(19) United States
(12) Patent Application Publication
(10) Pub. No.: US 2026/0167598 A1
(43) Pub. Date: Jun. 18, 2026

(54) PHOSPHINE LIGANDS AND USE THEREOF IN CARBONYLATION OF ACETYLENE/ETHYLENE TO SYNTHESIZE ACRYLATE/PROPIONATE

(71) Applicant: SHANGHAI INSTITUTE OF ORGANIC CHEMISTRY, CHINESE ACADEMY OF SCIENCES, Shanghai (CN)

(72) Inventors: Guosheng LIU, Shanghai (CN); Wencheng YANG, Shanghai (CN); Haihui PENG, Shanghai (CN); Pinhong CHEN, Shanghai (CN)

(21) Appl. No.: 19/158,427

(22) PCT Filed: Feb. 22, 2024
(86) PCT No.: PCT/CN2024/078075

§ 371 Date: Mar. 10, 2026

(30) Foreign Application Priority Data
Feb. 22, 2023 (CN) 202310151470.7
Feb. 22, 2023 (CN) 202310151489.1
Feb. 22, 2023 (CN) 202310151510.8
Feb. 22, 2023 (CN) 202310151550.2
Feb. 22, 2023 (CN) 202310151586.0
Feb. 22, 2023 (CN) 202310151621.9
Feb. 22, 2023 (CN) 202310151651.X
Feb. 22, 2023 (CN) 202310151696.7
Feb. 22, 2023 (CN) 202310151709.0
Feb. 22, 2023 (CN) 202310151719.4
Feb. 22, 2023 (CN) 202310151731.5
Feb. 22, 2023 (CN) 202310151744.2

(51) Int. Cl. C07C 67/38; C07F 9/58; C07F 9/6558; C07F 9/6568; C07F 9/6584

(52) U.S. Cl. CPC C07C 67/38; C07F 9/58; C07F 9/65583; C07F 9/65586; C07F 9/65683; C07F 9/6584

(57) ABSTRACT

The invention relates to phosphine ligands and their application in the carbonylation of acetylene/ethylene to synthesize acrylate/propionate. The phosphine ligands of the present invention are represented by Formula I, Formula II, Formula III, Formula IV, Formula V, Formula VI, Formula VII, and Formula VIII. The invention relates to the use of these phosphine ligands with Pd catalysts in the carbonylation reaction of acetylene/ethylene, thereby synthesizing acrylate/propionate. These methods feature high catalytic efficiency, high product yield, high selectivity, and simple operation.

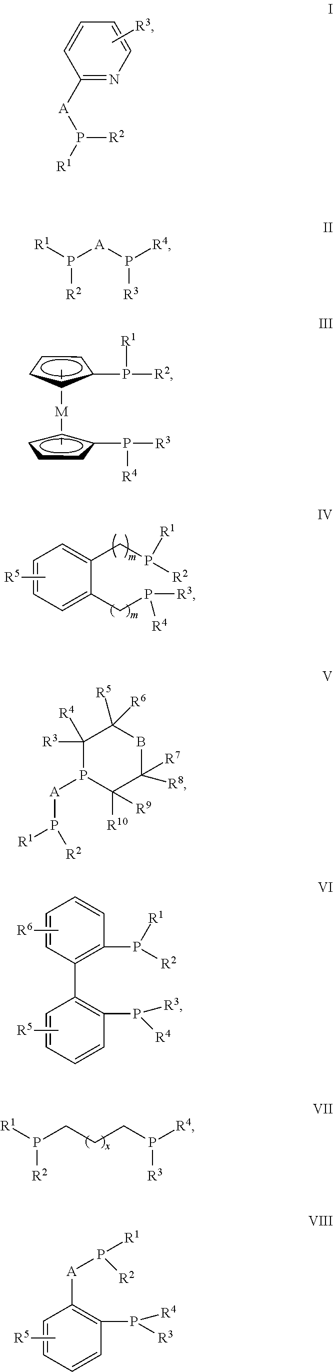

### FIELD

The invention relates to phosphine ligands and to the use thereof for the carbonylation of acetylene/ethylene to synthesize acrylate/propionate esters.

### BACKGROUND OF THE INVENTION

Acrylate esters are important industrial chemical materials primarily used as resin monomers, with extensive applications in coatings, adhesives, textiles, rubber and other industries. Driven by construction, textile and packaging sectors, acrylic acid (ester) consumption has maintained rapid growth. The propylene oxidation process has remained the dominant industrial method for acrylic acid production. However, with increasing depletion of petroleum resources and rising prices, alternative routes utilizing non-petrochemical feedstocks have attracted significant attention. The acetylene carbonylation route for acrylic acid (ester) synthesis, as a non-petroleum pathway, demonstrates competitive advantages and represents one of the key technological development trends in acrylic acid (ester) production.

Propionic acid and its ester derivatives constitute important fine chemical products and organic synthesis intermediates, widely used in agriculture, light industry/textiles, food, pharmaceuticals and related fields. Among the three major edible preservatives—benzoic acid, propionic acid and sorbic acid—propionic acid is universally recognized as the most cost-effective and safe option. W. Reppe first proposed the direct synthesis of methyl propionate and ethyl propionate via ethylene carbonylation (Chem. Rev. 2001, 101, 11, 3435). Ruthenium, nickel, cobalt and other catalysts also exhibit certain activity for this reaction, with over hundreds of catalyst systems having been reported to date.

The synthesis of acrylic acid via acetylene carbonylation was also developed by W. Reppe, who demonstrated the conversion of acetylene-carbon monoxide-water (alcohol) mixtures to acrylic acid (esters) in the presence of nickel tetracarbonyl (Liebigs Ann. Chem. 1953, 582, 161). This process required severe reaction conditions (>150° C., 1-3 MPa) and suffered from significant catalyst loss and high toxicity. Subsequent improvements by Rohm and Haas Company, Dow-Badische, and BASF enabled its industrial application. To date, over hundreds of catalyst systems have been reported for this reaction, predominantly based on nickel halides or copper halides. Although these halogen-containing catalysts demonstrate satisfactory yields and selectivity, they exhibit inherent drawbacks including prolonged reaction time, carbon deposition during the process, and severe equipment corrosion.

Alper et al. discovered that palladium acetate, when promoted by acid and phosphine ligands, could catalyze the hydrocarboxylation of alkynes to α,β-unsaturated acids (Organometallics 1993, 12, 712; J. Org. Chem. 1993, 58, 4739). Their work revealed the critical role of phosphine ligands in determining reaction activity and selectivity. In 2009, a system using palladium acetate as catalyst, sulfonic acid as co-catalyst, and 2-pyridyldiphenylphosphine as ligand, achieved the acetylene carbonylation under mild conditions (40° C., 5 MPa) (CN101768070A). However, this method showed limited acetylene conversion (<42%). In 2015, a Pd(II)/Xantphos catalytic system using formic acid as carbonyl source, achieved a maximum turnover number (TON) of 140 (Angew. Chem. Int. Ed. 2015, 54, 6302). While palladium-based systems operate under milder conditions with better selectivity, the current catalytic activity remains insufficient for industrial application due to relatively low TON values.

For the synthesis of propionate esters, the Shell process employs divalent palladium salts, triphenylphosphine, and acid as catalysts for the carbonylation of ethylene to methyl propionate (CN1041517C). A similar palladium acetate, when promoted by acid and phosphine ligands, can catalyze the carbonylation of ethylene to form propionate esters (Chem. Commun. 1999, 1877). Their research demonstrated the critical role of phosphine ligands in determining reaction activity and selectivity. Lucite developed an α-process for industrial use, wherein methyl propionate is first produced via ethylene carbonylation with carbon monoxide and methanol using a divalent palladium/phosphine ligand system, followed by condensation with formaldehyde to yield methyl methacrylate (MMA)(CN106854221, CN10553496). This catalytic system has attracted significant attention in both fundamental research and industrial applications. However, current technologies still require improvement in catalytic efficiency and selectivity.

Consequently, current catalyst systems for ethylene carbonylation to propionate esters predominantly utilize divalent palladium salts and phosphine ligands. While these systems operate under relatively mild conditions and demonstrate favorable reaction selectivity with high turnover numbers (TON), further improvements in catalytic efficiency and selectivity are urgently needed to reduce production costs and meet industrial-scale application requirements. There remains a critical need to develop highly efficient catalytic systems for ethylene carbonylation to propionate esters.

### SUMMARY OF THE INVENTION

The problem addressed by the present invention is that of providing catalysts for the carbonylation of acetylene/ethylene to prepare acrylate/propionate esters.

Another problem addressed by the present invention is that of providing a process for the carbonylation of acetylene to prepare acrylate, such as methyl acrylate, ethyl acrylate, butyl acrylate, or octyl acrylate.

Another problem addressed by the present invention is that of providing a process for the carbonylation of ethylene to prepare propionate esters, such as methyl propionate, ethyl propionate, butyl propionate, or octyl propionate.

In first aspect, the present invention provides a phosphine ligand for the catalytic preparation of acrylate esters from acetylene, wherein the phosphine ligand has a structure selected from Formula I

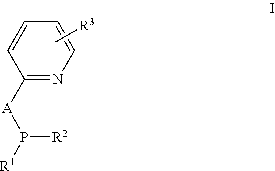

wherein
R¹ is selected from substituted or unsubstituted C₁₋₁₀ alkyl, C₃₋₁₂ cycloalkyl, optionally containing adamantyl, C₆₋₃₀ aryl;
R² is selected from the substituted or unsubstituted 5-20 membered heteroaryl;
R³ is selected from hydrogen, substituted or unsubstituted C₁₋₁₀ alkyl, substituted or unsubstituted C₁₋₁₀ alkoxy, substituted or unsubstituted C₂₋₁₀ ester group, cyano, COOH, benzenesulfonyl, trialkylsilyl (wherein said alkyl is C₁₋₄ alkyl), nitro, substituted or unsubstituted C₆₋₃₀ aryl, substituted or unsubstituted 5-30 membered heteroaryl; or two R³ groups attached to adjacent carbon atoms together with the carbon atoms to which they are attached form a group selected from the group consisting of: C₆₋₁₀ aryl, 5-12 membered heteroaryl;
A is selected from substituted and unsubstituted C₁₋₄ alkyl,

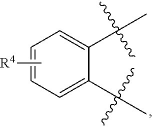

A and

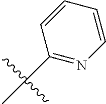 [SMILES: *c1ccccn1]

are linked to form

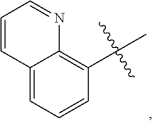

wherein, [symbol] is the attachment site linked to phosphorus;
and
A if it is

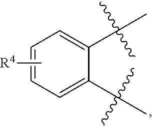

R⁴ is one or more substituents selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, and 5-30 membered heteroaryl; or R⁴ groups together with R³ and adjacent pyridine ring atoms form a fused 5-7 membered carbocyclic or heterocyclic ring;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, unless expressly stated otherwise.

In one embodiment, in the formula I, A is selected from CH₂,

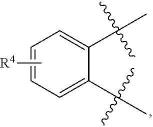

or A together with

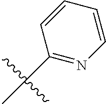 [SMILES: *c1ccccn1]

form

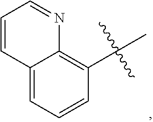 [SMILES: *c1cccc2cccnc12]

[symbol]

s the attachment site linked to phosphorus;
and R⁴ is one or more substituents selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl;

In one embodiment, in the formula I, R¹ is selected from substituted or unsubstituted C₁₋₁₀ alkyl, C₃₋₁₀ cycloalkyl;
R² is selected from the substituted or unsubstituted 5-10 membered heteroaryl;
R³ is selected from hydrogen, substituted or unsubstituted C₁₋₁₀ alkyl, C₁₋₁₀ alkoxy, C₂₋₁₀ ester, cyano, tri(C₁₋₄ alkyl)silyl, nitro, —CH(Ph)₂, C₆₋₃₀ aryl, 5-10 membered heteroaryl.

In one embodiment, in the formula I, R¹ is selected from methyl, ethyl, n-propyl, isopropyl, n-butyl, tert-butyl, isobutyl, cyclopropyl, cyclobutyl, cyclopentyl, cyclohexyl, adamantyl, phenyl.

R² is selected from pyridyl, pyrazinyl, pyrimidinyl, pyridazinyl, pyrrolyl, imidazolyl, pyrazolyl, thienyl, furyl, thiazolyl, triazolyl; may each be substituted by one or more substituents selected from C₁₋₄ alkyl.

R³ is selected from methyl, n-propyl, isopropyl, tert-butyl, methoxy, cyano, COOH, —CH(Ph)₂, benzenesulfonyl, trimethylsilyl.

In one embodiment, the phosphine ligand of formula I is selected from

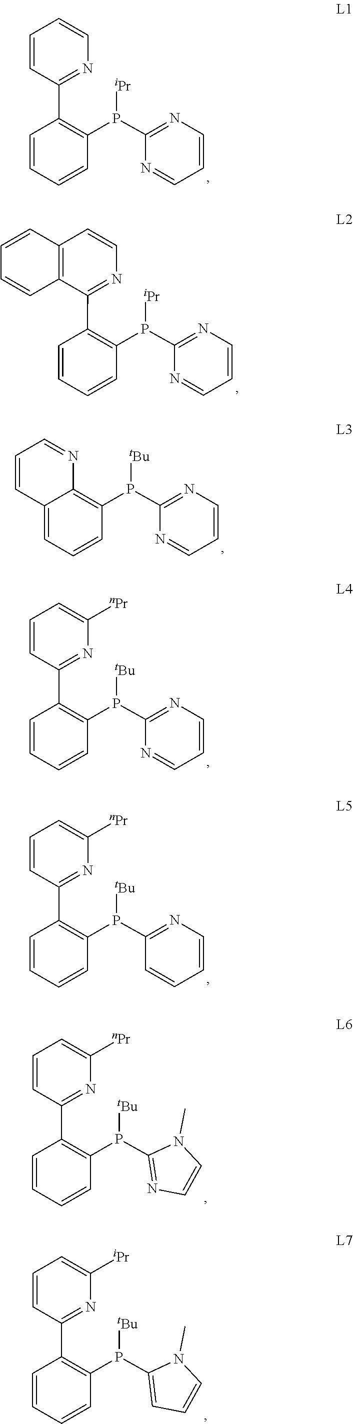

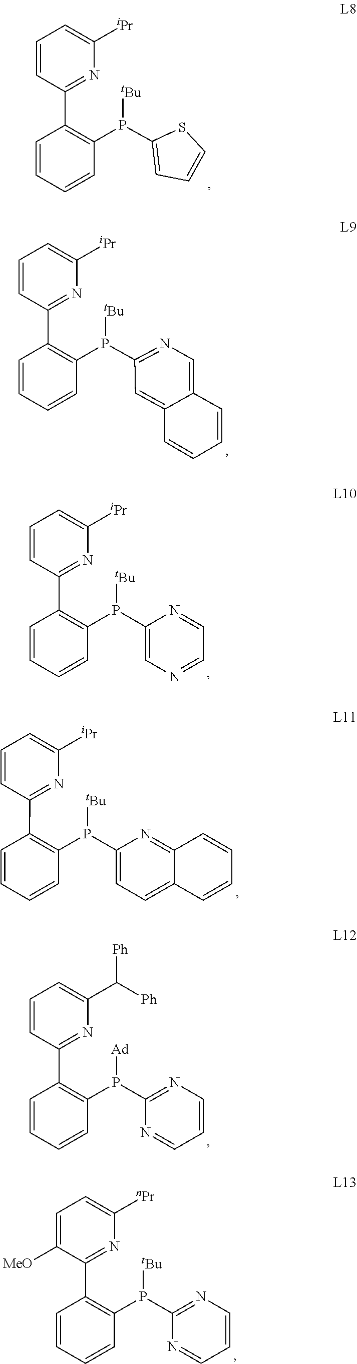

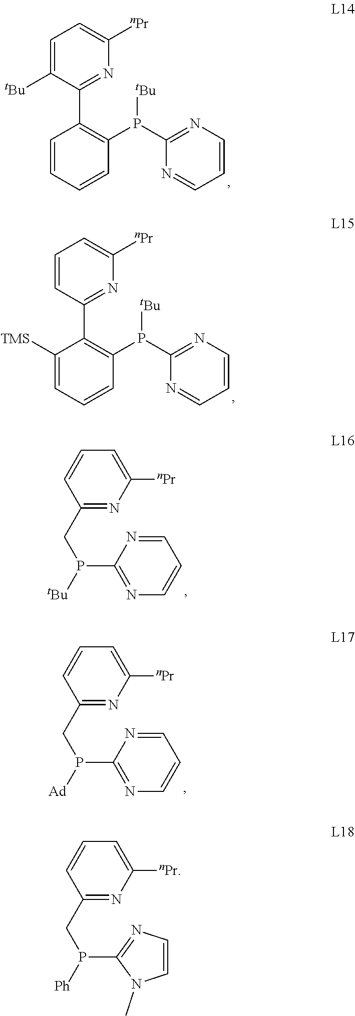

In second aspect, the present invention provides a method for palladium-catalyzed carbonylation of acetylene to synthesize acrylate esters, wherein said method comprising the steps of:
(a) In a reaction vessel, dissolving a palladium catalyst, a phosphine ligand, and an acid in an alcohol and an optional solvent; wherein said phosphine ligand is described as Formula I;
(b) Introducing acetylene into the vessel, followed by carbon monoxide, to initiate the reaction;
(c) Terminating the reaction and isolating the product.

In one embodiment, said reaction vessel is a high-pressure autoclave.

In one embodiment, the amount of said Formula I phosphine ligand is 0.00001-10 mol % relative to acetylene, more preferably 0.001-1 mol %.

In one embodiment, the reaction temperature is 0-200° C., preferably 60-120° C.

In one embodiment, the reaction is conducted under an inert gas atmosphere; preferably, said inert gas is nitrogen and/or argon.

In one embodiment, the reaction is carried out at 1-10 MPa, preferably 2-8 MPa.

In one embodiment, the reaction time is 0.5-72 hours, preferably 0.5-24 hours.

In one embodiment, the said alcohol is a C₁₋₁₂ alkyl alcohol; preferably, said alcohol is selected from the group consisting of: methanol, ethanol, propanol, butanol, octanol, or a combination thereof.

In one embodiment, the amount of said alcohol is 1-100 molar equivalents relative to acetylene, preferably 1-10 molar equivalents.

In one embodiment, said palladium catalyst is selected from the group consisting of: palladium acetate, palladium trifluoroacetate, palladium pivalate, palladium(II) tetraacetonitrile tetrafluoroborate, palladium(II) hexafluoroacetylacetonate, palladium(II) bis(acetylacetonate), palladium(II) tetraacetonitrile trifluoromethanesulfonate, palladium(II) neodecanoate, bis(dibenzylideneacetone)palladium(0), tris(dibenzylideneacetone)dipalladium(0), palladium(II) chloride, palladium(II) diacetonitrile dichloride, palladium(II) dibenzonitrile dichloride, or a combination thereof.

In one embodiment, said palladium catalyst is selected from the group consisting of: palladium acetate, palladium trifluoroacetate, palladium pivalate, tris(dibenzylideneacetone)dipalladium(0), palladium(II) chloride, palladium(II) diacetonitrile dichloride, or a combination thereof.

In one embodiment, the amount of said palladium catalyst is 0.00001-10 mol % relative to acetylene, more preferably 0.0001-1 mol %.

In the reaction, when said phosphine ligand is a nitrogen-phosphine ligand (i.e., a Formula I compound), the molar ratio of said palladium catalyst to said nitrogen-phosphine ligand is 1:1 to 1:30, more preferably 1:1 to 1:5.

In one embodiment, said acid is selected from the group consisting of: perchloric acid, sulfuric acid, phosphoric acid, sulfonic acid, alkylphosphonic acid, alkylsulfonic acid, alkylcarboxylic acid, perfluoroalkylsulfonic acid, perfluoroalkylcarboxylic acid (wherein said alkyl is a C₁₋₁₂ alkyl), or arylsulfonic acid (wherein said aryl is a C₆₋₁₀ aryl).

In one embodiment, said acid is selected from the group consisting of: methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid, dodecylsulfonic acid, sulfuric acid, sulfonic acid, formic acid, and trifluoroacetic acid.

In one embodiment, the amount of said acid is 0.00004-40 mol % relative to acetylene, preferably 0.0004-4 mol %.

In one embodiment, said solvent is selected from the group consisting of: alkane solvents, substituted aromatic solvents, ether solvents, ketone solvents, nitrile solvents, ester solvents, or a combination thereof.

In one embodiment, said alkane solvent is selected from the group consisting of: n-hexane, cyclohexane, or a combination thereof.

In one embodiment, said substituted aromatic solvent is selected from the group consisting of: chlorobenzene, toluene, xylene, and trifluorotoluene.

In one embodiment, said ether solvent is selected from the group consisting of: tetrahydrofuran, diethyl ether, methyl tert-butyl ether, ethyl tert-butyl ether, anisole, ethylene glycol dimethyl ether, 1,4-dioxane, or a combination thereof.

In one embodiment, said ketone solvent is acetone.

In one embodiment, said nitrile solvent is selected from the group consisting of: acetonitrile, propionitrile, benzonitrile, or a combination thereof.

In one embodiment, said ester solvent is ethyl acetate.

In one embodiment, said alcohol is methanol, and the reaction is carried out under the following conditions:
a) Dissolving palladium acetate, a phosphine ligand of Formula I, and an acid in methanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is ethanol, and the reaction is carried out under the following conditions:
a) Dissolving palladium acetate, a phosphine ligand of Formula I, and an acid in ethanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is butanol, and the reaction is carried out under the following conditions:
a) Dissolving palladium acetate, a phosphine ligand of Formula I, and an acid in butanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is octanol, and the reaction is carried out under the following conditions:
a) Dissolving palladium acetate, a phosphine ligand of Formula I, and an acid in octanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In third aspect, the present invention provides a method for palladium-catalyzed carbonylation of acetylene to synthesize acrylate esters, such as methyl acrylate, ethyl acrylate, butyl acrylate, or octyl acrylate, wherein said method comprising the steps of:
(a) In a reaction vessel, dissolving a palladium catalyst, a phosphine ligand, and an acid in an alcohol and an optional solvent; wherein said phosphine ligand is described as Formula II;
(b) Introducing acetylene into the vessel, followed by carbon monoxide, to initiate the reaction;
(c) Terminating the reaction and isolating the product.

The phosphine ligand according to the invention comprises the Formula II:

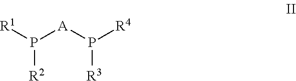

wherein
R¹, R⁴ are independently selected from substituted or unsubstituted C₁₋₁₂ alkyl, C₃₋₁₂ cycloalkyl, C₃₋₁₂ heterocycloalkyl, C₆₋₃₀ aryl;
R² and R³ are independently selected from 5-20 membered heteroaryl;
A is selected from

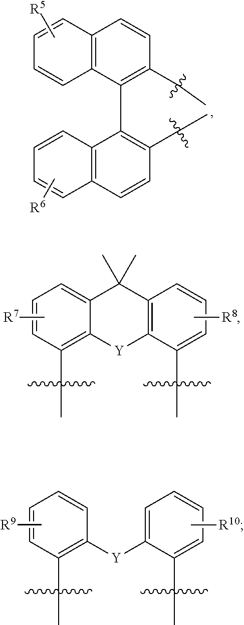

wherein, R⁵, R⁶, R⁷, R⁸, R⁹, R¹⁰ are independently selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, and 5-30 membered heteroaryl; two adjacent R⁵, R⁶, R⁷, R⁸, R⁹, R¹⁰ located on rings, together with the ring atoms to which they are attached, form a 5-7 membered carbocycle or heterocycle; Y is selected from O, NH, S;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, unless expressly stated otherwise.

In one embodiment, in the formula II,
R¹, R⁴ are independently selected from substituted or unsubstituted C₁₋₄ alkyl, C₃₋₁₀ cycloalkyl, optionally containing adamantyl, phenyl;
R² and R³ are independently selected from 5-10 membered heteroaryl;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, unless expressly stated otherwise.

In one embodiment, in the formula II,
R¹, R⁴ are independently selected from methyl, ethyl, n-propyl, isopropyl, n-butyl, tert-butyl, isobutyl, cyclopropyl, cyclobutyl, cyclopentyl, cyclohexyl, adamantyl, phenyl;
R², R³ are independently selected from pyridyl, pyrazinyl, pyrimidinyl, pyridazinyl, pyrrolyl, imidazolyl, pyrazolyl, thienyl, furyl, thiazolyl, triazolyl;
and
R⁵, R⁶, R⁷, R⁸, R⁹, R¹⁰ are independently selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₁₀ aryl, and 5-10 membered heteroaryl; two adjacent R⁵, R⁶, R⁷, R⁸, R⁹, R¹⁰ located on rings, together with the ring atoms to which they are attached, form a 5-7 membered carbocycle or heterocycle.

In one embodiment, the phosphine ligand of formula II is selected from

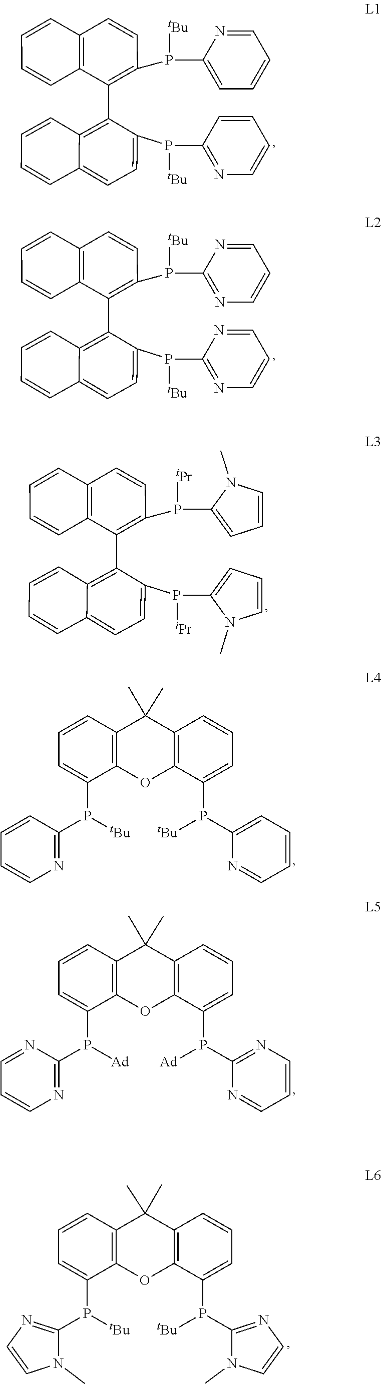

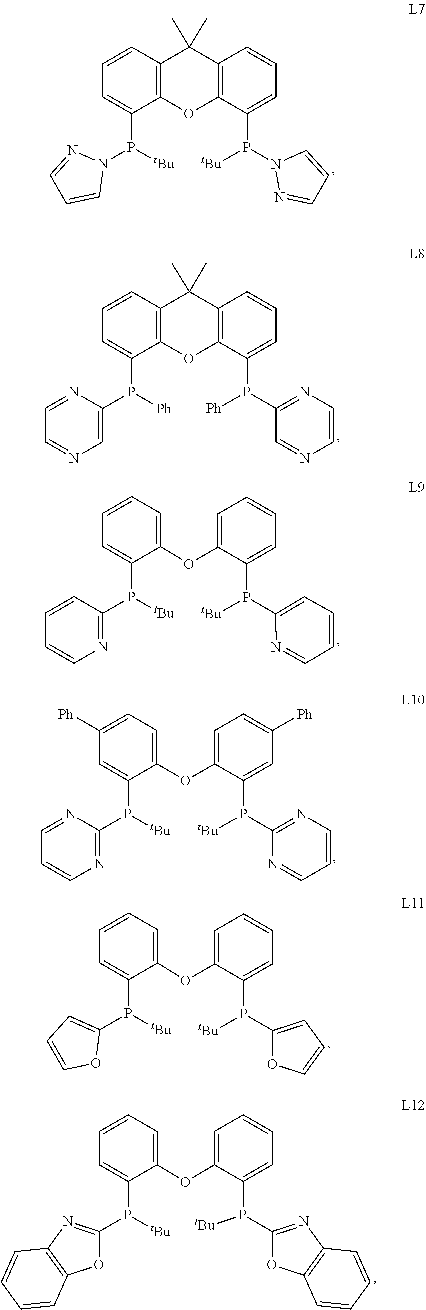 [SMILES: CC1(C)c2cccc(P(n3cccn3)C(C)(C)C)c2Oc2c(P(n3cccn3)C(C)(C)C)cccc21 ; CC1(C)c2cccc(P(c3ccccc3)c3cnccn3)c2Oc2c(P(c3ccccc3)c3cnccn3)cccc21 ; CC(C)(C)P(c1ccccn1)c1ccccc1Oc1ccccc1P(c1ccccn1)C(C)(C)C ; CC(C)(C)P(c1ncccn1)c1cc(-c2ccccc2)ccc1Oc1ccc(-c2ccccc2)cc1P(c1ncccn1)C(C)(C)C ; CC(C)(C)P(c1ccco1)c1ccccc1Oc1ccccc1P(c1ccco1)C(C)(C)C ; CC(C)(C)P(c1nc2ccccc2o1)c1ccccc1Oc1ccccc1P(c1nc2ccccc2o1)C(C)(C)C]

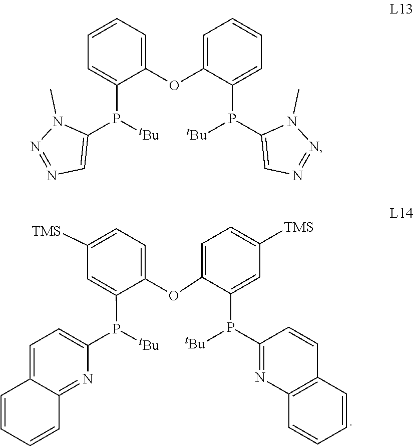 [SMILES: Cn1nncc1P(c1ccccc1Oc1ccccc1P(c1cnnn1C)C(C)(C)C)C(C)(C)C ; CC(C)(C)P(c1ccc2ccccc2n1)c1cc([Si](C)(C)C)ccc1Oc1ccc([Si](C)(C)C)cc1P(c1ccc2ccccc2n1)C(C)(C)C]

In one embodiment, said reaction vessel is a high-pressure autoclave.

In one embodiment, the amount of said Formula II phosphine ligand is 0.00001-10 mol % relative to acetylene, more preferably 0.0001-1 mol %.

In one embodiment, the reaction temperature is 0-200° C., preferably 60-130° C.

In one embodiment, the reaction is conducted under an inert gas atmosphere; preferably, said inert gas is nitrogen and/or argon.

In one embodiment, the reaction is carried out at 1-10 MPa, preferably 2-8 MPa.

In one embodiment, the reaction time is 0.5-72 hours, preferably 0.5-24 hours.

In one embodiment, the said alcohol is a C₁₋₁₂ alkyl alcohol; preferably, said alcohol is selected from the group consisting of: methanol, ethanol, propanol, butanol, octanol, or a combination thereof.

In one embodiment, the amount of said alcohol is 1-100 molar equivalents relative to acetylene, preferably 1-10 molar equivalents.

In one embodiment, said palladium catalyst is selected from the group consisting of: palladium acetate, palladium trifluoroacetate, palladium pivalate, palladium(II) tetraacetonitrile tetrafluoroborate, palladium(II) hexafluoroacetylacetonate, palladium(II) bis(acetylacetonate), palladium(II) tetraacetonitrile trifluoromethanesulfonate, palladium(II) neodecanoate, bis(dibenzylideneacetone)palladium(0), tris(dibenzylideneacetone)dipalladium(0), palladium(II) chloride, palladium(II) diacetonitrile dichloride, palladium(II) dibenzonitrile dichloride, or a combination thereof.

In one embodiment, said palladium catalyst is selected from the group consisting of: palladium acetate, palladium trifluoroacetate, palladium pivalate, tris(dibenzylideneacetone)dipalladium(0), palladium(II) chloride, palladium(II) diacetonitrile dichloride, or a combination thereof.

In one embodiment, the amount of said palladium catalyst is 0.00001-10 mol % relative to acetylene, more preferably 0.0001-1 mol %.

In the reaction, when said phosphine ligand is a nitrogen-phosphine ligand (i.e., a Formula I compound), the molar ratio of said palladium catalyst to said nitrogen-phosphine ligand is 1:1 to 1:30, more preferably 1:1 to 1:5.

In one embodiment, said acid is selected from the group consisting of: perchloric acid, sulfuric acid, phosphoric acid, sulfonic acid, alkylphosphonic acid, alkylsulfonic acid, alkylcarboxylic acid, perfluoroalkylsulfonic acid, perfluoroalkylcarboxylic acid (wherein said alkyl is a C₁₋₁₂ alkyl), or arylsulfonic acid (wherein said aryl is a C₆₋₁₀ aryl).

In one embodiment, said acid is selected from the group consisting of: methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid, dodecylsulfonic acid, sulfuric acid, sulfonic acid, formic acid, and trifluoroacetic acid.

In one embodiment, the amount of said acid is 0.00004-40 mol % relative to acetylene, preferably 0.0004-4 mol %.

In one embodiment, said solvent is selected from the group consisting of: alkane solvents, substituted aromatic solvents, ether solvents, ketone solvents, nitrile solvents, ester solvents, or a combination thereof.

In one embodiment, said alkane solvent is selected from the group consisting of: n-hexane, cyclohexane, or a combination thereof.

In one embodiment, said substituted aromatic solvent is selected from the group consisting of: chlorobenzene, toluene, xylene, and trifluorotoluene.

In one embodiment, said ether solvent is selected from the group consisting of: tetrahydrofuran, diethyl ether, methyl tert-butyl ether, ethyl tert-butyl ether, anisole, ethylene glycol dimethyl ether, 1,4-dioxane, or a combination thereof.

In one embodiment, said ketone solvent is acetone.

In one embodiment, said nitrile solvent is selected from the group consisting of: acetonitrile, propionitrile, benzonitrile, or a combination thereof.

In one embodiment, said ester solvent is ethyl acetate.

In one embodiment, said alcohol is methanol, and the reaction is carried out under the following conditions:
a) Dissolving palladium acetate, a phosphine ligand of Formula II, and an acid in methanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In one embodiment, said alcohol is ethanol, and the reaction is carried out under the following conditions:
a) Dissolving palladium acetate, a phosphine ligand of Formula II, and an acid in ethanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In one embodiment, said alcohol is butanol, and the reaction is carried out under the following conditions:
a) Dissolving palladium acetate, a phosphine ligand of Formula II, and an acid in butanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In one embodiment, said alcohol is octanol, and the reaction is carried out under the following conditions:
a) Dissolving palladium acetate, a phosphine ligand of Formula II, and an acid in octanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In fourth aspect, the present invention provides a method for palladium-catalyzed carbonylation of acetylene to synthesize acrylate esters, such as methyl acrylate, ethyl acrylate, butyl acrylate, or octyl acrylate, wherein said method comprising the steps of:
(a) In a reaction vessel, dissolving a palladium catalyst, a phosphine ligand, and an acid in an alcohol and an optional solvent;
(b) Introducing acetylene into the vessel, followed by carbon monoxide, to initiate the reaction;
(c) Terminating the reaction and isolating the product.

The phosphine ligand according to the invention comprises the Formula III:

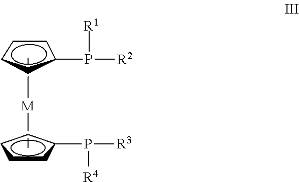

wherein,
R¹, R⁴ are independently selected from substituted or unsubstituted C₁₋₁₂ alkyl, C₃₋₁₂ cycloalkyl, C₃₋₁₂ heterocycloalkyl, C₆₋₃₀ aryl;
R² and R³ are independently selected from 5-20 membered heteroaryl;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, unless expressly stated otherwise.
M is selected from Fe, Co, Ru.
In one embodiment, in the formula III,
R¹, R⁴ are independently selected from substituted or unsubstituted C₁₋₁₀ alkyl, C₃₋₈ cycloalkyl, phenyl;
R² and R³ are independently selected from 5-10 membered heteroaryl.

In one embodiment, in the formula III,
R¹, R⁴ are independently selected from methyl, ethyl, n-propyl, isopropyl, n-butyl, tert-butyl, isobutyl, cyclopropyl, cyclobutyl, cyclopentyl, cyclohexyl, adamantyl, phenyl;
R², R³ are independently selected from pyridyl, pyrazinyl, pyrimidinyl, pyridazinyl, pyrrolyl, imidazolyl, pyrazolyl, thienyl, furyl, thiazolyl, triazolyl.

In one embodiment, the phosphine ligand of formula III is selected from

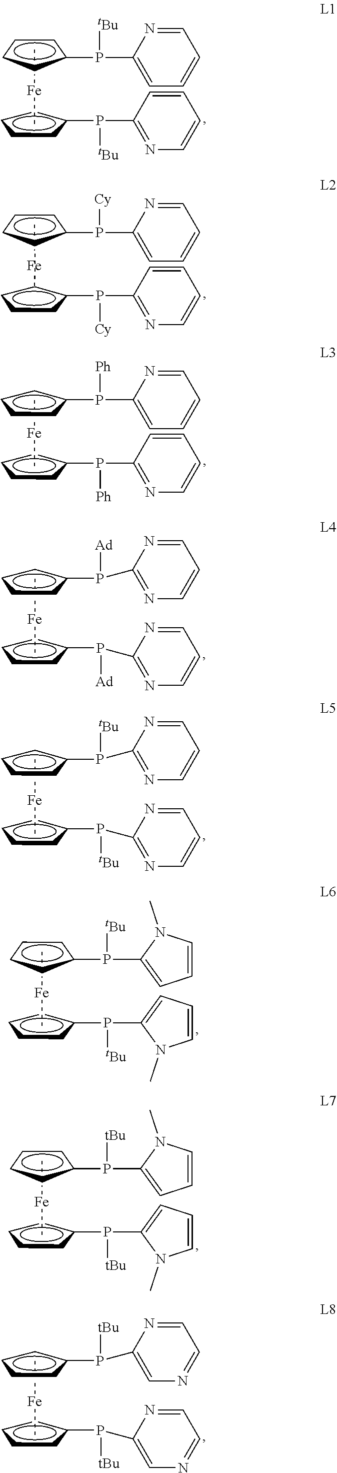

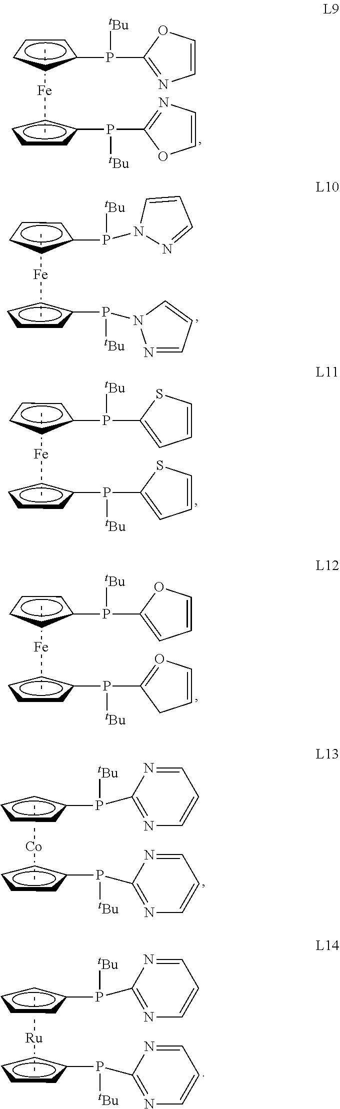

In one embodiment, said reaction vessel is a high-pressure autoclave.

In one embodiment, the amount of said Formula III phosphine ligand is 0.00001-10 mol % relative to acetylene, more preferably 0.001-1 mol %.

In one embodiment, the reaction temperature is 0-200° C., preferably 40-130° C.

In one embodiment, the reaction is conducted under an inert gas atmosphere; preferably, said inert gas is nitrogen and/or argon.

In one embodiment, the reaction is carried out at 1-10 MPa, preferably 2-8 MPa.

In one embodiment, the reaction time is 0.5-72 hours, preferably 0.5-24 hours.

In one embodiment, the said alcohol is a C₁₋₁₂ alkyl alcohol; preferably, said alcohol is selected from the group consisting of: methanol, ethanol, propanol, butanol, octanol, or a combination thereof.

In one embodiment, the amount of said alcohol is 1-100 molar equivalents relative to acetylene, preferably 1-10 molar equivalents.

In one embodiment, said palladium catalyst is selected from the group consisting of: palladium acetate, palladium trifluoroacetate, palladium pivalate, palladium(II) tetraacetonitrile tetrafluoroborate, palladium(II) hexafluoroacetylacetonate, palladium(II) bis(acetylacetonate), palladium(II) tetraacetonitrile trifluoromethanesulfonate, palladium(II) neodecanoate, bis(dibenzylideneacetone)palladium(0), tris(dibenzylideneacetone)dipalladium(0), palladium(II) chloride, palladium(II) diacetonitrile dichloride, palladium(II) dibenzonitrile dichloride, or a combination thereof.

In one embodiment, said palladium catalyst is selected from the group consisting of: palladium acetate, palladium trifluoroacetate, palladium pivalate, tris(dibenzylideneacetone)dipalladium(0), palladium(II) chloride, palladium(II) diacetonitrile dichloride, or a combination thereof.

In one embodiment, the amount of said palladium catalyst is 0.00001-10 mol % relative to acetylene, more preferably 0.0001-1 mol %.

In the reaction, when said phosphine ligand is a nitrogen-phosphine ligand (i.e., a Formula I compound), the molar ratio of said palladium catalyst to said nitrogen-phosphine ligand is 1:1 to 1:30, more preferably 1:1 to 1:5.

In one embodiment, said acid is selected from the group consisting of: perchloric acid, sulfuric acid, phosphoric acid, sulfonic acid, alkylphosphonic acid, alkylsulfonic acid, alkylcarboxylic acid, perfluoroalkylsulfonic acid, perfluoroalkylcarboxylic acid (wherein said alkyl is a C₁₋₁₂ alkyl), or arylsulfonic acid (wherein said aryl is a C₆₋₁₀ aryl).

In one embodiment, said acid is selected from the group consisting of: methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid, dodecylsulfonic acid, sulfuric acid, sulfonic acid, formic acid, and trifluoroacetic acid.

In one embodiment, the amount of said acid is 0.00004-40 mol % relative to acetylene, preferably 0.0004-4 mol %.

In one embodiment, said solvent is selected from the group consisting of: alkane solvents, substituted aromatic solvents, ether solvents, ketone solvents, nitrile solvents, ester solvents, or a combination thereof.

In one embodiment, said alkane solvent is selected from the group consisting of: n-hexane, cyclohexane, or a combination thereof.

In one embodiment, said substituted aromatic solvent is selected from the group consisting of: chlorobenzene, toluene, xylene, and trifluorotoluene.

In one embodiment, said ether solvent is selected from the group consisting of: tetrahydrofuran, diethyl ether, methyl tert-butyl ether, ethyl tert-butyl ether, anisole, ethylene glycol dimethyl ether, 1,4-dioxane, or a combination thereof.

In one embodiment, said ketone solvent is acetone.

In one embodiment, said nitrile solvent is selected from the group consisting of: acetonitrile, propionitrile, benzonitrile, or a combination thereof.

In one embodiment, said ester solvent is ethyl acetate.

In one embodiment, said alcohol is methanol, and the reaction is carried out under the following conditions:
a) Dissolving palladium acetate, a phosphine ligand of Formula III, and an acid in methanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In one embodiment, said alcohol is ethanol, and the reaction is carried out under the following conditions:
a) Dissolving palladium acetate, a phosphine ligand of Formula III, and an acid in ethanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In one embodiment, said alcohol is butanol, and the reaction is carried out under the following conditions:
a) Dissolving palladium acetate, a phosphine ligand of Formula III, and an acid in butanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In one embodiment, said alcohol is octanol, and the reaction is carried out under the following conditions:
a) Dissolving palladium acetate, a phosphine ligand of Formula III, and an acid in octanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In fifth aspect, the present invention provides a method for palladium-catalyzed carbonylation of acetylene to synthesize acrylate esters, such as methyl acrylate, ethyl acrylate, butyl acrylate, or octyl acrylate, wherein said method comprising the steps of:
(a) In a reaction vessel, dissolving a palladium catalyst, a phosphine ligand, and an acid in an alcohol and an optional solvent; wherein said phosphine ligand is described as Formula IV;
(b) Introducing acetylene into the vessel, followed by carbon monoxide, to initiate the reaction;
(c) Terminating the reaction and isolating the product.

The phosphine ligand according to the invention comprises the Formula IV:

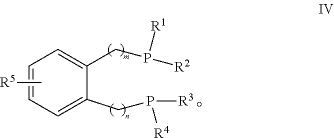

wherein
R¹, R⁴ are independently selected from substituted or unsubstituted C₁₋₁₂ alkyl, C₃₋₁₀ cycloalkyl, C₃₋₁₀ heterocycloalkyl, C₆₋₃₀ aryl;
R² and R³ are independently selected from 5-20 membered heteroaryl;
R⁵ is one or more substituents independently selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, and 5-30 membered heteroaryl; two adjacent R⁵ together with the ring atoms to which they are attached, form a 5-7 membered carbocycle or heterocycle;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, unless expressly stated otherwise.
m, n are independently selected from 0, 1, 2, 3.

In one embodiment, in the formula IV,
R¹, R⁴ are independently selected from substituted or unsubstituted C₁₋₁₀ alkyl, C₃₋₆ cycloalkyl, C₆₋₁₀ aryl;
R² and R³ are independently selected from 5-10 membered heteroaryl groups.

In one embodiment, in the formula IV,
R¹, R⁴ are independently selected from methyl, ethyl, n-propyl, isopropyl, n-butyl, tert-butyl, isobutyl, cyclopropyl, cyclobutyl, cyclopentyl, cyclohexyl, adamantyl, phenyl;
R², R³ are independently selected from pyridyl, pyrazinyl, pyrimidinyl, pyridazinyl, pyrrolyl, imidazolyl, pyrazolyl, thienyl, furyl, thiazolyl, triazolyl.

In one embodiment, the phosphine ligand of formula IV is selected from

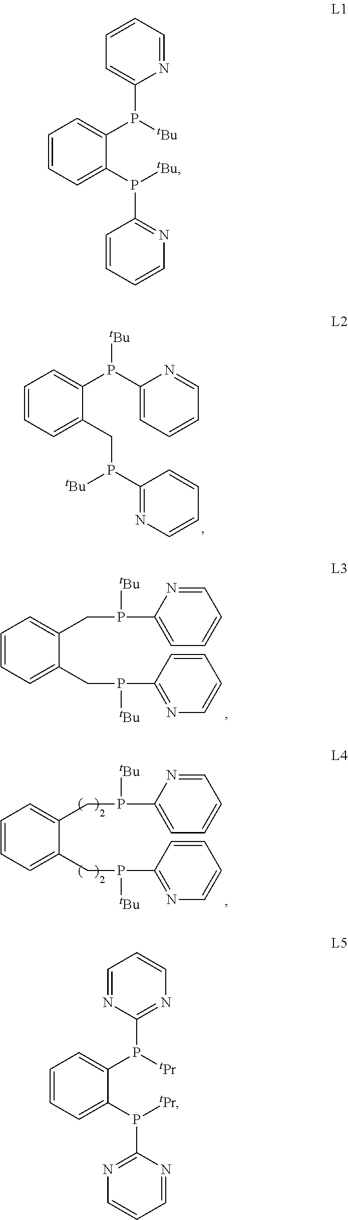

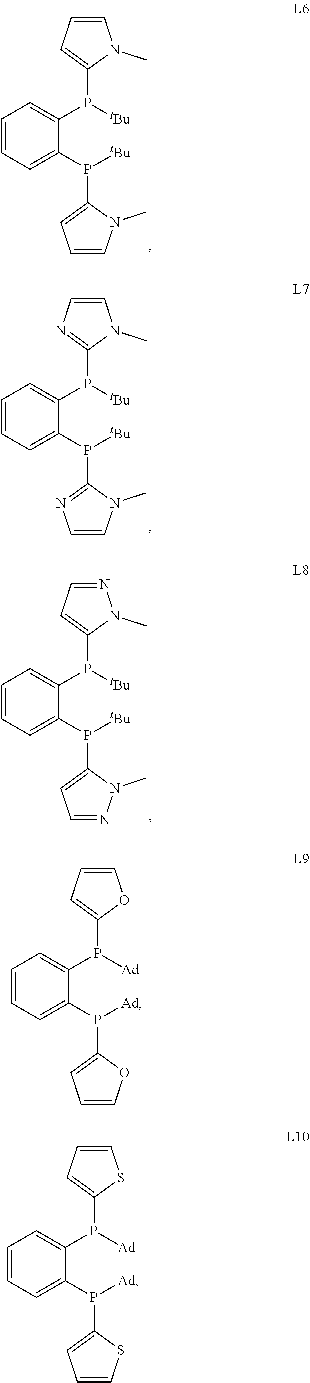

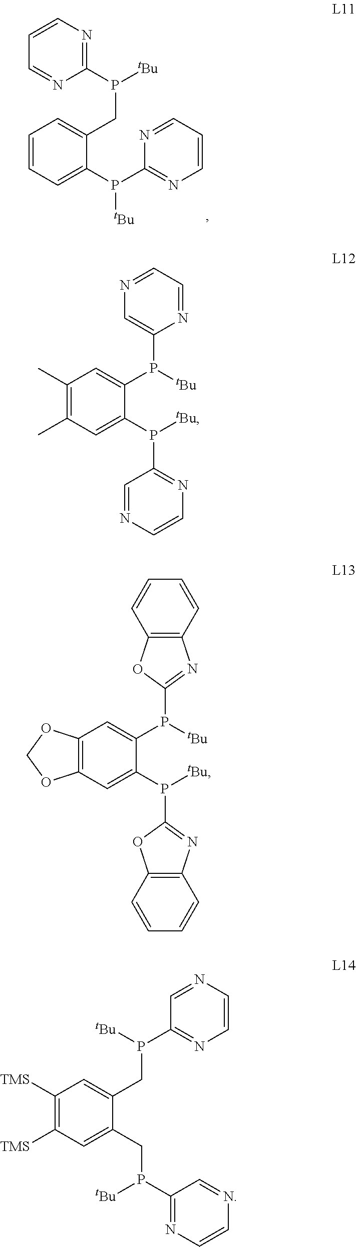

In one embodiment, said reaction vessel is a high-pressure autoclave.

In one embodiment, the amount of said Formula IV phosphine ligand is 0.00001-10 mol % relative to acetylene, more preferably 0.001-1 mol %.

In one embodiment, the reaction temperature is 0-200° C., preferably 40-130° C.

In one embodiment, the reaction is conducted under an inert gas atmosphere; preferably, said inert gas is nitrogen and/or argon.

In one embodiment, the reaction is carried out at 1-10 MPa, preferably 2-8 MPa.

In one embodiment, the reaction time is 0.5-72 hours, preferably 0.5-24 hours.

In one embodiment, the said alcohol is a C₁₋₁₂ alkyl alcohol; preferably, said alcohol is selected from the group consisting of: methanol, ethanol, propanol, butanol, octanol, or a combination thereof.

In one embodiment, the amount of said alcohol is 1-100 molar equivalents relative to acetylene, preferably 1-10 molar equivalents.

In one embodiment, said palladium catalyst is selected from the group consisting of: palladium acetate, palladium trifluoroacetate, palladium pivalate, palladium(II) tetraacetonitrile tetrafluoroborate, palladium(II) hexafluoroacetylacetonate, palladium(II) bis(acetylacetonate), palladium(II) tetraacetonitrile trifluoromethanesulfonate, palladium(II) neodecanoate, bis(dibenzylideneacetone)palladium(0), tris(dibenzylideneacetone)dipalladium(0), palladium(II) chloride, palladium(II) diacetonitrile dichloride, palladium(II) dibenzonitrile dichloride, or a combination thereof.

In one embodiment, said palladium catalyst is selected from the group consisting of: palladium acetate, palladium trifluoroacetate, palladium pivalate, tris(dibenzylideneacetone)dipalladium(0), palladium(II) chloride, palladium(II) diacetonitrile dichloride, or a combination thereof.

In one embodiment, the amount of said palladium catalyst is 0.00001-10 mol % relative to acetylene, more preferably 0.0001-1 mol %.

In the reaction, when said phosphine ligand is a nitrogen-phosphine ligand (i.e., a Formula I compound), the molar ratio of said palladium catalyst to said nitrogen-phosphine ligand is 1:1 to 1:30, more preferably 1:1 to 1:5.

In one embodiment, said acid is selected from the group consisting of: perchloric acid, sulfuric acid, phosphoric acid, sulfonic acid, alkylphosphonic acid, alkylsulfonic acid, alkylcarboxylic acid, perfluoroalkylsulfonic acid, perfluoroalkylcarboxylic acid (wherein said alkyl is a C₁₋₁₂ alkyl), or arylsulfonic acid (wherein said aryl is a C₆₋₁₀ aryl).

In one embodiment, said acid is selected from the group consisting of: methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid, dodecylsulfonic acid, sulfuric acid, sulfonic acid, formic acid, and trifluoroacetic acid.

In one embodiment, the amount of said acid is 0.00004-40 mol % relative to acetylene, preferably 0.0004-4 mol %.

In one embodiment, said solvent is selected from the group consisting of: alkane solvents, substituted aromatic solvents, ether solvents, ketone solvents, nitrile solvents, ester solvents, or a combination thereof.

In one embodiment, said alkane solvent is selected from the group consisting of: n-hexane, cyclohexane, or a combination thereof.

In one embodiment, said substituted aromatic solvent is selected from the group consisting of: chlorobenzene, toluene, xylene, and trifluorotoluene.

In one embodiment, said ether solvent is selected from the group consisting of: tetrahydrofuran, diethyl ether, methyl tert-butyl ether, ethyl tert-butyl ether, anisole, ethylene glycol dimethyl ether, 1,4-dioxane, or a combination thereof.

In one embodiment, said ketone solvent is acetone.

In one embodiment, said nitrile solvent is selected from the group consisting of: acetonitrile, propionitrile, benzonitrile, or a combination thereof.

In one embodiment, said ester solvent is ethyl acetate.

In one embodiment, said alcohol is methanol, and the reaction is carried out under the following conditions:
a) Dissolving palladium acetate, a phosphine ligand of Formula IV, and an acid in methanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In one embodiment, said alcohol is ethanol, and the reaction is carried out under the following conditions:
a) Dissolving palladium acetate, a phosphine ligand of Formula IV, and an acid in ethanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In one embodiment, said alcohol is butanol, and the reaction is carried out under the following conditions:
a) Dissolving palladium acetate, a phosphine ligand of Formula IV, and an acid in butanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In one embodiment, said alcohol is octanol, and the reaction is carried out under the following conditions:
a) Dissolving palladium acetate, a phosphine ligand of Formula IV, and an acid in octanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In sixth aspect, the present invention provides a phosphine ligand for the catalytic preparation of acrylate esters from acetylene, wherein the phosphine ligand has a structure selected from Formula V,

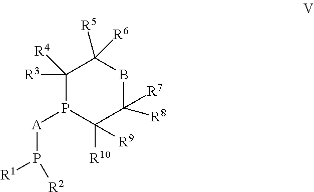

wherein
R¹ is selected from substituted or unsubstituted C₁₋₁₂ alkyl, C₃₋₁₀ cycloalkyl, C₆₋₃₀ aryl;
R² is selected from the substituted or unsubstituted 5-20 membered heteroaryl;
and
R¹ is selected from substituted or unsubstituted C₁₋₁₂ alkyl, C₃₋₁₀ cycloalkyl;
R² is selected from the substituted or unsubstituted C₆₋₃₀ heteroaryl;
R³, R⁴, R⁵, R⁶, R⁷, R⁸, R⁹, R¹⁰ are independently selected from hydrogen, hydroxyl, halogen, C₁₋₁₂ alkyl, C₃₋₁₂ cycloalkyl, C₃₋₁₀ heterocycloalkyl, C₂₋₁₀ alkenyl, C₆₋₃₀ aryl, 5-20 membered heteroaryl, C₁₋₁₀ alkoxyl, C₁₋₁₀ alkylamino, C₁₋₁₀ alkylamino, C₁₋₁₀ alkylthio; two adjacent R³, R⁴, R⁵, R⁶, R⁷, R⁸, R⁹, R¹⁰ located on rings, together with the ring atoms to which they are attached, form a 4-8 membered carbocycle or heterocycle;
A if it is -L¹-A¹-L²-, may each independently be selected from chemical bonds, O, NH, S, substituted or unsubstituted C₁₋₁₂ alkyl, C₃₋₈ cycloalkyl, C₃₋₈ heterocycloalkyl, C₆₋₃₀ aryl, 5-20 membered heteroaryl, biphenyl,

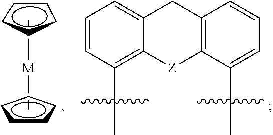

with the proviso that L¹, A¹ and L² are not all a chemical bond at the same time;
and
if the said aryl and heteroaryl are substituted by two or more substituents, any two of said substituents may, together with the ring atoms to which they are attached, form a 4-8 membered carbocycle or heterocycle;
M is selected from Fe, Co, Ru;
B is selected from substituted or unsubstituted methylene, ethylidene, ═CHR, ═NR, hydroxymethyl, carbonyl, thiocarbonyl,

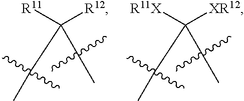

—C(O)—C(O)—; where R is selected from substituted or unsubstituted C₁₋₁₀ alkyl, C₆₋₃₀ aryl, and 5-20 membered heteroaryl;
R¹, R¹² are independently selected from hydrogen, hydroxyl, substituted or unsubstituted C₁₋₁₀ alkyl, or

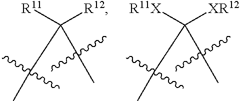

form 5-8 membered heterocycles, each may be unsubstituted or substituted by C₁₋₆ alkyl;
X is selected from O, S;
Z is selected from O, S, NH;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, benzyl, —CH(Ph)₂, unless expressly stated otherwise.

In one embodiment, in the formula V,
R¹ is selected from substituted or unsubstituted C₁₋₁₂ alkyl, C₃₋₁₀ cycloalkyl, C₆₋₂₀ aryl;
   R² is selected from the substituted or unsubstituted 5-12 membered heteroaryl;
and
R¹ is selected from substituted or unsubstituted C₁₋₁₂ alkyl, C₃₋₁₀ cycloalkyl; R² is selected from the substituted or unsubstituted C₆₋₂₀ aryl;

In one embodiment, in the formula V,
R³, R⁴, R⁵, R⁶, R⁷, R⁸, R⁹, R¹⁰ are independently selected from hydrogen, C₁₋₁₂ alkyl, C₃₋₁₂ cycloalkyl, C₃₋₁₀ heterocycloalkyl, C₂₋₁₀ alkenyl, C₁₋₁₀ alkoxyl, C₁₋₁₀ alkylamino, C₁₋₁₀ alkylamino, C₁₋₁₀ alkylthio; two adjacent R³, R¹⁰ located on rings, together with the ring atoms to which they are attached, form a 4-8 membered carbocycle or heterocycle;
B is selected from substituted or unsubstituted methylene, ethylidene, ═NR, hydroxymethyl, carbonyl,

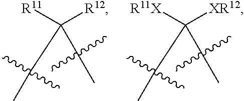

—C(O)—C(O)—; where R is selected from substituted or unsubstituted C₁₋₁₀ alkyl, phenyl;
R¹¹, R¹² are independently selected from hydrogen, hydroxyl, substituted or unsubstituted C₁₋₆ alkyl, or

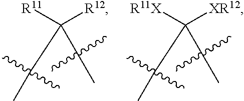

form 5-8 membered heterocycles, each may be unsubstituted or substituted by C₁₋₆ alkyl.

X is selected from O, S.

In one embodiment,
L¹ is selected from chemical bonds, substituted or unsubstituted C₁₋₆ alkyl;
A¹, L² are independently selected from substituted or unsubstituted C₁₋₆ alkyl, C₃₋₈ cycloalkyl, C₃₋₈ heterocycloalkyl, C₆₋₁₀ aryl, 5-10 membered heteroaryl, biphenyl,

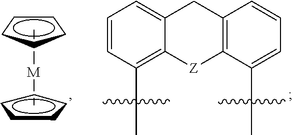

and
A¹, L² are independently be selected from chemical bonds, O, NH, S, substituted or unsubstituted C₁₋₁₂ alkyl, C₃₋₈ cycloalkyl, C₃₋₈ heterocycloalkyl, C₆₋₃₀ aryl, 5-20 membered heteroaryl;
and
A¹, if it is selected from O, NH, S, L¹ and L² each may independently be selected from substituted or unsubstituted C₆₋₁₀ aryl, 5-20 membered heteroaryl;
and
A¹, if it is O, L¹ and L² each may independently be selected from substituted or unsubstituted C₆₋₁₀ aryl, 5-10 membered heteroaryl;

In one embodiment, in the formula V, A is selected from

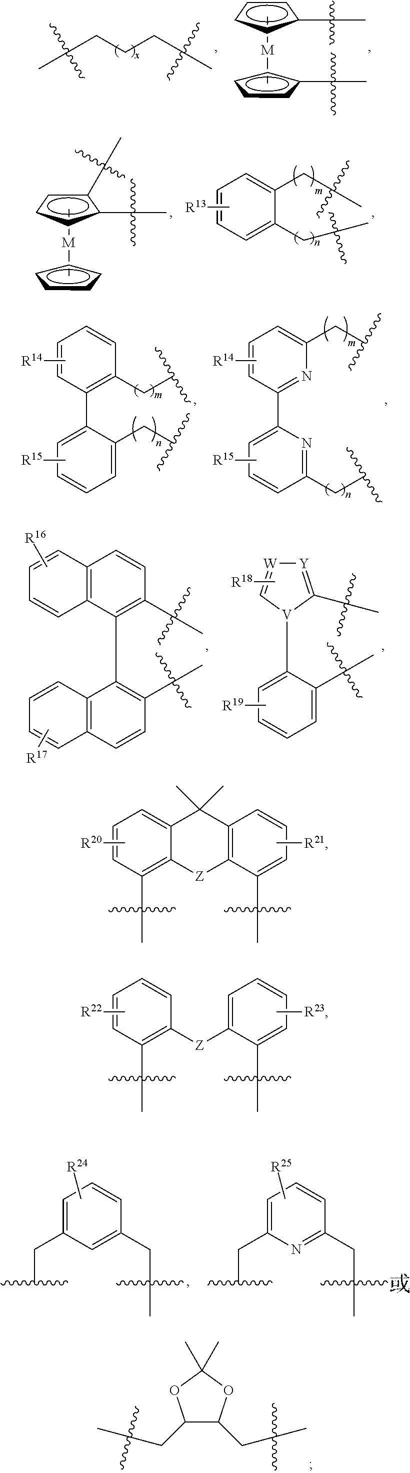

wherein
R¹³, R¹⁴, R¹⁵, R¹⁶, R¹⁷, R¹⁸, R¹⁹, R²⁰, R²¹, R²², R²³, R²⁴, R²⁵ are independently selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, and 5-30 membered heteroaryl; two adjacent R¹³, R¹⁴, R¹⁵, R¹⁶, R¹⁷, R¹⁸, R¹⁹, R²⁰, R²¹, R²², R²³ located on rings, together with the ring atoms to which they are attached, form a 5-7 membered carbocycle or heterocycle;
x is selected from 0, 1, 2, 3, 4, 5;
m, n are independently selected from 0, 1, 2, 3;
M is selected from Fe, Co, Ru;
V, W, Y are independently selected from N, CH; wherein any hydrogen atom on said CH may be substituted by R¹⁸;
and

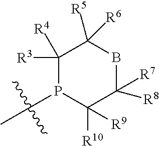

is selected from

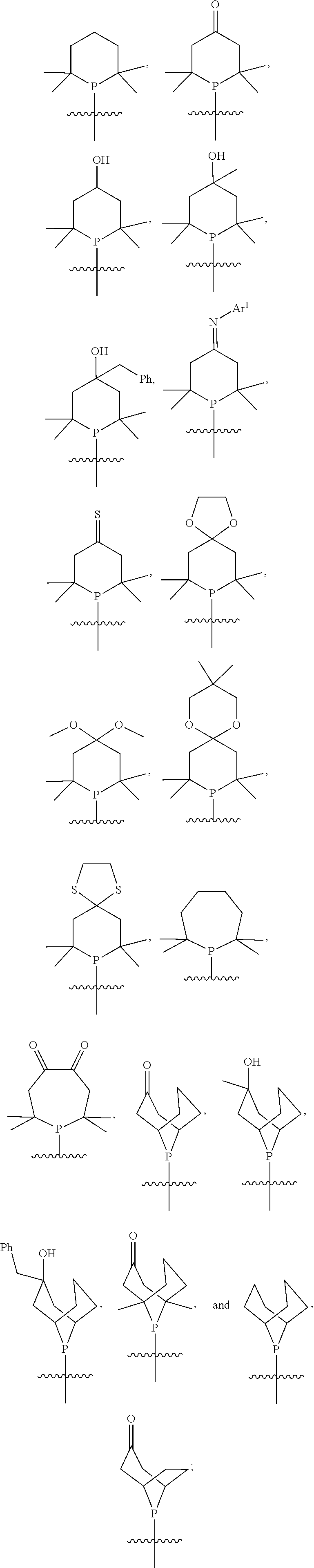

Ar¹ is selected from substituted or unsubstituted C₆₋₃₀ aryl, 5-20 membered heteroaryl.

In one embodiment, the phosphine ligand of formula V is selected from

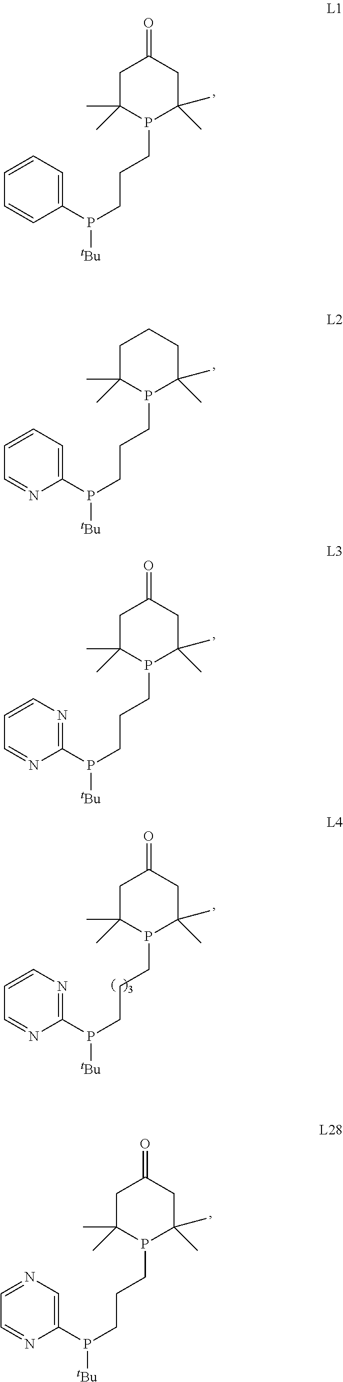

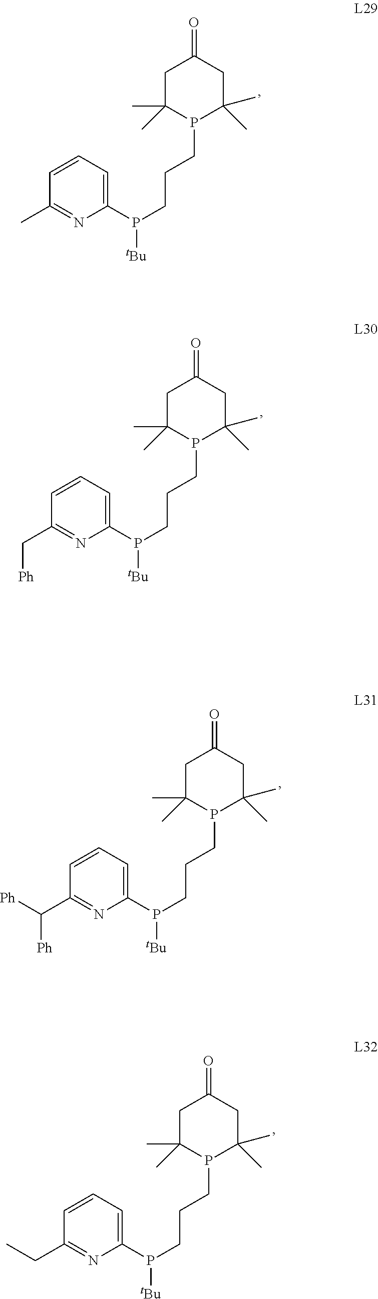

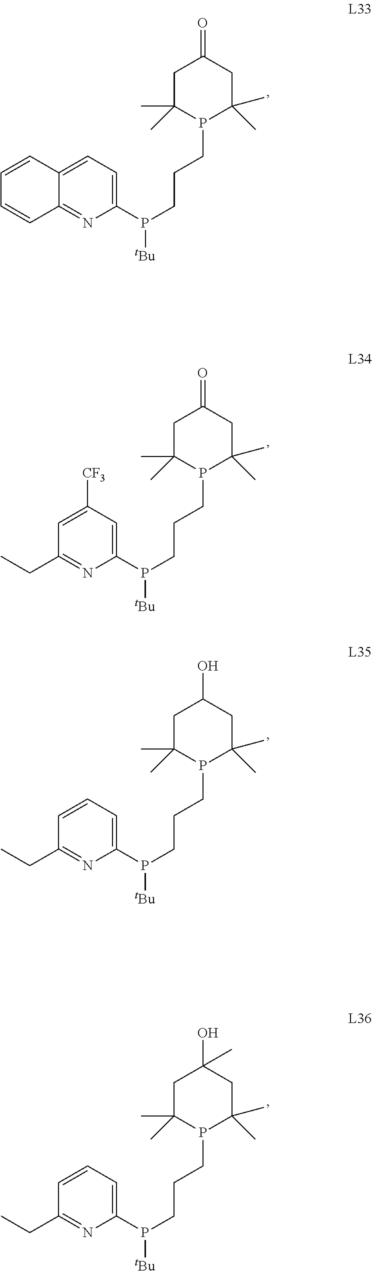

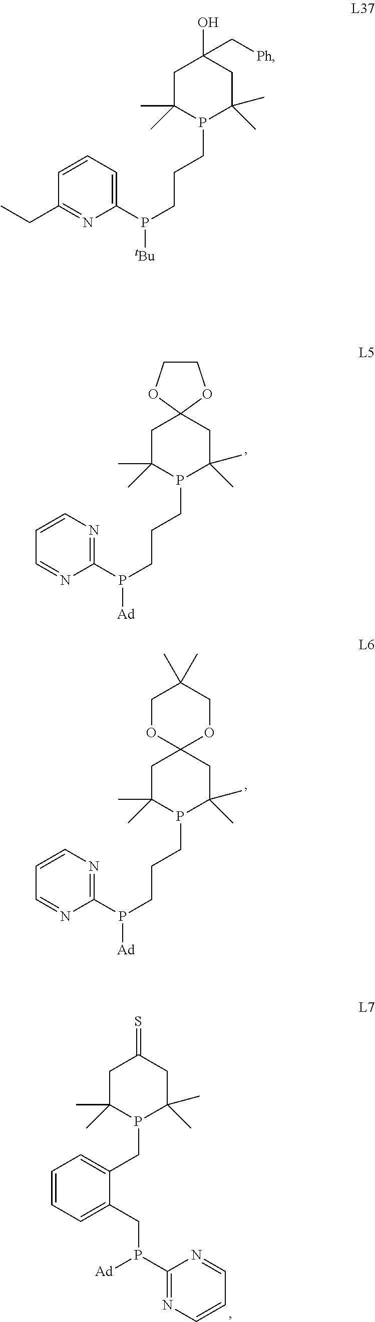

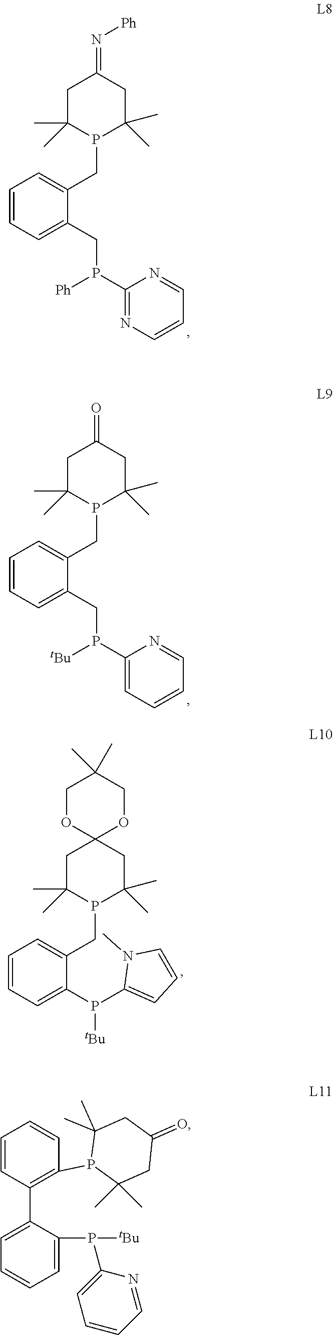 [SMILES: CC1(C)CC(=Nc2ccccc2)CC(C)(C)P1Cc1ccccc1CP(c1ccccc1)c1ncccn1 ; CC(C)(C)P(Cc1ccccc1CP1C(C)(C)CC(=O)CC1(C)C)c1ccccn1 ; Cn1cccc1P(c1ccccc1CP1C(C)(C)CC2(CC1(C)C)OCC(C)(C)CO2)C(C)(C)C ; CC(C)(C)P(c1ccccn1)c1ccccc1-c1ccccc1P1C(C)(C)CC(=O)CC1(C)C]

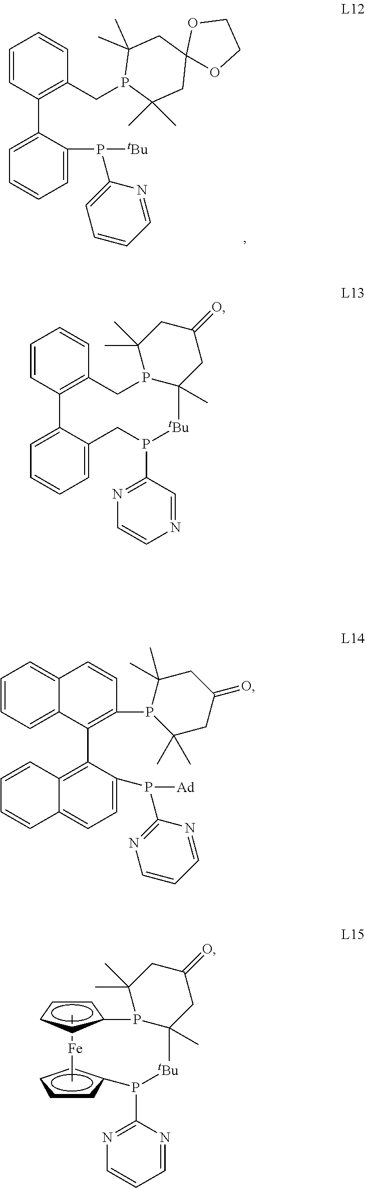

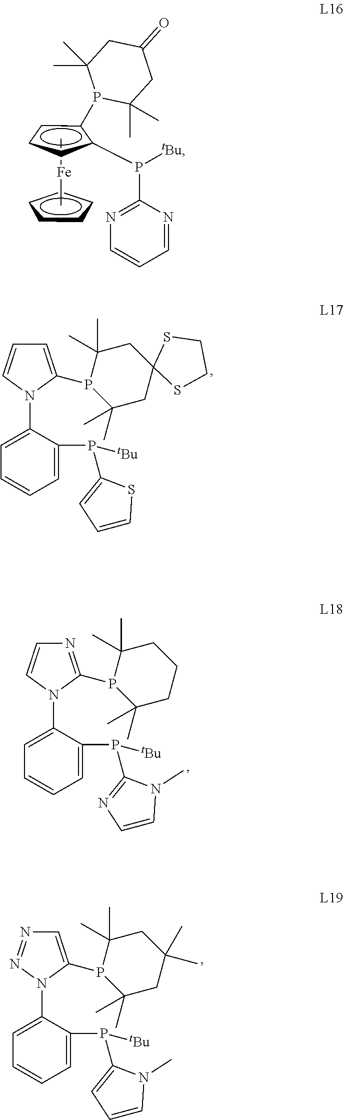

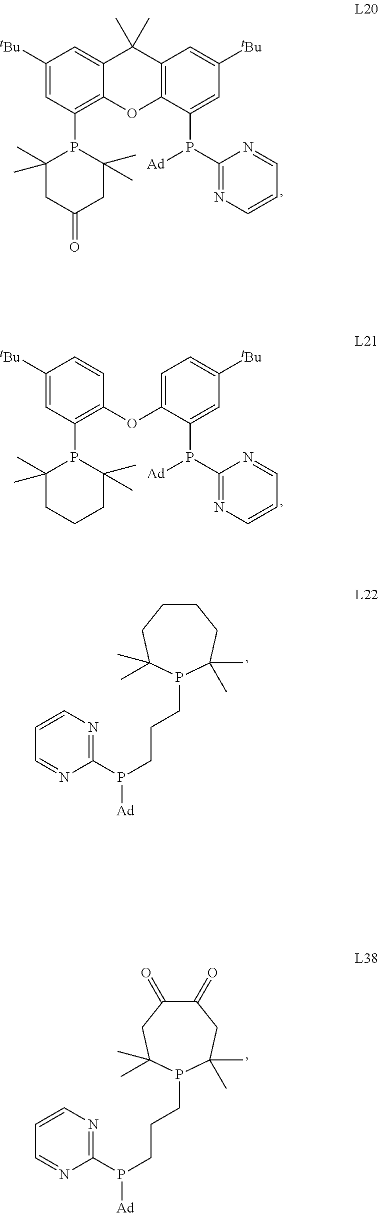

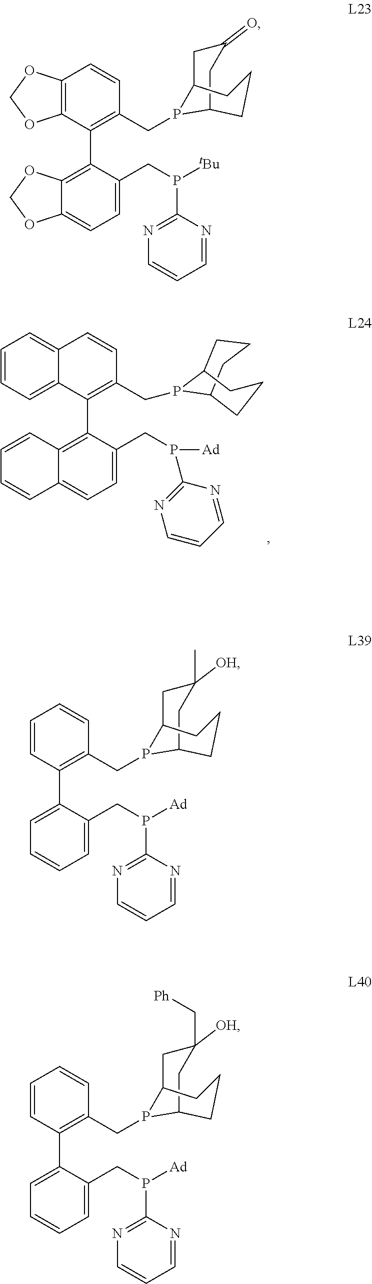

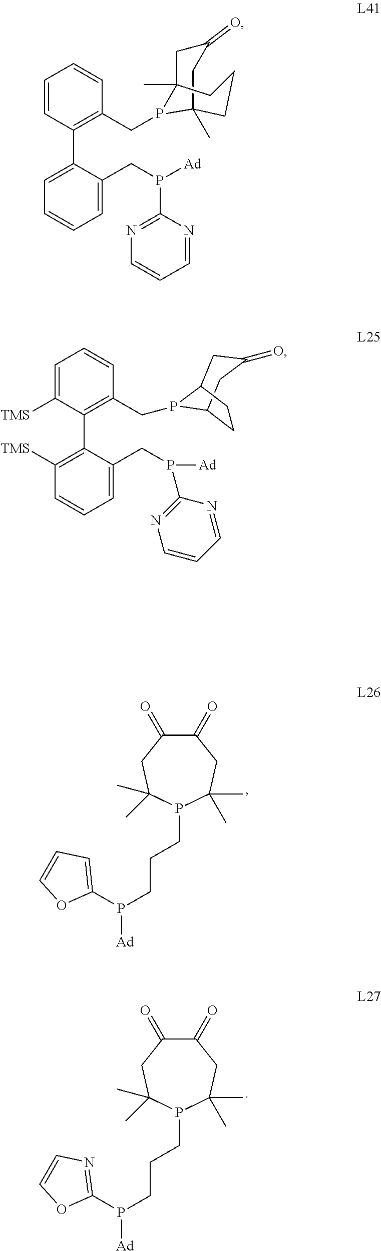

In seventh aspect, the present invention provides a method for palladium-catalyzed carbonylation of acetylene to synthesize acrylate esters, wherein said method comprising the steps of:
(a) In a reaction vessel, dissolving a palladium catalyst, a phosphine ligand, and an acid in an alcohol and an optional solvent; wherein said phosphine ligand is described as Formula V;
(b) Introducing acetylene into the vessel, followed by carbon monoxide, to initiate the reaction;
(c) Terminating the reaction and isolating the product.

In one embodiment, said reaction vessel is a high-pressure autoclave.

In one embodiment, the amount of said Formula V phosphine ligand is 0.00001-10 mol % relative to acetylene, more preferably 0.0001-1 mol %.

In one embodiment, the reaction temperature is 0-200° C., preferably 40-130° C.

In one embodiment, the reaction is conducted under an inert gas atmosphere; preferably, said inert gas is nitrogen and/or argon.

In one embodiment, the reaction is carried out at 1-10 MPa, preferably 2-8 MPa.

In one embodiment, the reaction time is 0.5-72 hours, preferably 0.5-24 hours.

In one embodiment, the said alcohol is a C₁₋₁₂ alkyl alcohol; preferably, said alcohol is selected from the group consisting of: methanol, ethanol, propanol, butanol, octanol, or a combination thereof.

In one embodiment, the amount of said alcohol is 1-100 molar equivalents relative to acetylene, preferably 1-10 molar equivalents.

In one embodiment, said palladium catalyst is selected from the group consisting of: palladium acetate, palladium trifluoroacetate, palladium pivalate, palladium(II) tetraacetonitrile tetrafluoroborate, palladium(II) hexafluoroacetylacetonate, palladium(II) bis(acetylacetonate), palladium(II) tetraacetonitrile trifluoromethanesulfonate, palladium(II) neodecanoate, bis(dibenzylideneacetone)palladium(0), tris(dibenzylideneacetone)dipalladium(0), palladium(II) chloride, palladium(II) diacetonitrile dichloride, palladium(II) dibenzonitrile dichloride, or a combination thereof.

In one embodiment, said palladium catalyst is selected from the group consisting of: palladium acetate, palladium trifluoroacetate, palladium pivalate, tris(dibenzylideneacetone)dipalladium(0), palladium(II) chloride, palladium(II) diacetonitrile dichloride, or a combination thereof.

In one embodiment, the amount of said palladium catalyst is 0.00001-10 mol % relative to acetylene, more preferably 0.0001-1 mol %.

In the reaction, when said phosphine ligand is a nitrogen-phosphine ligand (i.e., a Formula I compound), the molar ratio of said palladium catalyst to said nitrogen-phosphine ligand is 1:1 to 1:30, more preferably 1:1 to 1:5.

In one embodiment, said acid is selected from the group consisting of: perchloric acid, sulfuric acid, phosphoric acid, sulfonic acid, alkylphosphonic acid, alkylsulfonic acid, alkylcarboxylic acid, perfluoroalkylsulfonic acid, perfluoroalkylcarboxylic acid (wherein said alkyl is a C₁₋₁₂ alkyl), or arylsulfonic acid (wherein said aryl is a C₆₋₁₀ aryl).

In one embodiment, said acid is selected from the group consisting of: methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid, dodecylsulfonic acid, sulfuric acid, sulfonic acid, formic acid, and trifluoroacetic acid.

In one embodiment, the amount of said acid is 0.00004-40 mol % relative to acetylene, preferably 0.0004-4 mol %.

In one embodiment, said solvent is selected from the group consisting of: alkane solvents, substituted aromatic solvents, ether solvents, ketone solvents, nitrile solvents, ester solvents, or a combination thereof.

In one embodiment, said alkane solvent is selected from the group consisting of: n-hexane, cyclohexane, or a combination thereof.

In one embodiment, said substituted aromatic solvent is selected from the group consisting of: chlorobenzene, toluene, xylene, and trifluorotoluene.

In one embodiment, said ether solvent is selected from the group consisting of: tetrahydrofuran, diethyl ether, methyl tert-butyl ether, ethyl tert-butyl ether, anisole, ethylene glycol dimethyl ether, 1,4-dioxane, or a combination thereof.

In one embodiment, said ketone solvent is acetone.

In one embodiment, said nitrile solvent is selected from the group consisting of: acetonitrile, propionitrile, benzonitrile, or a combination thereof.

In one embodiment, said ester solvent is ethyl acetate.

In one embodiment, said alcohol is methanol, and the reaction is carried out under the following conditions:
a) Dissolving palladium acetate, a phosphine ligand of Formula V, and an acid in methanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is ethanol, and the reaction is carried out under the following conditions:
a) Dissolving palladium acetate, a phosphine ligand of Formula V, and an acid in ethanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is butanol, and the reaction is carried out under the following conditions:
a) Dissolving palladium acetate, a phosphine ligand of Formula V, and an acid in butanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is octanol, and the reaction is carried out under the following conditions:
a) Dissolving palladium acetate, a phosphine ligand of Formula V, and an acid in octanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In eighth aspect, the present invention provides a phosphine ligand for the catalytic preparation of acrylate esters from acetylene, wherein the phosphine ligand has a structure selected from Formula VI

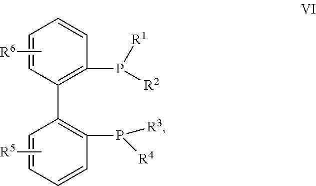

wherein
R¹, R⁴ are independently selected from substituted or unsubstituted C₁₋₁₀ alkyl, C₃₋₁₀ cycloalkyl, C₆₋₃₀ aryl;
R² and R³ are independently selected from 5-20 membered heteroaryl;
R⁵, R⁶ are independently selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, and 5-30 membered heteroaryl; two adjacent R⁵, R⁶ located on rings, together with the ring atoms to which they are attached, form a 5-7 membered carbocycle or heterocycle; wherein the heterocycles contain 1-3 heteroatoms selected from N, O, S;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, unless expressly stated otherwise.

In one embodiment, in the formula VI,
R¹, R⁴ are independently selected from substituted or unsubstituted C₁₋₄ alkyl, C₃₋₆ cycloalkyl, C₆₋₁₄ aryl;
R², R³ are independently selected from 5-10 membered heteroaryl;
and
R¹, R², R³, R⁴ are independently unsubstituted or substituted by one or more substituents selected from methyl, methoxyl, cyano, COOH, benzenesulfonyl, trimethylsilyl.

In one embodiment, in the formula VI,
R¹, R⁴ are independently selected from methyl, ethyl, n-propyl, isopropyl, n-butyl, tert-butyl, isobutyl, cyclopropyl, cyclobutyl, cyclopentyl, cyclohexyl, phenyl, adamantyl.
R², R³ are independently selected from pyridyl, pyrazinyl, pyrimidinyl, pyridazinyl, pyrrolyl, imidazolyl, pyrazolyl, thienyl, furyl, thiazolyl, triazolyl;
R⁵, R⁶ are independently selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, 5-30 membered heteroaryl; or two adjacent R⁵, R⁶ located on rings, together with the ring atoms to which they are attached, form a 5-7 membered carbocycle or heterocycle.

In one embodiment, the phosphine ligand of formula VI is selected from

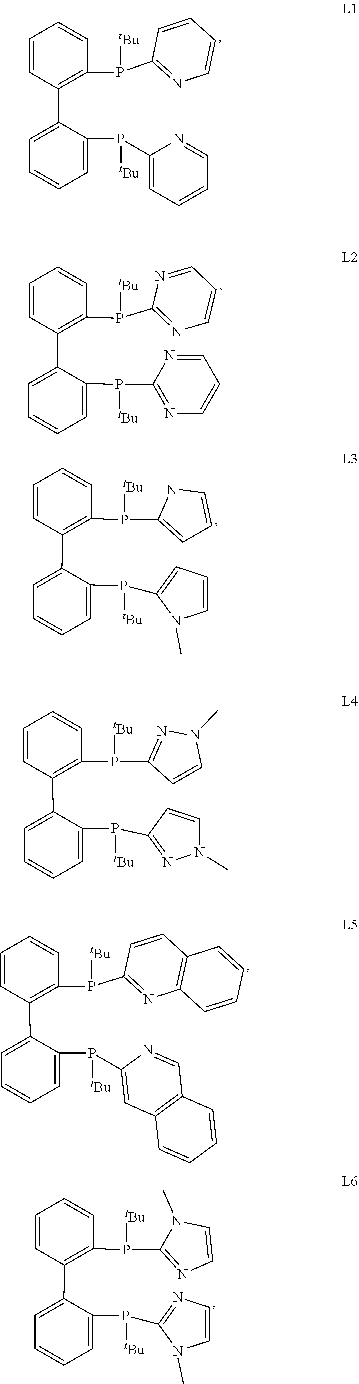

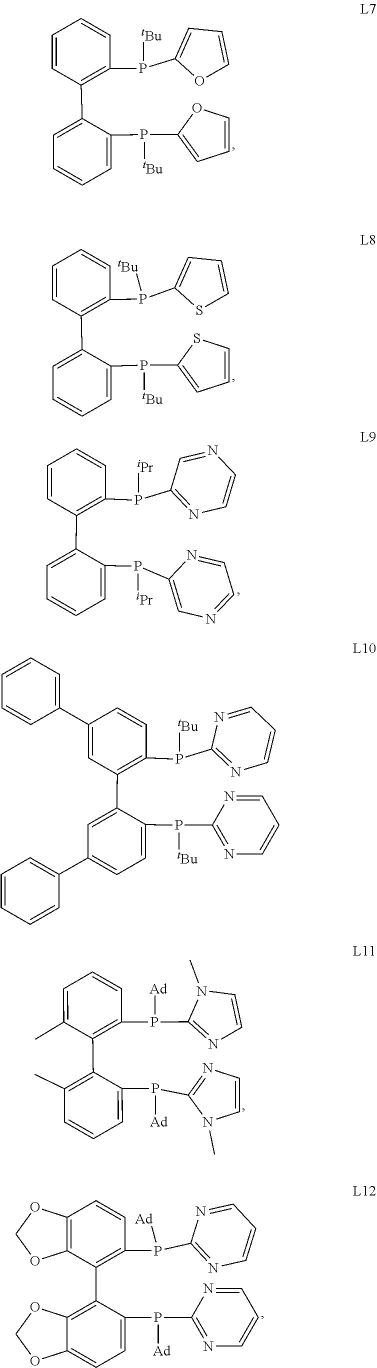

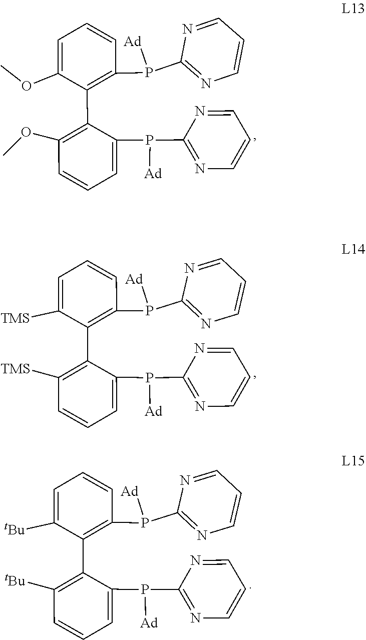

In ninth aspect, the present invention provides a method for palladium-catalyzed carbonylation of acetylene to synthesize acrylate esters, wherein said method comprising the steps of:
(a) In a reaction vessel, dissolving a palladium catalyst, a phosphine ligand, and an acid in an alcohol and an optional solvent; wherein said phosphine ligand is described as Formula VI;
(b) Introducing acetylene into the vessel, followed by carbon monoxide, to initiate the reaction;
(c) Terminating the reaction and isolating the product.

In one embodiment, said reaction vessel is a high-pressure autoclave.

In one embodiment, the amount of said Formula VI phosphine ligand is 0.00001-10 mol % relative to acetylene, more preferably 0.0001-1 mol %.

In one embodiment, the reaction temperature is 0-200° C., preferably rt-130° C.

In one embodiment, the reaction is conducted under an inert gas atmosphere; preferably, said inert gas is nitrogen and/or argon.

In one embodiment, the reaction is carried out at 1-10 MPa, preferably 2-8 MPa.

In one embodiment, the reaction time is 0.5-72 hours, preferably 0.5-24 hours.

In one embodiment, the said alcohol is a C₁₋₁₂ alkyl alcohol; preferably, said alcohol is selected from the group consisting of: methanol, ethanol, propanol, butanol, octanol, or a combination thereof.

In one embodiment, the amount of said alcohol is 1-100 molar equivalents relative to acetylene, preferably 1-10 molar equivalents.

In one embodiment, said palladium catalyst is selected from the group consisting of: palladium acetate, palladium trifluoroacetate, palladium pivalate, palladium(II) tetraacetonitrile tetrafluoroborate, palladium(II) hexafluoroacetylacetonate, palladium(II) bis(acetylacetonate), palladium(II) tetraacetonitrile trifluoromethanesulfonate, palladium(II) neodecanoate, bis(dibenzylideneacetone)palladium(0), tris(dibenzylideneacetone)dipalladium(0), palladium(II) chloride, palladium(II) diacetonitrile dichloride, palladium(II) dibenzonitrile dichloride, or a combination thereof.

In one embodiment, said palladium catalyst is selected from the group consisting of: palladium acetate, palladium trifluoroacetate, palladium pivalate, tris(dibenzylideneacetone)dipalladium(0), palladium(II) chloride, palladium(II) diacetonitrile dichloride, or a combination thereof.

In one embodiment, the amount of said palladium catalyst is 0.00001-10 mol % relative to acetylene, more preferably 0.0001-1 mol %.

In the reaction, when said phosphine ligand is a nitrogen-phosphine ligand (i.e., a Formula I compound), the molar ratio of said palladium catalyst to said nitrogen-phosphine ligand is 1:1 to 1:30, more preferably 1:1 to 1:5.

In one embodiment, said acid is selected from the group consisting of: perchloric acid, sulfuric acid, phosphoric acid, sulfonic acid, alkylphosphonic acid, alkylsulfonic acid, alkylcarboxylic acid, perfluoroalkylsulfonic acid, perfluoroalkylcarboxylic acid (wherein said alkyl is a C₁₋₁₂ alkyl), or arylsulfonic acid (wherein said aryl is a C₆₋₁₀ aryl).

In one embodiment, said acid is selected from the group consisting of: methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid, dodecylsulfonic acid, sulfuric acid, sulfonic acid, formic acid, and trifluoroacetic acid.

In one embodiment, the amount of said acid is 0.00004-100 mol % relative to acetylene, preferably 0.0004-4 mol %.

In one embodiment, said solvent is selected from the group consisting of: alkane solvents, substituted aromatic solvents, ether solvents, ketone solvents, nitrile solvents, ester solvents, or a combination thereof.

In one embodiment, said alkane solvent is selected from the group consisting of: n-hexane, cyclohexane, or a combination thereof.

In one embodiment, said substituted aromatic solvent is selected from the group consisting of: chlorobenzene, toluene, xylene, and trifluorotoluene.

In one embodiment, said ether solvent is selected from the group consisting of: tetrahydrofuran, diethyl ether, methyl tert-butyl ether, ethyl tert-butyl ether, anisole, ethylene glycol dimethyl ether, 1,4-dioxane, or a combination thereof.

In one embodiment, said ketone solvent is acetone.

In one embodiment, said nitrile solvent is selected from the group consisting of: acetonitrile, propionitrile, benzonitrile, or a combination thereof.

In one embodiment, said ester solvent is ethyl acetate.

In one embodiment, said alcohol is methanol, and the reaction is carried out under the following conditions:
a) Dissolving palladium acetate, a phosphine ligand of Formula VI, and an acid in methanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is ethanol, and the reaction is carried out under the following conditions:
a) Dissolving palladium acetate, a phosphine ligand of Formula VI, and an acid in ethanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is butanol, and the reaction is carried out under the following conditions:
a) Dissolving palladium acetate, a phosphine ligand of Formula VI, and an acid in butanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is octanol, and the reaction is carried out under the following conditions:
a) Dissolving palladium acetate, a phosphine ligand of Formula VI, and an acid in octanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In tenth aspect, the present invention provides a method for palladium-catalyzed carbonylation of acetylene to synthesize acrylate esters, such as methyl acrylate, ethyl acrylate, butyl acrylate, or octyl acrylate, wherein said method comprising the steps of:
(a) In a reaction vessel, dissolving a palladium catalyst, a phosphine ligand, and an acid in an alcohol and an optional solvent;
(b) Introducing acetylene into the vessel, followed by carbon monoxide, to initiate the reaction;
(c) Terminating the reaction and isolating the product.

The phosphine ligand according to the invention comprises the Formula VII:

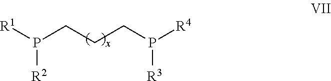

wherein
R¹, R⁴ are independently selected from substituted or unsubstituted C₁₋₁₂ alkyl, C₃₋₁₂ cycloalkyl, C₃₋₁₂ heterocycloalkyl, C₆₋₃₀ aryl;
R² and R³ are independently selected from 5-20 membered heteroaryl;
x is selected from 0, 1, 2, 3, 4, 5;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, unless expressly stated otherwise.

In one embodiment, in the formula VII,
R¹, R⁴ are independently selected from substituted or unsubstituted C₁₋₁₀ alkyl, C₃₋₆ cycloalkyl, C₆₋₁₀ aryl;
R² and R³ are independently selected from 5-20 membered heteroaryl;
and
R¹, R², R³, R⁴ are independently unsubstituted or substituted by one or more substituents selected from methyl, methoxyl, cyano, COOH, benzenesulfonyl, trimethylsilyl.

In one embodiment, in the formula VII,
R¹, R⁴ are independently selected from methyl, ethyl, n-propyl, isopropyl, n-butyl, tert-butyl, isobutyl, cyclopropyl, cyclobutyl, cyclopentyl, cyclohexyl, adamantyl, phenyl;
R², R³ are independently selected from pyridyl, pyrazinyl, pyrimidinyl, pyridazinyl, pyrrolyl, imidazolyl, pyrazolyl, thienyl, furyl, thiazolyl, triazolyl;
and
the said substituents are independently selected from C₁₋₄ alkyl, C₁₋₄ alkoxy, C₂₋₆ ester, cyano, COOH, benzenesulfonyl, tri(C₁₋₄ alkyl)silyl, nitro, C₆₋₁₀ aryl, 5-9 membered heteroaryl.

In one embodiment, the phosphine ligand of formula VII is selected from

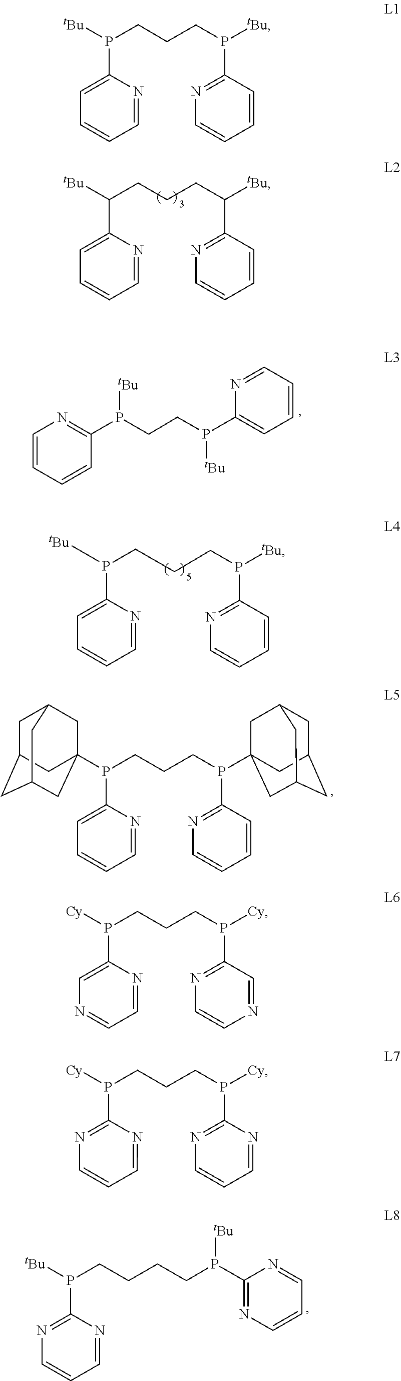

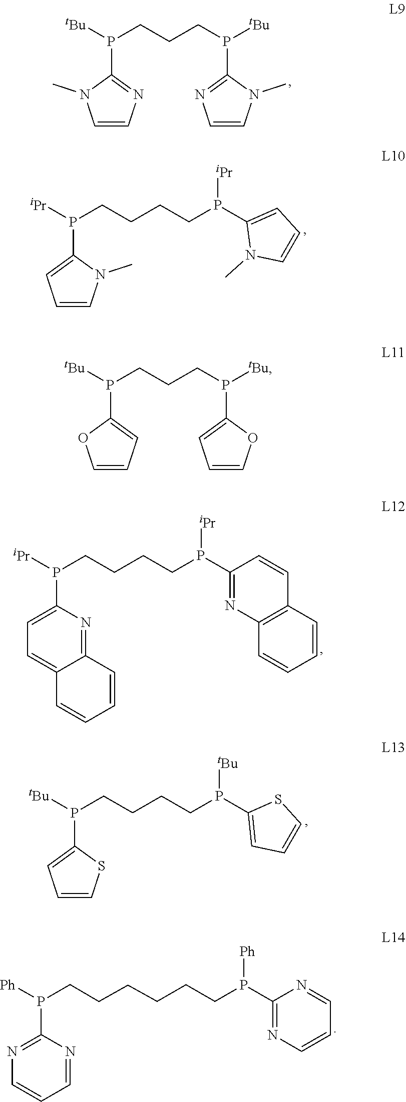

In one embodiment, said reaction vessel is a high-pressure autoclave.

In one embodiment, the amount of said Formula VII phosphine ligand is 0.00001-10 mol % relative to acetylene, more preferably 0.0001-1 mol %.

In one embodiment, the reaction temperature is 0-200° C., preferably 40-130° C.

In one embodiment, the reaction is conducted under an inert gas atmosphere; preferably, said inert gas is nitrogen and/or argon.

In one embodiment, the reaction is carried out at 1-10 MPa, preferably 2-8 MPa.

In one embodiment, the reaction time is 0.5-72 hours, preferably 0.5-24 hours.

In one embodiment, the said alcohol is a C₁₋₁₂ alkyl alcohol; preferably, said alcohol is selected from the group consisting of: methanol, ethanol, propanol, butanol, octanol, or a combination thereof.

In one embodiment, the amount of said alcohol is 1-100 molar equivalents relative to acetylene, preferably 1-10 molar equivalents.

In one embodiment, said palladium catalyst is selected from the group consisting of: palladium acetate, palladium trifluoroacetate, palladium pivalate, palladium(II) tetraacetonitrile tetrafluoroborate, palladium(II) hexafluoroacetylacetonate, palladium(II) bis(acetylacetonate), palladium(II) tetraacetonitrile trifluoromethanesulfonate, palladium(II) neodecanoate, bis(dibenzylideneacetone)palladium(0), tris(dibenzylideneacetone)dipalladium(0), palladium(II) chloride, palladium(II) diacetonitrile dichloride, palladium(II) dibenzonitrile dichloride, or a combination thereof.

In one embodiment, said palladium catalyst is selected from the group consisting of: palladium acetate, palladium trifluoroacetate, palladium pivalate, tris(dibenzylideneacetone)dipalladium(0), palladium(II) chloride, palladium(II) diacetonitrile dichloride, or a combination thereof.

In one embodiment, the amount of said palladium catalyst is 0.00001-10 mol % relative to acetylene, more preferably 0.0001-1 mol %.

In the reaction, when said phosphine ligand is a nitrogen-phosphine ligand (i.e., a Formula VII compound), the molar ratio of said palladium catalyst to said nitrogen-phosphine ligand is 1:1 to 1:30, more preferably 1:1 to 1:5.

In one embodiment, said acid is selected from the group consisting of: perchloric acid, sulfuric acid, phosphoric acid, sulfonic acid, alkylphosphonic acid, alkylsulfonic acid, alkylcarboxylic acid, perfluoroalkylsulfonic acid, perfluoroalkylcarboxylic acid (wherein said alkyl is a C₁₋₁₂ alkyl), or arylsulfonic acid (wherein said aryl is a C₆₋₁₀ aryl).

In one embodiment, said acid is selected from the group consisting of: methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid, dodecylsulfonic acid, sulfuric acid, sulfonic acid, formic acid, and trifluoroacetic acid.

In one embodiment, the amount of said acid is 0.00016-100 mol % relative to acetylene, preferably 0.0016-4 mol %.

In one embodiment, said solvent is selected from the group consisting of: alkane solvents, substituted aromatic solvents, ether solvents, ketone solvents, nitrile solvents, ester solvents, or a combination thereof.

In one embodiment, said alkane solvent is selected from the group consisting of: n-hexane, cyclohexane, or a combination thereof.

In one embodiment, said substituted aromatic solvent is selected from the group consisting of: chlorobenzene, toluene, xylene, and trifluorotoluene.

In one embodiment, said ether solvent is selected from the group consisting of: tetrahydrofuran, diethyl ether, methyl tert-butyl ether, ethyl tert-butyl ether, anisole, ethylene glycol dimethyl ether, 1,4-dioxane, or a combination thereof.

In one embodiment, said ketone solvent is acetone.

In one embodiment, said nitrile solvent is selected from the group consisting of: acetonitrile, propionitrile, benzonitrile, or a combination thereof.

In one embodiment, said ester solvent is ethyl acetate.

In one embodiment, said alcohol is methanol, and the reaction is carried out under the following conditions:
a) Dissolving palladium acetate, a phosphine ligand of Formula VII, and an acid in methanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In one embodiment, said alcohol is ethanol, and the reaction is carried out under the following conditions:
a) Dissolving palladium acetate, a phosphine ligand of Formula VII, and an acid in ethanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In one embodiment, said alcohol is butanol, and the reaction is carried out under the following conditions:
a) Dissolving palladium acetate, a phosphine ligand of Formula VII, and an acid in butanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In one embodiment, said alcohol is octanol, and the reaction is carried out under the following conditions:
a) Dissolving palladium acetate, a phosphine ligand of Formula VII, and an acid in octanol or a mixed solvent with an optional co-solvent;
b) Introducing acetylene into the reactor, followed by carbon monoxide to initiate the reaction, wherein the reaction proceeds at a temperature ranging from room temperature to 130° C.;
c) Terminating the reaction and isolating the product.

In eleventh aspect, the present invention provides a phosphine ligand, wherein the phosphine ligand has a structure as Formula VIII

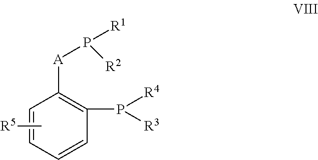

wherein
R¹, R⁴ are independently selected from substituted or unsubstituted C₁₋₁₀ alkyl, C₃₋₁₀ cycloalkyl, C₆₋₃₀ aryl;
R² and R³ are independently selected from 5-20 membered heteroaryl;
A is selected from 5-10 membered heteroaryl, may independently by one or more R⁶ substituents, where one or more heteroatoms are independently selected from O, N, S;
R⁵, R⁶ are independently selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, and 5-30 membered heteroaryl; two adjacent R⁵ located on rings, together with the ring atoms to which they are attached, form a 5-7 membered carbocycle or heterocycle;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, unless expressly stated otherwise.
with the proviso that phosphine ligand is not

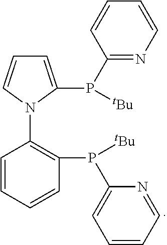 [SMILES: CC(C)(C)P(c1ccccn1)c1ccccc1-n1cccc1P(c1ccccn1)C(C)(C)C]

In one embodiment, when A is pyrrole, the compound satisfies any one to three of the following conditions (1)-(3):
(1) R⁵, R⁶ are not simultaneously hydrogen;
(2) R¹, R⁴ are not simultaneously tert-butyl;
(3) R², R³ are not simultaneously 2-pyridyl.

In one embodiment, in the formula VIII,
R¹, R⁴ are independently selected from substituted or unsubstituted C₁₋₄ alkyl, C₃₋₁₀ cycloalkyl, phenyl;
R² and R³ are independently selected from 5-20 membered heteroaryl;
and
R¹, R², R³, R⁴ are independently unsubstituted or substituted by one or more substituents selected from methyl, methoxyl, cyano, COOH, benzenesulfonyl, trimethylsilyl.

In one embodiment, in the formula VIII, A is a 5- to 9-membered heteroaryl group, unsubstituted or substituted with one or more R⁶, wherein the heteroatoms in the heteroaryl group are selected from O, N, and S, and may be the same or different.

In one embodiment, in the formula VIII,
R¹, R⁴ are independently selected from methyl, ethyl, n-propyl, isopropyl, n-butyl, tert-butyl, isobutyl, cyclopropyl, cyclobutyl, cyclopentyl, cyclohexyl, adamantyl, phenyl;
R², R³ are independently selected from pyridyl, pyrazinyl, pyrimidinyl, pyridazinyl, pyrrolyl, imidazolyl, pyrazolyl, thienyl, furyl, thiazolyl, triazolyl.
and
R¹, R², R³, R⁴ are independently unsubstituted or substituted by one or more substituents selected from methyl, methoxyl, cyano, COOH, benzenesulfonyl, trimethylsilyl.

In one embodiment, the phosphine ligand of formula VIII is selected from

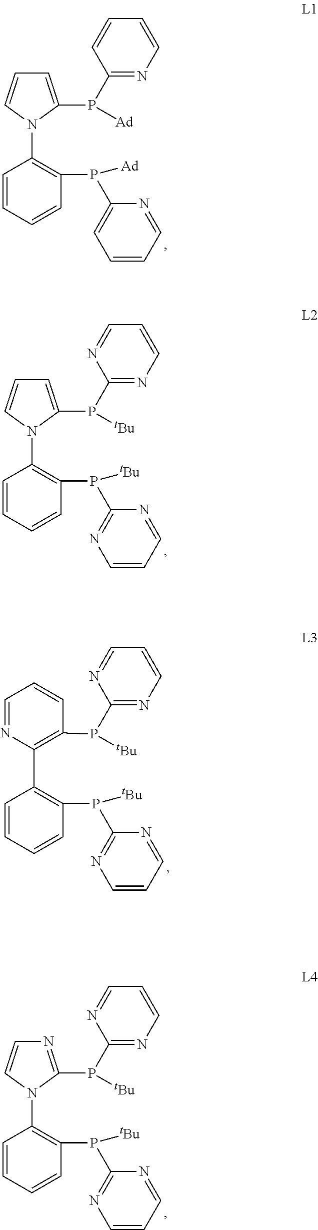

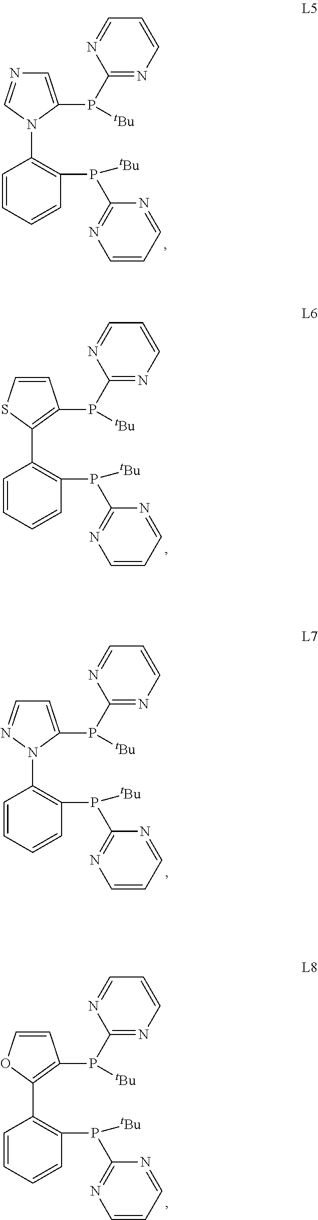 [SMILES: CC(C)(C)P(c1ncccn1)c1ccccc1-n1cncc1P(c1ncccn1)C(C)(C)C ; CC(C)(C)P(c1ncccn1)c1ccccc1-c1sccc1P(c1ncccn1)C(C)(C)C ; CC(C)(C)P(c1ncccn1)c1ccccc1-n1nccc1P(c1ncccn1)C(C)(C)C ; CC(C)(C)P(c1ncccn1)c1ccccc1-c1occc1P(c1ncccn1)C(C)(C)C]

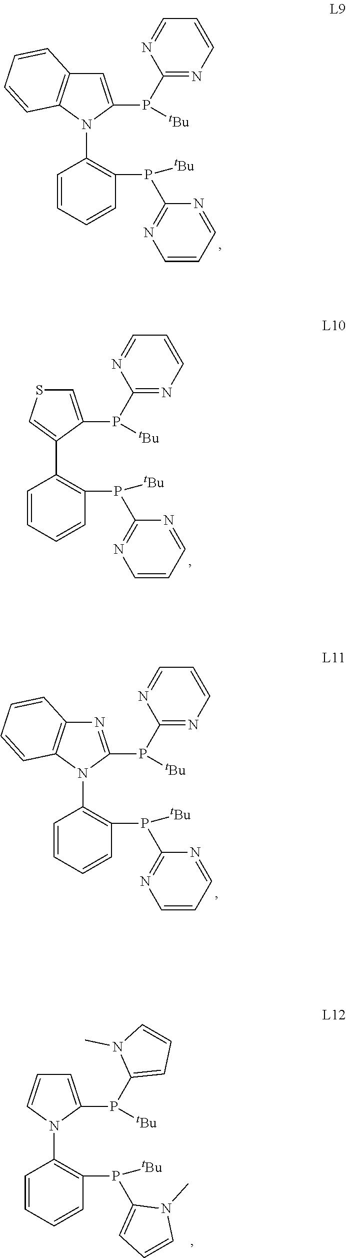 [SMILES: CC(C)(C)P(c1ncccn1)c1ccccc1-n1c(P(c2ncccn2)C(C)(C)C)cc2ccccc21 ; CC(C)(C)P(c1ncccn1)c1ccccc1-c1cscc1P(c1ncccn1)C(C)(C)C ; CC(C)(C)P(c1ncccn1)c1ccccc1-n1c(P(c2ncccn2)C(C)(C)C)nc2ccccc21 ; Cn1cccc1P(c1ccccc1-n1cccc1P(c1cccn1C)C(C)(C)C)C(C)(C)C]

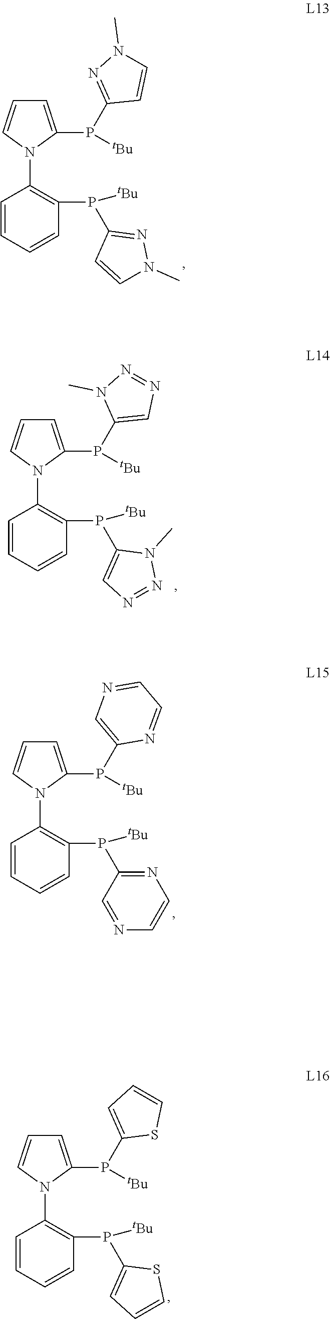 [SMILES: Cn1ccc(P(c2ccccc2-n2cccc2P(c2ccn(C)n2)C(C)(C)C)C(C)(C)C)n1 ; Cn1nncc1P(c1ccccc1-n1cccc1P(c1cnnn1C)C(C)(C)C)C(C)(C)C ; CC(C)(C)P(c1cnccn1)c1ccccc1-n1cccc1P(c1cnccn1)C(C)(C)C ; CC(C)(C)P(c1cccs1)c1ccccc1-n1cccc1P(c1cccs1)C(C)(C)C]

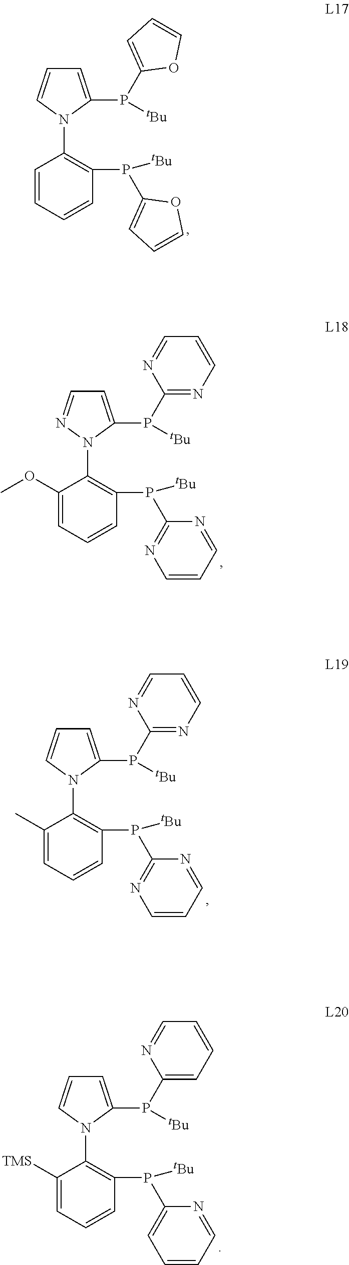 [SMILES: CC(C)(C)P(c1ccco1)c1ccccc1-n1cccc1P(c1ccco1)C(C)(C)C ; COc1cccc(P(c2ncccn2)C(C)(C)C)c1-n1nccc1P(c1ncccn1)C(C)(C)C ; Cc1cccc(P(c2ncccn2)C(C)(C)C)c1-n1cccc1P(c1ncccn1)C(C)(C)C ; CC(C)(C)P(c1ccccn1)c1cccc([Si](C)(C)C)c1-n1cccc1P(c1ccccn1)C(C)(C)C]

In twelfth aspect, the present invention provides a method for palladium-catalyzed carbonylation of acetylene to synthesize acrylate esters, wherein said method comprising the steps of:
(a) In a reaction vessel, dissolving a palladium catalyst, a phosphine ligand, and an acid in an alcohol and an optional solvent;
wherein said phosphine ligand is described as Formula VIII, or

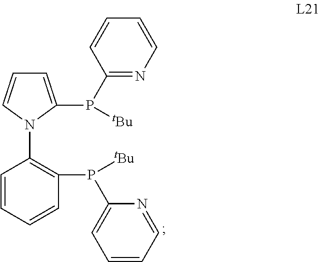 [SMILES: CC(C)(C)P(c1ccccn1)c1ccccc1-n1cccc1P(c1ccccn1)C(C)(C)C]

(b) Introducing acetylene into the vessel, followed by carbon monoxide, to initiate the reaction;
(c) Terminating the reaction and isolating the product

In one embodiment, said reaction vessel is a high-pressure autoclave.

In one embodiment, the amount of said Formula VIII phosphine ligand is 0.00001-10 mol % relative to acetylene, more preferably 0.0001-1 mol %.

In one embodiment, the reaction temperature is 0-200° C., preferably 40-130° C.

In one embodiment, the reaction is conducted under an inert gas atmosphere; preferably, said inert gas is nitrogen and/or argon.

In one embodiment, the reaction is carried out at 1-10 MPa, preferably 2-8 MPa.

In one embodiment, the reaction time is 0.5-72 hours, preferably 0.5-24 hours.

In one embodiment, the said alcohol is a C₁₋₁₂ alkyl alcohol; preferably, said alcohol is selected from the group consisting of: methanol, ethanol, propanol, butanol, octanol, or a combination thereof.

In one embodiment, the amount of said alcohol is 1-100 molar equivalents relative to acetylene, preferably 1-10 molar equivalents.

In one embodiment, said palladium catalyst is selected from the group consisting of: palladium acetate, palladium trifluoroacetate, palladium pivalate, palladium(II) tetraacetonitrile tetrafluoroborate, palladium(II) hexafluoroacetylacetonate, palladium(II) bis(acetylacetonate), palladium(II) tetraacetonitrile trifluoromethanesulfonate, palladium(II) neodecanoate, bis(dibenzylideneacetone)palladium(0), tris(dibenzylideneacetone)dipalladium(0), palladium(II) chloride, palladium(II) diacetonitrile dichloride, palladium(II) dibenzonitrile dichloride, or a combination thereof.

In one embodiment, said palladium catalyst is selected from the group consisting of: palladium acetate, palladium trifluoroacetate, palladium pivalate, tris(dibenzylideneacetone)dipalladium(0), palladium(II) chloride, palladium(II) diacetonitrile dichloride, or a combination thereof.

In one embodiment, the amount of said palladium catalyst is 0.00001-10 mol % relative to acetylene, more preferably 0.001-1 mol %.

In the reaction, when said phosphine ligand is a nitrogen-phosphine ligand (i.e., a Formula I compound), the molar ratio of said palladium catalyst to said nitrogen-phosphine ligand is 1:1 to 1:30, more preferably 1:1 to 1:5.

In one embodiment, said acid is selected from the group consisting of: perchloric acid, sulfuric acid, phosphoric acid, sulfonic acid, alkylphosphonic acid, alkylsulfonic acid, alkylcarboxylic acid, perfluoroalkylsulfonic acid, perfluoroalkylcarboxylic acid (wherein said alkyl is a C₁₋₁₂ alkyl), or arylsulfonic acid (wherein said aryl is a C₆₋₁₀ aryl).

In one embodiment, said acid is selected from the group consisting of: methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid, dodecylsulfonic acid, sulfuric acid, sulfonic acid, formic acid, and trifluoroacetic acid.

In one embodiment, the amount of said acid is 0.00004-40 mol % relative to acetylene, preferably 0.004-4 mol %.

In one embodiment, said solvent is selected from the group consisting of: alkane solvents, substituted aromatic solvents, ether solvents, ketone solvents, nitrile solvents, ester solvents, or a combination thereof.

In one embodiment, said alkane solvent is selected from the group consisting of: n-hexane, cyclohexane, or a combination thereof.

In one embodiment, said substituted aromatic solvent is selected from the group consisting of: chlorobenzene, toluene, xylene, and trifluorotoluene.

In one embodiment, said ether solvent is selected from the group consisting of: tetrahydrofuran, diethyl ether, methyl tert-butyl ether, ethyl tert-butyl ether, anisole, ethylene glycol dimethyl ether, 1,4-dioxane, or a combination thereof.

In one embodiment, said ketone solvent is acetone.

In one embodiment, said nitrile solvent is selected from the group consisting of: acetonitrile, propionitrile, benzonitrile, or a combination thereof.

In one embodiment, said ester solvent is ethyl acetate.

In one embodiment, said alcohol is methanol, and the reaction is carried out under the following conditions:
a) Dissolving palladium acetate, a phosphine ligand of Formula VIII, and an acid in methanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is ethanol, and the reaction is carried out under the following conditions:
a) Dissolving palladium acetate, a phosphine ligand of Formula VIII, and an acid in ethanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is butanol, and the reaction is carried out under the following conditions:
a) Dissolving palladium acetate, a phosphine ligand of Formula VIII, and an acid in butanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is octanol, and the reaction is carried out under the following conditions:
a) Dissolving palladium acetate, a phosphine ligand of Formula VIII, and an acid in octanol or a mixed solvent with an optional co-solvent;
b) Introducing acetylene into the reactor, followed by carbon monoxide to initiate the reaction, wherein the reaction proceeds at a temperature ranging from room temperature to 120° C.;
c) Terminating the reaction and isolating the product.

In thirteenth aspect, the present invention provides a method for palladium-catalyzed carbonylation of ethylene to synthesize propionate esters, wherein said method comprising the steps of:
(a) In a reaction vessel, dissolving a palladium catalyst, a phosphine ligand, and an acid in an alcohol and an optional solvent; wherein said phosphine ligand is described as Formula I;
(b) Introducing ethylene into the vessel, followed by carbon monoxide, to initiate the reaction;
(c) Terminating the reaction and isolating the product.
wherein the phosphine ligand has a structure as Formula I,

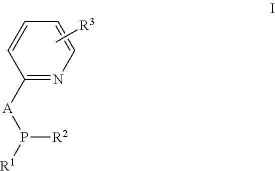

wherein
R¹ is selected from substituted or unsubstituted C₁₋₁₀ alkyl, C₃₋₁₂ cycloalkyl, C₆₋₃₀ aryl;
R² is selected from the substituted or unsubstituted 5-20 membered heteroaryl;
R³ is selected from hydrogen, substituted or unsubstituted C₁₋₁₀ alkyl, substituted or unsubstituted C₁₋₁₀ alkoxy, substituted or unsubstituted C₂₋₁₀ ester group, cyano, COOH, benzenesulfonyl, trialkylsilyl (wherein said alkyl is C₁₋₄ alkyl), nitro, substituted or unsubstituted C₆₋₃₀ aryl, substituted or unsubstituted 5-30 membered heteroaryl; or two R³ groups attached to adjacent carbon atoms together with the carbon atoms to which they are attached form a group selected from the group consisting of: C₆₋₁₀ aryl, 5-12 membered heteroaryl;
A is selected from substituted and unsubstituted C₁₋₄ alkyl,

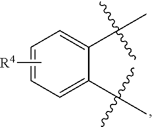

A and

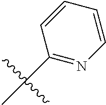 [SMILES: *c1ccccn1]

are linked to form

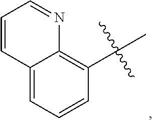

wherein, [symbol] is the attachment site linked to phosphorus;
and
A if it is

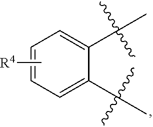

R⁴ is one or more substituents selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, and 5-30 membered heteroaryl; or R⁴ groups together with R³ and adjacent pyridine ring atoms form a fused 5-7 membered carbocyclic or heterocyclic ring;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, unless expressly stated otherwise.

In one embodiment, in the formula I,
A is selected from CH₂,

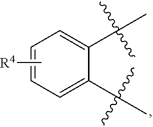

or A together with

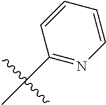 [SMILES: *c1ccccn1]

form

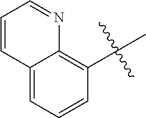 [SMILES: *c1cccc2cccnc12]

[symbol]

s the attachment site linked to phosphorus;
and
R⁴ is one or more substituents selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl.

In one embodiment, in the formula I,
R¹ is selected from substituted or unsubstituted C₁₋₁₀ alkyl, C₃₋₁₀ cycloalkyl;
R² is selected from the substituted or unsubstituted 5-10 membered heteroaryl;
R³ is selected from hydrogen, substituted or unsubstituted C₁₋₁₀ alkyl, C₁₋₁₀ alkoxy, C₂₋₁₀ ester, cyano, tri(C₁₋₄ alkyl)silyl, nitro, —CH(Ph)₂, C₆₋₃₀ aryl, 5-10 membered heteroaryl.

In one embodiment, in the formula I,
R¹ is selected from methyl, ethyl, n-propyl, isopropyl, n-butyl, tert-butyl, isobutyl, cyclopropyl, cyclobutyl, cyclopentyl, cyclohexyl, adamantyl, phenyl.

R² is selected from pyridyl, pyrazinyl, pyrimidinyl, pyridazinyl, pyrrolyl, imidazolyl, pyrazolyl, thienyl, furyl, thiazolyl, triazolyl; may each be substituted by one or more substituents selected from C₁₋₄ alkyl.

R³ is selected from methyl, n-propyl, isopropyl, tert-butyl, methoxy, cyano, COOH, —CH(Ph)₂, benzenesulfonyl, trimethylsilyl.

In one embodiment, the phosphine ligand of formula I is selected from

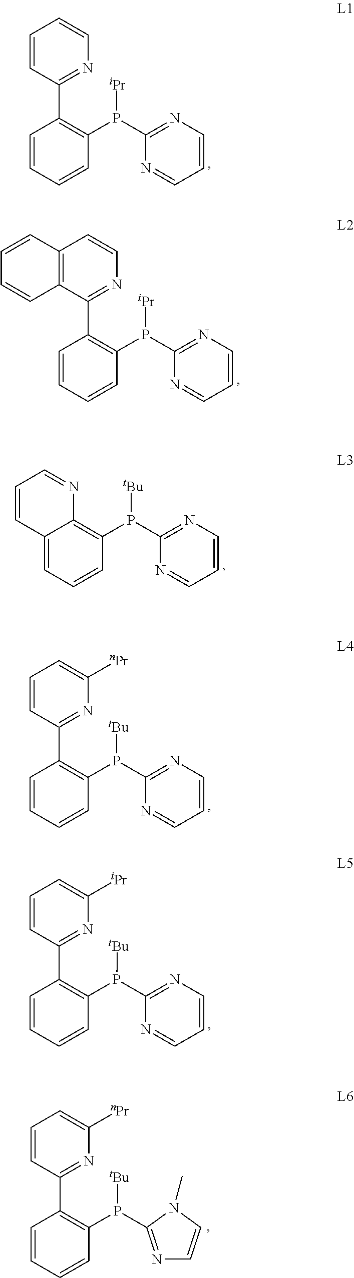

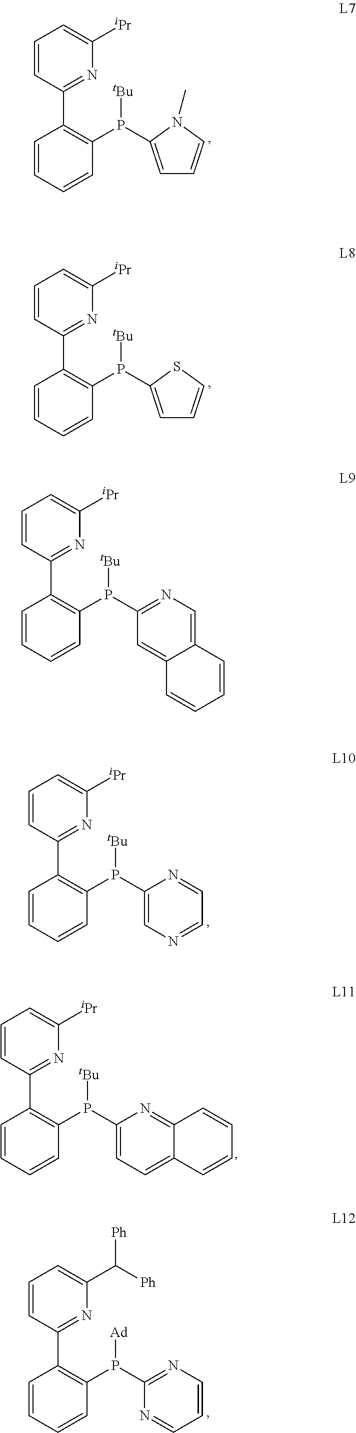

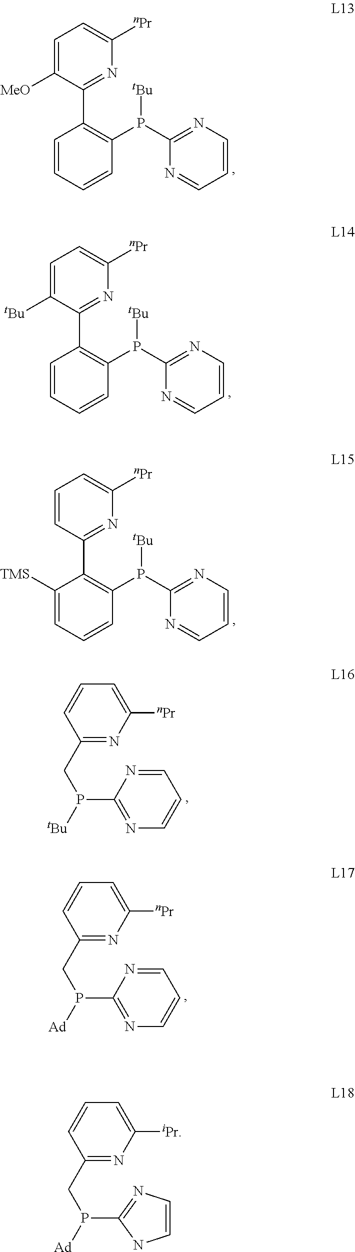

In one embodiment, said reaction vessel is a high-pressure autoclave.

In one embodiment, the amount of said Formula I phosphine ligand is 0.00001-10 mol % relative to ethylene, more preferably 0.0001-1 mol %.

In one embodiment, the reaction temperature is 0-200° C., preferably 60-150° C.

In one embodiment, the reaction is conducted under an inert gas atmosphere; preferably, said inert gas is nitrogen and/or argon.

In one embodiment, the reaction is carried out at 1-10 MPa, preferably 2-8 MPa.

In one embodiment, the reaction time is 0.2-72 hours, preferably 0.2-15 hours.

In one embodiment, the said alcohol is a C₁₋₁₂ alkyl alcohol; preferably, said alcohol is selected from the group consisting of: methanol, ethanol, propanol, butanol, octanol, or a combination thereof.

In one embodiment, the amount of said alcohol is 1-100 molar equivalents relative to ethylene, preferably 1-10 molar equivalents.

In one embodiment, said palladium catalyst is selected from the group consisting of: palladium acetate, palladium trifluoroacetate, palladium pivalate, palladium(II) tetraacetonitrile tetrafluoroborate, palladium(II) hexafluoroacetylacetonate, palladium(II) bis(acetylacetonate), palladium(II) tetraacetonitrile trifluoromethanesulfonate, palladium(II) neodecanoate, bis(dibenzylideneacetone)palladium(0), tris(dibenzylideneacetone)dipalladium(0), palladium(II) chloride, palladium(II) diacetonitrile dichloride, palladium(II) dibenzonitrile dichloride, or a combination thereof.

In one embodiment, said palladium catalyst is selected from the group consisting of: palladium acetate, palladium trifluoroacetate, palladium pivalate, tris(dibenzylideneacetone)dipalladium(0), palladium(II) chloride, palladium(II) diacetonitrile dichloride, or a combination thereof.

In one embodiment, the amount of said palladium catalyst is 0.00001-10 mol % relative to ethylene, more preferably 0.0001-1 mol %.

In the reaction, when said phosphine ligand is a nitrogen-phosphine ligand (i.e., a Formula I compound), the molar ratio of said palladium catalyst to said nitrogen-phosphine ligand is 1:1 to 1:30, more preferably 1:1 to 1:5.

In one embodiment, said acid is selected from the group consisting of: perchloric acid, sulfuric acid, phosphoric acid, sulfonic acid, alkylphosphonic acid, alkylsulfonic acid, alkylcarboxylic acid, perfluoroalkylsulfonic acid, perfluoroalkylcarboxylic acid (wherein said alkyl is a C₁₋₁₂ alkyl), or arylsulfonic acid (wherein said aryl is a C₆₋₁₀ aryl).

In one embodiment, said acid is selected from the group consisting of: methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid, dodecylsulfonic acid, sulfuric acid, sulfonic acid, formic acid, and trifluoroacetic acid.

In one embodiment, the amount of said acid is 0.00004-40 mol % relative to ethylene, preferably 0.0004-4 mol %.

In one embodiment, said solvent is selected from the group consisting of: alkane solvents, substituted aromatic solvents, ether solvents, ketone solvents, nitrile solvents, ester solvents, or a combination thereof.

In one embodiment, said alkane solvent is selected from the group consisting of: n-hexane, cyclohexane, or a combination thereof.

In one embodiment, said substituted aromatic solvent is selected from the group consisting of: chlorobenzene, toluene, xylene, and trifluorotoluene.

In one embodiment, said ether solvent is selected from the group consisting of: tetrahydrofuran, diethyl ether, methyl tert-butyl ether, ethyl tert-butyl ether, anisole, ethylene glycol dimethyl ether, 1,4-dioxane, or a combination thereof.

In one embodiment, said ketone solvent is acetone.

In one embodiment, said nitrile solvent is selected from the group consisting of: acetonitrile, propionitrile, benzonitrile, or a combination thereof.

In one embodiment, said ester solvent is ethyl acetate.

In one embodiment, said alcohol is methanol, and the reaction is carried out under the following conditions:
a) Dissolving palladium acetate, a phosphine ligand of Formula I, and an acid in methanol or a mixed solvent with an optional co-solvent;
b) Introducing ethylene into the reactor, followed by carbon monoxide to initiate the reaction, wherein the reaction proceeds at a temperature ranging from room temperature to 60-100° C.;
c) Terminating the reaction and isolating the product.

In one embodiment, said alcohol is ethanol, and the reaction is carried out under the following conditions:
a) Dissolving palladium acetate, a phosphine ligand of Formula I, and an acid in ethanol or a mixed solvent with an optional co-solvent;
b) Introducing ethylene into the reactor, followed by carbon monoxide to initiate the reaction, wherein the reaction proceeds at a temperature ranging from room temperature to 60-100° C.;
c) Terminating the reaction and isolating the product.

In one embodiment, said alcohol is butanol, and the reaction is carried out under the following conditions:
a) Dissolving palladium acetate, a phosphine ligand of Formula I, and an acid in butanol or a mixed solvent with an optional co-solvent;
b) Introducing ethylene into the reactor, followed by carbon monoxide to initiate the reaction, wherein the reaction proceeds at a temperature ranging from room temperature to 60-100° C.;
c) Terminating the reaction and isolating the product.

In one embodiment, said alcohol is octanol, and the reaction is carried out under the following conditions:
a) Dissolving palladium acetate, a phosphine ligand of Formula I, and an acid in octanol or a mixed solvent with an optional co-solvent;
b) Introducing ethylene into the reactor, followed by carbon monoxide to initiate the reaction, wherein the reaction proceeds at a temperature ranging from room temperature to 60-100° C.;
c) Terminating the reaction and isolating the product.

In fourteenth aspect, the present invention provides a method for the catalytic preparation of propionate esters from ethylene, wherein said method comprising the steps of:
(a) In a reaction vessel, dissolving a palladium catalyst, a phosphine ligand, and an acid in an alcohol and an optional solvent;
(b) Introducing ethylene into the vessel, followed by carbon monoxide, to initiate the reaction;
(c) Terminating the reaction and isolating the product.
wherein the phosphine ligand has a structure as Formula V

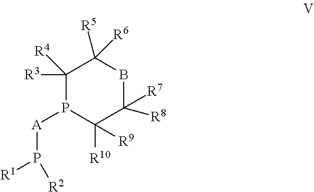

R¹ is selected from substituted or unsubstituted C₁₋₁₂ alkyl, C₃₋₁₀ cycloalkyl, C₆₋₃₀ aryl;
R² is selected from the substituted or unsubstituted 5-20 membered heteroaryl;
and
R¹ is selected from substituted or unsubstituted C₁₋₁₂ alkyl, C₃₋₁₀ cycloalkyl, C₆₋₃₀ aryl;
R² is selected from the substituted or unsubstituted C₆₋₃₀ aryl;
R³, R⁴, R⁵, R⁶, R⁷, R⁸, R⁹, R¹⁰ are independently selected from hydrogen, hydroxyl, halogen, C₁₋₁₂ alkyl, C₃₋₁₂ cycloalkyl, C₃₋₁₀ heterocycloalkyl, C₂₋₁₀ alkenyl, C₆₋₃₀ aryl, 5-20 membered heteroaryl, C₁₋₁₀ alkoxyl, C₁₋₁₀ alkylamino, C₁₋₁₀ alkylamino, C₁₋₁₀ alkylthio; two adjacent R³, R⁴, R⁵, R⁶, R⁷, R⁸, R⁹, R¹⁰ located on rings, together with the ring atoms to which they are attached, form a 4-8 membered carbocycle or heterocycle;
A if it is -L¹-A¹-L²-, may each independently be selected from chemical bonds, O, NH, S, substituted or unsubstituted C₁₋₁₂ alkyl, C₃₋₈ cycloalkyl, C₃₋₈ heterocycloalkyl, C₆₋₃₀ aryl, 5-20 membered heteroaryl, biphenyl,

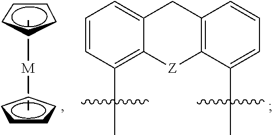

with the proviso that L¹, A¹ and L² are not all a chemical bond at the same time;
and
if the said aryl and heteroaryl are substituted by two or more substituents, any two of said substituents may, together with the ring atoms to which they are attached, form a 4-8 membered carbocycle or heterocycle;
M is selected from Fe, Co, Ru;
B is selected from substituted or unsubstituted methylene, ethylidene, ═CHR, ═NR, hydroxymethyl, carbonyl, thiocarbonyl,

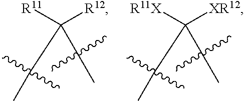

—C(O)—C(O)—; where R is selected from substituted or unsubstituted C₁₋₁₀ alkyl, C₆₋₃₀ aryl, and 5-20 membered heteroaryl;
R¹¹, R¹² are independently selected from hydrogen, hydroxyl, substituted or unsubstituted C₁₋₁₀ alkyl,

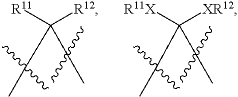

may selected from substituted and unsubstituted 5-8 membered heterocycles;
X is selected from O, S;
Z is selected from O, S, NH;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, benzyl, —CH(Ph)₂ unless expressly stated otherwise.

In one embodiment, in the formula V,
R¹ is selected from substituted or unsubstituted C₁₋₁₂ alkyl, C₃₋₁₀ cycloalkyl, C₆₋₂₀ aryl;
R² is selected from the substituted or unsubstituted 5-12 membered heteroaryl;
and
R¹ is selected from substituted or unsubstituted C₁₋₁₂ alkyl, C₃₋₁₀ cycloalkyl;
R² is selected from the substituted or unsubstituted C₆₋₂₀ aryl.

In one embodiment, in the formula V,
R³, R⁴, R⁵, R⁶, R⁷, R⁸, R⁹, R¹⁰ are independently selected from hydrogen, C₁₋₁₂ alkyl, C₃₋₁₂ cycloalkyl, C₃₋₁₀ heterocycloalkyl, C₂₋₁₀ alkenyl, C₁₋₁₀ alkoxyl, C₁₋₁₀ alkylamino, C₁₋₁₀ alkylamino, C₁₋₁₀ alkylthio; two adjacent R³, R¹⁰ located on rings, together with the ring atoms to which they are attached, form a 4-8 membered carbocycle or heterocycle;
B is selected from substituted or unsubstituted methylene, ethylidene, ═NR, hydroxymethyl, carbonyl,

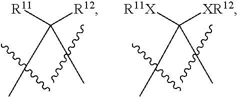

—C(O)—C(O)—; where R is selected from substituted or unsubstituted C₁₋₁₀ alkyl, phenyl;
R¹¹, R¹² are independently selected from hydrogen, hydroxyl, substituted or unsubstituted C₁₋₆ alkyl,

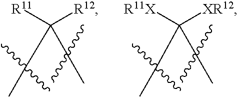

may selected from substituted and unsubstituted 5-8 membered heterocycles, and said heterocycle may be unsubstituted or substituted by C₁₋₆ alkyl.

X is selected from O, S.

In one embodiment,
L¹ is selected from chemical bonds, substituted or unsubstituted C₁₋₆ alkyl;
A¹, L² are independently selected from substituted or unsubstituted C₁₋₆ alkyl, C₃₋₈ cycloalkyl, C₃₋₈ heterocycloalkyl, C₆₋₁₀ aryl, 5-10 membered heteroaryl, biphenyl,

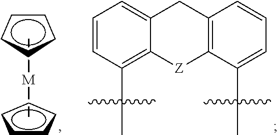

and
A¹, L² are independently be selected from chemical bonds, O, NH, S, substituted or unsubstituted C₁₋₁₂ alkyl, C₃₋₈ cycloalkyl, C₃₋₈ heterocycloalkyl, C₆₋₃₀ aryl, 5-20 membered heteroaryl;
and
A¹, if it is selected from O, NH, S, L¹ and L² each may independently be selected from substituted or unsubstituted C₆₋₁₀ aryl, 5-20 membered heteroaryl;
and
A¹, if it is O, L¹ and L² each may independently be selected from substituted or unsubstituted C₆₋₁₀ aryl, 5-10 membered heteroaryl;

In one embodiment, in the formula V, A is selected from

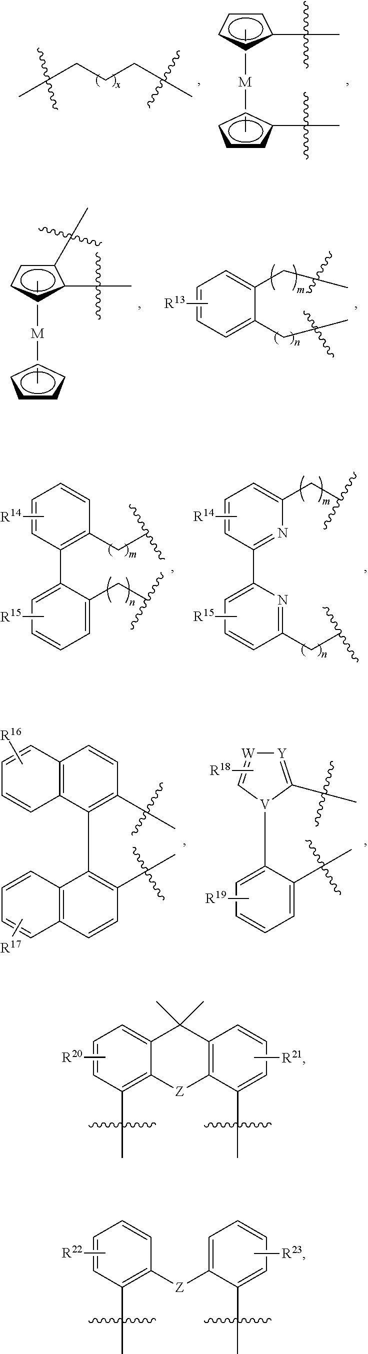

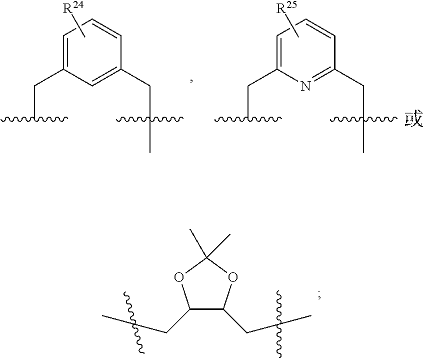

where

R¹³, R¹⁴, R¹⁵, R¹⁶, R¹⁷, R¹⁸, R¹⁹, R²⁰, R²¹, R²², R²³, R²⁴, R²⁵ are independently selected from C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, and 5-30 membered heteroaryl; or two adjacent R¹³, R¹⁴, R¹⁵, R¹⁶, R¹⁷, R¹⁸, R¹⁹, R²⁰, R²¹, R²², R²³ located on rings, together with the ring atoms to which they are attached, form a 5-7 membered carbocycle or heterocycle;
x is selected from 0, 1, 2, 3, 4, 5;
m, n are independently selected from 0, 1, 2, 3;
M is selected from Fe, Co, Ru;
V, W, Y are independently selected from N, CH; wherein any hydrogen atom on said CH may be substituted by R¹⁸;

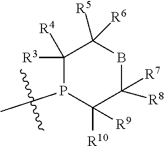

is selected from

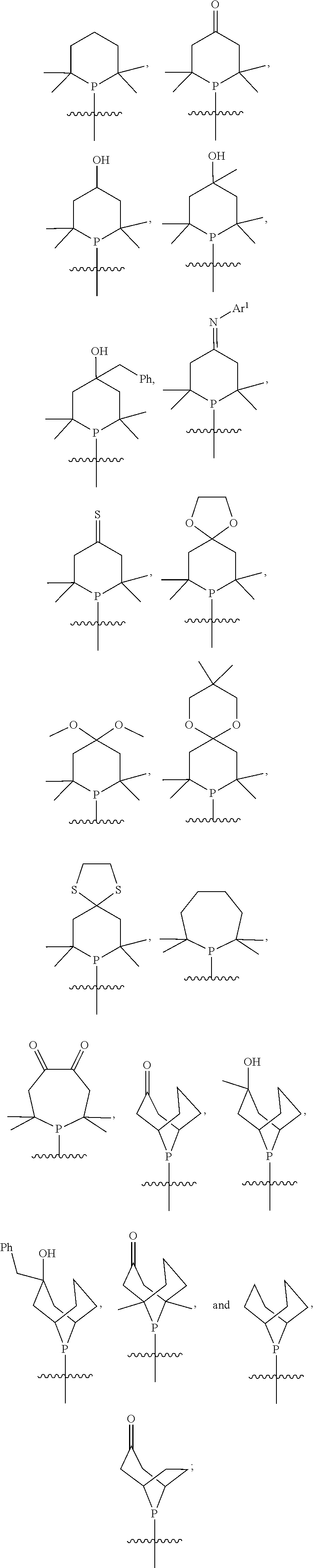

Ar¹ is selected from substituted or unsubstituted C₆₋₃₀ aryl, 5-20 membered heteroaryl.

In one embodiment, the phosphine ligand of formula V is selected from

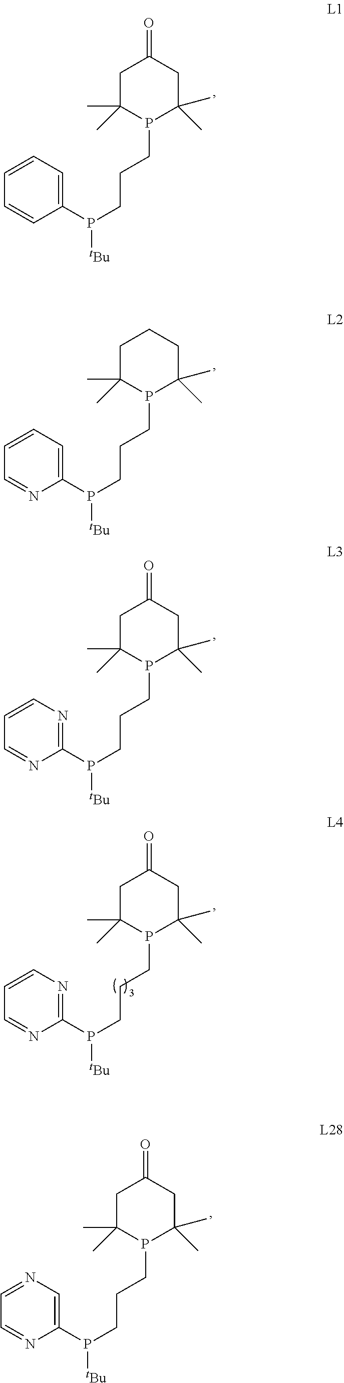

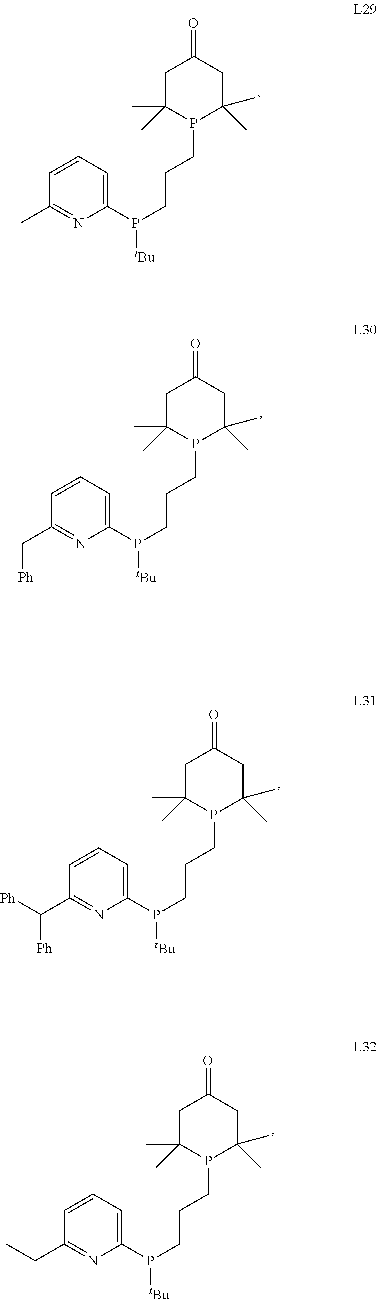

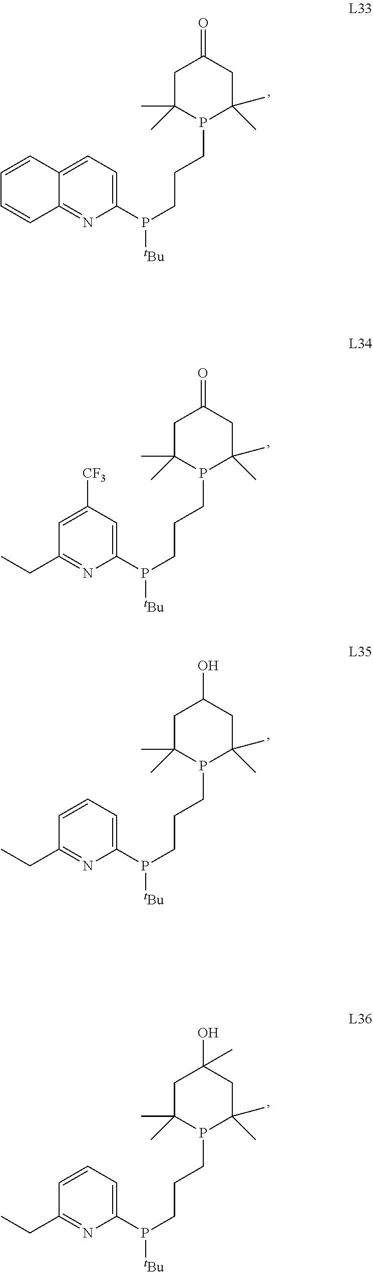

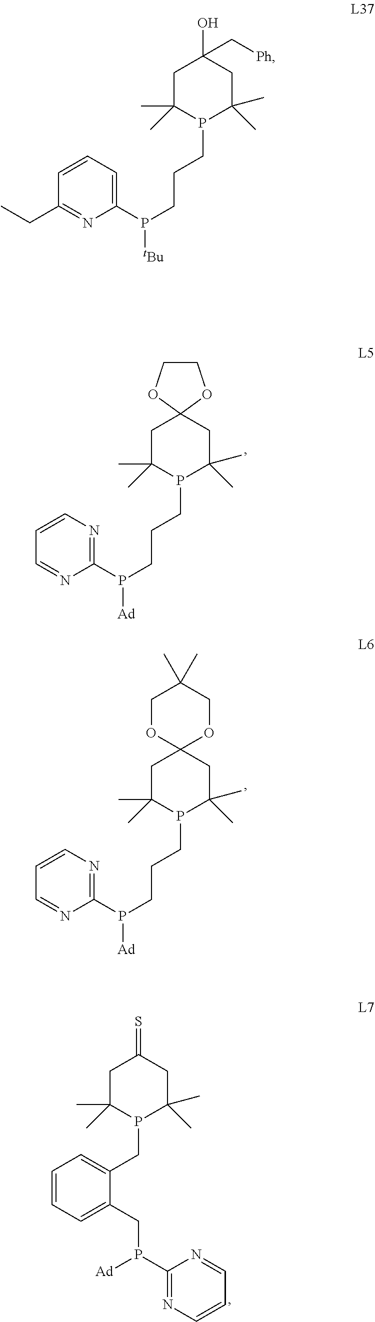

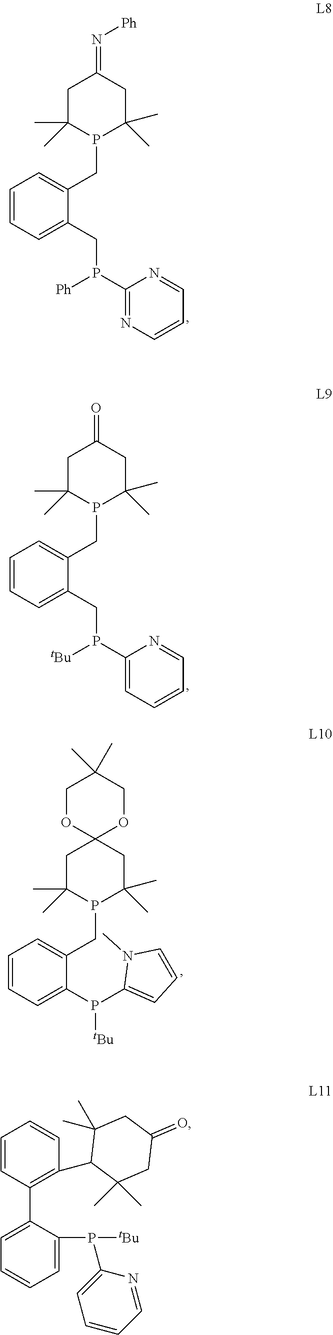 [SMILES: CC1(C)CC(=Nc2ccccc2)CC(C)(C)P1Cc1ccccc1CP(c1ccccc1)c1ncccn1 ; CC(C)(C)P(Cc1ccccc1CP1C(C)(C)CC(=O)CC1(C)C)c1ccccn1 ; Cn1cccc1P(c1ccccc1CP1C(C)(C)CC2(CC1(C)C)OCC(C)(C)CO2)C(C)(C)C ; CC1(C)CC(=O)CC(C)(C)C1c1ccccc1-c1ccccc1P(c1ccccn1)C(C)(C)C]

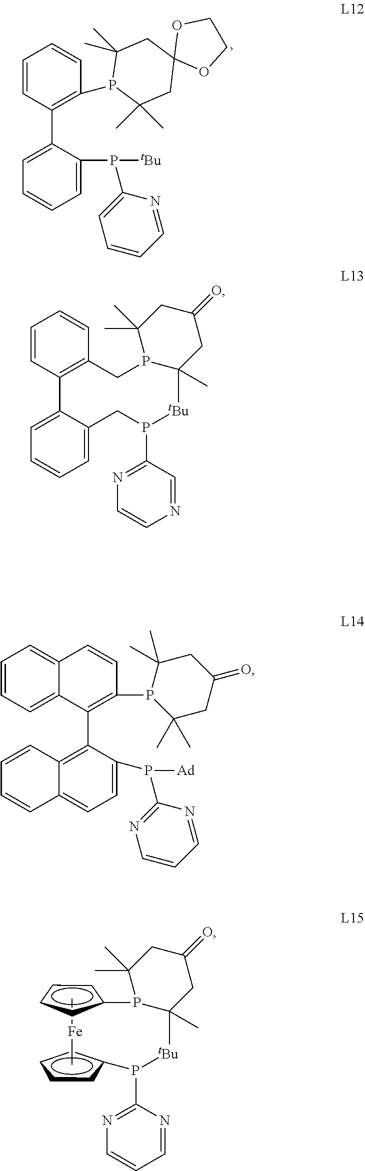

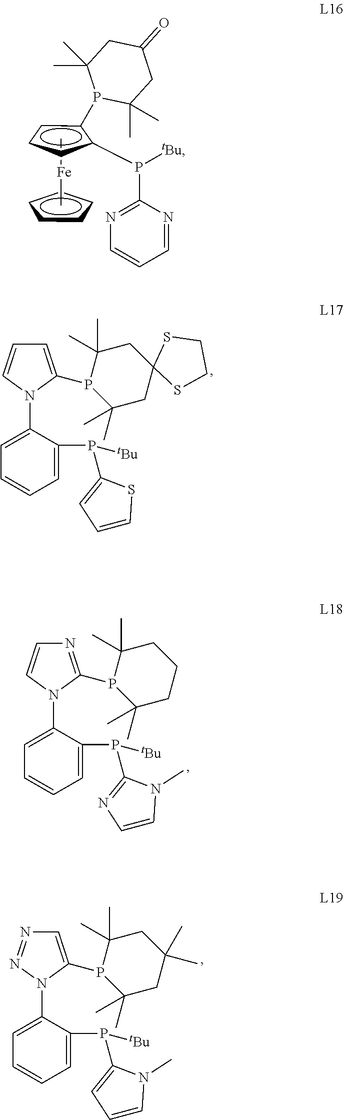

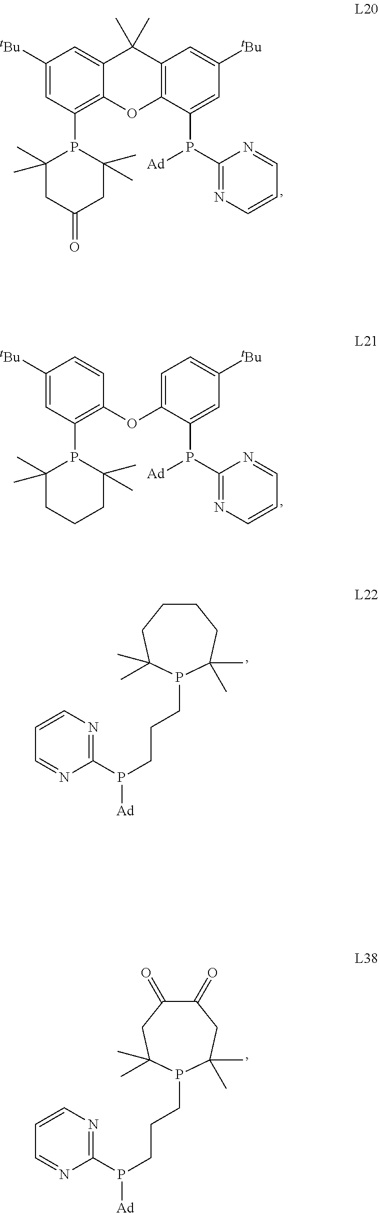

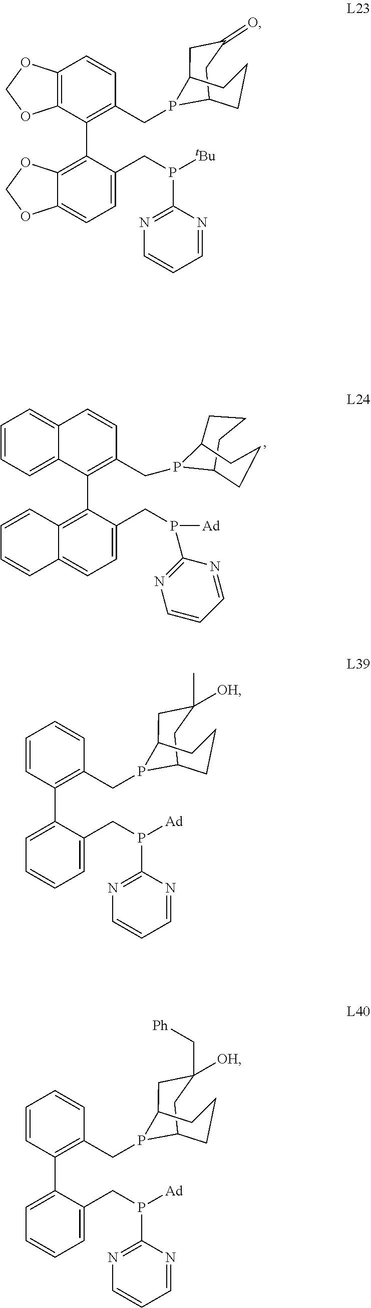

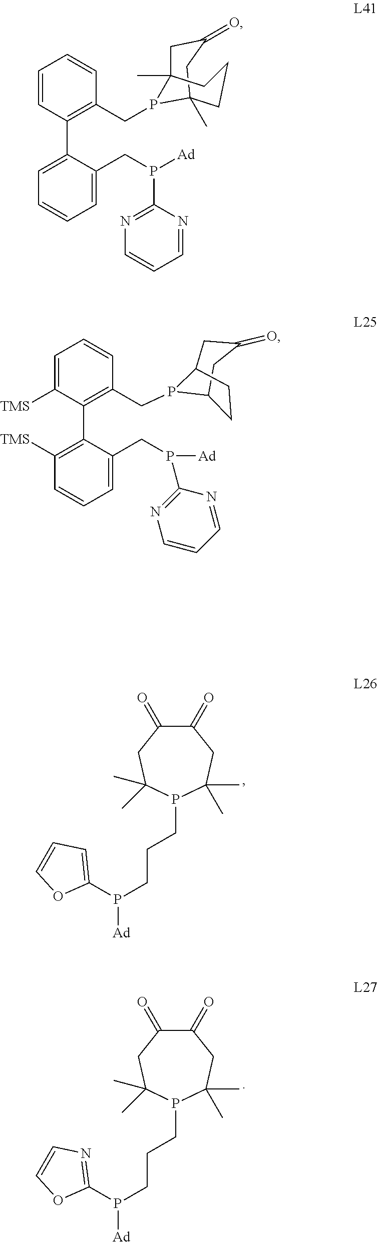

In one embodiment, said reaction vessel is a high-pressure autoclave.

In one embodiment, the amount of said Formula V phosphine ligand is 0.00001-10 mol % relative to ethylene, more preferably 0.0001-1 mol %.

In one embodiment, the reaction temperature is 0-200° C., preferably 40-150° C.

In one embodiment, the reaction is conducted under an inert gas atmosphere; preferably, said inert gas is nitrogen and/or argon.

In one embodiment, the reaction is carried out at 1-10 MPa, preferably 2-8 MPa.

In one embodiment, the reaction time is 0.2-72 hours, preferably 0.2-24 hours.

In one embodiment, the said alcohol is a C₁₋₁₂ alkyl alcohol; preferably, said alcohol is selected from the group consisting of: methanol, ethanol, propanol, butanol, octanol, or a combination thereof.

In one embodiment, the amount of said alcohol is 1-100 molar equivalents relative to ethylene, preferably 1-10 molar equivalents.

In one embodiment, said palladium catalyst is selected from the group consisting of: palladium acetate, palladium trifluoroacetate, palladium pivalate, palladium(II) tetraacetonitrile tetrafluoroborate, palladium(II) hexafluoroacetylacetonate, palladium(II) bis(acetylacetonate), palladium(II) tetraacetonitrile trifluoromethanesulfonate, palladium(II) neodecanoate, bis(dibenzylideneacetone)palladium(0), tris(dibenzylideneacetone)dipalladium(0), palladium(II) chloride, palladium(II) diacetonitrile dichloride, palladium(II) dibenzonitrile dichloride, or a combination thereof.

In one embodiment, said palladium catalyst is selected from the group consisting of: palladium acetate, palladium trifluoroacetate, palladium pivalate, tris(dibenzylideneacetone)dipalladium(0), palladium(II) chloride, palladium(II) diacetonitrile dichloride, or a combination thereof.

In one embodiment, the amount of said palladium catalyst is 0.00001-10 mol % relative to ethylene, more preferably 0.0001-1 mol %.

In the reaction, when said phosphine ligand is a nitrogen-phosphine ligand (i.e., a Formula I compound), the molar ratio of said palladium catalyst to said nitrogen-phosphine ligand is 1:1 to 1:30, more preferably 1:1 to 1:5.

In one embodiment, said acid is selected from the group consisting of: perchloric acid, sulfuric acid, phosphoric acid, sulfonic acid, alkylphosphonic acid, alkylsulfonic acid, alkylcarboxylic acid, perfluoroalkylsulfonic acid, perfluoroalkylcarboxylic acid (wherein said alkyl is a C₁₋₁₂ alkyl), or arylsulfonic acid (wherein said aryl is a C₆₋₁₀ aryl).

In one embodiment, said acid is selected from the group consisting of: methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid, dodecylsulfonic acid, sulfuric acid, sulfonic acid, formic acid, and trifluoroacetic acid.

In one embodiment, the amount of said acid is 0.00004-40 mol % relative to ethylene, preferably 0.0004-4 mol %.

In one embodiment, said solvent is selected from the group consisting of: alkane solvents, substituted aromatic solvents, ether solvents, ketone solvents, nitrile solvents, ester solvents, or a combination thereof.

In one embodiment, said alkane solvent is selected from the group consisting of: n-hexane, cyclohexane, or a combination thereof.

In one embodiment, said substituted aromatic solvent is selected from the group consisting of: chlorobenzene, toluene, xylene, and trifluorotoluene.

In one embodiment, said ether solvent is selected from the group consisting of: tetrahydrofuran, diethyl ether, methyl tert-butyl ether, ethyl tert-butyl ether, anisole, ethylene glycol dimethyl ether, 1,4-dioxane, or a combination thereof.

In one embodiment, said ketone solvent is acetone.

In one embodiment, said nitrile solvent is selected from the group consisting of: acetonitrile, propionitrile, benzonitrile, or a combination thereof.

In one embodiment, said ester solvent is ethyl acetate.

In one embodiment, said alcohol is methanol, and the reaction is carried out under the following conditions:
a) Dissolving palladium acetate, a phosphine ligand of Formula V, and an acid in methanol or a mixed solvent with an optional co-solvent;
b) Introducing ethylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is ethanol, and the reaction is carried out under the following conditions:
a) Dissolving palladium acetate, a phosphine ligand of Formula V, and an acid in ethanol or a mixed solvent with an optional co-solvent;
b) Introducing ethylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is butanol, and the reaction is carried out under the following conditions:
a) Dissolving palladium acetate, a phosphine ligand of Formula V, and an acid in butanol or a mixed solvent with an optional co-solvent;
b) Introducing ethylene into the reactor, followed by carbon monoxide to initiate the reaction, wherein the reaction proceeds at a temperature ranging from room temperature to 120° C.;
c) Terminating the reaction and isolating the product.

In one embodiment, said alcohol is octanol, and the reaction is carried out under the following conditions:
a) Dissolving palladium acetate, a phosphine ligand of Formula V, and an acid in octanol or a mixed solvent with an optional co-solvent;
b) Introducing ethylene into the reactor, followed by carbon monoxide to initiate the reaction, wherein the reaction proceeds at a temperature ranging from room temperature to 120° C.;
c) Terminating the reaction and isolating the product.

In fifth aspect, the present invention provides a method for the catalytic preparation of propionate esters, such as methyl propionate, ethyl propionate, butyl propionate, or octyl propionate, wherein said method comprising the steps of:
(a) In a reaction vessel, dissolving a palladium catalyst, a phosphine ligand, and an acid in an alcohol and an optional solvent;
(b) Introducing ethylene into the vessel, followed by carbon monoxide, to initiate the reaction;
(c) Terminating the reaction and isolating the product.
wherein the phosphine ligand has a structure as Formula VI

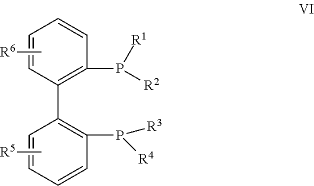

wherein
R¹, R⁴ are independently selected from substituted or unsubstituted C₁₋₁₀ alkyl, C₃₋₁₀ cycloalkyl, C₆₋₃₀ aryl;
R² and R³ are independently selected from 5-20 membered heteroaryl;
R⁵, R⁶ are independently selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, and 5-30 membered heteroaryl; two adjacent R⁵, R⁶ located on rings, together with the ring atoms to which they are attached, form a 5-7 membered carbocycle or heterocycle; wherein the heterocycles contain 1-3 heteroatoms selected from N, O, S;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, unless expressly stated otherwise.

In one embodiment, in the formula VI,
R¹, R⁴ are independently selected from substituted or unsubstituted C₁₋₄ alkyl, C₃₋₆ cycloalkyl, C₆₋₁₄ aryl;
R², R³ are independently selected from 5-20 membered heteroaryl;
and
R¹, R², R³, R⁴ are independently unsubstituted or substituted by one or more substituents selected from methyl, methoxyl, cyano, COOH, benzenesulfonyl, trimethylsilyl.

In one embodiment, in the formula VI,
R¹, R⁴ are independently selected from methyl, ethyl, n-propyl, isopropyl, n-butyl, tert-butyl, isobutyl, cyclopropyl, cyclobutyl, cyclopentyl, cyclohexyl, phenyl, adamantyl.
R², R³ are independently selected from pyridyl, pyrazinyl, pyrimidinyl, pyridazinyl, pyrrolyl, imidazolyl, pyrazolyl, thienyl, furyl, thiazolyl, triazolyl.

In one embodiment, the phosphine ligand of formula VI is selected from

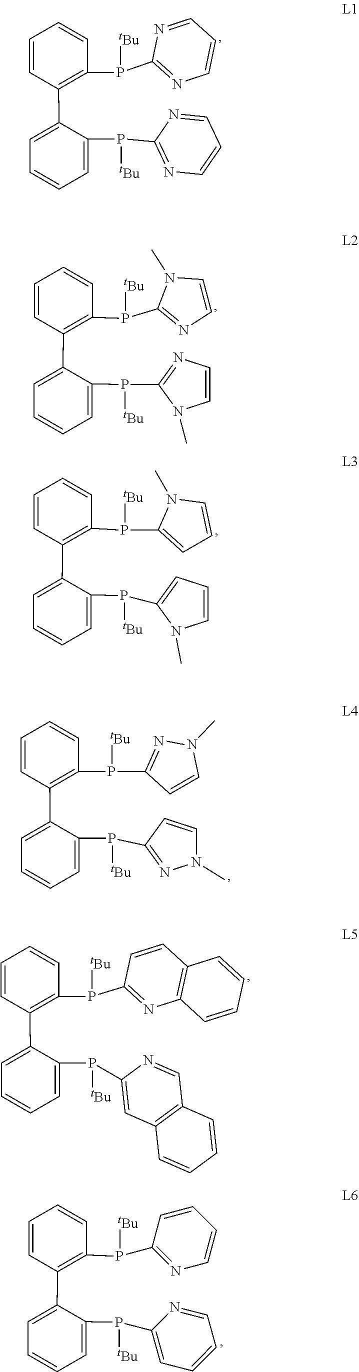

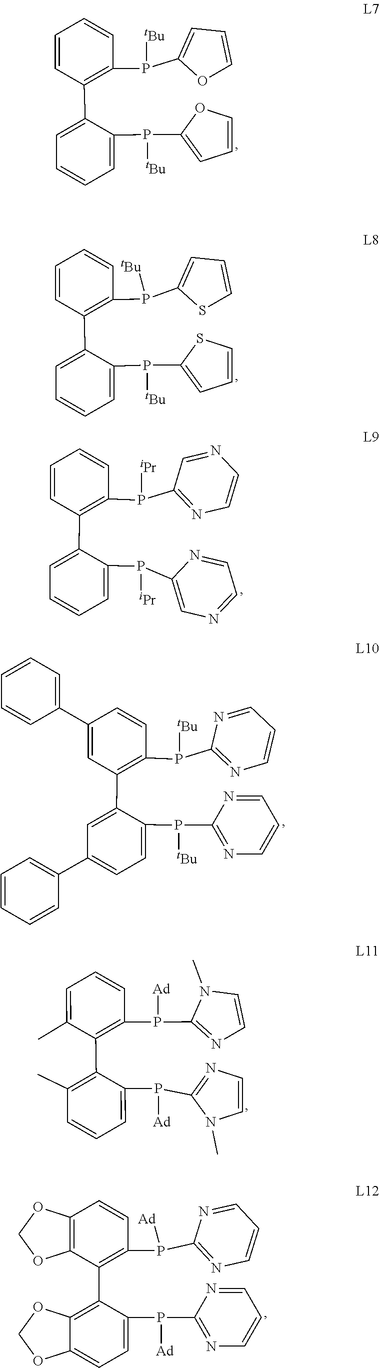

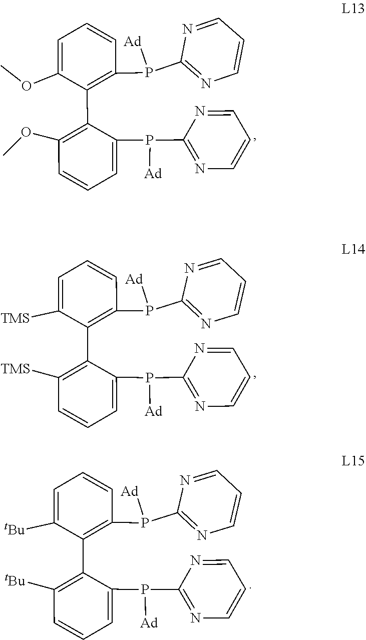

In one embodiment, said reaction vessel is a high-pressure autoclave.

In one embodiment, the amount of said Formula VI phosphine ligand is 0.00001-10 mol % relative to ethylene, more preferably 0.0001-1 mol %.

In one embodiment, the reaction temperature is 0-200° C., preferably 60-150° C.

In one embodiment, the reaction is conducted under an inert gas atmosphere; preferably, said inert gas is nitrogen and/or argon.

In one embodiment, the reaction is carried out at 1-10 MPa, preferably 2-8 MPa.

In one embodiment, the reaction time is 0.2-72 hours, preferably 0.2-24 hours.

In one embodiment, the said alcohol is a C₁₋₁₂ alkyl alcohol; preferably, said alcohol is selected from the group consisting of: methanol, ethanol, propanol, butanol, octanol, or a combination thereof.

In one embodiment, the amount of said alcohol is 1-100 molar equivalents relative to ethylene, preferably 1-10 molar equivalents.

In one embodiment, said palladium catalyst is selected from the group consisting of: palladium acetate, palladium trifluoroacetate, palladium pivalate, palladium(II) tetraacetonitrile tetrafluoroborate, palladium(II) hexafluoroacetylacetonate, palladium(II) bis(acetylacetonate), palladium(II) tetraacetonitrile trifluoromethanesulfonate, palladium(II) neodecanoate, bis(dibenzylideneacetone)palladium(0), tris(dibenzylideneacetone)dipalladium(0), palladium(II) chloride, palladium(II) diacetonitrile dichloride, palladium(II) dibenzonitrile dichloride, or a combination thereof.

In one embodiment, said palladium catalyst is selected from the group consisting of: palladium acetate, palladium trifluoroacetate, palladium pivalate, tris(dibenzylideneacetone)dipalladium(0), palladium(II) chloride, palladium(II) diacetonitrile dichloride, or a combination thereof.

In one embodiment, the amount of said palladium catalyst is 0.00001-10 mol % relative to ethylene, more preferably 0.0001-1 mol %.

In the reaction, when said phosphine ligand is a nitrogen-phosphine ligand (i.e., a Formula I compound), the molar ratio of said palladium catalyst to said nitrogen-phosphine ligand is 1:1 to 1:30, more preferably 1:1 to 1:5.

In one embodiment, said acid is selected from the group consisting of: perchloric acid, sulfuric acid, phosphoric acid, sulfonic acid, alkylphosphonic acid, alkylsulfonic acid, alkylcarboxylic acid, perfluoroalkylsulfonic acid, perfluoroalkylcarboxylic acid (wherein said alkyl is a C₁₋₁₂ alkyl), or arylsulfonic acid (wherein said aryl is a C₆₋₁₀ aryl).

In one embodiment, said acid is selected from the group consisting of: methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid, dodecylsulfonic acid, sulfuric acid, sulfonic acid, formic acid, and trifluoroacetic acid.

In one embodiment, the amount of said acid is 0.00004-40 mol % relative to ethylene, preferably 0.0004-4 mol %.

In one embodiment, said solvent is selected from the group consisting of: alkane solvents, substituted aromatic solvents, ether solvents, ketone solvents, nitrile solvents, ester solvents, or a combination thereof.

In one embodiment, said alkane solvent is selected from the group consisting of: n-hexane, cyclohexane, or a combination thereof.

In one embodiment, said substituted aromatic solvent is selected from the group consisting of: chlorobenzene, toluene, xylene, and trifluorotoluene.

In one embodiment, said ether solvent is selected from the group consisting of: tetrahydrofuran, diethyl ether, methyl tert-butyl ether, ethyl tert-butyl ether, anisole, ethylene glycol dimethyl ether, 1,4-dioxane, or a combination thereof.

In one embodiment, said ketone solvent is acetone.

In one embodiment, said nitrile solvent is selected from the group consisting of: acetonitrile, propionitrile, benzonitrile, or a combination thereof.

In one embodiment, said ester solvent is ethyl acetate.

In one embodiment, said alcohol is methanol, and the reaction is carried out under the following conditions:
a) Dissolving palladium acetate, a phosphine ligand of Formula VI, and an acid in methanol or a mixed solvent with an optional co-solvent;
b) Introducing ethylene into the reactor, followed by carbon monoxide to initiate the reaction, wherein the reaction proceeds at a temperature ranging from room temperature to 60-100° C.;
c) Terminating the reaction and isolating the product.

In one embodiment, said alcohol is ethanol, and the reaction is carried out under the following conditions:
a) Dissolving palladium acetate, a phosphine ligand of Formula VI, and an acid in ethanol or a mixed solvent with an optional co-solvent;
b) Introducing ethylene into the reactor, followed by carbon monoxide to initiate the reaction, wherein the reaction proceeds at a temperature ranging from room temperature to 60-100° C.;
c) Terminating the reaction and isolating the product.

In one embodiment, said alcohol is butanol, and the reaction is carried out under the following conditions:
a) Dissolving palladium acetate, a phosphine ligand of Formula VI, and an acid in butanol or a mixed solvent with an optional co-solvent;
b) Introducing ethylene into the reactor, followed by carbon monoxide to initiate the reaction, wherein the reaction proceeds at a temperature ranging from room temperature to 60-100° C.;
c) Terminating the reaction and isolating the product.

In one embodiment, said alcohol is octanol, and the reaction is carried out under the following conditions:
a) Dissolving palladium acetate, a phosphine ligand of Formula VI, and an acid in octanol or a mixed solvent with an optional co-solvent;
b) Introducing ethylene into the reactor, followed by carbon monoxide to initiate the reaction, wherein the reaction proceeds at a temperature ranging from room temperature to 60-100° C.;
c) Terminating the reaction and isolating the product.

In sixteenth aspect, the present invention provides a method for the catalytic preparation of propionate esters, such as methyl propionate, ethyl propionate, butyl propionate, or octyl propionate, wherein said method comprising the steps of:
(a) In a reaction vessel, dissolving a palladium catalyst, a phosphine ligand, and an acid in an alcohol and an optional solvent;
(b) Introducing ethylene into the vessel, followed by carbon monoxide, to initiate the reaction;
(c) Terminating the reaction and isolating the product.
wherein the phosphine ligand has a structure as Formula VIII

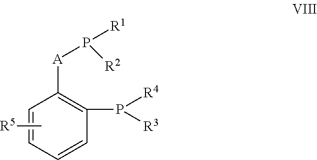

wherein
R¹, R⁴ are independently selected from substituted or unsubstituted C₁₋₁₀ alkyl, C₃₋₁₀ cycloalkyl, C₆₋₃₀ aryl;
R² and R³ are independently selected from 5-20 membered heteroaryl;
A is selected from 5-10 membered heteroaryl, may independently by one or more R⁶ substituents, where one or more heteroatoms are independently selected from O, N, S;
R⁵, R⁶ are independently selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, and 5-30 membered heteroaryl; two adjacent R⁵ located on rings, together with the ring atoms to which they are attached, form a 5-7 membered carbocycle or heterocycle;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, unless expressly stated otherwise.
with the proviso that phosphine ligand is not

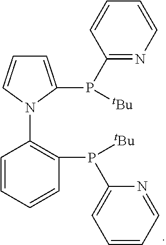 [SMILES: CC(C)(C)P(c1ccccn1)c1ccccc1-n1cccc1P(c1ccccn1)C(C)(C)C]

In one embodiment, when A is pyrrole, the compound satisfies any one to three of the following conditions (1)-(3):
(1) R⁵, R⁶ are not simultaneously hydrogen;
(2) R¹, R⁴ are not simultaneously tert-butyl;
(3) R², R³ are not simultaneously 2-pyridyl.

In one embodiment, in the formula VIII,
R¹, R⁴ are independently selected from substituted or unsubstituted C₁₋₄ alkyl, C₃₋₁₀ cycloalkyl, phenyl;
R² and R³ are independently selected from 5-20 membered heteroaryl;
and
R¹, R², R³, R⁴ are independently unsubstituted or substituted by one or more substituents selected from methyl, methoxyl, cyano, COOH, benzenesulfonyl, trimethylsilyl.

In one embodiment, in the formula VIII, A is a 5-9 membered heteroaryl group, unsubstituted or substituted with one or more R⁶, wherein the heteroatoms in the heteroaryl group are selected from O, N, and S, and may be the same or different.

In one embodiment, in the formula VIII,
R¹, R⁴ are independently selected from methyl, ethyl, n-propyl, isopropyl, n-butyl, tert-butyl, isobutyl, cyclopropyl, cyclobutyl, cyclopentyl, cyclohexyl, adamantyl, phenyl;
R², R³ are independently selected from pyridyl, pyrazinyl, pyrimidinyl, pyridazinyl, pyrrolyl, imidazolyl, pyrazolyl, thienyl, furyl, thiazolyl, triazolyl.
and
R¹, R², R³, R⁴ are independently unsubstituted or substituted by one or more substituents selected from methyl, methoxyl, cyano, COOH, benzenesulfonyl, trimethylsilyl.

In one embodiment, the phosphine ligand of formula VIII is selected from

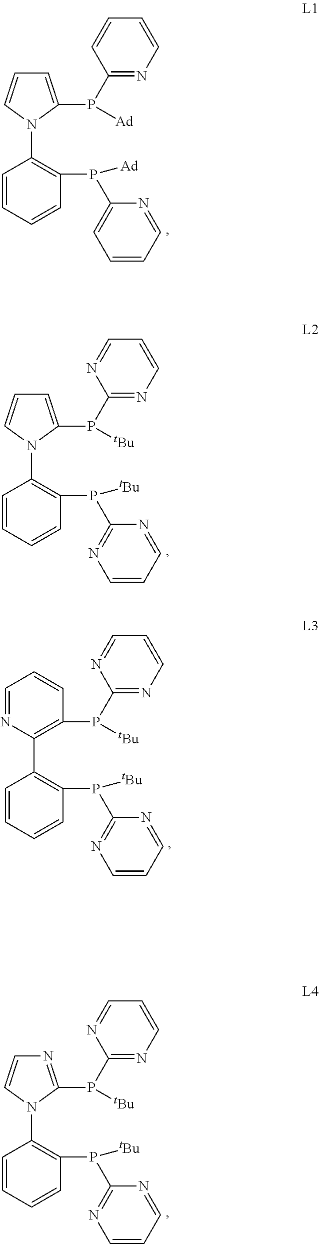

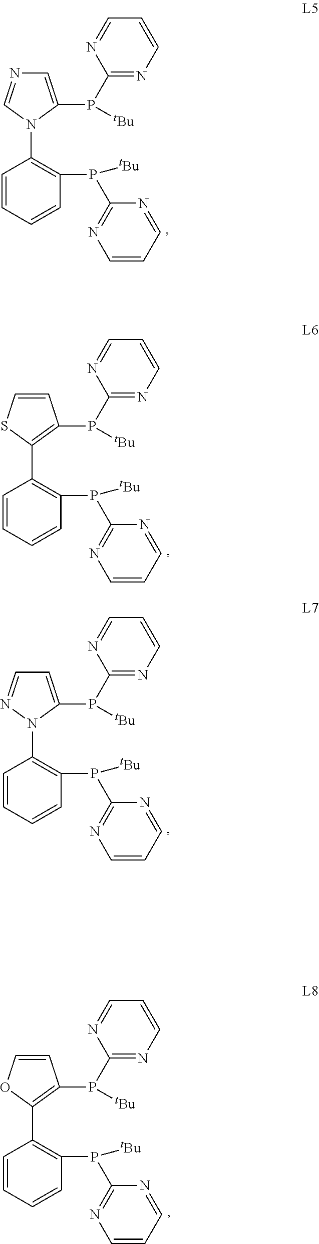

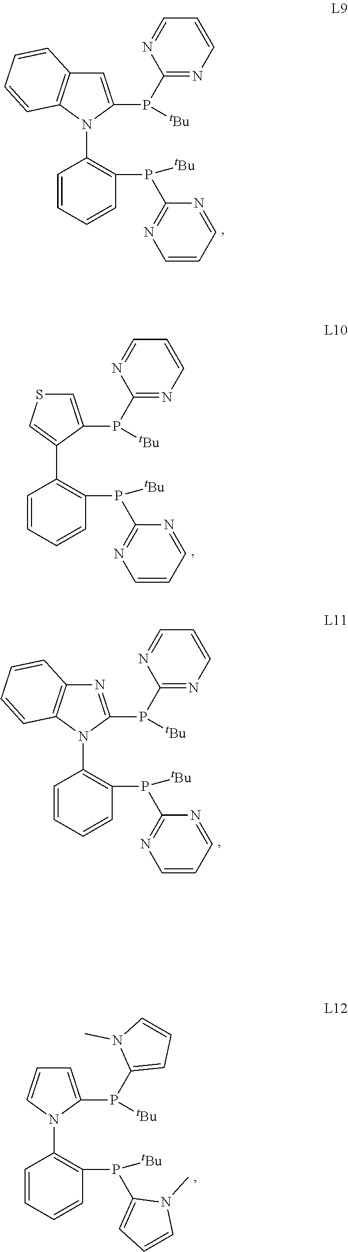 [SMILES: CC(C)(C)P(c1ncccn1)c1ccccc1-n1c(P(c2ncccn2)C(C)(C)C)cc2ccccc21 ; CC(C)(C)P(c1ncccn1)c1ccccc1-c1cscc1P(c1ncccn1)C(C)(C)C ; CC(C)(C)P(c1ncccn1)c1ccccc1-n1c(P(c2ncccn2)C(C)(C)C)nc2ccccc21 ; Cn1cccc1P(c1ccccc1-n1cccc1P(c1cccn1C)C(C)(C)C)C(C)(C)C]

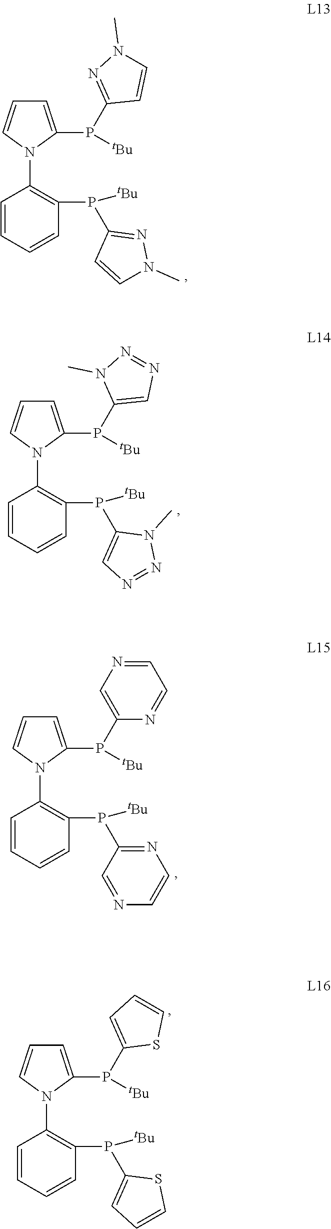 [SMILES: Cn1ccc(P(c2ccccc2-n2cccc2P(c2ccn(C)n2)C(C)(C)C)C(C)(C)C)n1 ; Cn1nncc1P(c1ccccc1-n1cccc1P(c1cnnn1C)C(C)(C)C)C(C)(C)C ; CC(C)(C)P(c1cnccn1)c1ccccc1-n1cccc1P(c1cnccn1)C(C)(C)C ; CC(C)(C)P(c1cccs1)c1ccccc1-n1cccc1P(c1cccs1)C(C)(C)C]

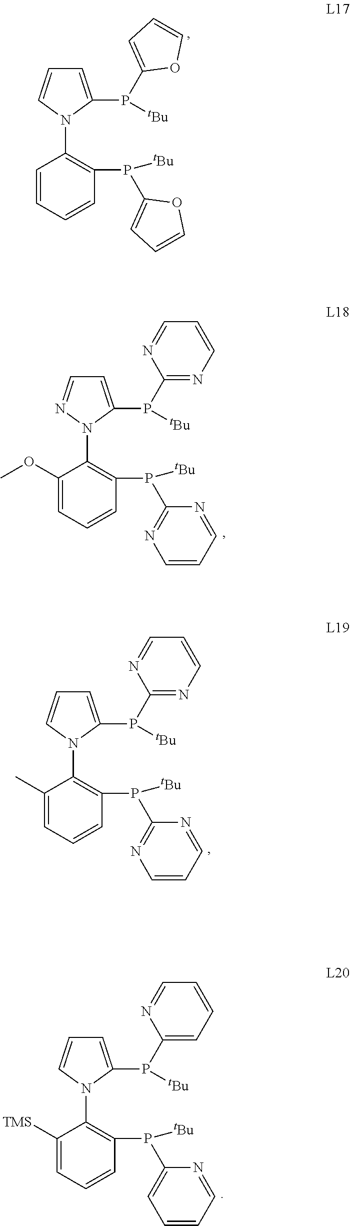 [SMILES: CC(C)(C)P(c1ccco1)c1ccccc1-n1cccc1P(c1ccco1)C(C)(C)C ; COc1cccc(P(c2ncccn2)C(C)(C)C)c1-n1nccc1P(c1ncccn1)C(C)(C)C ; Cc1cccc(P(c2ncccn2)C(C)(C)C)c1-n1cccc1P(c1ncccn1)C(C)(C)C ; CC(C)(C)P(c1ccccn1)c1cccc([Si](C)(C)C)c1-n1cccc1P(c1ccccn1)C(C)(C)C]

In one embodiment, said reaction vessel is a high-pressure autoclave.

In one embodiment, the amount of said Formula VIII phosphine ligand is 0.00001-10 mol % relative to ethylene, more preferably 0.0001-1 mol %.

In one embodiment, the reaction temperature is 0-200° C., preferably 60-150° C.

In one embodiment, the reaction is conducted under an inert gas atmosphere; preferably, said inert gas is nitrogen and/or argon.

In one embodiment, the reaction is carried out at 1-10 MPa, preferably 2-8 MPa.

In one embodiment, the reaction time is 0.2-72 hours, preferably 0.2-24 hours.

In one embodiment, the said alcohol is a C₁₋₁₂ alkyl alcohol; preferably, said alcohol is selected from the group consisting of: methanol, ethanol, propanol, butanol, octanol, or a combination thereof.

In one embodiment, the amount of said alcohol is 1-100 molar equivalents relative to ethylene, preferably 1-10 molar equivalents.

In one embodiment, said palladium catalyst is selected from the group consisting of: palladium acetate, palladium trifluoroacetate, palladium pivalate, palladium(II) tetraacetonitrile tetrafluoroborate, palladium(II) hexafluoroacetylacetonate, palladium(II) bis(acetylacetonate), palladium(II) tetraacetonitrile trifluoromethanesulfonate, palladium(II) neodecanoate, bis(dibenzylideneacetone)palladium(0), tris(dibenzylideneacetone)dipalladium(0), palladium(II) chloride, palladium(II) diacetonitrile dichloride, palladium(II) dibenzonitrile dichloride, or a combination thereof.

In one embodiment, said palladium catalyst is selected from the group consisting of: palladium acetate, palladium trifluoroacetate, palladium pivalate, tris(dibenzylideneacetone)dipalladium(0), palladium(II) chloride, palladium(II) diacetonitrile dichloride, or a combination thereof.

In one embodiment, the amount of said palladium catalyst is 0.00001-10 mol % relative to ethylene, more preferably 0.0001-1 mol %.

In the reaction, when said phosphine ligand is a nitrogen-phosphine ligand (i.e., a Formula I compound), the molar ratio of said palladium catalyst to said nitrogen-phosphine ligand is 1:1 to 1:30, more preferably 1:1 to 1:5.

In one embodiment, said acid is selected from the group consisting of: perchloric acid, sulfuric acid, phosphoric acid, sulfonic acid, alkylphosphonic acid, alkylsulfonic acid, alkylcarboxylic acid, perfluoroalkylsulfonic acid, perfluoroalkylcarboxylic acid (wherein said alkyl is a C₁₋₁₂ alkyl), or arylsulfonic acid (wherein said aryl is a C₆₋₁₀ aryl).

In one embodiment, said acid is selected from the group consisting of: methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid, dodecylsulfonic acid, sulfuric acid, sulfonic acid, formic acid, and trifluoroacetic acid.

In one embodiment, the amount of said acid is 0.00004-40 mol % relative to ethylene, preferably 0.0004-4 mol %.

In one embodiment, said solvent is selected from the group consisting of: alkane solvents, substituted aromatic solvents, ether solvents, ketone solvents, nitrile solvents, ester solvents, or a combination thereof.

In one embodiment, said alkane solvent is selected from the group consisting of: n-hexane, cyclohexane, or a combination thereof.

In one embodiment, said substituted aromatic solvent is selected from the group consisting of: chlorobenzene, toluene, xylene, and trifluorotoluene.

In one embodiment, said ether solvent is selected from the group consisting of: tetrahydrofuran, diethyl ether, methyl tert-butyl ether, ethyl tert-butyl ether, anisole, ethylene glycol dimethyl ether, 1,4-dioxane, or a combination thereof.

In one embodiment, said ketone solvent is acetone.

In one embodiment, said nitrile solvent is selected from the group consisting of: acetonitrile, propionitrile, benzonitrile, or a combination thereof.

In one embodiment, said ester solvent is ethyl acetate.

In one embodiment, said alcohol is methanol, and the reaction is carried out under the following conditions:
a) Dissolving palladium acetate, a phosphine ligand of Formula VIII, and an acid in methanol or a mixed solvent with an optional co-solvent;
b) Introducing ethylene into the reactor, followed by carbon monoxide to initiate the reaction, wherein the reaction proceeds at a temperature ranging from room temperature to 80-100° C.;
c) Terminating the reaction and isolating the product.

In one embodiment, said alcohol is ethanol, and the reaction is carried out under the following conditions:
a) Dissolving palladium acetate, a phosphine ligand of Formula VIII, and an acid in ethanol or a mixed solvent with an optional co-solvent;
b) Introducing ethylene into the reactor, followed by carbon monoxide to initiate the reaction, wherein the reaction proceeds at a temperature ranging from room temperature to 80-100° C.;
c) Terminating the reaction and isolating the product.

In one embodiment, said alcohol is butanol, and the reaction is carried out under the following conditions:
a) Dissolving palladium acetate, a phosphine ligand of Formula VIII, and an acid in butanol or a mixed solvent with an optional co-solvent;
b) Introducing ethylene into the reactor, followed by carbon monoxide to initiate the reaction, wherein the reaction proceeds at a temperature ranging from room temperature to 80-100° C.;
c) Terminating the reaction and isolating the product.

In one embodiment, said alcohol is octanol, and the reaction is carried out under the following conditions:
a) Dissolving palladium acetate, a phosphine ligand of Formula VIII, and an acid in octanol or a mixed solvent with an optional co-solvent;
b) Introducing ethylene into the reactor, followed by carbon monoxide to initiate the reaction, wherein the reaction proceeds at a temperature ranging from room temperature to 80-100° C.;
c) Terminating the reaction and isolating the product.

It should be understood that, within the scope of the present invention, the various technical features described above and those specifically described below (e.g., in the embodiments) may be combined with one another to form new or preferred technical solutions. For brevity, these combinations are not exhaustively detailed herein.

### DETAILED DESCRIPTION

The inventors have prepared a series of novel phosphine ligands and a method for synthesizing acrylate/propionate via acetylene/ethylene carbonylation based on these ligands, following extensive and in-depth research. This method improves the catalytic efficiency of acetylene/ethylene carbonylation reactions, enhances the conversion rate of acetylene/ethylene and product selectivity, and features mild reaction conditions and simple operation. Based on these findings, the inventors have accomplished the present invention.

### Definitions

In the present invention, “room temperature” refers to 10-30° C.

In the present invention, the term “alkyl” refers to a straight-chain or branched saturated hydrocarbon group, preferably a C₁₋₁₀ alkyl (e.g., C₁₋₈ alkyl, C₁₋₆ alkyl, or C₁₋₄ alkyl).

In the present invention, the term “cycloalkyl” refers to a saturated monocyclic or polycyclic carbocyclic substituent comprising fused, bridged, or spiro ring systems, preferably a C₃₋₈ cycloalkyl (e.g., C₃₋₆ cycloalkyl).

In the present invention, the term “alkoxy” refers to a cyclic or acyclic alkyl group connected via an oxygen bridge, wherein the definitions of alkyl and cycloalkyl are as described above, preferably a C₁₋₁₀ alkoxy (e.g., C₁₋₈ alkoxy, C₁₋₆ alkoxy, or C₁₋₄ alkoxy).

Unless otherwise specified, the term “aryl” in the present invention refers to a monocyclic or polycyclic (e.g., bicyclic or tricyclic) 4n+2 aromatic ring system (e.g., having 6, 10, or 14 shared π electrons in a cyclic array) with 6-30 (preferably 6-14) ring carbon atoms and zero heteroatoms, preferably a C₆₋₁₄ aryl, more preferably a C₆₋₁₀ aryl.

Unless otherwise specified, the term “heteroaryl” in the present invention refers to a monocyclic or polycyclic (e.g., bicyclic or tricyclic) 4n+2 aromatic ring system (e.g., having 6, 10, or 14 shared π electrons in a cyclic array) with 5-30 (preferably 5-20, more preferably 5-14) ring atoms (which may be carbon atoms or heteroatoms), preferably a 5-15-membered heteroaryl, more preferably a 5-9 membered heteroaryl.

Without departing from common knowledge in the field, the above preferred conditions may be combined in any manner to obtain various preferred embodiments of the present invention.

The reagents and raw materials used in the present invention are all commercially available.

The following specific examples are provided to further illustrate the present invention. It should be understood that these examples are intended to demonstrate the invention without limiting its scope. Unless otherwise specified, experimental methods without detailed conditions in the following examples are generally performed under conventional conditions or as recommended by the manufacturer. Unless stated otherwise, percentages and parts are by weight.

In the following embodiments, where reference is made to a specific category of catalysts, the catalyst structure numbering corresponds to the numbering system for that particular category of catalyst structures.

### Carbonylation of Acetylene

### Category I: Synthesis and Application of Formula I

### Example 1 Preparation of Ligand Represented by Formula I Using L1

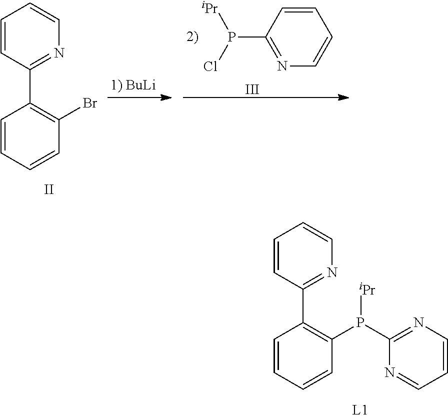

In a 100 mL three-necked flask equipped with a thermometer, magnetic stirrer, and reflux condenser, bromide 11 (10 mmol) was dissolved in 50 mL of dry tetrahydrofuran. The solution was cooled to −78° C., and n-butyllithium (12 mL, 1.6 M in hexane) was added via syringe. After stirring at low temperature for 3 hours, a solution of chlorophosphine III (10 mmol) in 10 mL of dry tetrahydrofuran was added dropwise. The reaction mixture was allowed to warm to room temperature naturally and stirred overnight. Upon completion of the reaction, the mixture was quenched with 2 mL of degassed water, concentrated to remove the solvent, and then treated with oxygen-free water. The resulting mixture was extracted with diethyl ether, and the organic layer was separated, dried over anhydrous magnesium sulfate, and concentrated under reduced pressure to remove the ether. The crude product was recrystallized from methanol to afford a white or pale yellow solid powder.

Partial ligand data are as follows:

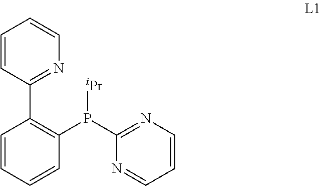 [SMILES: CC(C)P(c1ncccn1)c1ccccc1-c1ccccn1]

¹H NMR (400 MHz, CDCl₃) δ 8.58 (d, J=7.6 Hz, 2H), 8.42 (d, J=8.0 Hz, 1H), 8.12 (d, J=7.6 Hz, 1H), 7.53-7.47 (m, 3H), 7.34-7.30 (m, 2H), 7.14-7.11 (m, 1H), 7.00-6.97 (m, 1H), 1.72-1.68 (m, 1H), 0.98 (d, J=6.8 Hz, 6H).

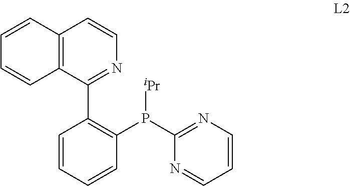 [SMILES: CC(C)P(c1ncccn1)c1ccccc1-c1nccc2ccccc12]

¹H NMR (400 MHz, CDCl₃) δ 8.63 (d, J=7.6 Hz, 2H), 8.46 (d, J=8.0 Hz, 1H), 8.12 (d, J=7.6 Hz, 1H), 8.00 (d, J=7.6 Hz, 1H), 7.64-7.47 (m, 7H), 7.34-7.30 (m, 1H), 1.72-1.68 (m, 1H), 1.01 (d, J=6.8 Hz, 6H).

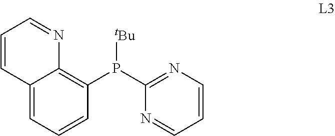 [SMILES: CC(C)(C)P(c1ncccn1)c1cccc2cccnc12]

¹H NMR (400 MHz, CDCl₃) δ 9.01 (d, J=7.6 Hz, 1H), 8.71 (d, J=7.6 Hz, 2H), 8.49 (d, J=7.6 Hz, 1H), 8.09 (d, J=7.6 Hz, 1H), 7.83-7.67 (m, 4H), 1.08 (s, 9H).

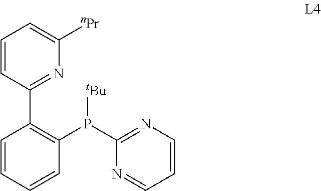

¹H NMR (400 MHz, CDCl₃) δ 9.01 (d, J=7.6 Hz, 1H), 8.71 (d, J=7.6 Hz, 2H), 8.49 (d, J=7.6 Hz, 1H), 8.09 (d, J=7.6 Hz, 1H), 7.83-7.67 (m, 4H), 1.08 (s, 9H).

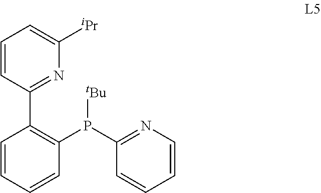 [SMILES: CC(C)c1cccc(-c2ccccc2P(c2ccccn2)C(C)(C)C)n1]

¹H NMR (400 MHz, CDCl₃) δ 8.56 (d, J=7.6 Hz, 1H), 8.17 (d, J=7.6 Hz, 1H), 7.66-7.53 (m, 3H), 7.35-7.30 (m, 4H), 6.87-6.78 (m, 2H), 3.22-3.18 (m, 1H), 1.28 (d, J=7.2 Hz, 6H), 1.09 (s, 9H).

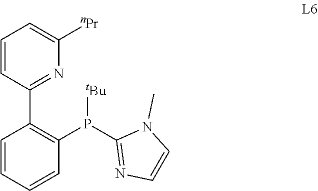

¹H NMR (400 MHz, CDCl₃) δ 8.18 (d, J=7.6 Hz, 1H), 7.52-7.49 (m, 2H), 7.35-7.30 (m, 2H), 7.02 (d, J=7.2 Hz, 1H), 6.81 (d, J=7.2 Hz, 1H), 3.68 (s, 3H), 3.00 (t, J=7.2 Hz, 2H), 1.76-1.73 (m, 2H), 1.08 (s, 9H), 0.93 (t, J=7.2 Hz, 3H).

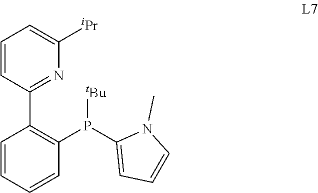 [SMILES: CC(C)c1cccc(-c2ccccc2P(c2cccn2C)C(C)(C)C)n1]

¹H NMR (400 MHz, CDCl₃) δ 8.20 (d, J=8.0 Hz, 1H), 7.52-7.49 (m, 2H), 7.35-7.26 (m, 3H), 7.13 (d, J=7.2 Hz, 1H), 6.82 (d, J=7.2 Hz, 1H), 6.20-6.17 (m, 2H), 3.65 (s, 3H), 3.35-3.30 (m, 1H), 1.29 (d, J=7.2 Hz, 6H), 1.07 (s, 9H).

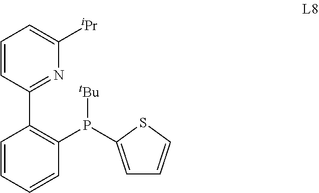 [SMILES: CC(C)c1cccc(-c2ccccc2P(c2cccs2)C(C)(C)C)n1]

¹H NMR (400 MHz, CDCl₃) δ 8.15 (d, J=7.6 Hz, 1H), 7.55-7.53 (m, 2H), 7.35-7.30 (m, 2H), 7.08-6.97 (m, 4H), 6.82 (d, J=7.2 Hz, 1H), 3.39-3.36 (m, 1H), 1.28 (d, J=7.2 Hz, 6H), 1.09 (s, 9H).

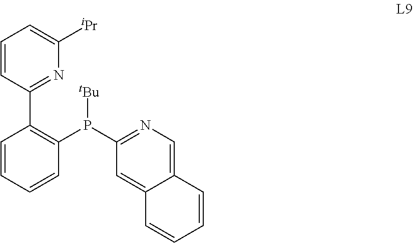 [SMILES: CC(C)c1cccc(-c2ccccc2P(c2cc3ccccc3cn2)C(C)(C)C)n1]

¹H NMR (400 MHz, CDCl₃) δ 9.30 (s, 1H), 8.17 (d, J=7.6 Hz, 1H), 8.02 (d, J=7.6 Hz, 1H), 7.60-7.48 (m, 6H), 7.35-7.30 (m, 2H), 7.01 (d, J=7.6 Hz, 1H), 6.82 (d, J=7.2 Hz, 1H), 3.37-3.33 (m, 1H), 1.28 (d, J=7.2 Hz, 6H), 1.09 (s, 9H).

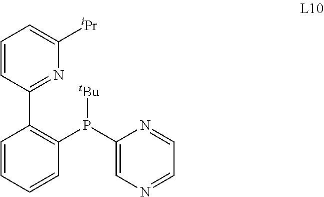 [SMILES: CC(C)c1cccc(-c2ccccc2P(c2cnccn2)C(C)(C)C)n1]

¹H NMR (400 MHz, CDCl₃) δ 8.85-8.76 (m, 3H), 8.18 (d, J=7.6 Hz, 1H), 7.55-7.50 (m, 2H), 7.34-7.29 (m, 2H), 6.98 (d, J=7.6 Hz, 1H), 6.75 (d, J=7.2 Hz, 1H), 3.37-3.33 (m, 1H), 1.30 (d, J=7.2 Hz, 6H), 1.08 (s, 9H).

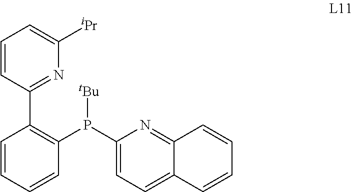 [SMILES: CC(C)c1cccc(-c2ccccc2P(c2ccc3ccccc3n2)C(C)(C)C)n1]

¹H NMR (400 MHz, CDCl₃) δ 8.55 (d, J=8.0 Hz, 1H), 8.15 (d, J=7.6 Hz, 1H), 8.02-7.97 (m, 2H), 7.85-7.80 (m, 1H), 7.55-7.50 (m, 4H), 7.34-7.29 (m, 2H), 7.00 (d, J=7.6 Hz, 1H), 6.70 (d, J=7.2 Hz, 1H), 3.37-3.33 (m, 1H), 1.27 (d, J=7.2 Hz, 6H), 1.07 (s, 9H).

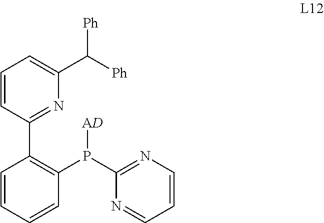

¹H NMR (400 MHz, CDCl₃) δ 8.63 (d, J=8.0 Hz, 2H), 8.13 (d, J=7.6 Hz, 1H), 7.55-7.50 (m, 3H), 7.33-7.15 (m, 12H), 7.00-6.95 (m, 2H), 5.53 (s, 1H), 2.03-1.98 (m, 3H), 1.80-1.71 (m, 12H).

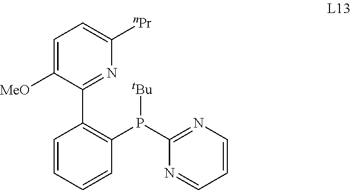

¹H NMR (400 MHz, CDCl₃) δ 8.65 (d, J=8.0 Hz, 2H), 8.17 (d, J=7.6 Hz, 1H), 7.52-7.35 (m, 4H), 7.02-6.97 (m, 2H), 3.98 (s, 3H), 3.05 (t, J=7.2 Hz, 2H), 1.76-1.73 (m, 2H), 1.08 (s, 9H), 0.95 (t, J=7.2 Hz, 3H).

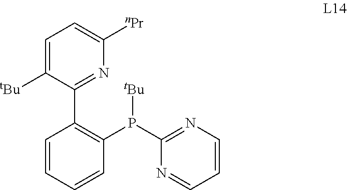

¹H NMR (400 MHz, CDCl₃) δ 8.68 (d, J=8.0 Hz, 2H), 8.15 (d, J=7.6 Hz, 1H), 7.60-7.30 (m, 4H), 7.12 (d, J=7.6 Hz, 1H), 6.72 (d, J=7.6 Hz, 1H), 3.02 (t, J=7.2 Hz, 2H), 1.76-1.70 (m, 2H), 1.40 (s, 9H), 1.08 (s, 9H), 0.95 (t, J=7.2 Hz, 3H).

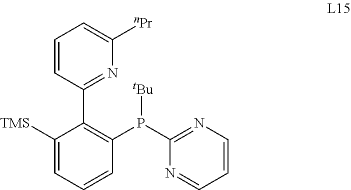

¹H NMR (400 MHz, CDCl₃) δ 8.65 (d, J=7.6 Hz, 2H), 7.65-7.30 (m, 5H), 7.02 (d, J=7.6 Hz, 1H), 6.81 (d, J=7.6 Hz, 1H), 3.02 (t, J=7.2 Hz, 2H), 1.76-1.70 (m, 2H), 1.08 (s, 9H), 0.96 (t, J=7.2 Hz, 3H), 0.12 (s, 9H).

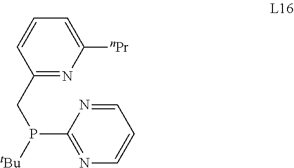

¹H NMR (400 MHz, CDCl₃) δ 8.75 (d, J=7.6 Hz, 2H), 7.65-7.58 (m, 2H), 7.02-6.97 (m, 2H), 4.00-3.96 (m, 1H), 3.70-3.66 (m, 1H), 3.02 (t, J=7.2 Hz, 2H), 1.82-1.79 (m, 2H), 1.06 (s, 9H), 0.98 (t, J=7.2 Hz, 3H).

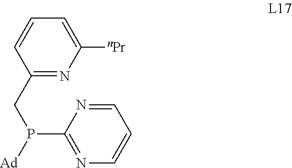

¹H NMR (400 MHz, CDCl₃) δ 8.73 (d, J=7.6 Hz, 2H), 7.60-7.53 (m, 2H), 7.06-7.00 (m, 2H), 4.03-3.97 (m, 1H), 3.74-3.69 (m, 1H), 3.02 (t, J=7.2 Hz, 2H), 2.03-1.98 (m, 3H), 1.80-1.71 (m, 14H), 0.98 (t, J=7.2 Hz, 3H).

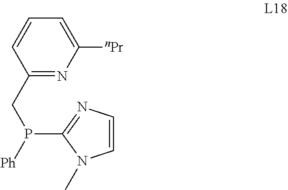

¹H NMR (400 MHz, CDCl₃) δ 7.57 (t, J=7.6 Hz, 1H), 7.46-7.38 (m, 3H), 7.18-7.14 (m, 3H), 7.02-6.98 (m, 2H), 6.88 (d, J=7.6 Hz, 1H), 4.02-3.98 (m, 1H), 3.72-3.68 (m, 1H), 3.54 (s, 3H), 3.45-3.42 (m, 1H), 1.36 (d, J=7.2 Hz, 6H).

### Example 2

A 300 mL Parr autoclave was charged under nitrogen atmosphere with Pd(OAc)₂ (2.2 mg, 0.01 mmol), nitrogen-phosphorus ligand L1 of Formula I (6.2 mg, 0.02 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 87% yield with >90% selectivity.

### Example 3

A 300 mL Parr autoclave was charged under nitrogen atmosphere with Pd(OAc)₂ (2.2 mg, 0.01 mmol), nitrogen-phosphorus ligand L2 of Formula I (7.1 mg, 0.02 mmol), methanesulfonic acid (6.5 μL, 0.10 mmol), methanol (20 mL), and a stir bar. The system was purged with acetylene and charged with acetylene (62 mmol) via flow meter under cooling, followed by pressurization with carbon monoxide to 4 MPa. The mixture was rapidly heated to 120° C. and stirred for 5 hours. After cooling, NMR analysis indicated methyl acrylate was obtained in 85% yield with >90% selectivity.

### Example 4

A 300 mL Parr autoclave was charged under nitrogen atmosphere with Pd(OAc)₂ (2.2 mg, 0.01 mmol), nitrogen-phosphorus ligand L3 of Formula I (5.9 mg, 0.02 mmol), methanesulfonic acid (6.5 μL, 0.10 mmol), methanol (10 mL), and a stir bar. The system was purged with acetylene and charged with acetylene (62 mmol) via flow meter under cooling, followed by pressurization with carbon monoxide to 4 MPa. The mixture was rapidly heated to 120° C. and stirred for 5 hours. After cooling, NMR analysis indicated methyl acrylate was obtained in 64% yield with >90% selectivity.

### Example 5

A 300 mL Parr autoclave was charged under nitrogen atmosphere with Pd(OAc)₂ (2.2 mg, 0.01 mmol), nitrogen-phosphorus ligand L4 of Formula I (14.5 mg, 0.02 mmol), methanesulfonic acid (10.4 μL, 0.16 mmol), methanol (10 mL), and a stir bar. The system was purged with acetylene and charged with acetylene (62 mmol) via flow meter under cooling, followed by pressurization with carbon monoxide to 6 MPa. The mixture was rapidly heated to 120° C. and stirred for 6 hours. After cooling, NMR analysis indicated methyl acrylate was obtained in 90% yield with >90% selectivity.

### Example 6

A 300 mL Parr autoclave was charged under nitrogen atmosphere with Pd(OAc)₂ (2.2 mg, 0.01 mmol), nitrogen-phosphorus ligand L5 of Formula I (14.4 mg, 0.04 mmol), methanesulfonic acid (10.4 μL, 0.16 mmol), methanol (10 mL), and a stir bar. The system was purged with acetylene and charged with acetylene (100 mmol) via flow meter under cooling, followed by pressurization with carbon monoxide to 6 MPa. The mixture was rapidly heated to 120° C. and stirred for 6 hours. After cooling, NMR analysis indicated methyl acrylate was obtained in 85% yield with >90% selectivity.

### Example 7

A 300 mL Parr autoclave was charged under nitrogen atmosphere with Pd(OAc)₂ (2.2 mg, 0.01 mmol), nitrogen-phosphorus ligand L6 of Formula I (14.6 mg, 0.04 mmol), p-toluenesulfonic acid (17.2 mg, 0.10 mmol), methanol (10 mL), and a stir bar. The system was purged with acetylene and charged with acetylene (100 mmol) via flow meter under cooling, followed by pressurization with carbon monoxide to 6 MPa. The mixture was rapidly heated to 120° C. and stirred for 5 hours. After cooling, NMR analysis indicated methyl acrylate was obtained in 78% yield with >90% selectivity.

### Example 8

A 300 mL Parr autoclave was charged under nitrogen atmosphere with PdCl₂ (1.8 mg, 0.01 mmol), nitrogen-phosphorus ligand L7 of Formula I (14.5 mg, 0.04 mmol), trifluoromethanesulfonic acid (14.2 μL, 0.16 mmol), butanol (5 mL), and THF (10 mL), and a stir bar. The system was purged with acetylene, charged with acetylene (62 mmol) via flow meter under cooling, then pressurized with carbon monoxide to 4 MPa. The mixture was rapidly heated to 120° C. and stirred for 6 hours. After cooling, NMR analysis indicated butyl acrylate was obtained in 65% yield with >90% selectivity.

### Example 9

A 300 mL Parr autoclave was charged under nitrogen atmosphere with Pd(OAc)₂ (2.2 mg, 0.01 mmol), nitrogen-phosphorus ligand L8 of Formula I (7.3 mg, 0.02 mmol), dodecylsulfonic acid (26.1 mg, 0.08 mmol) in methanol (20 mL), and a stir bar. The system was purged with acetylene, charged with acetylene (62 mmol) via flow meter under cooling, then pressurized with carbon monoxide to 4 MPa. The mixture was rapidly heated to 120° C. and stirred for 6 hours. After cooling, NMR analysis indicated methyl acrylate was obtained in 60% yield with >90% selectivity.

### Example 10

A 300 mL Parr autoclave was charged under nitrogen atmosphere with Pd(OAc)₂ (2.2 mg, 0.01 mmol), nitrogen-phosphorus ligand L9 of Formula I (8.2 mg, 0.02 mmol), methanesulfonic acid (10.4 μL, 0.16 mmol), octanol (10 mL), and hexane (10 mL), and a stir bar. The system was purged with acetylene, charged with acetylene (62 mmol) via flow meter under cooling, then pressurized with carbon monoxide to 4 MPa. The mixture was rapidly heated to 120° C. and stirred for 5 hours. After cooling, NMR analysis indicated octyl acrylate was obtained in 81% yield with >90% selectivity.

### Example 11

A 300 mL Parr autoclave was charged under nitrogen atmosphere with Pd(OAc)₂ (2.2 mg, 0.01 mmol), nitrogen-phosphorus ligand L10 of Formula I (7.3 mg, 0.02 mmol), methanesulfonic acid (10.4 μL, 0.16 mmol), methanol (20 mL), and a stir bar. The system was purged with acetylene, charged with acetylene (62 mmol) via flow meter under cooling, then pressurized with carbon monoxide to 4 MPa. The mixture was rapidly heated to 120° C. and stirred for 5 hours. After cooling, NMR analysis indicated methyl acrylate was obtained in 77% yield with >90% selectivity.

### Example 12

A 300 mL Parr autoclave was charged under nitrogen atmosphere with Pd(OAc)₂ (2.2 mg, 0.01 mmol), nitrogen-phosphorus ligand L12 of Formula I (2.3 mg, 0.004 mmol), methanesulfonic acid (10.4 μL, 0.16 mmol), methanol (20 mL), and a stir bar. The system was purged with acetylene, charged with acetylene (62 mmol) via flow meter under cooling, then pressurized with carbon monoxide to 4 MPa. The mixture was rapidly heated to 120° C. and stirred for 5 hours. After cooling, NMR analysis indicated methyl acrylate was obtained in 85% yield with >90% selectivity.

### Example 13

A 300 mL Parr autoclave was charged under nitrogen atmosphere with Pd(OAc)₂ (2.2 mg, 0.01 mmol), nitrogen-phosphorus ligand L13 of Formula I (7.8 mg, 0.02 mmol), methanesulfonic acid (10.4 μL, 0.16 mmol), methanol (20 mL), and a stir bar. The system was purged with acetylene, charged with acetylene (62 mmol) via flow meter under cooling, then pressurized with carbon monoxide to 4 MPa. The mixture was rapidly heated to 120° C. and stirred for 5 hours. After cooling, NMR analysis indicated methyl acrylate was obtained in 80% yield with >90% selectivity.

### Example 14

A 300 mL Parr autoclave was charged under nitrogen atmosphere with Pd(OAc)₂ (2.2 mg, 0.01 mmol), nitrogen-phosphorus ligand L15 of Formula I (17.4 mg, 0.04 mmol), methanesulfonic acid (10.4 μL, 0.16 mmol), methanol (20 mL), and a stir bar. The system was purged with acetylene, charged with acetylene (62 mmol) via flow meter under cooling, then pressurized with carbon monoxide to 4 MPa. The mixture was rapidly heated to 120° C. and stirred for 5 hours. After cooling, NMR analysis indicated methyl acrylate was obtained in 82% yield with >90% selectivity.

### Example 15

A 300 mL Parr autoclave was charged under nitrogen atmosphere with Pd(OAc)₂ (2.2 mg, 0.01 mmol), nitrogen-phosphorus ligand L16 of Formula I (12.0 mg, 0.04 mmol), methanesulfonic acid (10.4 μL, 0.16 mmol), methanol (20 mL), and a stir bar. The system was purged with acetylene, charged with acetylene (62 mmol) via flow meter under cooling, then pressurized with carbon monoxide to 4 MPa. The mixture was rapidly heated to 120° C. and stirred for 5 hours. After cooling, NMR analysis indicated methyl acrylate was obtained in 65% yield with >90% selectivity.

### Example 16

A 300 mL Parr autoclave was charged under nitrogen atmosphere with Pd(OAc)₂ (2.2 mg, 0.01 mmol), nitrogen-phosphorus ligand L17 of Formula I (15.2 mg, 0.04 mmol), methanesulfonic acid (10.4 μL, 0.16 mmol), methanol (20 mL), and a stir bar. The system was purged with acetylene, charged with acetylene (62 mmol) via flow meter under cooling, then pressurized with carbon monoxide to 4 MPa. The mixture was rapidly heated to 120° C. and stirred for 5 hours. After cooling, NMR analysis indicated methyl acrylate was obtained in 61% yield with >90% selectivity.

### Example 17

A 300 mL Parr autoclave was charged under nitrogen atmosphere with Pd(OAc)₂ (2.2 mg, 0.01 mmol), nitrogen-phosphorus ligand L18 of Formula I (12.9 mg, 0.04 mmol), methanesulfonic acid (10.4 μL, 0.16 mmol), methanol (20 mL), and a stir bar. The system was purged with acetylene, charged with acetylene (62 mmol) via flow meter under cooling, then pressurized with carbon monoxide to 4 MPa. The mixture was rapidly heated to 120° C. and stirred for 5 hours. After cooling, NMR analysis indicated methyl acrylate was obtained in 76% yield with >90% selectivity.

### Example 18

A 300 mL Parr autoclave was charged under nitrogen atmosphere with Pd(CH₃CN)₄(BF₄)₂ (4.4 mg, 0.01 mmol), nitrogen-phosphorus ligand L1 of Formula I (12.3 mg, 0.04 mmol), methanesulfonic acid (10.4 μL, 0.16 mmol), methanol (10 mL) 1,4-dioxane (10 mL), and a stir bar. The system was purged with acetylene, charged with acetylene (62 mmol) via flow meter under cooling, then pressurized with carbon monoxide to 4 MPa. The mixture was rapidly heated to 100° C. and stirred for 5 hours. After cooling, NMR analysis indicated methyl acrylate was obtained in 80% yield with >90% selectivity.

### Example 19

A 300 mL Parr autoclave was charged under nitrogen atmosphere with Pd(hfacac)₂ (3.1 mg, 0.01 mmol), nitrogen-phosphorus ligand L1 of Formula I (12.3 mg, 0.04 mmol), methanesulfonic acid (10.4 μL, 0.16 mmol), butanol (5 mL), THF (15 mL), and a stir bar. The system was purged with acetylene, charged with acetylene (62 mmol) via flow meter under cooling, then pressurized with carbon monoxide to 4 MPa. The mixture was rapidly heated to 120° C. and stirred for 5 hours. After cooling, NMR analysis indicated butyl acrylate was obtained in 92% yield with >93% selectivity.

### Example 20

A 300 mL Parr autoclave was charged under nitrogen atmosphere with Pd(OTFA)₂ (4.0 mg, 0.01 mmol), nitrogen-phosphorus ligand L1 of Formula I (12.3 mg, 0.04 mmol), p-toluenesulfonic acid (17.5 mg, 0.10 mmol), octanol (5 mL), THF (15 mL), and a stir bar. The system was purged with acetylene, charged with acetylene (100 mmol) via flow meter under cooling, then pressurized with carbon monoxide to 6 MPa. The mixture was rapidly heated to 120° C. and stirred for 5 hours. After cooling, NMR analysis indicated octyl acrylate was obtained in 86% yield with >90% selectivity.

### Example 21

A 300 mL Parr autoclave was charged under nitrogen atmosphere with Pd(OPiv)₂ (0.3 mg, 0.001 mmol), nitrogen-phosphorus ligand L1 of Formula I (1.2 mg, 0.004 mmol), methanesulfonic acid (1.0 μL, 0.016 mmol), methanol (20 mL), and a stir bar. The system was purged with acetylene, charged with acetylene (100 mmol) via flow meter under cooling, then pressurized with carbon monoxide to 6 MPa. The mixture was rapidly heated to 80° C. and stirred for 10 hours. After cooling, NMR analysis indicated methyl acrylate was obtained in 89% yield with >90% selectivity.

### Example 22

A 300 mL Parr autoclave was charged under nitrogen atmosphere with Pd₂(dba)₃ (4.6 mg, 0.005 mmol), nitrogen-phosphorus ligand L1 of Formula I (12.3 mg, 0.04 mmol), trifluoroacetic acid (12 μL, 0.16 mmol), methanol (20 mL), and a stir bar. The system was purged with acetylene, charged with acetylene (100 mmol) via flow meter under cooling, then pressurized with carbon monoxide to 6 MPa. The mixture was rapidly heated to 40° C. and stirred for 6 hours. After cooling, NMR analysis indicated methyl acrylate was obtained in 85% yield with >90% selectivity.

### Example 23

A 300 mL Parr autoclave was charged under nitrogen atmosphere with Pd(OAc)₂ (2.24 mg, 0.01 mmol), nitrogen-phosphorus ligand L1 of Formula I (12.3 mg, 0.04 mmol), methanesulfonic acid (10.4 μL, 0.16 mmol), methanol (20 mL), and a stir bar. The system was purged with acetylene, charged with acetylene (100 mmol) via flow meter under cooling, then pressurized with carbon monoxide to 6 MPa. The mixture was rapidly heated to 40° C. and stirred for 6 hours. After cooling, NMR analysis indicated methyl acrylate was obtained in 85% yield with >90% selectivity.

### Category II: Application of Formula II

### Example 24

A 300 mL Parr autoclave was charged under nitrogen atmosphere with Pd(OAc)₂ (2.2 mg, 0.01 mmol), bisphosphorus ligand L1 of Formula II (23.4 mg, 0.04 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 87% yield with >90% selectivity.

### Example 25

A 300 mL Parr autoclave was charged under nitrogen atmosphere with Pd(OAc)₂ (2.2 mg, 0.01 mmol), bisphosphorus ligand L2 of Formula II (23.4 mg, 0.04 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 85% yield with >90% selectivity.

### Example 26

A 300 mL Parr autoclave was charged under nitrogen atmosphere with Pd(OAc)₂ (2.2 mg, 0.01 mmol), bisphosphorus ligand L3 of Formula II (22.4 mg, 0.04 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 78% yield with >90% selectivity.

### Example 27

A 300 mL Parr autoclave was charged under nitrogen atmosphere with Pd(OAc)₂ (2.2 mg, 0.01 mmol), bisphosphorus ligand L4 of Formula II (21.6 mg, 0.04 mmol), p-toluenesulfonic acid (17.2 mg, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 82% yield with >90% selectivity.

### Example 28

A 300 mL Parr autoclave was charged under nitrogen atmosphere with Pd(OAc)₂ (2.2 mg, 0.01 mmol), bisphosphorus ligand L5 of Formula II (14.0 mg, 0.02 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 78% yield with >90% selectivity.

### Example 29

A 300 mL Parr autoclave was charged under nitrogen atmosphere with Pd(OAc)₂ (2.2 mg, 0.01 mmol), bisphosphorus ligand L6 of Formula II (21.8 mg, 0.04 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 75% yield with >90% selectivity.

### Example 30

A 300 mL Parr autoclave was charged under nitrogen atmosphere with Pd(OAc)₂ (2.2 mg, 0.01 mmol), bisphosphorus ligand L8 of Formula II (101.6 mg, 0.02 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 80% yield with >90% selectivity.

### Example 31

A 300 mL Parr autoclave was charged under nitrogen atmosphere with Pd(OAc)₂ (2.2 mg, 0.01 mmol), bisphosphorus ligand L9 of Formula II (20.0 mg, 0.04 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 85% yield with >90% selectivity.

### Example 32

A 300 mL Parr autoclave was charged under nitrogen atmosphere with Pd(OTFA)₂ (4.0 mg, 0.01 mmol), bisphosphorus ligand L10 of Formula II (26.2 mg, 0.04 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 86% yield with >90% selectivity.

### Example 33

A 300 mL Parr autoclave was charged under nitrogen atmosphere with Pd(OPiv)₂ (3.1 mg, 0.01 mmol), bisphosphorus ligand L11 of Formula II (19.1 mg, 0.04 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 6 hours. After completion, the system was cooled to room temperature. NMR analysis indicated methyl acrylate was obtained in 65% yield with >90% selectivity.

### Example 34

A 300 mL Parr autoclave was charged under nitrogen atmosphere with Pd₂(dba)₃ (4.6 mg, 0.005 mmol), bisphosphorus ligand L12 of Formula II (11.6 mg, 0.02 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 74% yield with >90% selectivity.

### Example 35

A 300 mL Parr autoclave was charged under nitrogen atmosphere with PdCl₂ (1.8 mg, 0.01 mmol), bisphosphorus L14 of Formula II (29.8 mg, 0.04 mmol), trifluoromethanesulfonic acid (14.2 μL, 0.10 mmol), methanol (10 mL), THF (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated methyl acrylate was obtained in 83% yield with >90% selectivity.

### Example 36

A 300 mL Parr autoclave was charged under nitrogen atmosphere with Pd(OAc)₂ (2.2 mg, 0.01 mmol), bisphosphorus ligand L2 of Formula II (11.7 mg, 0.02 mmol), methanesulfonic acid (10.4 μL, 0.16 mmol), ethanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 100° C. and stirred for 6 hours. After completion, the system was cooled to room temperature. NMR analysis indicated ethyl acrylate was obtained in 83% yield with >90% selectivity.

### Example 37

A 300 mL Parr autoclave was charged under nitrogen atmosphere with Pd(OAc)₂ (2.2 mg, 0.01 mmol), bisphosphorus ligand L2 of Formula II (23.4 mg, 0.04 mmol), methanesulfonic acid (10.4 μL, 0.16 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 80° C. and stirred for 6 hours. After completion, the system was cooled to room temperature. NMR analysis indicated methyl acrylate was obtained in 82% yield with >90% selectivity.

### Example 38

A 300 mL Parr autoclave was charged under nitrogen atmosphere with Pd(hfacac)₂ (3.1 mg, 0.01 mmol), bisphosphorus ligand L2 of Formula II (23.4 mg, 0.04 mmol), methanesulfonic acid (10.4 μL, 0.16 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 60° C. and stirred for 12 hours. After completion, the system was cooled to room temperature. NMR analysis indicated methyl acrylate was obtained in 80% yield with >90% selectivity.

### Example 39

A 300 mL Parr autoclave was charged under nitrogen atmosphere with Pd(OAc)₂ (2.2 mg, 0.01 mmol), bisphosphorus ligand L2 of Formula II (23.4 mg, 0.04 mmol), dodecylbenzenesulfonic acid (26.1 mg, 0.08 mmol), methanol (10 mL), 1,4-dioxane (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 40° C. and stirred for 16 hours. After completion, the system was cooled to room temperature. NMR analysis indicated methyl acrylate was obtained in 80% yield with >90% selectivity.

### Example 40

A 300 mL Parr autoclave was charged under nitrogen atmosphere with Pd(OAc)₂ (1.1 mg, 0.005 mmol), bisphosphorus ligand L2 of Formula II (2.3 mg, 0.004 mmol), methanesulfonic acid (10.4 μL, 0.16 mmol), butanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 80° C. and stirred for 6 hours. After completion, the system was cooled to room temperature. NMR analysis indicated butyl acrylate was obtained in 82% yield with >90% selectivity.

### Example 41

A 300 mL Parr autoclave was charged under nitrogen atmosphere with Pd(OAc)₂ (11.2 mg, 0.05 mmol), bisphosphorus ligand L2 of Formula II (23.4 mg, 0.04 mmol), methanesulfonic acid (10.4 μL, 0.16 mmol), propanol (10 mL), n-hexane (10 mL), and a stir bar. The system was purged with acetylene gas and then charged with acetylene (62 mmol) via flow meter under cooling, followed by pressurization with carbon monoxide to 4 MPa.

The reaction mixture was stirred at rt for 16 hours. After completion, NMR analysis indicated propyl acrylate was obtained in 78% yield with >90% selectivity.

### Example 42

A 300 mL Parr autoclave was charged under nitrogen atmosphere with Pd₂(dba)₃ (4.6 mg, 0.005 mmol), bisphosphorus ligand L2 of Formula II (23.4 mg, 0.04 mmol), trifluoroacetic acid (12 μL, 0.16 mmol), octanol (10 mL), THF (10 mL) and a stir bar. The system was purged with acetylene gas and then charged with acetylene (100 mmol) via flow meter under cooling, followed by pressurization with carbon monoxide to 6 MPa. The reaction mixture was rapidly heated to 100° C. and stirred for 5 hours. After completion, the system was cooled to room temperature. NMR analysis indicated octyl acrylate was obtained in 85% yield with >90% selectivity.

### Category III: Application of Formula III

### Example 43

A 300 mL Parr autoclave was charged under nitrogen atmosphere with Pd(OAc)₂ (2.2 mg, 0.01 mmol), bisphosphorus ligand L1 of Formula III (7.4 mg, 0.02 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 4 hours. After completion, the system was cooled to room temperature. NMR analysis indicated methyl acrylate was obtained in 89% yield with >90% selectivity.

### Example 44

A 300 mL Parr autoclave was charged under nitrogen atmosphere with Pd(OAc)₂ (2.2 mg, 0.01 mmol), bisphosphorus ligand L2 of Formula III (8.4 mg, 0.02 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 4 hours. After completion, the system was cooled to room temperature. NMR analysis indicated methyl acrylate was obtained in 82% yield with >90% selectivity.

### Example 45

A 300 mL Parr autoclave was charged under nitrogen atmosphere with Pd(OAc)₂ (0.2 mg, 0.001 mmol), bisphosphorus ligand L3 of Formula III (2.2 mg, 0.004 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 4 hours. After completion, the system was cooled to room temperature. NMR analysis indicated methyl acrylate was obtained in 76% yield with >90% selectivity.

### Example 46

A 300 mL Parr autoclave was charged under nitrogen atmosphere with Pd(OTFA)₂ (4.0 mg, 0.01 mmol), bisphosphorus ligand L4 of Formula III (13.5 mg, 0.02 mmol), p-toluenesulfonic acid (17.2 mg, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 4 hours. After completion, the system was cooled to room temperature. NMR analysis indicated methyl acrylate was obtained in 91% yield with >90% selectivity.

### Example 47

A 300 mL Parr autoclave was charged under nitrogen atmosphere with Pd(OPiv)₂ (3.1 mg, 0.01 mmol), bisphosphorus ligand L5 of Formula III (20.7 mg, 0.04 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 4 hours. After completion, the system was cooled to room temperature. NMR analysis indicated methyl acrylate was obtained in 90% yield with >90% selectivity.

### Example 48

A 300 mL Parr autoclave was charged under nitrogen atmosphere with Pd₂(dba)₃ (4.6 mg, 0.005 mmol), bisphosphorus ligand L6 of Formula III (8.0 mg, 0.02 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 4 hours. After completion, the system was cooled to room temperature. NMR analysis indicated methyl acrylate was obtained in 83% yield with >90% selectivity.

### Example 49

A 300 mL Parr autoclave was charged under nitrogen atmosphere with PdCl₂ (1.8 mg, 0.01 mmol), bisphosphorus ligand L8 of Formula III (20.7 mg, 0.04 mmol), trifluoromethanesulfonic acid (14.2 μL, 0.16 mmol), ethanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 4 hours. After completion, the system was cooled to room temperature. NMR analysis indicated ethyl acrylate was obtained in 78% yield with >90% selectivity.

### Example 50

A 300 mL Parr autoclave was charged under nitrogen atmosphere with Pd(OAc)₂ (2.2 mg, 0.01 mmol), bisphosphorus ligand L9 of Formula III (7.6 mg, 0.02 mmol), methanesulfonic acid (10.4 μL, 0.16 mmol), methanol (20 mL), THF (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 4 hours. After completion, the system was cooled to room temperature. NMR analysis indicated methyl acrylate was obtained in 80% yield with >90% selectivity.

### Example 51

A 300 mL Parr autoclave was charged under nitrogen atmosphere with Pd(OAc)₂ (2.2 mg, 0.01 mmol), bisphosphorus ligand L10 of Formula III (7.5 mg, 0.02 mmol), methanesulfonic acid (10.4 μL, 0.16 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 4 hours. After completion, the system was cooled to room temperature. NMR analysis indicated methyl acrylate was obtained in 80% yield with >90% selectivity.

### Example 52

A 300 mL Parr autoclave was charged under nitrogen atmosphere with Pd(OAc)₂ (2.2 mg, 0.01 mmol), bisphosphorus ligand L11 of Formula III (8.2 mg, 0.02 mmol), methanesulfonic acid (10.4 μL, 0.16 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 4 hours. After completion, the system was cooled to room temperature. NMR analysis indicated methyl acrylate was obtained in 76% yield with >90% selectivity.

### Example 53

A 300 mL Parr autoclave was charged under nitrogen atmosphere with Pd(OAc)₂ (2.2 mg, 0.01 mmol), bisphosphorus ligand L13 of Formula III (10.4 mg, 0.02 mmol), dodecylbenzenesulfonic acid (26.1 mg, 0.08 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 4 hours. After completion, the system was cooled to room temperature. NMR analysis indicated methyl acrylate was obtained in 86% yield with >90% selectivity.

### Example 54

A 300 mL Parr autoclave was charged under nitrogen atmosphere with Pd(OAc)₂ (11.2 mg, 0.05 mmol), bishosphorus ligand L14 of Formula III (22.6 mg, 0.04 mmol), methanesulfonic acid (10.4 μL, 0.16 mmol), butanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 4 hours. After completion, the system was cooled to room temperature. NMR analysis indicated butyl acrylate was obtained in 85% yield with >90% selectivity.

### Example 55

A 300 mL Parr autoclave was charged under nitrogen atmosphere with Pd(OAc)₂ (11.2 mg, 0.05 mmol), bisphosphorus ligand L5 of Formula III (20.7 mg, 0.04 mmol), methanesulfonic acid (10.4 μL, 0.16 mmol), butanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 80° C. and stirred for 6 hours. After completion, the system was cooled to room temperature. NMR analysis indicated butyl acrylate was obtained in 85% yield with >90% selectivity.

### Example 56

A 300 mL Parr autoclave was charged under nitrogen atmosphere with Pd(hfacac)₂ (3.1 mg, 0.01 mmol), bisphosphorus ligand L5 of Formula III (20.7 mg, 0.04 mmol), methanesulfonic acid (10.4 μL, 0.16 mmol), octanol (10 mL), n-hexane (10 mL), and a stir bar. The system was purged with acetylene gas and then charged with acetylene (62 mmol) via flow meter under cooling, followed by pressurization with carbon monoxide to 4 MPa.

The reaction mixture was rapidly heated to 120° C. and stirred for 4 hours. After completion, the system was cooled to room temperature. NMR analysis indicated octyl acrylate was obtained in 90% yield with >90% selectivity.

### Example 57

A 300 mL Parr autoclave was charged under nitrogen atmosphere with Pd(CH₃CN)₄(BF₄)₂ (4.4 mg, 0.01 mmol), bisphosphorus ligand L5 of Formula III (20.7 mg, 0.04 mmol), methanesulfonic acid (10.4 μL, 0.16 mmol), methanol (10 mL), 1,4-dioxane (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 100° C. and stirred for 5 hours. After completion, the system was cooled to room temperature. NMR analysis indicated methyl acrylate was obtained in 90% yield with >90% selectivity.

### Example 58

A 300 mL Parr autoclave was charged under nitrogen atmosphere with Pd₂(dba)₃ (4.6 mg, 0.005 mmo), bisphosphorus ligand L5 of Formula III (10.4 mg, 0.02 mmol), p-toluenesulfonic acid (17.5 mg, 0.10 mmol), methanol (10 mL), THF (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 100° C. and stirred for 5 hours. After completion, the system was cooled to room temperature. NMR analysis indicated methyl acrylate was obtained in 90% yield with >90% selectivity.

### Example 59

A 300 mL Parr autoclave was charged under nitrogen atmosphere with Pd(OAc)₂ (11.2 mg, 0.05 mmol), bisphosphorus ligand L5 of Formula III (20.7 mg, 0.04 mmol), methanesulfonic acid (10.4 μL, 0.16 mmol), methanol (10 mL), toluene (10 mL), and a stir bar. The system was purged with acetylene gas and then charged with acetylene (62 mmol) via flow meter under cooling, followed by pressurization with carbon monoxide to 4 MPa. The reaction mixture was stirred at rt for 12 hours. After completion, NMR analysis indicated methyl acrylate was obtained in 87% yield with >90% selectivity.

### Example 60

A 300 mL Parr autoclave was charged under nitrogen atmosphere with Pd(dba)₂ (5.7 mg, 0.01 mmol), bisphosphorus ligand L5 of Formula III (20.7 mg, 0.04 mmol), trifluoroacetic acid (12 μL, 0.16 mmol), methanol (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 100° C. and stirred for 5 hours. After completion, the system was cooled to room temperature. NMR analysis indicated methyl acrylate was obtained in 84% yield with >90% selectivity.

### Category IV: Application of Formula IV

### Example 61

A 300 mL Parr autoclave was charged under nitrogen atmosphere with Pd(OAc)₂ (2.2 mg, 0.01 mmol), bisphosphorus ligand L1 of Formula IV (16.3 mg, 0.04 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 90% yield with >90% selectivity.

### Example 62

A 300 mL Parr autoclave was charged under nitrogen atmosphere with Pd(OAc)₂ (2.2 mg, 0.01 mmol), bisphosphorus ligand L2 of Formula IV (16.9 mg, 0.04 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 85% yield with >90% selectivity.

### Example 63

A 300 mL Parr autoclave was charged under nitrogen atmosphere with Pd(OAc)₂ (2.2 mg, 0.01 mmol), bisphosphorus ligand L3 of Formula IV (17.4 mg, 0.04 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 88% yield with >90% selectivity.

### Example 64

A 300 mL Parr autoclave was charged under nitrogen atmosphere with Pd(OAc)₂ (2.2 mg, 0.01 mmol), bisphosphorus ligand L4 of Formula IV (19.6 mg, 0.04 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 83% yield with >90% selectivity.

### Example 65

A 300 mL Parr autoclave was charged under nitrogen atmosphere with Pd(OTFA)₂ (4.0 mg, 0.01 mmol), bisphosphorus ligand L5 of Formula IV (15.3 mg, 0.04 mmol), p-toluenesulfonic acid (17.2 mg, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 93% yield with >90% selectivity.

### Example 66

A 300 mL Parr autoclave was charged under nitrogen atmosphere with Pd(OPiv)₂ (3.1 mg, 0.01 mmol), bisphosphorus ligand L6 of Formula IV (16.5 mg, 0.04 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 6 hours. After completion, the system was cooled to room temperature. NMR analysis indicated methyl acrylate was obtained in 81% yield with >90% selectivity.

### Example 67

A 300 mL Parr autoclave was charged under nitrogen atmosphere with Pd₂(dba)₃ (4.6 mg, 0.005 mmol), bisphosphorus ligand L7 of Formula IV (16.5 mg, 0.04 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 73% yield with >90% selectivity.

### Example 68

A 300 mL Parr autoclave was charged under nitrogen atmosphere with PdCl₂ (1.8 mg, 0.01 mmol), bisphosphorus ligand L8 of Formula IV (16.6 mg, 0.04 mmol), trifluoromethanesulfonic acid (14.2 μL, 0.16 mmol), methanol (10 mL), THF (10 mL), and a stir bar. The system was purged with acetylene gas and then charged with acetylene (62 mmol) via flow meter under cooling, followed by pressurization with carbon monoxide to 6 MPa. The reaction mixture was rapidly heated to 120° C. and stirred for 6 hours. After completion, the system was cooled to room temperature. NMR analysis indicated methyl acrylate was obtained in 78% yield with >90% selectivity.

### Example 69

A 300 mL Parr autoclave was charged under nitrogen atmosphere with Pd(OAc)₂ (2.2 mg, 0.01 mmol), bisphosphorus ligand L9 of Formula IV (10.8 mg, 0.02 mmol), dodecylbenzenesulfonic acid (26.1 mg, 0.08 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated methyl acrylate was obtained in 65% yield with >90% selectivity.

### Example 70

A 300 mL Parr autoclave was charged under nitrogen atmosphere with Pd(hfacac)₂ (3.1 mg, 0.01 mmol), bisphosphorus ligand L10 of Formula IV (23.0 mg, 0.04 mmol), methanesulfonic acid (10.4 μL, 0.16 mmol), octanol (10 mL), n-hexane (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated octyl acrylate was obtained in 81% yield with >90% selectivity.

### Example 71

A 300 mL Parr autoclave was charged under nitrogen atmosphere with Pd(CH₃CN)₄(BF₄)₂ (4.4 mg, 0.01 mmol), bisphosphorus ligand L11 of Formula IV (17.0 mg, 0.04 mmol), methanesulfonic acid (10.4 μL, 0.16 mmol), methanol (10 mL), 1,4-dioxane (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated methyl acrylate was obtained in 68% yield with >90% selectivity.

### Example 72

A 300 mL Parr autoclave was charged under nitrogen atmosphere with Pd(OAc)₂ (11.2 mg, 0.05 mmol), bisphosphorus ligand L12 of Formula IV (8.9 mg, 0.02 mmol), methanesulfonic acid (10.4 μL, 0.16 mmol), butanol (10 mL), THF (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 8 hours. After completion, the system was cooled to room temperature. NMR analysis indicated methyl acrylate was obtained in 78% yield with >90% selectivity.

### Example 73

A 300 mL Parr autoclave was charged under nitrogen atmosphere with Pd(OAc)₂ (2.2 mg, 0.01 mmol), bisphosphorus ligand L13 of Formula IV (21.3 mg, 0.04 mmol), p-toluenesulfonic acid (17.5 mg, 0.10 mmol), methanol (10 mL), THF (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 82% yield with >90% selectivity.

### Example 74

A 300 mL Parr autoclave was charged under nitrogen atmosphere with Pd(OAc)₂ (2.2 mg, 0.01 mmol), bisphosphorus ligand L14 of Formula IV (23.3 mg, 0.04 mmol), p-toluenesulfonic acid (17.5 mg, 0.10 mmol), methanol (10 mL), THF (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 100° C. and stirred for 6 hours. After completion, the system was cooled to room temperature. NMR analysis indicated methyl acrylate was obtained in 85% yield with >90% selectivity.

### Example 75

A 300 mL Parr autoclave was charged under nitrogen atmosphere with Pd(dba)₂ (0.6 mg, 0.001 mmol), bisphosphorus ligand L15 of Formula IV (1.5 mg, 0.004 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 80° C. and stirred for 10 hours. After completion, the system was cooled to room temperature. NMR analysis indicated methyl acrylate was obtained in 84% yield with >90% selectivity.

### Example 76

A 300 mL Parr autoclave was charged under nitrogen atmosphere with Pd(dba)₂ (5.7 mg, 0.01 mmol), bisphosphorus ligand L5 of Formula IV (15.3 mg, 0.04 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture stirred at rt for 16 hours. After completion, NMR analysis indicated methyl acrylate was obtained in 80% yield with >90% selectivity.

### Category V: Synthesis and Application of Formula V

### Example 77 Preparation of Ligand Represented by Formula V Using L1

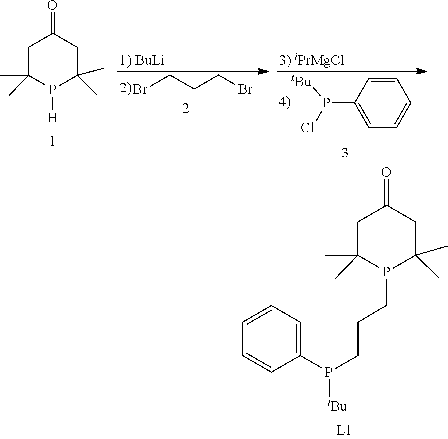

In a 100 mL three-necked flask equipped with a thermometer and magnetic stirrer, cyclic phosphine compound 1 (1.72 g, 10 mmol) was dissolved in anhydrous tetrahydrofuran (50 mL) under nitrogen atmosphere. The solution was cooled to −78° C., and n-butyllithium (6.3 mL, 1.6 M in hexane) was added dropwise via syringe. After complete addition, the reaction mixture was warmed to room temperature and stirred for 1 hour. The solution was recooled to −78° C., and a solution of 1,3-dibromopropane 2 (2.02 g, 10 mmol) in anhydrous THF (10 mL) was added dropwise. The reaction was then allowed to warm to room temperature and stirred for 5 hours. The mixture was cooled to −20° C. (ice-salt bath), and isopropylmagnesium chloride (5 mL, 2 M in THF) was added dropwise. After stirring at room temperature for 1 hour, chlorophosphine 3 (2.0 g, 10 mmol) was introduced. The reaction was stirred overnight at room temperature. The reaction was quenched with degassed water (2 mL), and the solvent was removed under reduced pressure. The residue was treated with oxygen-free water and extracted with diethyl ether. The combined organic layers were dried over anhydrous magnesium sulfate, filtered, and concentrated. The crude product was recrystallized from methanol to afford the target compound as a white to pale yellow solid powder.

Partial ligand data are as follows:

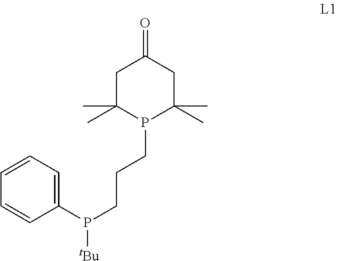 [SMILES: CC(C)(C)P(CCCP1C(C)(C)CC(=O)CC1(C)C)c1ccccc1]

¹H NMR (400 MHz, CDCl₃) δ 7.46-7.35 (m, 2H), 7.33-7.30 (m, 3H), 2.87-2.85 (m, 2H), 2.31-2.27 (m, 2H), 1.45-1.38 (m, 6H), 1.28-0.84 (m, 21H).

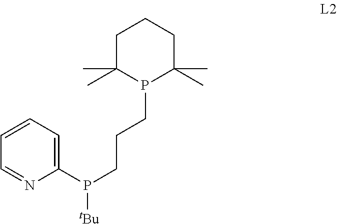 [SMILES: CC(C)(C)P(CCCP1C(C)(C)CCCC1(C)C)c1ccccn1]

¹H NMR (400 MHz, CDCl₃) δ 8.55-8.53 (m, 1H), 7.52-7.47 (m, 1H), 7.42-7.40 (m, 1H), 7.13-7.10 (m, 1H), 1.47-1.38 (m, 12H), 1.13-0.97 (m, 21H).

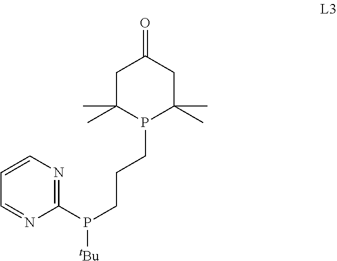 [SMILES: CC(C)(C)P(CCCP1C(C)(C)CC(=O)CC1(C)C)c1ncccn1]

¹H NMR (400 MHz, CDCl₃) δ 8.88-8.85 (m, 2H), 7.66-7.63 (m, 1H), 2.87-2.85 (m, 2H), 2.31-2.27 (m, 2H), 1.45-1.38 (m, 6H), 1.13-0.97 (m, 21H).

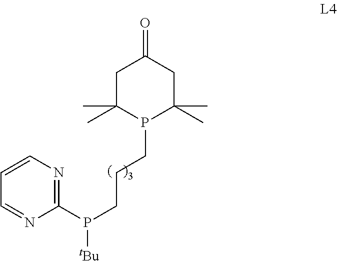

¹H NMR (400 MHz, CDCl₃) δ 8.88-8.85 (m, 2H), 7.66-7.63 (m, 1H), 2.87-2.85 (m, 2H), 2.31-2.27 (m, 2H), 1.45-1.38 (m, 4H), 1.13-0.97 (m, 27H).

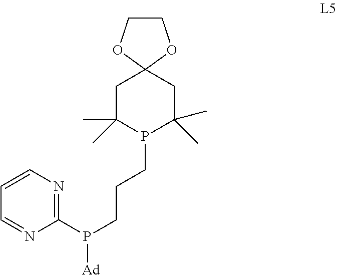

¹H NMR (400 MHz, CDCl₃) δ 8.88-8.85 (m, 2H), 7.66-7.63 (m, 1H), 4.05-3.97 (m, 4H), 2.45-2.38 (m, 4H), 2.11-2.03 (m, 2H), 2.03-1.97 (m, 3H), 1.73-1.67 (m, 12H), 1.38-0.94 (m, 12H).

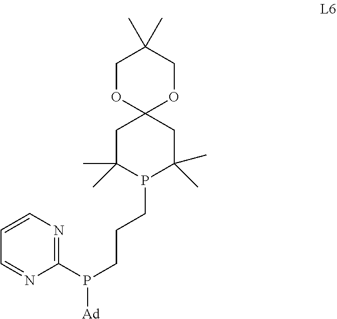

¹H NMR (400 MHz, CDCl₃) δ 8.88-8.85 (m, 2H), 7.66-7.63 (m, 1H), 4.00-3.94 (m, 4H), 2.45-2.38 (m, 4H), 2.11-2.03 (m, 2H), 2.03-1.97 (m, 3H), 1.73-1.67 (m, 12H), 1.38-0.94 (m, 16H), 0.90-0.86 (m, 6H).

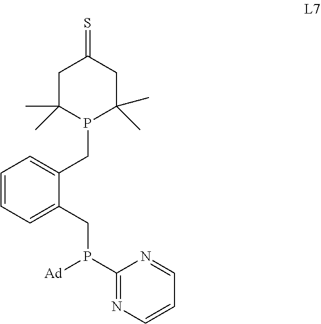

¹H NMR (400 MHz, CDCl₃) δ 8.85-8.82 (m, 2H), 7.51-7.41 (m, 1H), 7.30 (t, J=8.0 Hz, 1H), 6.97-6.89 (m, 2H), 6.70-6.57 (m, 1H), 3.45-3.34 (m, 4H), 2.40-2.18 (m, 4H), 2.03-1.97 (m, 3H), 1.73-1.67 (m, 12H), 1.38-0.94 (m, 12H).

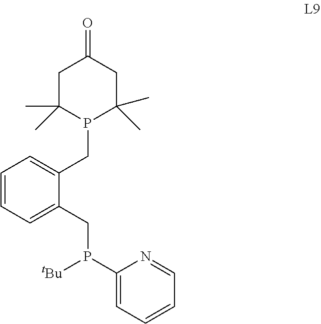 [SMILES: CC(C)(C)P(Cc1ccccc1CP1C(C)(C)CC(=O)CC1(C)C)c1ccccn1]

¹H NMR (400 MHz, CDCl₃) δ 8.85-8.82 (m, 2H), 7.51-7.41 (m, 2H), 7.30 (t, J=8.0 Hz, 1H), 6.94-6.89 (m, 2H), 6.70-6.57 (m, 1H), 3.45-3.34 (m, 4H), 2.40-2.18 (m, 4H), 1.38-0.94 (m, 21H).

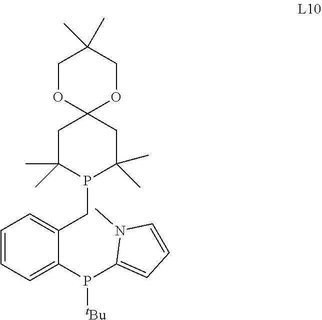 [SMILES: Cn1cccc1P(c1ccccc1CP1C(C)(C)CC2(CC1(C)C)OCC(C)(C)CO2)C(C)(C)C]

¹H NMR (400 MHz, CDCl₃) δ 7.51-7.41 (m, 2H), 6.98-6.92 (m, 3H), 6.20-6.16 (m, 2H), 3.86-3.84 (m, 4H), 3.45-3.42 (m, 2H), 2.40-2.18 (m, 4H), 1.38-0.94 (m, 21H), 0.97-0.90 (m, 6H).

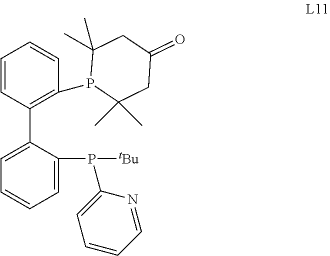 [SMILES: CC(C)(C)P(c1ccccn1)c1ccccc1-c1ccccc1P1C(C)(C)CC(=O)CC1(C)C]

¹H NMR (400 MHz, CDCl₃) δ 8.65-8.62 (m, 1H), 7.76-7.30 (m, 11H), 2.86-2.83 (m, 2H), 2.25-2.23 (m, 2H), 1.20-0.94 (m, 21H).

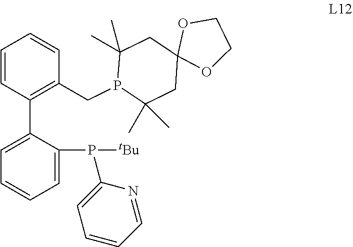 [SMILES: CC(C)(C)P(c1ccccn1)c1ccccc1-c1ccccc1CP1C(C)(C)CC2(CC1(C)C)OCCO2]

¹H NMR (400 MHz, CDCl₃) δ 8.65-8.62 (m, 1H), 7.86 (d, J=8.0 Hz, 1H), 7.58-7.22 (m, 10H), 3.96-3.90 (m, 4H), 2.98 (d, J=14.5 Hz, 2H), 2.04 (t, J=14.0 Hz, 2H), 1.94-1.74 (m, 2H), 1.53-1.31 (m, 21H).

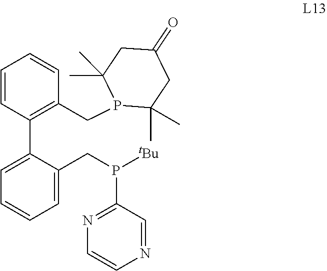 [SMILES: CC(C)(C)P(Cc1ccccc1-c1ccccc1CP1C(C)(C)CC(=O)CC1(C)C)c1cnccn1]

¹H NMR (400 MHz, CDCl₃) δ 8.87-8.71 (m, 5H), 7.45-7.22 (m, 6H), 3.28-2.23 (m, 4H), 2.91-2.87 (m, 2H), 2.10-2.09 (m, 2H), 1.53-1.31 (m, 21H).

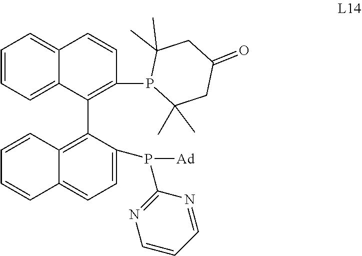

¹H NMR (400 MHz, CDCl₃) δ 8.87-8.71 (m, 3H), 8.25-8.04 (m, 4H), 7.65-7.43 (m, 6H), 2.86-2.83 (m, 2H), 2.25-2.23 (m, 2H), 2.03-1.97 (m, 3H), 1.73-1.67 (m, 12H), 1.38-0.94 (m, 12H).

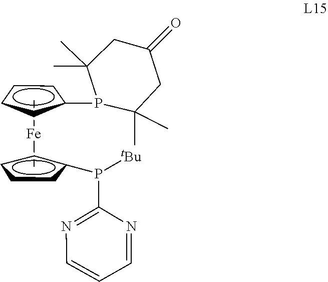

¹H NMR (400 MHz, CDCl₃) δ 8.64-8.58 (m, 2H), 7.50-7.48 (m, 1H), 4.63-4.58 (m, 2H), 4.23-4.19 (m, 6H), 2.86-2.83 (m, 2H), 2.25-2.23 (m, 2H), 1.53-1.31 (m, 21H).

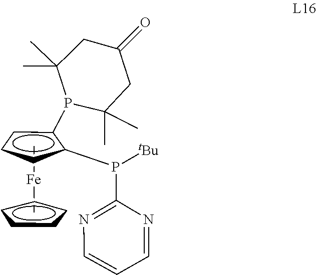

¹H NMR (400 MHz, CDCl₃) δ 8.64-8.58 (m, 2H), 7.50-7.48 (m, 1H), 4.52-4.48 (m, 2H), 4.23-4.19 (m, 5H), 2.86-2.83 (m, 2H), 2.25-2.23 (m, 2H), 1.53-1.31 (m, 21H).

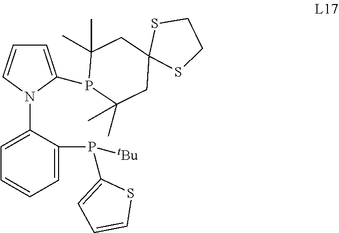 [SMILES: CC(C)(C)P(c1cccs1)c1ccccc1-n1cccc1P1C(C)(C)CC2(CC1(C)C)SCCS2]

¹H NMR (400 MHz, CDCl₃) δ 7.74-7.68 (m, 2H), 7.39-7.02 (m, 1H), 4.52-4.48 (m, 5H), 6.52-6.46 (m, 2H), 2.93-2.89 (m, 4H), 2.86-2.83 (m, 2H), 2.25-2.23 (m, 2H), 1.34-1.28 (m, 21H).

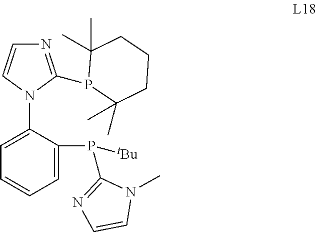 [SMILES: Cn1ccnc1P(c1ccccc1-n1ccnc1P1C(C)(C)CCCC1(C)C)C(C)(C)C]

¹H NMR (400 MHz, CDCl₃) δ 7.64-7.52 (m, 4H), 7.21-7.16 (m, 3H), 6.87 (d, J=7.6 Hz, 1H), 3.84 (s, 3H), 1.93-1.89 (m, 2H), 1.78-1.73 (m, 2H), 1.52-1.48 (m, 2H), 1.34-1.18 (m, 21H).

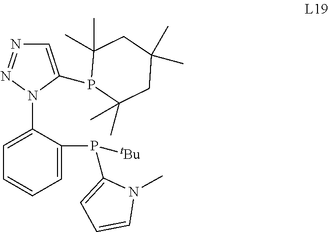 [SMILES: Cn1cccc1P(c1ccccc1-n1nncc1P1C(C)(C)CC(C)(C)CC1(C)C)C(C)(C)C]

¹H NMR (400 MHz, CDCl₃) δ 8.64 (s, 1H), 7.56-7.18 (m, 5H), 6.21-6.16 (m, 2H), 3.95 (s, 3H), 1.34-1.18 (m, 27H).

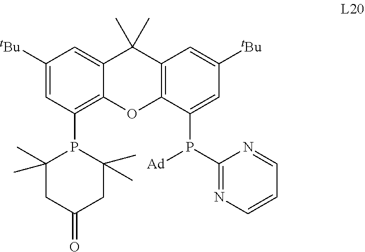

¹H NMR (400 MHz, CDCl₃) δ 8.64-8.58 (m, 2H), 7.50-7.48 (m, 1H), 7.23-7.18 (m, 4H), 3.95 (s, 3H), 2.86-2.83 (m, 2H), 2.25-2.23 (m, 2H), 2.03-1.97 (m, 3H), 1.73-1.64 (m, 18H), 1.34-1.18 (m, 30H).

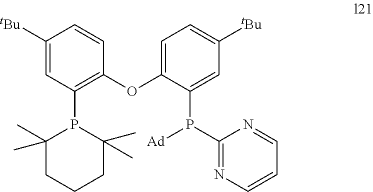

¹H NMR (400 MHz, CDCl₃) δ 8.73-8.68 (m, 2H), 7.53-7.48 (m, 3H), 7.32 (s, 2H), 6.95-6.90 (m, 2H), 2.03-1.97 (m, 3H), 1.93-1.89 (m, 2H), 1.76-1.64 (m, 14H), 1.52-1.48 (m, 2H), 1.34-1.18 (m, 30H).

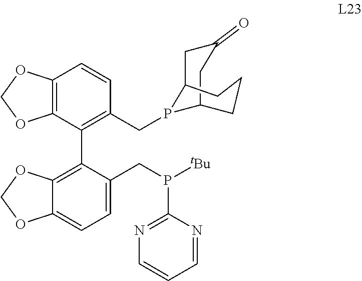 [SMILES: CC(C)(C)P(Cc1ccc2c(c1-c1c(CP3C4CCCC3CC(=O)C4)ccc3c1OCO3)OCO2)c1ncccn1]

¹H NMR (400 MHz, CDCl₃) δ 8.83-8.77 (m, 2H), 7.65-7.62 (m, 1H), 7.03-6.97 (m, 4H), 6.07 (s, 4H), 3.03-2.99 (m, 4H), 2.56-2.43 (m, 4H), 1.67-1.65 (m, 2H), 1.26-1.20 (m, 6H), 1.12-1.08 (m, 9H).

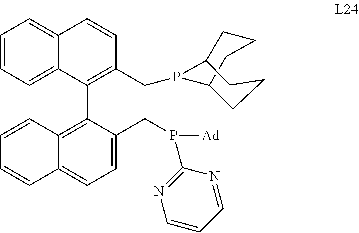

¹H NMR (400 MHz, CDCl₃) δ 8.90-8.77 (m, 4H), 8.10-8.09 (m, 2H), 7.89-7.85 (m, 2H), 7.63-7.34 (m, 5H), 7.10 (d, J=7.6 Hz, 2H), 3.03-2.99 (m, 4H), 2.06-2.03 (m, 3H), 1.80-1.65 (m, 12H), 1.26-1.16 (m, 14H).

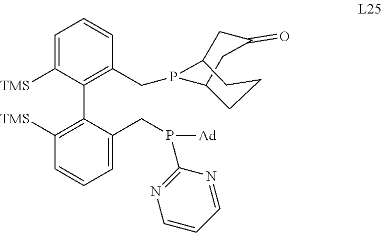

¹H NMR (400 MHz, CDCl₃) δ 8.86-8.80 (m, 2H), 7.65-7.36 (m, 7H), 3.03-2.99 (m, 4H), 2.45-2.38 (m, 4H), 2.06-2.03 (m, 3H), 1.80-1.65 (m, 14H), 1.43-1.38 (m, 2H), 1.12-1.08 (m, 2H), 0.12-0.09 (m, 18H).

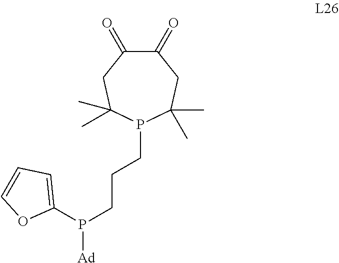

¹H NMR (400 MHz, CDCl₃) δ 7.79-7.75 (m, 1H), 6.98-6.96 (m, 1H), 6.52-4.49 (m, 1H), 2.45-2.43 (m, 4H), 2.06-2.03 (m, 3H), 1.80-1.65 (m, 12H), 1.48-1.38 (m, 4H), 1.18-1.10 (m, 14H).

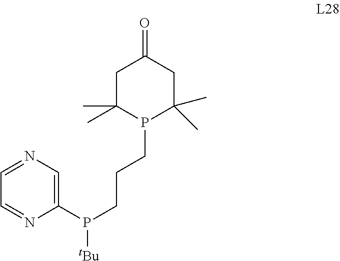 [SMILES: CC(C)(C)P(CCCP1C(C)(C)CC(=O)CC1(C)C)c1cnccn1]

¹H NMR (400 MHz, CDCl₃) δ 8.82-8.74 (m, 3H), 2.40-2.18 (m, 4H), 2.01-1.86 (m, 4H), 1.38-0.94 (m, 23H).

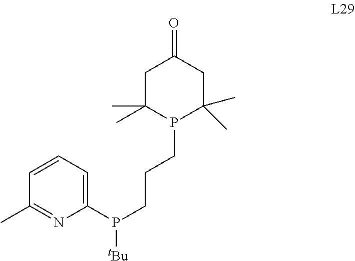

¹H NMR (400 MHz, CDCl₃) δ 7.68-7.62 (m, 1H), 7.23-7.10 (m, 2H), 2.61 (s, 3H), 2.40-2.18 (m, 4H), 2.01-1.86 (m, 4H), 1.38-0.94 (m, 23H).

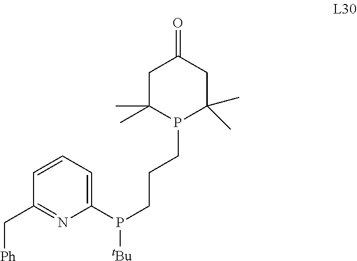 [SMILES: CC(C)(C)P(CCCP1C(C)(C)CC(=O)CC1(C)C)c1cccc(Cc2ccccc2)n1]

¹H NMR (400 MHz, CDCl₃) δ 7.86-7.82 (m, 1H), 7.45-7.41 (m, 1H), 7.35-7.25 (m, 5H), 7.10-7.06 (m, 1H), 4.33 (s, 2H), 2.40-2.18 (m, 4H), 2.01-1.86 (m, 4H), 1.38-0.94 (m, 23H).

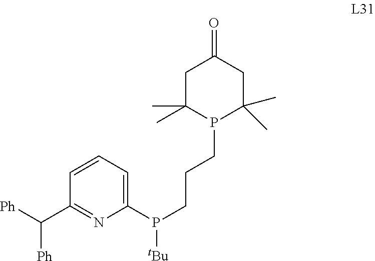 [SMILES: CC(C)(C)P(CCCP1C(C)(C)CC(=O)CC1(C)C)c1cccc(C(c2ccccc2)c2ccccc2)n1]

¹H NMR (400 MHz, CDCl₃) δ 7.89-7.842 (m, 1H), 7.35-7.22 (m, 9H), 7.14-7.10 (m, 2H), 4.36 (s, 1H), 2.40-2.18 (m, 4H), 2.01-1.86 (m, 4H), 1.38-0.94 (m, 23H).

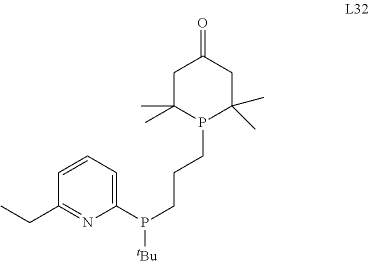 [SMILES: CCc1cccc(P(CCCP2C(C)(C)CC(=O)CC2(C)C)C(C)(C)C)n1]

¹H NMR (400 MHz, CDCl₃) δ 7.68-7.62 (m, 1H), 7.43-7.39 (m, 1H), 7.13-7.10 (m, 1H), 2.58 (t, J=7.2 Hz, 1H), 2.40-2.18 (m, 4H), 2.01-1.86 (m, 4H), 1.38-0.90 (m, 26H).

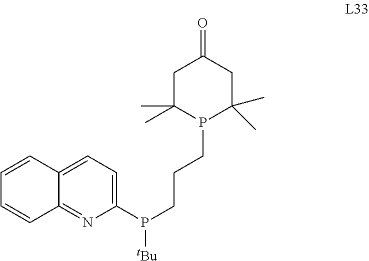 [SMILES: CC(C)(C)P(CCCP1C(C)(C)CC(=O)CC1(C)C)c1ccc2ccccc2n1]

¹H NMR (400 MHz, CDCl₃) δ 8.55-8.43 (m, 1H), 8.00-7.94 (m, 2H), 7.68-7.53 (m, 3H), 2.40-2.18 (m, 4H), 2.01-1.86 (m, 4H), 1.38-0.90 (m, 23H).

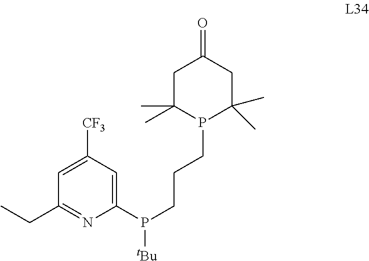 [SMILES: CCc1cc(C(F)(F)F)cc(P(CCCP2C(C)(C)CC(=O)CC2(C)C)C(C)(C)C)n1]

¹H NMR (400 MHz, CDCl₃) δ 7.62 (s, 1H), 7.13-7.10 (m, 1H), 3.23 (t, J=7.2 Hz, 1H), 2.40-2.18 (m, 4H), 2.04-1.86 (m, 4H), 1.38-0.90 (m, 26H).

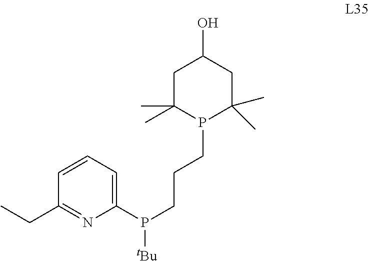 [SMILES: CCc1cccc(P(CCCP2C(C)(C)CC(O)CC2(C)C)C(C)(C)C)n1]

¹H NMR (400 MHz, CDCl₃) δ 7.68-7.62 (m, 1H), 7.43-7.39 (m, 1H), 7.13-7.10 (m, 1H), 3.52-3.48 (m, 1H), 3.01 (t, J=7.2 Hz, 2H), 1.87-1.67 (m, 4H), 1.48-1.39 (m, 4H), 1.38-0.90 (m, 26H).

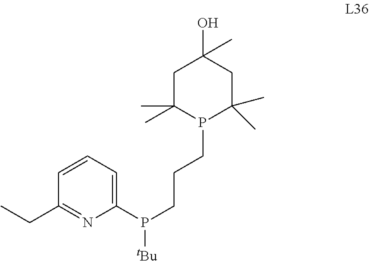 [SMILES: CCc1cccc(P(CCCP2C(C)(C)CC(C)(O)CC2(C)C)C(C)(C)C)n1]

¹H NMR (400 MHz, CDCl₃) δ 7.68-7.62 (m, 1H), 7.43-7.39 (m, 1H), 7.13-7.10 (m, 1H), 2.99 (t, J=7.2 Hz, 2H), 1.87-1.67 (m, 4H), 1.48-1.39 (m, 4H), 1.38-0.90 (m, 29H).

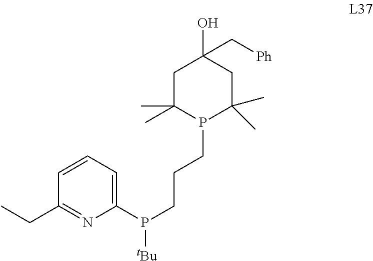 [SMILES: CCc1cccc(P(CCCP2C(C)(C)CC(O)(Cc3ccccc3)CC2(C)C)C(C)(C)C)n1]

¹H NMR (400 MHz, CDCl₃) δ 7.71-7.67 (m, 1H), 7.40-7.36 (m, 1H), 7.26-7.19 (m, 5H), 7.12-7.08 (m, 1H), 3.06 (d, J=11.2 Hz, 1H), 3.00 (d, J=11.2 Hz, 1H), 2.96 (t, J=7.2 Hz, 2H), 1.89-1.70 (m, 4H), 1.48-1.39 (m, 4H), 1.38-0.90 (m, 26H).

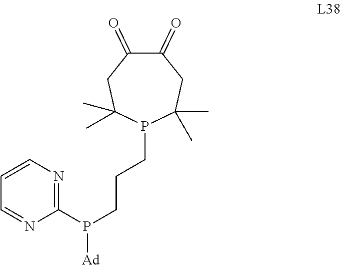

¹H NMR (400 MHz, CDCl₃) δ 8.85-8.82 (m, 2H), 7.68-7.65 (m, 1H), 2.45-2.43 (m, 4H), 2.06-2.03 (m, 7H), 1.80-1.65 (m, 12H), 1.48-1.38 (m, 4H), 1.18-1.10 (m, 14H).

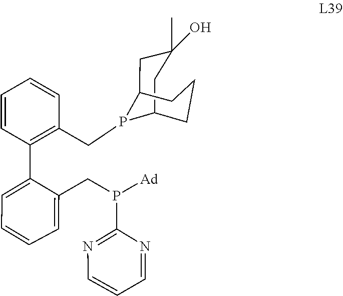

¹H NMR (400 MHz, CDCl₃) δ 8.85-8.82 (m, 2H), 7.78-7.65 (m, 3H), 7.46-7.38 (m, 6H), 2.86-2.83 (m, 2H), 2.53-2.49 (m, 2H), 2.04-2.00 (m, 3H), 1.82-1.72 (m, 12H), 1.20-1.02 (m, 15H).

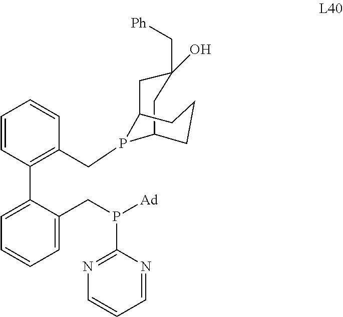

¹H NMR (400 MHz, CDCl₃) δ 8.87-8.84 (m, 2H), 7.76-7.66 (m, 3H), 7.46-7.38 (m, 6H), 7.25-7.16 (m, 5H), 3.03 (d, J=10.8 Hz, 1H), 2.96 (d, J=10.8 Hz, 1H), 2.86-2.83 (m, 2H), 2.53-2.49 (m, 2H), 2.04-2.00 (m, 3H), 1.82-1.72 (m, 12H), 1.20-1.02 (m, 12H).

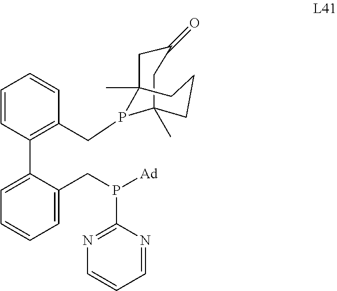

¹H NMR (400 MHz, CDCl₃) δ 8.86-8.80 (m, 2H), 7.75-7.66 (m, 3H), 7.46-7.38 (m, 6H), 3.03-2.99 (m, 4H), 2.45-2.38 (m, 4H), 2.06-2.03 (m, 3H), 1.80-1.65 (m, 12H), 1.43-1.38 (m, 4H), 1.12-1.08 (m, 8H).

### Example 78

A 300 mL Parr autoclave was charged under nitrogen atmosphere with Pd(OAc)₂ (2.2 mg, 0.01 mmol), bisphosphorus ligand L1 of Formula V (15.1 mg, 0.04 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 78% yield with >90% selectivity.

### Example 79

A 300 mL Parr autoclave was charged under nitrogen atmosphere with Pd(OAc)₂ (2.2 mg, 0.01 mmol), bisphosphorus ligand L2 of Formula V (14.6 mg, 0.04 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 82% yield with >90% selectivity.

### Example 80

A 300 mL Parr autoclave was charged under nitrogen atmosphere with Pd(OTFA)₂ (4.0 mg, 0.01 mmol), bisphosphorus ligand L3 of Formula V (15.2 mg, 0.04 mmol), methanesulfonic acid (6.5 μL, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 98% yield with >90% selectivity.

### Example 81

A 300 mL Parr autoclave was charged under nitrogen atmosphere with Pd(OPiv)₂ (3.1 mg, 0.01 mmol), bisphosphorus ligand L4 of Formula V (16.2 mg, 0.04 mmol), methanesulfonic acid (10.4 μL, 0.16 mmol), methanol (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 6 hours. After completion, the system was cooled to room temperature. NMR analysis indicated methyl acrylate was obtained in 87% yield with >90% selectivity.

### Example 82

A 300 mL Parr autoclave was charged under nitrogen atmosphere with Pd₂(dba)₃ (4.6 mg, 0.005 mmol), bisphosphorus ligand L5 of Formula V (20.1 mg, 0.04 mmol), methanesulfonic acid (10.4 μL, 0.16 mmol), ethanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated ethyl acrylate was obtained in 83% yield with >90% selectivity.

### Example 83

A 300 mL Parr autoclave was charged under nitrogen atmosphere with Pd(CH₃CN)₄(BF₄)₂ (4.4 mg, 0.01 mmol), bisphosphorus ligand L6 of Formula V (21.8 mg, 0.04 mmol), methanesulfonic acid (10.4 μL, 0.16 mmol), methanol (10 mL), 1,4-dioxane (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated methyl acrylate was obtained in 80% yield with >90% selectivity.

### Example 84

A 300 mL Parr autoclave was charged under nitrogen atmosphere with Pd(dba)₂ (5.7 mg, 0.01 mmol), bisphosphorus ligand L7 of Formula V (24.1 mg, 0.04 mmol), trifluoromethanesulfonic acid (14.2 μL, 0.16 mmol), butanol (10 mL), tetrahydrofuran (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated butyl acrylate was obtained in 85% yield with >90% selectivity.

### Example 85

A 300 mL Parr autoclave was charged under nitrogen atmosphere with PdCl₂ (0.2 mg, 0.001 mmol), bisphosphorus ligand L9 of Formula V (1.7 mg, 0.004 mmol), dodecyl sulfonic acid (26.1 mg, 0.08 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 8 hours. After completion, the system was cooled to room temperature. NMR analysis indicated methyl acrylate was obtained in 95% yield with >90% selectivity.

### Example 86

A 300 mL Parr autoclave was charged under nitrogen atmosphere with Pd(hfacac)₂ (3.1 mg, 0.01 mmol), bisphosphorus ligand L11 of Formula V (19.6 mg, 0.04 mmol), methanesulfonic acid (10.4 μL, 0.16 mmol), methanol (20 mL), n-hexane (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated methyl acrylate was obtained in 92% yield with >90% selectivity.

### Example 87

A 300 mL Parr autoclave was charged under nitrogen atmosphere with Pd(OAc)₂ (1.1 mg, 0.005 mmol), bisphosphorus ligand L12 of Formula V (21.9 mg, 0.04 mmol), methanesulfonic acid (10.4 μL, 0.16 mmol), methanol (10 mL), THF (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 8 hours. After completion, the system was cooled to room temperature. NMR analysis indicated methyl acrylate was obtained in 83% yield with >90% selectivity.

### Example 88

A 300 mL Parr autoclave was charged under nitrogen atmosphere with Pd(OAc)₂ (2.2 mg, 0.01 mmol), bisphosphorus ligand L13 of Formula V (10.4 mg, 0.02 mmol), p-toluenesulfonic acid (17.5 mg, 0.10 mmol), methanol (10 mL), THF (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 90% yield with >90% selectivity.

### Example 89

A 300 mL Parr autoclave was charged under nitrogen atmosphere with Pd(OAc)₂ (2.2 mg, 0.01 mmol), bisphosphorus ligand L14 of Formula V (13.4 mg, 0.02 mmol), methanesulfonic acid (10.4 μL, 0.16 mmol), methanol (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 86% yield with >90% selectivity.

### Example 90

A 300 mL Parr autoclave was charged under nitrogen atmosphere with Pd(OAc)₂ (2.2 mg, 0.01 mmol), bisphosphorus ligand L15 of Formula V (20.9 mg, 0.04 mmol), trifluoroacetic acid (12 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 86% yield with >90% selectivity.

### Example 91

A 300 mL Parr autoclave was charged under nitrogen atmosphere with Pd(OAc)₂ (2.2 mg, 0.01 mmol), bisphosphorus ligand L16 of Formula V (19.1 mg, 0.04 mmol), methanesulfonic acid (10.4 μL, 0.16 mmol), octanol (10 mL), acetone (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 100° C. and stirred for 5 hours. After completion, the system was cooled to room temperature. NMR analysis indicated octyl acrylate was obtained in 80% yield with >90% selectivity.

### Example 92

A 300 mL Parr autoclave was charged under nitrogen atmosphere with Pd(OAc)₂ (2.2 mg, 0.01 mmol), bisphosphorus ligand L18 of Formula V (18.7 mg, 0.04 mmol), methanesulfonic acid (10.4 μL, 0.16 mmol), butanol (10 mL), tetrahydrofuran (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 60° C. and stirred for 8 hours. After completion, the system was cooled to room temperature. NMR analysis indicated butyl acrylate was obtained in 73% yield with >90% selectivity.

### Example 93

A 300 mL Parr autoclave was charged under nitrogen atmosphere with Pd(OAc)₂ (2.2 mg, 0.01 mmol), bisphosphorus ligand L19 of Formula V (20.0 mg, 0.04 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 75% yield with >90% selectivity.

### Example 94

A 300 mL Parr autoclave was charged under nitrogen atmosphere with Pd(OAc)₂ (2.2 mg, 0.01 mmol), bisphosphorus ligand L20 of Formula V (29.5 mg, 0.04 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was stirred at rt for 10 hours. After completion, NMR analysis indicated methyl acrylate was obtained in 70% yield with >90% selectivity.

### Example 95

A 300 mL Parr autoclave was charged under nitrogen atmosphere with Pd(OAc)₂ (2.2 mg, 0.01 mmol), bisphosphorus ligand L21 of Formula V (27.3 mg, 0.04 mmol), methanesulfonic acid (10.4 μL, 0.16 mmol), butanol (10 mL), THF (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 80° C. and stirred for 5 hours. After completion, the system was cooled to room temperature. NMR analysis indicated butyl acrylate was obtained in 82% yield with >95% selectivity.

### Example 96

A 300 mL Parr autoclave was charged under nitrogen atmosphere with Pd(OAc)₂ (2.2 mg, 0.01 mmol), bisphosphorus ligand L23 of Formula V (26.3 mg, 0.04 mmol), methanesulfonic acid (10.4 μL, 0.16 mmol), methanol (50 mL), and a stir bar. The system was purged with acetylene gas and then charged with acetylene (100 mmol) via flow meter under cooling, followed by pressurization with carbon monoxide to 6 MPa. The reaction mixture was rapidly heated to 60° C. and stirred for 10 hours. After completion, the system was cooled to room temperature. NMR analysis indicated methyl acrylate was obtained in 78% yield with >95% selectivity.

### Example 97

A 300 mL Parr autoclave was charged under nitrogen atmosphere with Pd(OAc)₂ (2.2 mg, 0.01 mmol), bisphosphorus ligand L25 of Formula V (28.4 mg, 0.04 mmol), methanesulfonic acid (10.4 μL, 0.16 mmol), methanol (50 mL), and a stir bar. The system was purged with acetylene gas and then charged with acetylene (62 mmol) via flow meter under cooling, followed by pressurization with carbon monoxide to 4 MPa. The reaction mixture was rapidly heated to 80° C. and stirred for 5 hours. After completion, the system was cooled to room temperature. NMR analysis indicated methyl acrylate was obtained in 93% yield with >95% selectivity.

### Example 98

A 300 mL Parr autoclave was charged under nitrogen atmosphere with Pd(OPiv)₂ (3.1 mg, 0.01 mmol), bisphosphorus ligand L26 of Formula V (19.0 mg, 0.04 mmol), methanesulfonic acid (10.4 μL, 0.16 mmol), methanol (50 mL), and a stir bar. The system was purged with acetylene gas and then charged with acetylene (62 mmol) via flow meter under cooling, followed by pressurization with carbon monoxide to 4 MPa. The reaction mixture was rapidly heated to 80° C. and stirred for 5 hours. After completion, the system was cooled to room temperature. NMR analysis indicated methyl acrylate was obtained in 90% yield with >95% selectivity.

### Example 99

A 300 mL Parr autoclave was charged under nitrogen atmosphere with Pd(OAc)₂ (2.2 mg, 0.01 mmol), bisphosphorus ligand L28 of Formula V (15.2 mg, 0.04 mmol), methanesulfonic acid (6.5 μL, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 90% yield with >95% selectivity.

### Example 100

A 300 mL Parr autoclave was charged under nitrogen atmosphere with Pd(OAc)₂ (2.2 mg, 0.01 mmol), bisphosphorus ligand L29 of Formula V (15.7 mg, 0.04 mmol), methanesulfonic acid (6.5 μL, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 86% yield with >90% selectivity.

### Example 101

A 300 mL Parr autoclave was charged under nitrogen atmosphere with Pd(OAc)₂ (2.2 mg, 0.01 mmol), bisphosphorus ligand L30 of Formula V (18.8 mg, 0.04 mmol), methanesulfonic acid (6.5 μL, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 80% yield with >90% selectivity.

### Example 102

A 300 mL Parr autoclave was charged under nitrogen atmosphere with Pd(OAc)₂ (2.2 mg, 0.01 mmol), bisphosphorus ligand L31 of Formula V (21.8 mg, 0.04 mmol), methanesulfonic acid (6.5 μL, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 80% yield with >90% selectivity.

### Example 103

A 300 mL Parr autoclave was charged under nitrogen atmosphere with Pd(OAc)₂ (2.2 mg, 0.01 mmol), bisphosphorus ligand L32 of Formula V (16.3 mg, 0.04 mmol), methanesulfonic acid (6.5 μL, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 90% yield with >90% selectivity.

### Example 104

A 300 mL Parr autoclave was charged under nitrogen atmosphere with Pd(OAc)₂ (2.2 mg, 0.01 mmol), bisphosphorus ligand L35 of Formula V (16.4 mg, 0.04 mmol), methanesulfonic acid (6.5 μL, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 80% yield with >95% selectivity.

### Example 105

A 300 mL Parr autoclave was charged under nitrogen atmosphere with Pd(OAc)₂ (2.2 mg, 0.01 mmol), bisphosphorus ligand L36 of Formula V (16.9 mg, 0.04 mmol), methanesulfonic acid (6.5 μL, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 84% yield with >95% selectivity.

### Example 106

A 300 mL Parr autoclave was charged under nitrogen atmosphere with Pd(OAc)₂ (2.2 mg, 0.01 mmol), bisphosphorus ligand L37 of Formula V (20.0 mg, 0.04 mmol), methanesulfonic acid (6.5 μL, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 81% yield with >95% selectivity.

### Example 107

A 300 mL Parr autoclave was charged under nitrogen atmosphere with Pd(OAc)₂ (2.2 mg, 0.01 mmol), bisphosphorus ligand L39 of Formula V (23.8 mg, 0.04 mmol), methanesulfonic acid (6.5 μL, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 89% yield with >95% selectivity.

### Example 108

A 300 mL Parr autoclave was charged under nitrogen atmosphere with Pd(OAc)₂ (2.2 mg, 0.01 mmol), bisphosphorus ligand L40 of Formula V (26.9 mg, 0.04 mmol), methanesulfonic acid (6.5 μL, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 90% yield with >95% selectivity.

### Example 109

A 300 mL Parr autoclave was charged under nitrogen atmosphere with Pd(OAc)₂ (2.2 mg, 0.01 mmol), bisphosphorus ligand L41 of Formula V (24.3 mg, 0.04 mmol), methanesulfonic acid (6.5 μL, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 92% yield with >95% selectivity.

### Category VI: Synthesis and Application of Formula VI

### Example 110 Preparation of Ligand Represented by Formula VI Using L1

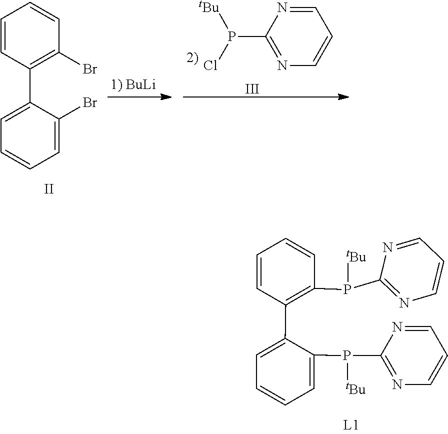

In a 100 mL three-necked flask equipped with a thermometer, magnetic stirrer, and reflux condenser, dibromide II (10 mmol) was dissolved in anhydrous tetrahydrofuran (THF, 50 mL) under nitrogen atmosphere. The solution was cooled to −78° C. using a dry ice/acetone bath, and n-butyllithium (25 mL, 1.6 M in hexane) was added dropwise via syringe. After stirring at −78° C. for 3 hours, a solution of chlorophosphine III (20 mmol) in anhydrous THF (20 mL) was added slowly. The reaction mixture was then allowed to warm to room temperature naturally and stirred overnight. Upon completion, the reaction was quenched with degassed water (2 mL). The mixture was concentrated under reduced pressure to remove the solvent, followed by the addition of oxygen-free water. The aqueous layer was extracted with diethyl ether, and the combined organic phases were dried over anhydrous magnesium sulfate. After filtration and removal of the ether, the crude product was recrystallized from methanol to afford the target compound as a white to pale yellow solid powder.

Partial ligand data are as follows:

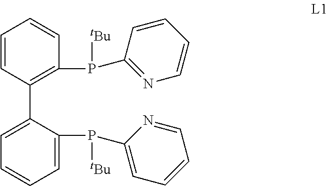 [SMILES: CC(C)(C)P(c1ccccn1)c1ccccc1-c1ccccc1P(c1ccccn1)C(C)(C)C]

¹H NMR (400 MHz, CDCl₃) δ 8.59-8.48 (m, 2H), 7.60 (d, J=8.0 Hz, 2H), 7.53 (d, J=7.6 Hz, 2H), 7.45-7.36 (m, 2H), 7.30-7.25 (m, 6H), 6.99-6.90 (m, 2H), 1.17-0.97 (m, 18H).

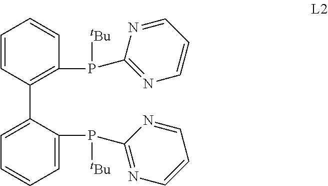 [SMILES: CC(C)(C)P(c1ncccn1)c1ccccc1-c1ccccc1P(c1ncccn1)C(C)(C)C]

¹H NMR (400 MHz, CDCl₃) δ 8.60 (d, J=7.6 Hz, 4H), 7.55 (d, J=7.6 Hz, 2H), 7.45-7.38 (m, 2H), 7.30-7.26 (m, 4H), 7.02-6.97 (m, 2H), 1.18-0.99 (m, 18H).

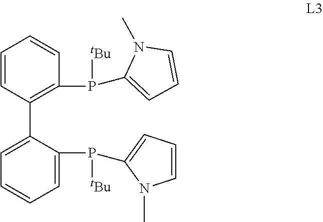 [SMILES: Cn1cccc1P(c1ccccc1-c1ccccc1P(c1cccn1C)C(C)(C)C)C(C)(C)C]

¹H NMR (400 MHz, CDCl₃) 7.45-7.38 (m, 2H), 7.30-7.26 (m, 4H), 7.02-6.97 (m, 2H), 6.75-6.67 (m, 2H), 6.55-6.50 (m, 2H), 6.38-6.30 (m, 2H), 3.55 (s, 6H), 1.20-0.99 (m, 18H).

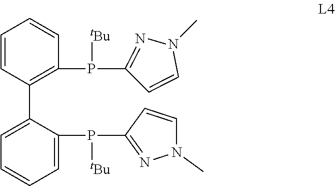 [SMILES: Cn1ccc(P(c2ccccc2-c2ccccc2P(c2ccn(C)n2)C(C)(C)C)C(C)(C)C)n1]

¹H NMR (400 MHz, CDCl₃) 7.75-7.70 (m, 2H), 7.45-7.38 (m, 2H), 7.30-7.26 (m, 4H), 7.15-7.10 (m, 2H), 6.55-6.50 (m, 2H), 3.72 (s, 3H), 3.68 (s, 3H), 1.20-0.99 (m, 18H).

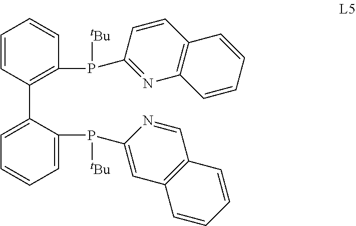 [SMILES: CC(C)(C)P(c1cc2ccccc2cn1)c1ccccc1-c1ccccc1P(c1ccc2ccccc2n1)C(C)(C)C]

¹H NMR (400 MHz, CDCl₃) 8.32-8.27 (m, 4H), 7.78-7.70 (m, 6H), 7.56-7.36 (m, 6H), 7.30-7.26 (m, 4H), 1.17-1.00 (m, 18H).

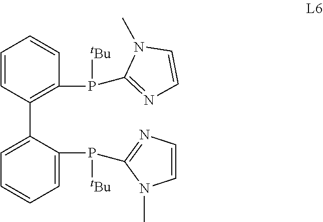 [SMILES: Cn1ccnc1P(c1ccccc1-c1ccccc1P(c1nccn1C)C(C)(C)C)C(C)(C)C]

¹H NMR (400 MHz, CDCl₃) δ 7.62 (d, J=8.0 Hz, 2H), 7.45-7.38 (m, 2H), 7.30-7.26 (m, 4H), 7.02-6.97 (m, 2H), 6.45 (d, J=8.0 Hz, 2H), 3.55 (s, 6H), 1.20-0.99 (m, 18H).

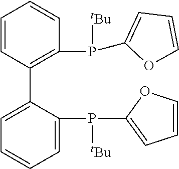 [SMILES: CC(C)(C)P(c1ccco1)c1ccccc1-c1ccccc1P(c1ccco1)C(C)(C)C]

¹H NMR (400 MHz, CDCl₃) δ 7.78-7.60 (m, 2H), 7.53 (d, J=7.6 Hz, 2H), 7.45-7.36 (m, 2H), 7.30-7.25 (m, 4H), 6.93-6.89 (m, 2H), 6.54-6.49 (m, 2H), 1.10-0.95 (m, 18H).

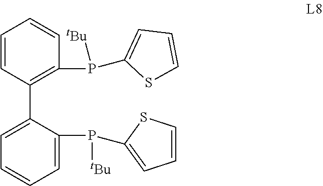 [SMILES: CC(C)(C)P(c1cccs1)c1ccccc1-c1ccccc1P(c1cccs1)C(C)(C)C]

¹H NMR (400 MHz, CDCl₃) δ 7.96-7.90 (m, 2H), 7.60-6.55 (m, 4H), 7.45-7.36 (m, 2H), 7.30-7.25 (m, 6H), 1.10-0.91 (m, 18H).

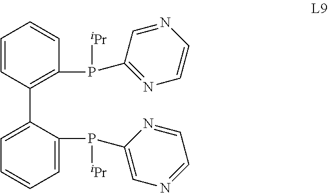 [SMILES: CC(C)P(c1cnccn1)c1ccccc1-c1ccccc1P(c1cnccn1)C(C)C]

¹H NMR (400 MHz, CDCl₃) δ 8.85-8.75 (m, 6H), 7.60-6.55 (m, 2H), 7.45-7.36 (m, 2H), 7.30-7.25 (m, 4H), 1.78-1.70 (m, 2H), 1.03-0.90 (m, 12H).

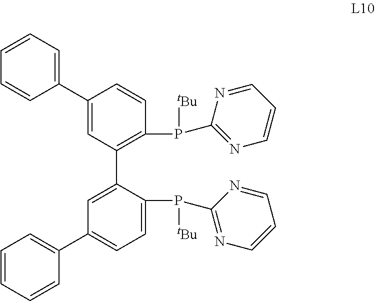 [SMILES: CC(C)(C)P(c1ncccn1)c1ccc(-c2ccccc2)cc1-c1cc(-c2ccccc2)ccc1P(c1ncccn1)C(C)(C)C]

¹H NMR (400 MHz, CDCl₃) δ 8.60 (d, J=7.6 Hz, 4H), 8.02 (s, 2H), 7.87-7.83 (m, 2H), 7.78-7.73 (m, 4H), 7.62-7.51 (m, 10H), 1.16-1.02 (m, 18H).

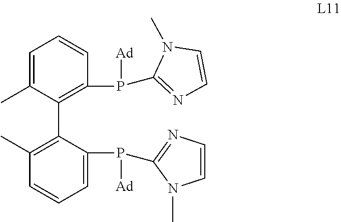

¹H NMR (400 MHz, CDCl₃) δ 7.45-7.37 (m, 4H), 7.27-7.24 (m, 2H), 7.16-7.14 (m, 2H), 6.56-6.50 (m, 2H), 3.67-3.65 (m, 6H), 2.58-2.54 (m, 6H), 2.03-1.96 (m, 6H), 1.73-1.58 (m, 24H).

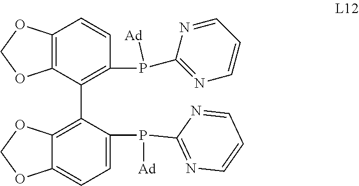

¹H NMR (400 MHz, CDCl₃) δ 8.60-8.52 (m, 4H), 7.72-7.67 (m, 2H), 7.12-7.06 (m, 4H), 6.03 (s, 4H), 2.03-1.96 (m, 6H), 1.75-1.55 (m, 24H).

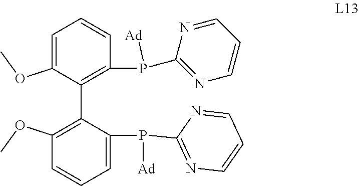

¹H NMR (400 MHz, CDCl₃) δ 8.60-8.52 (m, 4H), 7.72-7.67 (m, 4H), 7.21-7.18 (m, 2H), 7.12-7.06 (m, 2H), 3.82 (s, 6H), 2.03-1.96 (m, 6H), 1.83-1.71 (m, 24H).

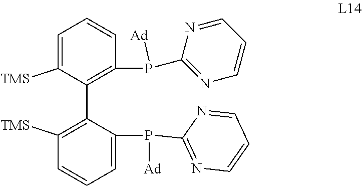

¹H NMR (400 MHz, CDCl₃) δ 8.63-8.54 (m, 4H), 7.72-7.62 (m, 4H), 7.41-7.32 (m, 2H), 7.27-7.22 (m, 2H), 2.03-1.96 (m, 6H), 1.83-1.71 (m, 24H), 0.13 (s, 18H).

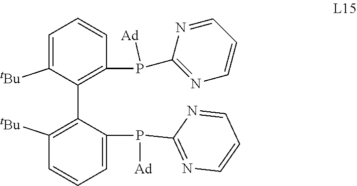

¹H NMR (400 MHz, CDCl₃) δ 8.56-8.51 (m, 4H), 7.70-7.66 (m, 4H), 7.38-7.28 (m, 4H), 2.03-1.96 (m, 6H), 1.36-1.32 (m, 18H), 1.83-1.71 (m, 24H).

### Example 111

A 300 mL Parr autoclave was charged under nitrogen atmosphere with PdCl₂ (1.8 mg, 0.01 mmol), bisphosphorus ligand L1 of Formula VI (19.4 mg, 0.04 mmol), trifluoromethanesulfonic acid (14.2 μL, 0.16 mmol), butanol (10 mL), THF (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated butyl acrylate was obtained in 83% yield with >90% selectivity.

### Example 112

A 300 mL Parr autoclave was charged under nitrogen atmosphere with Pd(OAc)₂ (2.2 mg, 0.01 mmol), bisphosphorus ligand L2 of Formula VI (9.7 mg, 0.02 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 91% yield with >90% selectivity.

### Example 113

A 300 mL Parr autoclave was charged under nitrogen atmosphere with Pd(OTFA)₂ (4.0 mg, 0.01 mmol), bisphosphorus ligand L3 of Formula VI (19.5 mg, 0.04 mmol), methanesulfonic acid (6.5 μL, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 82% yield with >90% selectivity.

### Example 114

A 300 mL Parr autoclave was charged under nitrogen atmosphere with Pd(OPiv)₂ (3.1 mg, 0.01 mmol), bisphosphorus ligand L4 of Formula VI (11.7 mg, 0.02 mmol), methanesulfonic acid (10.4 μL, 0.16 mmol), methanol (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 92% yield with >90% selectivity.

### Example 115

A 300 mL Parr autoclave was charged under nitrogen atmosphere with Pd₂(dba)₃ (4.6 mg, 0.005 mmol), bisphosphorus ligand L5 of Formula VI (9.7 mg, 0.02 mmol), p-toluenesulfonic acid (17.2 mg, 0.10 mmol), methanol (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 96% yield with >90% selectivity.

### Example 116

A 300 mL Parr autoclave was charged under nitrogen atmosphere with Pd(OAc)₂ (2.2 mg, 0.01 mmol), bisphosphorus ligand L6 of Formula VI (19.6 mg, 0.04 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 80% yield with >90% selectivity.

### Example 117

A 300 mL Parr autoclave was charged under nitrogen atmosphere with Pd(OAc)₂ (2.2 mg, 0.01 mmol), bisphosphorus ligand L7 of Formula VI (9.2 mg, 0.02 mmol), dodecyl sulfonic acid (26.1 mg, 0.08 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 57% yield with >90% selectivity.

### Example 118

A 300 mL Parr autoclave was charged under nitrogen atmosphere with Pd(hfacac)₂ (3.1 mg, 0.01 mmol), bisphosphorus ligand L8 of Formula VI (19.7 mg, 0.04 mmol), methanesulfonic acid (10.4 μL, 0.16 mmol), octanol (10 mL), n-hexane (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated octyl acrylate was obtained in 87% yield with >90% selectivity.

### Example 119

A 300 mL Parr autoclave was charged under nitrogen atmosphere with Pd(CH₃CN)₄(BF₄)₂ (4.4 mg, 0.01 mmol), bisphosphorus ligand L9 of Formula VI (18.3 mg, 0.04 mmol), methanesulfonic acid (10.4 μL, 0.16 mmol), methanol (10 mL), 1,4-dioxane (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 86% yield with >90% selectivity.

### Example 120

A 300 mL Parr autoclave was charged under nitrogen atmosphere with Pd(OAc)₂ (0.2 mg, 0.001 mmol), bisphosphorus ligand L10 of Formula VI (1.3 mg, 0.002 mmol), methanesulfonic acid (10.4 μL, 0.16 mmol), butanol (10 mL), THF (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 7 hours. After completion, the system was cooled to room temperature. NMR analysis indicated butyl acrylate was obtained in 81% yield with >90% selectivity.

### Example 121

A 300 mL Parr autoclave was charged under nitrogen atmosphere with Pd(OAc)₂ (2.2 mg, 0.01 mmol), bisphosphorus ligand L11 of Formula VI (13.5 mg, 0.02 mmol), p-toluenesulfonic acid (17.5 mg, 0.10 mmol), methanol (10 mL), THF (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 68% yield with >90% selectivity.

### Example 122

A 300 mL Parr autoclave was charged under nitrogen atmosphere with Pd(OAc)₂ (2.2 mg, 0.01 mmol), bisphosphorus ligand L12 of Formula VI (14.6 mg, 0.02 mmol), methanesulfonic acid (10.4 μL, 0.16 mmol), methanol (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 92% yield with >90% selectivity.

### Example 123

A 300 mL Parr autoclave was charged under nitrogen atmosphere with Pd(OAc)₂ (2.24 mg, 0.01 mmol), bisphosphorus ligand L14 of Formula VI (15.7 mg, 0.02 mmol), trifluoroacetic acid (12 μL, 0.16 mmol), methanol (20 mL), THF (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 75% yield with >90% selectivity.

### Example 124

A 300 mL Parr autoclave was charged under nitrogen atmosphere with Pd(OAc)₂ (2.2 mg, 0.01 mmol), bisphosphorus ligand L15 of Formula VI (15.1 mg, 0.02 mmol), methanesulfonic acid (10.4 μL, 0.16 mmol), methanol (20 mL), THF (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 82% yield with >90% selectivity.

### Example 125

A 300 mL Parr autoclave was charged under nitrogen atmosphere with Pd(OAc)₂ (2.2 mg, 0.01 mmol), bisphosphorus ligand L1 of Formula VI (19.4 mg, 0.04 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 100° C. and stirred for 5 hours. After completion, the system was cooled to room temperature. NMR analysis indicated methyl acrylate was obtained in 90% yield with >90% selectivity.

### Example 126

A 300 mL Parr autoclave was charged under nitrogen atmosphere with Pd(OAc)₂ (2.2 mg, 0.01 mmol), bisphosphorus ligand L1 of Formula VI (19.4 mg, 0.04 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 80° C. and stirred for 5 hours. After completion, the system was cooled to room temperature. NMR analysis indicated methyl acrylate was obtained in 85% yield with >90% selectivity.

### Example 127

A 300 mL Parr autoclave was charged under nitrogen atmosphere with Pd(OAc)₂ (2.2 mg, 0.01 mmol), bisphosphorus ligand L1 of Formula VI (19.4 mg, 0.04 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 40° C. and stirred for 8 hours. After completion, the system was cooled to room temperature. NMR analysis indicated methyl acrylate was obtained in 84% yield with >90% selectivity.

### Category VII: Application of Formula VII

### Example 128

A 300 mL Parr autoclave was charged under nitrogen atmosphere with Pd(OAc)₂ (2.2 mg, 0.01 mmol), bisphosphorus ligand L1 of Formula VII (7.5 mg, 0.02 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 91% yield with >90% selectivity.

### Example 129

A 300 mL Parr autoclave was charged under nitrogen atmosphere with Pd(OAc)₂ (2.2 mg, 0.01 mmol), bisphosphorus ligand L2 of Formula VII (8.0 mg, 0.02 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 88% yield with >90% selectivity.

### Example 130

A 300 mL Parr autoclave was charged under nitrogen atmosphere with Pd(OAc)₂ (2.2 mg, 0.01 mmol), bisphosphorus ligand L3 of Formula VII (7.2 mg, 0.02 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 86% yield with >90% selectivity.

### Example 131

A 300 mL Parr autoclave was charged under nitrogen atmosphere with Pd(OTFA)₂ (0.4 mg, 0.001 mmol), bisphosphorus ligand L5 of Formula VII (1.1 mg, 0.002 mmol), p-toluenesulfonic acid (17.2 mg, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 8 hours. After completion, the system was cooled to room temperature. NMR analysis indicated methyl acrylate was obtained in 86% yield with >90% selectivity.

### Example 132

A 300 mL Parr autoclave was charged under nitrogen atmosphere with Pd(OPiv)₂ (3.1 mg, 0.01 mmol), bisphosphorus ligand L7 of Formula VII (17.1 mg, 0.04 mmol), methanesulfonic acid (10.4 μL, 0.16 mmol), methanol (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 6 hours. After completion, the system was cooled to room temperature. NMR analysis indicated methyl acrylate was obtained in 92% yield with >90% selectivity.

### Example 133

A 300 mL Parr autoclave was charged under nitrogen atmosphere with Pd₂(dba)₃ (4.6 mg, 0.005 mmol), bisphosphorus ligand L9 of Formula VII (7.6 mg, 0.02 mmol), methanesulfonic acid (10.4 μL, 0.16 mmol), methanol (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 81% yield with >90% selectivity.

### Example 134

A 300 mL Parr autoclave was charged under nitrogen atmosphere with PdCl₂ (1.8 mg, 0.01 mmol), bisphosphorus ligand L10 of Formula VII (14.6 mg, 0.04 mmol), trifluoromethanesulfonic acid (14.2 μL, 0.16 mmol), ethanol (10 mL), THF (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated methyl acrylate was obtained in 80% yield with >90% selectivity.

### Example 135

A 300 mL Parr autoclave was charged under nitrogen atmosphere with Pd(OAc)₂ (2.2 mg, 0.01 mmol), bisphosphorus ligand L12 of Formula VII (9.2 mg, 0.02 mmol), methanesulfonic acid (10.4 μL, 0.16 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated methyl acrylate was obtained in 86% yield with >90% selectivity.

### Example 136

A 300 mL Parr autoclave was charged under nitrogen atmosphere with Pd(OAc)₂ (2.2 mg, 0.01 mmol), bisphosphorus ligand L13 of Formula VII (8.0 mg, 0.02 mmol), methanesulfonic acid (10.4 μL, 0.16 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated methyl acrylate was obtained in 78% yield with >90% selectivity.

### Example 137

A 300 mL Parr autoclave was charged under nitrogen atmosphere with Pd(OAc)₂ (2.2 mg, 0.01 mmol), bisphosphorus ligand L14 of Formula VII (9.2 mg, 0.02 mmol), methanesulfonic acid (10.4 μL, 0.16 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated methyl acrylate was obtained in 76% yield with >90% selectivity.

### Example 138

A 300 mL Parr autoclave was charged under nitrogen atmosphere with Pd(OAc)₂ (2.2 mg, 0.01 mmol), bisphosphorus ligand L8 of Formula VII (7.8 mg, 0.02 mmol), dodecyl sulfonic acid (26.1 mg, 0.08 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated methyl acrylate was obtained in 82% yield with >90% selectivity.

### Example 139

A 300 mL Parr autoclave was charged under nitrogen atmosphere with Pd(OAc)₂ (11.2 mg, 0.05 mmol), bisphosphorus ligand L8 of Formula VII (7.8 mg, 0.02 mmol), methanesulfonic acid (10.4 μL, 0.16 mmol), butanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 80° C. and stirred for 6 hours. After completion, the system was cooled to room temperature. NMR analysis indicated butyl acrylate was obtained in 91% yield with >90% selectivity.

### Example 140

A 300 mL Parr autoclave was charged under nitrogen atmosphere with Pd(OAc)₂ (1.1 mg, 0.005 mmol), bisphosphorus ligand L8 of Formula VII (0.8 mg, 0.002 mmol), methanesulfonic acid (10.4 μL, 0.16 mmol), octanol (10 mL), n-hexane (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 60° C. and stirred for 16 hours. After completion, the system was cooled to room temperature. NMR analysis indicated octyl acrylate was obtained in 89% yield with >90% selectivity.

### Example 141

A 300 mL Parr autoclave was charged under nitrogen atmosphere with Pd(hfacac)₂ (3.1 mg, 0.01 mmol), bisphosphorus ligand L8 of Formula VII (7.8 mg, 0.02 mmol), methanesulfonic acid (10.4 μL, 0.16 mmol), butanol (20 mL), and a stir bar. The system was purged with acetylene gas and then charged with acetylene (62 mmol) via flow meter under cooling, followed by pressurization with carbon monoxide to 4 MPa. The reaction mixture was stirred at rt for 12 hours. After completion, the system was cooled to room temperature. After completion, NMR analysis indicated butyl acrylate was obtained in 90% yield with >90% selectivity.

### Example 142

A 300 mL Parr autoclave was charged under nitrogen atmosphere with Pd(CH₃CN)₄(BF₄)₂ (4.4 mg, 0.01 mmol), bisphosphorus ligand L8 of Formula VII (7.8 mg, 0.02 mmol), methanesulfonic acid (10.4 μL, 0.16 mmol), methanol (10 mL), 1,4-dioxane (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 100° C. and stirred for 5 hours. After completion, the system was cooled to room temperature. NMR analysis indicated butyl acrylate was obtained in 90% yield with >90% selectivity.

### Example 143

A 300 mL Parr autoclave was charged under nitrogen atmosphere with Pd₂(dba)₃ (4.6 mg, 0.005 mmol), bisphosphorus ligand L8 of Formula VII (7.8 mg, 0.02 mmol), p-toluenesulfonic acid (17.5 mg, 0.10 mmol), methanol (10 mL), THF (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 100° C. and stirred for 5 hours. After completion, the system was cooled to room temperature. NMR analysis indicated butyl acrylate was obtained in 90% yield with >90% selectivity.

### Example 144

A 300 mL Parr autoclave was charged under nitrogen atmosphere with Pd(dba)₂ (5.7 mg, 0.01 mmol), bisphosphorus ligand L8 of Formula VII (15.6 mg, 0.04 mmol), trifluoroacetic acid (12 μL, 0.16 mmol), methanol (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 100° C. and stirred for 5 hours. After completion, the system was cooled to room temperature. NMR analysis indicated butyl acrylate was obtained in 87% yield with >90% selectivity.

### Category VIII: Synthesis and Application of Formula VIII

### Example 145 Preparation of Ligand Represented by Formula VIII Using L1

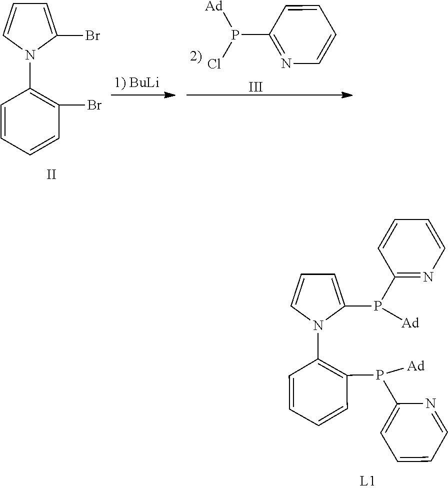

In a 100 mL three-necked flask equipped with a thermometer, magnetic stirrer, and reflux condenser, dibromide II (10 mmol) was dissolved in 50 mL of anhydrous tetrahydrofuran (THF) under nitrogen atmosphere. The solution was cooled to −78° C., and n-butyllithium (25 mL, 1.6 M in hexane) was added dropwise via syringe. After stirring at −78° C. for 3 hours, a solution of chlorophosphine III (20 mmol) in 20 mL of anhydrous THF was added slowly. The reaction mixture was then allowed to warm to room temperature naturally and stirred overnight. Upon completion, the reaction was quenched with 2 mL of degassed water, and the solvent was removed under reduced pressure. The residue was treated with oxygen-free water and extracted with diethyl ether. The organic layer was separated, dried over anhydrous magnesium sulfate, and concentrated to remove the ether. The crude product was purified by recrystallization from methanol to afford the target compound as a white to pale yellow solid powder.

Partial ligand data are as follows:

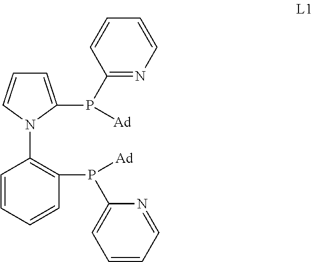

¹H NMR (400 MHz, CDCl₃) δ 8.61-8.46 (m, 2H), 7.70-7.61 (m, 4H), 7.40-7.21 (m, 7H), 6.52-6.42 (m, 2H), 2.05-1.98 (m, 6H), 1.80-1.71 (m, 24H).

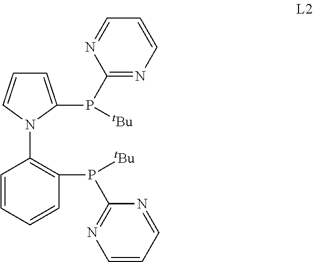 [SMILES: CC(C)(C)P(c1ncccn1)c1ccccc1-n1cccc1P(c1ncccn1)C(C)(C)C]

¹H NMR (400 MHz, CDCl₃) δ 8.75-8.68 (m, 4H), 7.73-7.61 (m, 4H), 7.38-7.25 (m, 3H), 6.53-6.41 (m, 2H), 1.18-1.10 (m, 18H).

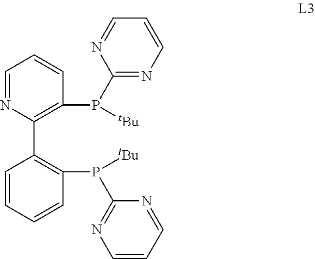 [SMILES: CC(C)(C)P(c1ncccn1)c1ccccc1-c1ncccc1P(c1ncccn1)C(C)(C)C]

¹H NMR (400 MHz, CDCl₃) δ 8.79-8.70 (m, 4H), 8.42 ((d, J=7.6 Hz, 1H), 8.34 (d, J=7.6 Hz, 1H), 7.83-7.38 (m, 6H), 7.06-7.00 (m, 1H), 1.18-1.10 (m, 18H).

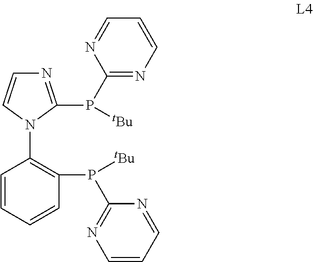 [SMILES: CC(C)(C)P(c1ncccn1)c1ccccc1-n1ccnc1P(c1ncccn1)C(C)(C)C]

¹H NMR (400 MHz, CDCl₃) δ 8.76-8.68 (m, 4H), 7.65-7.13 (m, 8H), 1.16-1.07 (m, 18H).

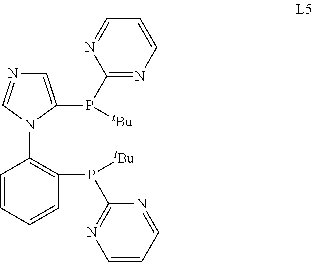 [SMILES: CC(C)(C)P(c1ncccn1)c1ccccc1-n1cncc1P(c1ncccn1)C(C)(C)C]

¹H NMR (400 MHz, CDCl₃) δ 8.76-8.68 (m, 4H), 7.65-7.13 (m, 8H), 1.16-1.07 (m, 18H)

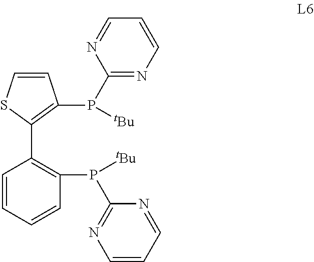 [SMILES: CC(C)(C)P(c1ncccn1)c1ccccc1-c1sccc1P(c1ncccn1)C(C)(C)C]

¹H NMR (400 MHz, CDCl₃) δ 8.85-8.75 (m, 4H), 7.80-7.56 (m, 6H), 7.38-7.35 (m, 1H), 7.13-7.10 (m, 1H), 1.21-1.08 (m, 18H).

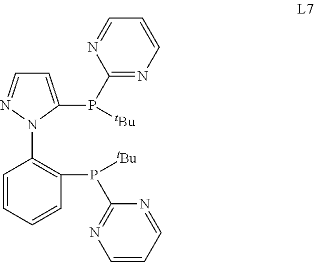 [SMILES: CC(C)(C)P(c1ncccn1)c1ccccc1-n1nccc1P(c1ncccn1)C(C)(C)C]

¹H NMR (400 MHz, CDCl₃) δ 8.85-8.75 (m, 4H), 7.75-7.40 (m, 6H), 7.18-7.15 (m, 1H), 6.92-6.89 (m, 1H), 1.18-1.08 (m, 18H).

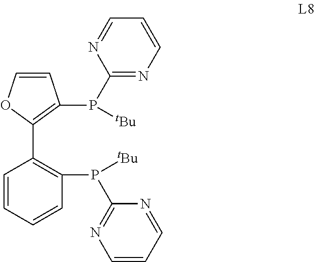 [SMILES: CC(C)(C)P(c1ncccn1)c1ccccc1-c1occc1P(c1ncccn1)C(C)(C)C]

¹H NMR (400 MHz, CDCl₃) δ 8.90-8.73 (m, 4H), 7.82-7.50 (m, 6H), 7.36-7.32 (m, 1H), 6.61-6.58 (m, 1H), 1.18-1.06 (m, 18H).

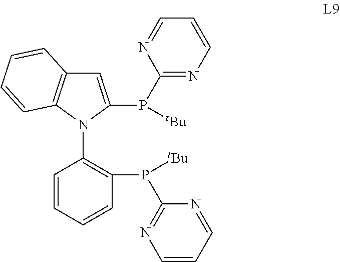 [SMILES: CC(C)(C)P(c1ncccn1)c1ccccc1-n1c(P(c2ncccn2)C(C)(C)C)cc2ccccc21]

¹H NMR (400 MHz, CDCl₃) δ 8.79-8.65 (m, 4H), 7.95-7.90 (m, 2H), 7.78-7.59 (m, 4H), 7.39-7.28 (m, 3H), 6.88-6.80 (m, 2H), 1.17-1.05 (m, 18H).

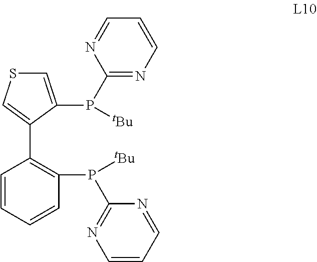 [SMILES: CC(C)(C)P(c1ncccn1)c1ccccc1-c1cscc1P(c1ncccn1)C(C)(C)C]

¹H NMR (400 MHz, CDCl₃) δ 8.90-8.75 (m, 4H), 7.98-7.34 (m, 8H), 1.20-1.06 (m, 18H).

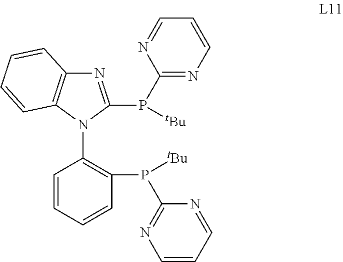 [SMILES: CC(C)(C)P(c1ncccn1)c1ccccc1-n1c(P(c2ncccn2)C(C)(C)C)nc2ccccc21]

¹H NMR (400 MHz, CDCl₃) δ 8.86-8.71 (m, 4H), 8.51-8.49 (m, 1H), 7.68-7.15 (m, 9H), 1.20-1.06 (m, 18H).

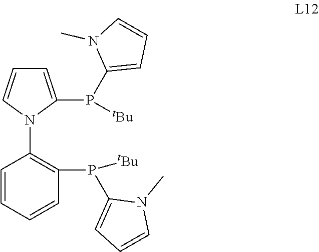 [SMILES: Cn1cccc1P(c1ccccc1-n1cccc1P(c1cccn1C)C(C)(C)C)C(C)(C)C]

¹H NMR (400 MHz, CDCl₃) δ 7.75-7.68 (m, 2H), 7.40-7.21 (m, 5H), 6.48-6.41 (m, 2H), 6.21-6.12 (m, 4H), 3.62-3.59 (m, 6H), 1.20-1.06 (m, 18H).

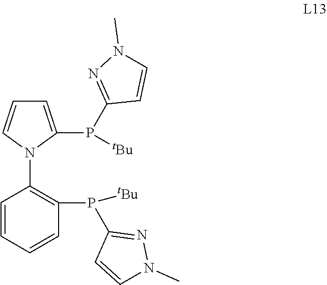 [SMILES: Cn1ccc(P(c2ccccc2-n2cccc2P(c2ccn(C)n2)C(C)(C)C)C(C)(C)C)n1]

¹H NMR (400 MHz, CDCl₃) δ 7.85-7.70 (m, 4H), 7.40-7.26 (m, 3H), 6.65-6.60 (m, 2H), 6.46-6.40 (m, 2H), 3.95-3.90 (m, 6H), 1.20-1.06 (m, 18H).

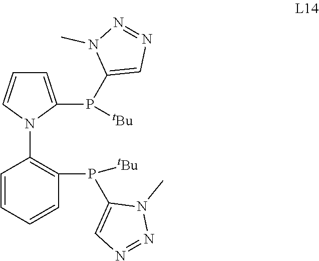 [SMILES: Cn1nncc1P(c1ccccc1-n1cccc1P(c1cnnn1C)C(C)(C)C)C(C)(C)C]

¹H NMR (400 MHz, CDCl₃) δ 8.21 (s, 1H), 7.76-7.70 (m, 2H), 7.34-7.29 (m, 3H), 6.52-6.47 (m, 2H), 3.85-3.80 (m, 6H), 1.18-1.09 (m, 18H).

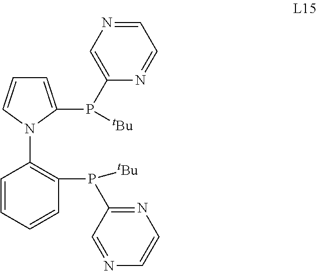 [SMILES: CC(C)(C)P(c1cnccn1)c1ccccc1-n1cccc1P(c1cnccn1)C(C)(C)C]

¹H NMR (400 MHz, CDCl₃) δ 8.92-8.76 (m, 6H), 7.73-7.68 (m, 2H), 7.40-7.31 (m, 3H), 6.52-6.47 (m, 2H), 1.16-1.07 (m, 18H).

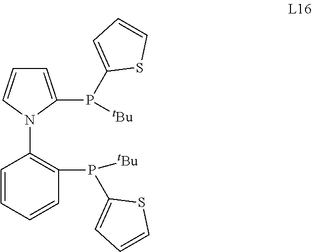 [SMILES: CC(C)(C)P(c1cccs1)c1ccccc1-n1cccc1P(c1cccs1)C(C)(C)C]

¹H NMR (400 MHz, CDCl₃) δ 7.75-7.52 (m, 4H), 7.40-7.31 (m, 3H), 7.12-7.00 (m, 4H), 6.54-6.48 (m, 2H), 1.16-1.07 (m, 18H).

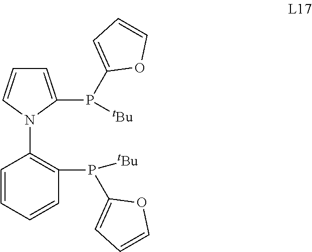 [SMILES: CC(C)(C)P(c1ccco1)c1ccccc1-n1cccc1P(c1ccco1)C(C)(C)C]

¹H NMR (400 MHz, CDCl₃) δ 7.85-7.70 (m, 4H), 7.40-7.29 (m, 3H), 6.60-6.43 (m, 6H), 1.18-1.07 (m, 18H).

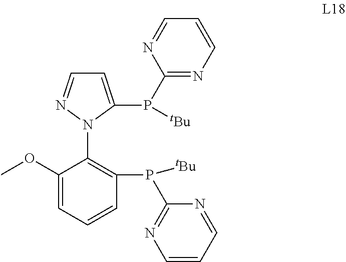 [SMILES: COc1cccc(P(c2ncccn2)C(C)(C)C)c1-n1nccc1P(c1ncccn1)C(C)(C)C]

¹H NMR (400 MHz, CDCl₃) δ 8.87-8.83 (m, 4H), 7.66-7.34 (m, 4H), 7.00-6.87 (m, 3H), 3.85-3.83 (m, 3H), 1.16-1.06 (m, 18H).

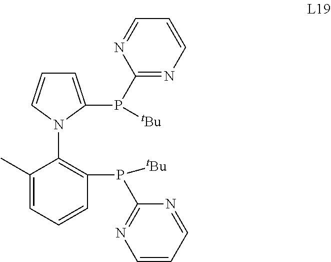 [SMILES: Cc1cccc(P(c2ncccn2)C(C)(C)C)c1-n1cccc1P(c1ncccn1)C(C)(C)C]

¹H NMR (400 MHz, CDCl₃) δ 8.87-8.83 (m, 4H), 7.65-7.53 (m, 3H), 7.28-7.18 (m, 3H), 6.55-6.49 (m, 2H), 2.12-2.08 (m, 3H), 1.16-1.06 (m, 18H).

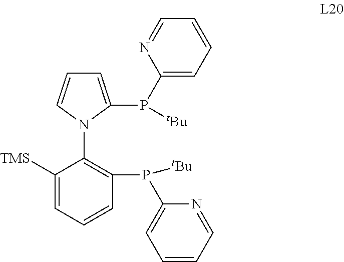 [SMILES: CC(C)(C)P(c1ccccn1)c1cccc([Si](C)(C)C)c1-n1cccc1P(c1ccccn1)C(C)(C)C]

¹H NMR (400 MHz, CDCl₃) δ 8.47-8.43 (m, 2H), 7.68-7.25 (m, 10H), 6.48-6.44 (m, 2H), 1.20-1.11 (m, 18H), −0.08-−0.12 (m, 9H).

### Example 146

A 300 mL Parr autoclave was charged under nitrogen atmosphere with Pd(OAc)₂ (2.2 mg, 0.01 mmol), bisphosphorus ligand L1 of Formula VIII (12.6 mg, 0.02 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 78% yield with >90% selectivity.

### Example 147

A 300 mL Parr autoclave was charged under nitrogen atmosphere with Pd(OAc)₂ (0.2 mg, 0.001 mmol), bisphosphorus ligand L2 of Formula VIII (0.9 mg, 0.002 mmol), methanesulfonic acid (6.5 μL, 0.10 mmol), methanol (20 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 8 hours. After completion, the system was cooled to room temperature. NMR analysis indicated methyl acrylate was obtained in 85% yield with >90% selectivity.

### Example 148

A 300 mL Parr autoclave was charged under nitrogen atmosphere with Pd(OTFA)₂ (4.0 mg, 0.01 mmol), bisphosphorus ligand L4 of Formula VIII (9.3 mg, 0.02 mmol), methanesulfonic acid (6.5 μL, 0.10 mmol), methanol (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 5 hours. After completion, the system was cooled to room temperature. NMR analysis indicated methyl acrylate was obtained in 80% yield with >90% selectivity.

### Example 149

A 300 mL Parr autoclave was charged under nitrogen atmosphere with Pd(OPiv)₂ (3.1 mg, 0.01 mmol), bisphosphorus ligand L5 of Formula VIII (18.6 mg, 0.04 mmol), methanesulfonic acid (10.4 μL, 0.16 mmol), methanol (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 6 hours. After completion, the system was cooled to room temperature. NMR analysis indicated methyl acrylate was obtained in 85% yield with >90% selectivity.

### Example 150

A 300 mL Parr autoclave was charged under nitrogen atmosphere with Pd₂(dba)₃ (4.6 mg, 0.005 mmol), bisphosphorus ligand L6 of Formula VIII (9.8 mg, 0.02 mmol), methanesulfonic acid (10.4 μL, 0.16 mmol), ethanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated ethyl acrylate was obtained in 68% yield with >90% selectivity.

### Example 151

A 300 mL Parr autoclave was charged under nitrogen atmosphere with Pd(dba)₂ (5.7 mg, 0.01 mmol), bisphosphorus ligand L8 of Formula VIII (19.0 mg, 0.04 mmol), trifluoromethanesulfonic acid (14.2 μL, 0.16 mmol), butanol (10 mL) and THF (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated butyl acrylate was obtained in 87% yield with >90% selectivity.

### Example 152

A 300 mL Parr autoclave was charged under nitrogen atmosphere with PdCl₂ (1.8 mg, 0.01 mmol), bisphosphorus ligand L9 of Formula VIII (10.5 mg, 0.02 mmol), dodecyl sulfonic acid (26.1 mg, 0.08 mmol), methanol (20 mL), and THF (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated methyl acrylate was obtained in 78% yield with >90% selectivity.

### Example 153

A 300 mL Parr autoclave was charged under nitrogen atmosphere with Pd(hfacac)₂ (3.1 mg, 0.01 mmol), bisphosphorus ligand L10 of Formula VIII (19.7 mg, 0.04 mmol), methanesulfonic acid (10.4 μL, 0.16 mmol), octanol (10 mL) and n-hexane (10 mL), and a stir bar. The system was purged with acetylene gas and then charged with acetylene (62 mmol) via flow meter under cooling, followed by pressurization with carbon monoxide to 4 MPa. The reaction mixture was stirred at rt for 24 hours. After completion, NMR analysis indicated octyl acrylate was obtained in 73% yield with >90% selectivity.

### Example 154

A 300 mL Parr autoclave was charged under nitrogen atmosphere with Pd(CH₃CN)₄(BF₄)₂ (4.4 mg, 0.01 mmol), bisphosphorus ligand L11 of Formula VIII (21.0 mg, 0.04 mmol), methanesulfonic acid (10.4 μL, 0.16 mmol), methanol (10 mL) and 1,4-dioxane (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 6 hours. After completion, the system was cooled to room temperature. NMR analysis indicated methyl acrylate was obtained in 78% yield with >90% selectivity.

### Example 155

A 300 mL Parr autoclave was charged under nitrogen atmosphere with Pd(OAc)₂ (11.2 mg, 0.05 mmol), bisphosphorus ligand L12 of Formula VIII (19.1 mg, 0.04 mmol), methanesulfonic acid (10.4 μL, 0.16 mmol), butanol (10 mL) and THF (10 mL), and a stir bar. The system was purged with acetylene gas and then charged with acetylene (62 mmol) via flow meter under cooling, followed by pressurization with carbon monoxide to 4 MPa. The reaction mixture was rapidly heated to 120° C. and stirred for 8 hours. After completion, the system was cooled to room temperature. NMR analysis indicated butyl acrylate was obtained in 75% yield with >90% selectivity.

### Example 156

A 300 mL Parr autoclave was charged under nitrogen atmosphere with Pd(OAc)₂ (2.2 mg, 0.01 mmol), bisphosphorus ligand L13 of Formula VIII (9.5 mg, 0.02 mmol), p-toluenesulfonic acid (17.5 mg, 0.10 mmol), methanol (10 mL) and THF (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 64% yield with >90% selectivity.

### Example 157

A 300 mL Parr autoclave was charged under nitrogen atmosphere with Pd(OAc)₂ (2.2 mg, 0.01 mmol), bisphosphorus ligand L14 of Formula VIII (9.6 mg, 0.02 mmol), methanesulfonic acid (10.4 μL, 0.16 mmol), methanol (10 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 67% yield with >90% selectivity.

### Example 158

A 300 mL Parr autoclave was charged under nitrogen atmosphere with Pd(OAc)₂ (2.24 mg, 0.01 mmol), bisphosphorus ligand L15 of Formula VIII (9.5 mg, 0.02 mmol), trifluoroacetic acid (12 μL, 0.16 mmol) with methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 82% yield with >90% selectivity.

### Example 159

A 300 mL Parr autoclave was charged under nitrogen atmosphere with Pd(OAc)₂ (2.24 mg, 0.01 mmol), bisphosphorus ligand L16 of Formula VIII (9.7 mg, 0.02 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 100° C. and stirred for 5 hours. After completion, the system was cooled to room temperature. NMR analysis indicated methyl acrylate was obtained in 63% yield with >90% selectivity.

### Example 160

A 300 mL Parr autoclave was charged under nitrogen atmosphere with Pd(OAc)₂ (2.2 mg, 0.01 mmol), bisphosphorus ligand L17 of Formula VIII (18.0 mg, 0.04 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 60° C. and stirred for 8 hours. After completion, the system was cooled to room temperature. NMR analysis indicated methyl acrylate was obtained in 58% yield with >90% selectivity.

### Example 161

A 300 mL Parr autoclave was charged under nitrogen atmosphere with Pd(OAc)₂ (2.2 mg, 0.01 mmol), bisphosphorus ligand L18 of Formula VIII (20.2 mg, 0.04 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 120° C. and stirred for 5 hours. After completion, the system was cooled to room temperature. NMR analysis indicated methyl acrylate was obtained in 84% yield with >90% selectivity.

### Example 162

A 300 mL Parr autoclave was charged under nitrogen atmosphere with Pd(OAc)₂ (2.2 mg, 0.01 mmol), bisphosphorus ligand L19 of Formula VIII (19.6 mg, 0.04 mmol), methanesulfonic acid (10.4 μL, 0.16 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was stirred at rt for 10 hours. After completion, NMR analysis indicated methyl acrylate was obtained in 81% yield with >90% selectivity.

### Example 163

A 300 mL Parr autoclave was charged under nitrogen atmosphere with Pd(OAc)₂ (0.2 mg, 0.001 mmol), bisphosphorus ligand L20 of Formula VIII (2.0 mg, 0.004 mmol), methanesulfonic acid (1.0 μL, 0.016 mmol), methanol (20 mL), and a stir bar. The system was purged with acetylene gas and then charged with acetylene (100 mmol) via flow meter under cooling, followed by pressurization with carbon monoxide to 6 MPa. The reaction mixture was rapidly heated to 100° C. and stirred for 8 hours. After completion, the system was cooled to room temperature. NMR analysis indicated methyl acrylate was obtained in 81% yield with >90% selectivity.

The phosphine ligands of the present invention, when used for catalyzing the preparation of acrylate esters, can provide the acrylate ester products in high yields (>55%, optimally >80% in preferred Examples) with excellent selectivity, thereby demonstrating potential for industrial applications.

Furthermore, the phosphine ligands of the present invention exhibit superior catalytic turnover, enabling the completion of the catalytic reaction at low loadings (<10⁻⁴ equivalents, preferably <10⁻⁵ equivalents, more preferably <10⁻⁶ equivalents).

### Carbonylation of Ethylene

### Category I: Application of Formula I

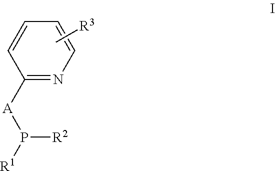

### Example 1

A 300 mL Parr autoclave was charged under nitrogen atmosphere with Pd(OAc)₂ (2.2 mg, 0.01 mmol), nitrogen-phosphorus ligand L1 of Formula I (12.4 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 3 hours. After completion, the system was cooled to room temperature. NMR analysis indicated methyl propionate was obtained in 82% yield with >95% selectivity.

### Example 2

A 300 mL Parr autoclave was charged under nitrogen atmosphere with Pd(OAc)₂ (2.2 mg, 0.01 mmol), nitrogen-phosphorus ligand L2 of Formula I (14.2 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 3 hours. After completion, the system was cooled to room temperature. NMR analysis indicated methyl propionate was obtained in 84% yield with >95% selectivity.

### Example 3

A 300 mL Parr autoclave was charged under nitrogen atmosphere with Pd(OAc)₂ (2.2 mg, 0.01 mmol), nitrogen-phosphorus ligand L3 of Formula I (11.8 mg, 0.04 mmol), methanesulfonic acid (6.5 μL, 0.10 mmol), methanol (30 mL), toluene (30 mL), and a stir bar. The system was purged with nitrogen, then pressurized with ethylene gas to 2.0 MPa, followed by pressurization with carbon monoxide to 6.0 MPa. The reaction mixture was rapidly heated to 100° C. and stirred for 2 hours. After completion, the system was cooled to room temperature. NMR analysis indicated methyl propionate was obtained in 67% yield with >95% selectivity.

### Example 4

A 300 mL Parr autoclave was charged under nitrogen atmosphere with Pd(OAc)₂ (0.02 mg, 0.0001 mmol), nitrogen-phosphorus ligand L4 of Formula I (0.15 mg, 0.0004 mmol), methanesulfonic acid (0.7 μL, 0.01 mmol), methanol (30 mL), acetone (20 mL), and a stir bar. The system was purged with nitrogen, then pressurized with ethylene gas to 2.0 MPa, followed by pressurization with carbon monoxide to 6.0 MPa. The reaction mixture was rapidly heated to 60° C. and stirred for 6 hours. After completion, the system was cooled to room temperature. NMR analysis indicated methyl propionate was obtained in 92% yield with >95% selectivity.

### Example 5

A 300 mL Parr autoclave was charged under nitrogen atmosphere with Pd(OAc)₂ (0.2 mg, 0.001 mmol), nitrogen-phosphorus ligand L5 of Formula I (1.4 mg, 0.004 mmol), methanesulfonic acid (6.5 μL, 0.10 mmol), methanol (20 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 3 hours. After completion, the system was cooled to room temperature. NMR analysis indicated methyl propionate was obtained in 90% yield with >95% selectivity.

### Example 6

A 300 mL Parr autoclave was charged under nitrogen atmosphere with Pd(OAc)₂ (2.2 mg, 0.01 mmol), nitrogen-phosphorus ligand L6 of Formula I (14.6 mg, 0.04 mmol), methanesulfonic acid (6.5 μL, 0.10 mmol), methanol (30 mL), acetonitrile (20 mL), and a stir bar. The system was purged with nitrogen, then pressurized with ethylene gas to 2.0 MPa, followed by pressurization with carbon monoxide to 6.0 MPa. The reaction mixture was rapidly heated to 60° C. and stirred for 3 hours. After completion, the system was cooled to room temperature. NMR analysis indicated methyl propionate was obtained in 87% yield with >95% selectivity.

### Example 7

A 300 mL Parr autoclave was charged under nitrogen atmosphere with Pd(OAc)₂ (2.2 mg, 0.01 mmol), nitrogen-phosphorus ligand L7 of Formula I (14.5 mg, 0.04 mmol), methanesulfonic acid (6.5 μL, 0.10 mmol), methanol (30 mL), THF (20 mL), and a stir bar. The system was purged with nitrogen, then pressurized with ethylene gas to 2.0 MPa, followed by pressurization with carbon monoxide to 6.0 MPa. The reaction mixture was rapidly heated to 50° C. and stirred for 5 hours. After completion, the system was cooled to room temperature. NMR analysis indicated methyl propionate was obtained in 78% yield with >95% selectivity.

### Example 8

A 300 mL Parr autoclave was charged under nitrogen atmosphere with Pd(OAc)₂ (2.2 mg, 0.01 mmol), nitrogen-phosphorus ligand L8 of Formula I (14.6 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 60° C. and stirred for 5 hours. After completion, the system was cooled to room temperature. NMR analysis indicated methyl propionate was obtained in 72% yield with >95% selectivity.

### Example 9

A 300 mL Parr autoclave was charged under nitrogen atmosphere with Pd(OAc)₂ (2.2 mg, 0.01 mmol), nitrogen-phosphorus ligand L9 of Formula I (16.4 mg, 0.04 mmol), methanesulfonic acid (6.5 μL, 0.10 mmol), methanol (30 mL), 1-hexane (20 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 82% yield with >95% selectivity.

### Example 10

A 300 mL Parr autoclave was charged under nitrogen atmosphere with Pd₂(dba)₃ (4.6 mg, 0.005 mmol), nitrogen-phosphorus ligand L10 of Formula I (14.6 mg, 0.04 mmol), methanesulfonic acid (6.5 μL, 0.10 mmol), methanol (30 mL), ethyl acetate (20 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 85% yield with >95% selectivity.

### Example 11

A 300 mL Parr autoclave was charged under nitrogen atmosphere with Pd(dba)₂ (5.7 mg, 0.01 mmol), nitrogen-phosphorus ligand L11 of Formula I (16.5 mg, 0.04 mmol), trifluoroacetic acid (14.2 μL, 0.16 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 80% yield with >95% selectivity.

### Example 12

A 300 mL Parr autoclave was charged under nitrogen atmosphere with PdCl₂ (0.2 mg, 0.001 mmol), nitrogen-phosphorus ligand L12 of Formula I (1.1 mg, 0.002 mmol), dodecylsulfonic acid (2.6 mg, 0.008 mmol), methanol (50 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 80% yield with >95% selectivity.

### Example 13

A 300 mL Parr autoclave was charged under nitrogen atmosphere with Pd(hfacac)₂ (3.1 mg, 0.01 mmol), nitrogen-phosphorus ligand L13 of Formula I (15.6 mg, 0.04 mmol), methanesulfonic acid (10.4 μL, 0.16 mmol), methanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 88% yield with >95% selectivity.

### Example 14

A 300 mL Parr autoclave was charged under nitrogen atmosphere with Pd(CH₃CN)₄(BF₄)₂ (4.4 mg, 0.01 mmol), nitrogen-phosphorus ligand L14 of Formula I (8.4 mg, 0.02 mmol), methanesulfonic acid (10.4 μL, 0.16 mmol), propanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated propyl propionate was obtained in 85% yield with >95% selectivity.

### Example 15

A 300 mL Parr autoclave was charged under nitrogen atmosphere with Pd(OAc)₂ (11.2 mg, 0.05 mmol), nitrogen-phosphorus ligand L15 of Formula I (17.4 mg, 0.04 mmol), methanesulfonic acid (10.4 μL, 0.16 mmol), butanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated butyl propionate was obtained in 90% yield with >95% selectivity.

### Example 16

A 300 mL Parr autoclave was charged under nitrogen atmosphere with Pd(OAc)₂ (2.2 mg, 0.01 mmol), nitrogen-phosphorus ligand L16 of Formula I (12.0 mg, 0.04 mmol), p-toluenesulfonic acid (17.5 mg, 0.10 mmol), ethanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated ethyl propionate was obtained in 82% yield with >95% selectivity.

### Example 17

A 300 mL Parr autoclave was charged under nitrogen atmosphere with Pd(OAc)₂ (2.2 mg, 0.01 mmol), nitrogen-phosphorus ligand L17 of Formula I (15.2 mg, 0.04 mmol), methanesulfonic acid (10.4 μL, 0.16 mmol), butanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated butyl propionate was obtained in 81% yield with >95% selectivity.

### Example 18

A 300 mL Parr autoclave was charged under nitrogen atmosphere with Pd(OAc)₂ (2.24 mg, 0.01 mmol), nitrogen-phosphorus ligand L18 of Formula I (12.9 mg, 0.04 mmol), trifluoroacetic acid (12 μL, 0.16 mmol), octanol (30 mL), THF (20 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated octyl propionate was obtained in 67% yield with >90% selectivity.

### Category II: Application of Formula V

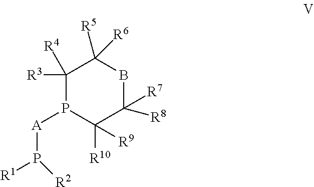

### Example 20

A 300 mL Parr autoclave was charged under nitrogen atmosphere with Pd(OAc)₂ (2.2 mg, 0.01 mmol), bisphosphorus ligand L1 of Formula V (15.1 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 100° C. and stirred for 5 hours. After completion, the system was cooled to room temperature. NMR analysis indicated methyl propionate was obtained in 68% yield with >95% selectivity.

### Example 21

A 300 mL Parr autoclave was charged under nitrogen atmosphere with Pd(OAc)₂ (2.2 mg, 0.01 mmol), bisphosphorus ligand L2 of Formula V (14.6 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 3 hours. After completion, the system was cooled to room temperature. NMR analysis indicated methyl propionate was obtained in 89% yield with >95% selectivity.

### Example 22

A 300 mL Parr autoclave was charged under nitrogen atmosphere with Pd(OAc)₂ (2.2 mg, 0.01 mmol), bisphosphorus ligand L3 of Formula V (15.2 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 60° C. and stirred for 3 hours. After completion, the system was cooled to room temperature. NMR analysis indicated methyl propionate was obtained in 92% yield with >95% selectivity.

### Example 23

A 300 mL Parr autoclave was charged under nitrogen atmosphere with Pd(OAc)₂ (2.2 mg, 0.01 mmol), bisphosphorus ligand L4 of Formula V (16.2 mg, 0.04 mmol), methanesulfonic acid (6.5 μL, 0.10 mmol), methanol (20 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 3 hours. After completion, the system was cooled to room temperature. NMR analysis indicated methyl propionate was obtained in 95% yield with >95% selectivity.

### Example 24

A 300 mL Parr autoclave was charged under nitrogen atmosphere with Pd(OAc)₂ (2.2 mg, 0.01 mmol), bisphosphorus ligand L5 of Formula V (20.1 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 60° C. and stirred for 3 hours. After completion, the system was cooled to room temperature. NMR analysis indicated methyl propionate was obtained in 88% yield with >95% selectivity.

### Example 25

A 300 mL Parr autoclave was charged under nitrogen atmosphere with Pd(OAc)₂ (2.2 mg, 0.01 mmol), bisphosphorus ligand L7 of Formula V (24.1 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 60° C. and stirred for 3 hours. After completion, the system was cooled to room temperature. NMR analysis indicated methyl propionate was obtained in 94% yield with >95% selectivity.

### Example 26

A 300 mL Parr autoclave was charged under nitrogen atmosphere with Pd(OAc)₂ (2.2 mg, 0.01 mmol), bisphosphorus ligand L9 of Formula V (17.6 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 60° C. and stirred for 5 hours. After completion, the system was cooled to room temperature. NMR analysis indicated methyl propionate was obtained in 91% yield with >95% selectivity.

### Example 27

A 300 mL Parr autoclave was charged under nitrogen atmosphere with Pd(OAc)₂ (2.2 mg, 0.01 mmol), bisphosphorus ligand L11 of Formula V (19.6 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 90% yield with >95% selectivity.

### Example 28

A 300 mL Parr autoclave was charged under nitrogen atmosphere with Pd₂(dba)₃ (4.6 mg, 0.005 mmol), bisphosphorus ligand L6 of Formula V (21.8 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 85% yield with >95% selectivity.

### Example 29

A 300 mL Parr autoclave was charged under nitrogen atmosphere with Pd(dba)₂ (5.7 mg, 0.01 mmol), bisphosphorus ligand L12 of Formula V (21.9 mg, 0.04 mmol), trifluoromethanesulfonic acid (14.2 μL, 0.16 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 3 hours. After completion, the system was cooled to room temperature. NMR analysis indicated methyl propionate was obtained in 89% yield with >95% selectivity.

### Example 30

A 300 mL Parr autoclave was charged under nitrogen atmosphere with PdCl₂ (1.8 mg, 0.01 mmol), bisphosphorus ligand L13 of Formula V (10.4 mg, 0.04 mmol), dodecylsulfonic acid (26.1 mg, 0.08 mmol), methanol (50 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 90% yield with >95% selectivity.

### Example 31

A 300 mL Parr autoclave was charged under nitrogen atmosphere with Pd(hfacac)₂ (3.1 mg, 0.01 mmol), bisphosphorus ligand L14 of Formula V (13.6 mg, 0.04 mmol), methanesulfonic acid (10.4 μL, 0.16 mmol), methanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 89% yield with >95% selectivity.

### Example 32

A 300 mL Parr autoclave was charged under nitrogen atmosphere with Pd(CH₃CN)₄(BF₄)₂ (4.4 mg, 0.01 mmol), bisphosphorus ligand L15 of Formula V (20.9 mg, 0.04 mmol), methanesulfonic acid (10.4 μL, 0.16 mmol), methanol (50 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 85% yield with >95% selectivity.

### Example 33

A 300 mL Parr autoclave was charged under nitrogen atmosphere with Pd(OAc)₂ (1.1 mg, 0.005 mmol), bisphosphorus ligand L16 of Formula V (9.6 mg, 0.02 mmol), methanesulfonic acid (10.4 μL, 0.16 mmol), butanol (30 mL), 1-hexane (20 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated butyl propionate was obtained in 75% yield with >95% selectivity.

### Example 34

A 300 mL Parr autoclave was charged under nitrogen atmosphere with Pd(OAc)₂ (2.2 mg, 0.01 mmol), bisphosphorus ligand L18 of Formula V (18.7 mg, 0.04 mmol), p-toluenesulfonic acid (17.5 mg, 0.10 mmol), ethanol (30 mL), THF (30 mL), and a stir bar.

The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated ethyl propionate was obtained in 82% yield with >95% selectivity.

### Example 35

A 300 mL Parr autoclave was charged under nitrogen atmosphere with Pd(OAc)₂ (2.2 mg, 0.01 mmol), bisphosphorus ligand L19 of Formula V (20.0 mg, 0.04 mmol), methanesulfonic acid (10.4 μL, 0.16 mmol), buanol (30 mL), acetonitrile (20 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated butyl propionate was obtained in 78% yield with >95% selectivity.

### Example 36

A 300 mL Parr autoclave was charged under nitrogen atmosphere with Pd(OAc)₂ (0.02 mg, 0.0001 mmol), bisphosphorus ligand L20 of Formula V (0.3 mg, 0.0004 mmol), trifluoroacetic acid (1.2 μL, 0.016 mmol), octanol (30 mL), acetone (20 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 8 hours. After completion, the system was cooled to room temperature. NMR analysis indicated octyl propionate was obtained in 61% yield with >90% selectivity.

### Example 37

A 300 mL Parr autoclave was charged under nitrogen atmosphere with Pd(OAc)₂ (2.2 mg, 0.01 mmol), bisphosphorus ligand L21 of Formula V (27.3 mg, 0.04 mmol), methanesulfonic acid (10.4 μL, 0.16 mmol), propanol (30 mL), acetonitrile (20 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated propyl propionate was obtained in 82% yield with >95% selectivity.

### Example 38

A 300 mL Parr autoclave was charged under nitrogen atmosphere with Pd(OAc)₂ (0.2 mg, 0.001 mmol), bisphosphorus ligand L23 of Formula V (2.6 mg, 0.004 mmol), methanesulfonic acid (1.0 μL, 0.016 mmol), methanol (50 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 8 hours. After completion, the system was cooled to room temperature. NMR analysis indicated methyl propionate was obtained in 83% yield with >95% selectivity.

### Example 39

A 300 mL Parr autoclave was charged under nitrogen atmosphere with Pd(OAc)₂ (2.2 mg, 0.01 mmol), bisphosphorus ligand L25 of Formula V (28.4 mg, 0.04 mmol), methanesulfonic acid (10.4 μL, 0.16 mmol), methanol (30 mL), methyl propionate (20 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 93% yield with >95% selectivity.

### Example 40

A 300 mL Parr autoclave was charged under nitrogen atmosphere with Pd(OAc)₂ (2.2 mg, 0.01 mmol), bisphosphorus ligand L26 of Formula V (19.0 mg, 0.04 mmol), methanesulfonic acid (10.4 μL, 0.16 mmol), methanol (50 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 90% yield with >95% selectivity.

### Example 41

A 300 mL Parr autoclave was charged under nitrogen atmosphere with Pd(OAc)₂ (2.2 mg, 0.01 mmol), bisphosphorus ligand L28 of Formula V (15.2 mg, 0.04 mmol), p-toluenesulfonic acid (17.5 mg, 0.10 mmol), methanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 83% yield with >95% selectivity.

### Example 42

A 300 mL Parr autoclave was charged under nitrogen atmosphere with Pd(OAc)₂ (2.2 mg, 0.01 mmol), bisphosphorus ligand L29 of Formula V (15.7 mg, 0.04 mmol), p-toluenesulfonic acid (17.5 mg, 0.10 mmol), methanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 87% yield with >95% selectivity.

### Example 43

A 300 mL Parr autoclave was charged under nitrogen atmosphere with Pd(OAc)₂ (2.2 mg, 0.01 mmol), bisphosphorus ligand L30 of Formula V (18.8 mg, 0.04 mmol), p-toluenesulfonic acid (17.5 mg, 0.10 mmol), methanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 82% yield with >95% selectivity.

### Example 44

A 300 mL Parr autoclave was charged under nitrogen atmosphere with Pd(OAc)₂ (2.2 mg, 0.01 mmol), bisphosphorus ligand L31 of Formula V (21.8 mg, 0.04 mmol), p-toluenesulfonic acid (17.5 mg, 0.10 mmol), methanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 82% yield with >95% selectivity.

### Example 45

A 300 mL Parr autoclave was charged under nitrogen atmosphere with Pd(OAc)₂ (2.2 mg, 0.01 mmol), bisphosphorus ligand L32 of Formula V (16.3 mg, 0.04 mmol), p-toluenesulfonic acid (17.5 mg, 0.10 mmol), methanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 89% yield with >95% selectivity.

### Example 46

A 300 mL Parr autoclave was charged under nitrogen atmosphere with Pd(OAc)₂ (2.2 mg, 0.01 mmol), bisphosphorus ligand L33 of Formula V (17.2 mg, 0.04 mmol), p-toluenesulfonic acid (17.5 mg, 0.10 mmol), methanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 81% yield with >95% selectivity.

### Example 47

A 300 mL Parr autoclave was charged under nitrogen atmosphere with Pd(OAc)₂ (2.2 mg, 0.01 mmol), bisphosphorus ligand L35 of Formula V (16.4 mg, 0.04 mmol), p-toluenesulfonic acid (17.5 mg, 0.10 mmol), methanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 87% yield with >95% selectivity.

### Example 48

A 300 mL Parr autoclave was charged under nitrogen atmosphere with Pd(OAc)₂ (2.2 mg, 0.01 mmol), bisphosphorus ligand L36 of Formula V (16.9 mg, 0.04 mmol), p-toluenesulfonic acid (17.5 mg, 0.10 mmol), methanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 85% yield with >95% selectivity.

### Example 49

A 300 mL Parr autoclave was charged under nitrogen atmosphere with Pd(OAc)₂ (2.2 mg, 0.01 mmol), bisphosphorus ligand L37 of Formula V (20.0 mg, 0.04 mmol), p-toluenesulfonic acid (17.5 mg, 0.10 mmol), methanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 85% yield with >95% selectivity.

### Example 50

A 300 mL Parr autoclave was charged under nitrogen atmosphere with Pd(OAc)₂ (2.2 mg, 0.01 mmol), bisphosphorus ligand L39 of Formula V (23.8 mg, 0.04 mmol), p-toluenesulfonic acid (17.5 mg, 0.10 mmol), methanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 90% yield with >95% selectivity.

### Example 51

A 300 mL Parr autoclave was charged under nitrogen atmosphere with Pd(OAc)₂ (2.2 mg, 0.01 mmol), bisphosphorus ligand L40 of Formula V (26.9 mg, 0.04 mmol), p-toluenesulfonic acid (17.5 mg, 0.10 mmol), methanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 92% yield with >95% selectivity.

### Example 52

A 300 mL Parr autoclave was charged under nitrogen atmosphere with Pd(OAc)₂ (2.2 mg, 0.01 mmol), bisphosphorus ligand L41 of Formula V (24.3 mg, 0.04 mmol), p-toluenesulfonic acid (17.5 mg, 0.10 mmol), methanol (30 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 95% yield with >95% selectivity.

### Category III: Application of Formula VI

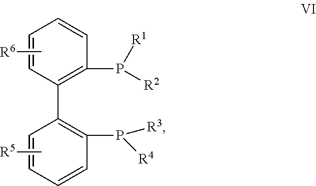

### Example 53

A 300 mL Parr autoclave was charged under nitrogen atmosphere with Pd(OAc)₂ (2.2 mg, 0.01 mmol), bisphosphorus ligand L1 of Formula VI (19.4 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 100° C. and stirred for 2 hours. After completion, the system was cooled to room temperature. NMR analysis indicated methyl propionate was obtained in 94% yield with >95% selectivity.

### Example 54

A 300 mL Parr autoclave was charged under nitrogen atmosphere with Pd(OAc)₂ (2.2 mg, 0.01 mmol), bisphosphorus ligand L2 of Formula VI (19.6 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 6 hours. After completion, the system was cooled to room temperature. NMR analysis indicated methyl propionate was obtained in 86% yield with >95% selectivity.

### Example 55

A 300 mL Parr autoclave was charged under nitrogen atmosphere with Pd(OAc)₂ (2.2 mg, 0.01 mmol), bisphosphorus ligand L3 of Formula VI (19.5 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 100° C. and stirred for 2 hours. After completion, the system was cooled to room temperature. NMR analysis indicated methyl propionate was obtained in 85% yield with >95% selectivity.

### Example 56

A 300 mL Parr autoclave was charged under nitrogen atmosphere with Pd(OAc)₂ (2.2 mg, 0.01 mmol), bisphosphorus ligand L4 of Formula VI (11.7 mg, 0.02 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 60° C. and stirred for 8 hours. After completion, the system was cooled to room temperature. NMR analysis indicated methyl propionate was obtained in 78% yield with >95% selectivity.

### Example 57

A 300 mL Parr autoclave was charged under nitrogen atmosphere with Pd(OAc)₂ (2.2 mg, 0.01 mmol), bisphosphorus ligand L5 of Formula VI (19.4 mg, 0.04 mmol), methanesulfonic acid (6.5 μL, 0.10 mmol), methanol (20 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 3 hours. After completion, the system was cooled to room temperature. NMR analysis indicated methyl propionate was obtained in 90% yield with >95% selectivity.

### Example 58

A 300 mL Parr autoclave was charged under nitrogen atmosphere with Pd(OAc)₂ (2.2 mg, 0.01 mmol), bisphosphorus ligand L6 of Formula VI (19.4 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 3 hours. After completion, the system was cooled to room temperature. NMR analysis indicated methyl propionate was obtained in 90% yield with >95% selectivity.

### Example 59

A 300 mL Parr autoclave was charged under nitrogen atmosphere with Pd(OAc)₂ (2.2 mg, 0.01 mmol), bisphosphorus ligand L7 of Formula VI (18.4 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 100° C. and stirred for 10 hours. After completion, the system was cooled to room temperature. NMR analysis indicated methyl propionate was obtained in 84% yield with >95% selectivity.

### Example 60

A 300 mL Parr autoclave was charged under nitrogen atmosphere with Pd(OAc)₂ (2.2 mg, 0.01 mmol), bisphosphorus ligand L8 of Formula VI (9.9 mg, 0.02 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 100° C. and stirred for 5 hours. After completion, the system was cooled to room temperature. NMR analysis indicated methyl propionate was obtained in 87% yield with >95% selectivity.

### Example 61

A 300 mL Parr autoclave was charged under nitrogen atmosphere with Pd(OAc)₂ (2.2 mg, 0.01 mmol), bisphosphorus ligand L9 of Formula VI (18.3 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 88% yield with >95% selectivity.

### Example 62

A 300 mL Parr autoclave was charged under nitrogen atmosphere with Pd₂(dba)₃ (4.6 mg, 0.005 mmol), bisphosphorus ligand L10 of Formula VI (12.8 mg, 0.02 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 85% yield with >95% selectivity.

### Example 63

A 300 mL Parr autoclave was charged under nitrogen atmosphere with Pd(dba)₂ (5.7 mg, 0.01 mmol), bisphosphorus ligand L11 of Formula VI (13.5 mg, 0.02 mmol), trifluoromethanesulfonic acid (14.2 μL, 0.16 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated methyl propionate was obtained in 84% yield with >95% selectivity.

### Example 64

A 300 mL Parr autoclave was charged under nitrogen atmosphere with PdCl₂ (1.8 mg, 0.01 mmol), bisphosphorus ligand L12 of Formula VI (29.2 mg, 0.04 mmol), dodecylsulfonic acid (26.1 mg, 0.08 mmol), methanol (50 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 8 hours. After completion, the system was cooled to room temperature. NMR analysis indicated methyl propionate was obtained in 87% yield with >95% selectivity.

### Example 65

A 300 mL Parr autoclave was charged under nitrogen atmosphere with Pd(hfacac)₂ (3.1 mg, 0.01 mmol), bisphosphorus ligand L13 of Formula VI (28.1 mg, 0.04 mmol), methanesulfonic acid (10.4 μL, 0.16 mmol), methanol (30 mL), 1-hexane (20 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 8 hours. After completion, the system was cooled to room temperature. NMR analysis indicated methyl propionate was obtained in 85% yield with >95% selectivity.

### Example 66

A 300 mL Parr autoclave was charged under nitrogen atmosphere with Pd(CH₃CN)₄(BF₄)₂ (4.4 mg, 0.01 mmol), bisphosphorus ligand L14 of Formula VI (15.7 mg, 0.02 mmol), methanesulfonic acid (10.4 μL, 0.16 mmol), propanol (30 mL), acetonitrile (20 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 8 hours. After completion, the system was cooled to room temperature. NMR analysis indicated propyl propionate was obtained in 80% yield with >95% selectivity.

### Example 67

A 300 mL Parr autoclave was charged under nitrogen atmosphere with Pd(OAc)₂ (11.2 mg, 0.05 mmol), bisphosphorus ligand L15 of Formula VI (30.2 mg, 0.04 mmol), methanesulfonic acid (10.4 μL, 0.16 mmol), butanol (30 mL), toluene (20 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 8 hours. After completion, the system was cooled to room temperature. NMR analysis indicated butyl propionate was obtained in 82% yield with >95% selectivity.

### Example 68

A 300 mL Parr autoclave was charged under nitrogen atmosphere with Pd(OAc)₂ (0.2 mg, 0.001 mmol), bisphosphorus ligand L1 of Formula VI (1.9 mg, 0.004 mmol), p-toluenesulfonic acid (1.8 mg, 0.01 mmol), ethanol (30 mL), THF (20 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 8 hours. After completion, the system was cooled to room temperature. NMR analysis indicated ethyl propionate was obtained in 86% yield with >95% selectivity.

### Example 69

A 300 mL Parr autoclave was charged under nitrogen atmosphere with Pd(OAc)₂ (2.2 mg, 0.01 mmol), bisphosphorus ligand L1 of Formula VI (19.4 mg, 0.04 mmol), trifluoroacetic acid (12 μL, 0.16 mmol), octanol (30 mL), THF (20 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 5 hours. After completion, the system was cooled to room temperature. NMR analysis indicated octyl propionate was obtained in 87% yield with >90% selectivity.

### Category IV: Application of Formula VIII

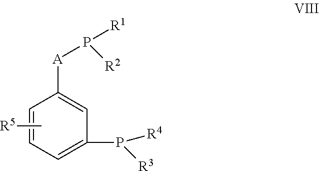

### Example 70

A 300 mL Parr autoclave was charged under nitrogen atmosphere with Pd(OAc)₂ (2.2 mg, 0.01 mmol), bisphosphorus ligand L1 of Formula VIII (25.2 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 100° C. and stirred for 10 hours. After completion, the system was cooled to room temperature. NMR analysis indicated methyl propionate was obtained in 87% yield with >95% selectivity.

### Example 71

A 300 mL Parr autoclave was charged under nitrogen atmosphere with Pd(OAc)₂ (0.2 mg, 0.001 mmol), bisphosphorus ligand L2 of Formula VIII (1.9 mg, 0.004 mmol), methanesulfonic acid (0.6 μmL, 0.010 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 12 hours. After completion, the system was cooled to room temperature. NMR analysis indicated methyl propionate was obtained in 93% yield with >95% selectivity.

### Example 72

A 300 mL Parr autoclave was charged under nitrogen atmosphere with Pd(OAc)₂ (0.02 mg, 0.0001 mmol), bisphosphorus ligand L3 of Formula VIII (0.1 mg, 0.0002 mmol), p-toluenesulfonic acid (0.2 mg, 0.001 mmol), methanol (30 mL), acetone (20 mL), and a stir bar. The system was purged with nitrogen, then pressurized with ethylene gas to 2.0 MPa, followed by pressurization with carbon monoxide to 6.0 MPa. The reaction mixture was rapidly heated to 100° C. and stirred for 12 hours. After completion, the system was cooled to room temperature. NMR analysis indicated methyl propionate was obtained in 86% yield with >95% selectivity.

### Example 73

A 300 mL Parr autoclave was charged under nitrogen atmosphere with Pd(OAc)₂ (2.2 mg, 0.01 mmol), bisphosphorus ligand L4 of Formula VIII (18.6 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 100° C. and stirred for 10 hours. After completion, the system was cooled to room temperature. NMR analysis indicated methyl propionate was obtained in 80% yield with >95% selectivity.

### Example 74

A 300 mL Parr autoclave was charged under nitrogen atmosphere with Pd(OAc)₂ (2.2 mg, 0.01 mmol), bisphosphorus ligand L5 of Formula VIII (18.6 mg, 0.04 mmol), methanesulfonic acid (6.5 μL, 0.10 mmol), methanol (30 mL), methyl propionate (20 mL), and a stir bar. The system was purged with nitrogen, then pressurized with ethylene gas to 2.0 MPa, followed by pressurization with carbon monoxide to 6.0 MPa. The reaction mixture was rapidly heated to 100° C. and stirred for 10 hours. After completion, the system was cooled to room temperature. NMR analysis indicated methyl propionate was obtained in 88% yield with >95% selectivity.

### Example 75

A 300 mL Parr autoclave was charged under nitrogen atmosphere with Pd(OAc)₂ (2.2 mg, 0.01 mmol), bisphosphorus ligand L6 of Formula VIII (19.6 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 10 hours. After completion, the system was cooled to room temperature. NMR analysis indicated methyl propionate was obtained in 78% yield with >95% selectivity.

### Example 76

A 300 mL Parr autoclave was charged under nitrogen atmosphere with Pd(OAc)₂ (2.2 mg, 0.01 mmol), bisphosphorus ligand L7 of Formula VIII (18.6 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 100° C. and stirred for 10 hours. After completion, the system was cooled to room temperature. NMR analysis indicated methyl propionate was obtained in 84% yield with >95% selectivity.

### Example 77

A 300 mL Parr autoclave was charged under nitrogen atmosphere with Pd(OAc)₂ (2.2 mg, 0.01 mmol), bisphosphorus ligand L8 of Formula VIII (19.0 mg, 0.02 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 100° C. and stirred for 10 hours. After completion, the system was cooled to room temperature. NMR analysis indicated methyl propionate was obtained in 77% yield with >95% selectivity.

### Example 78

A 300 mL Parr autoclave was charged under nitrogen atmosphere with Pd(OAc)₂ (2.2 mg, 0.01 mmol), bisphosphorus ligand L9 of Formula VIII (18.3 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 10 hours. After completion, the system was cooled to room temperature. NMR analysis indicated methyl propionate was obtained in 88% yield with >95% selectivity.

### Example 79

A 300 mL Parr autoclave was charged under nitrogen atmosphere with Pd₂(dba)₃ (4.6 mg, 0.005 mmol), bisphosphorus ligand L10 of Formula VIII (19.7 mg, 0.04 mmol), methanesulfonic acid (6.5 μL, 0.10 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 100° C. and stirred for 10 hours. After completion, the system was cooled to room temperature. NMR analysis indicated methyl propionate was obtained in 75% yield with >95% selectivity.

### Example 80

A 300 mL Parr autoclave was charged under nitrogen atmosphere with Pd(dba)₂ (5.7 mg, 0.01 mmol), bisphosphorus ligand L11 of Formula VIII (21.0 mg, 0.02 mmol), trifluoromethanesulfonic acid (14.2 μL, 0.16 mmol), methanol (50 mL), and a stir bar. The system was purged with nitrogen, then pressurized with ethylene gas to 2.0 MPa, followed by pressurization with carbon monoxide to 6.0 MPa. The reaction mixture was rapidly heated to 80° C. and stirred for 10 hours. After completion, the system was cooled to room temperature. NMR analysis indicated methyl propionate was obtained in 84% yield with >95% selectivity.

### Example 81

A 300 mL Parr autoclave was charged under nitrogen atmosphere with PdCl₂ (1.8 mg, 0.01 mmol), bisphosphorus ligand L12 of Formula VIII (19.1 mg, 0.04 mmol), dodecylsulfonic acid (26.1 mg, 0.08 mmol), methanol (50 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 10 hours. After completion, the system was cooled to room temperature. NMR analysis indicated methyl propionate was obtained in 88% yield with >95% selectivity.

### Example 82

A 300 mL Parr autoclave was charged under nitrogen atmosphere with Pd(hfacac)₂ (3.1 mg, 0.01 mmol), bisphosphorus ligand L13 of Formula VIII (9.5 mg, 0.04 mmol), trifluoroacetic acid (12.0 μL, 0.16 mmol), methanol (30 mL), THF (20 mL), and a stir bar. The system was purged with ethylene, then pressurized with ethylene gas to 2.0 MPa, followed by pressurization with carbon monoxide to 6.0 MPa. The reaction mixture was rapidly heated to 100° C. and stirred for 10 hours. After completion, the system was cooled to room temperature. NMR analysis indicated methyl propionate was obtained in 84% yield with >95% selectivity.

### Example 83

A 300 mL Parr autoclave was charged under nitrogen atmosphere with Pd(CH₃CN)₄(BF₄)₂ (4.4 mg, 0.01 mmol), bisphosphorus ligand L14 of Formula VIII (9.6 mg, 0.02 mmol), methanesulfonic acid (10.4 μL, 0.16 mmol), propanol (30 mL), acetonitrile (20 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 10 hours. After completion, the system was cooled to room temperature. NMR analysis indicated propyl propionate was obtained in 70% yield with >95% selectivity.

### Example 84

A 300 mL Parr autoclave was charged under nitrogen atmosphere with Pd(OAc)₂ (11.2 mg, 0.05 mmol), bisphosphorus ligand L15 of Formula VIII (19.0 mg, 0.04 mmol), methanesulfonic acid (10.4 μL, 0.16 mmol), butanol (30 mL), methyl tert-butyl ether (20 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 10 hours. After completion, the system was cooled to room temperature. NMR analysis indicated butyl propionate was obtained in 89% yield with >95% selectivity.

### Example 85

A 300 mL Parr autoclave was charged under nitrogen atmosphere with Pd(OAc)₂ (2.2 mg, 0.01 mmol), bisphosphorus ligand L16 of Formula VIII (19.4 mg, 0.04 mmol), p-toluenesulfonic acid (17.5 mg, 0.10 mmol), ethanol (30 mL), xylene (20 mL), and a stir bar. The system was purged with ethylene, then pressurized with ethylene gas to 2.0 MPa, followed by pressurization with carbon monoxide to 6.0 MPa. The reaction mixture was rapidly heated to 100° C. and stirred for 10 hours. After completion, the system was cooled to room temperature. NMR analysis indicated ethyl propionate was obtained in 83% yield with >95% selectivity.

### Example 86

A 300 mL Parr autoclave was charged under nitrogen atmosphere with Pd(OAc)₂ (2.2 mg, 0.01 mmol), bisphosphorus ligand L17 of Formula VIII (18.0 mg, 0.04 mmol), trifluoroacetic acid (12 μL, 0.16 mmol), methanol (30 mL), chlorobenzene (20 mL), and a stir bar. The system was purged with ethylene, then pressurized with ethylene gas to 2.0 MPa, followed by pressurization with carbon monoxide to 6.0 MPa. The reaction mixture was rapidly heated to 100° C. and stirred for 10 hours. After completion, the system was cooled to room temperature. NMR analysis indicated methyl propionate was obtained in 80% yield with >90% selectivity.

### Example 87

A 300 mL Parr autoclave was charged under nitrogen atmosphere with Pd(OAc)₂ (2.24 mg, 0.01 mmol), bisphosphorus ligand L18 of Formula VIII (20.2 mg, 0.04 mmol), methanesulfonic acid (10.4 μL, 0.16 mmol), octanol (50 mL), and a stir bar. The system was purged with ethylene, then pressurized with ethylene gas to 2.0 MPa, followed by pressurization with carbon monoxide to 6.0 MPa. The reaction mixture was rapidly heated to 100° C. and stirred for 10 hours. After completion, the system was cooled to room temperature. NMR analysis indicated octyl propionate was obtained in 83% yield with >90% selectivity.

### Example 88

A 300 mL Parr autoclave was charged under nitrogen atmosphere with Pd(OAc)₂ (2.24 mg, 0.01 mmol), bisphosphorus ligand L19 of Formula VIII (19.6 mg, 0.04 mmol), methanesulfonic acid (10.4 μL, 0.16 mmol), butanol (50 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 10 hours. After completion, the system was cooled to room temperature. NMR analysis indicated butyl propionate was obtained in 88% yield with >90% selectivity.

### Example 89

A 300 mL Parr autoclave was charged under nitrogen atmosphere with Pd(OAc)₂ (2.24 mg, 0.01 mmol), bisphosphorus ligand L20 of Formula VIII (21.8 mg, 0.04 mmol), methanesulfonic acid (10.4 μL, 0.16 mmol), butanol (30 mL), THF (20 mL), and a stir bar. The system was purged with ethylene, then pressurized with ethylene gas to 2.0 MPa, followed by pressurization with carbon monoxide to 6.0 MPa. The reaction mixture was rapidly heated to 80° C. and stirred for 10 hours. After completion, the system was cooled to room temperature. NMR analysis indicated butyl propionate was obtained in 83% yield with >90% selectivity.

The phosphine ligands of the present invention, when used for catalyzing the preparation of propionate esters, can provide the propionate ester products in high yields (>65%, optimally >90% in preferred examples) with excellent selectivity, thereby demonstrating potential for industrial applications.

Furthermore, the phosphine ligands of the present invention exhibit superior catalytic turnover, enabling the completion of the catalytic reaction at low loadings (<10⁻⁴ equivalents, preferably <10⁻⁵ equivalents, more preferably <10⁻⁶ equivalents).

All documents mentioned herein are incorporated by reference as if each were individually incorporated by reference. Furthermore, it should be understood that after reading the above teachings of the present invention, those skilled in the art may make various modifications or alterations to the invention. Such equivalent forms shall likewise fall within the scope defined by the appended claims of this application.

1-34. (canceled)

35. A phosphine ligand comprising a structure represented by Formula I, V, or VI,
wherein the phosphine ligand of Formula I has the following structure: 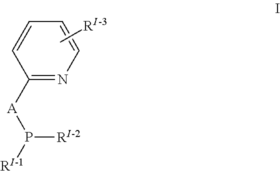
wherein
R^{I-1} is selected from substituted or unsubstituted C₁₋₁₀ alkyl, C₃₋₁₂ cycloalkyl, optionally containing adamantyl, C₆₋₃₀ aryl;
R^{I-2} is selected from the substituted or unsubstituted 5-20 membered heteroaryl;
R^{I-3} is selected from hydrogen, substituted or unsubstituted C₁₋₁₀ alkyl, substituted or unsubstituted C₁₋₁₀ alkoxy, substituted or unsubstituted C₂₋₁₀ ester group, cyano, COOH, benzenesulfonyl, trialkylsilyl (wherein said alkyl is C₁₋₄ alkyl), nitro, substituted or unsubstituted C₆₋₃₀ aryl, substituted or unsubstituted 5-30 membered heteroaryl; or two R³ groups attached to adjacent carbon atoms together with the carbon atoms to which they are attached form a group selected from the group consisting of: C₆₋₁₀ aryl, 5-12 membered heteroaryl;
A is selected from substituted and unsubstituted C₁₋₄ alkyl, 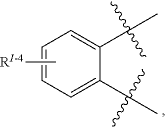
A and 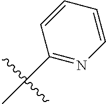 [SMILES: *c1ccccn1]
are linked to form 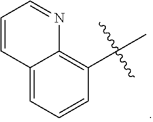
wherein, [symbol] is the attachment site linked to phosphorus;
and
when A is 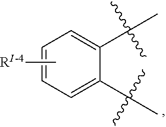
then R^{I-4} is one or more substituents selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, and 5-30 membered heteroaryl; or R^{I-4} groups together with R^{I-3} and adjacent pyridine ring atoms form a fused 5-7 membered carbocyclic or heterocyclic ring;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, unless expressly stated otherwise;
wherein the phosphine ligand of Formula V has the following structure: 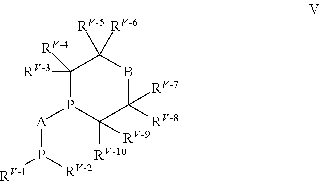
wherein
R^{V-1} is selected from substituted or unsubstituted C₁₋₁₂ alkyl, C₃₋₁₀ cycloalkyl, C₆₋₃₀ aryl;
R^{V-2} is selected from the substituted or unsubstituted 5-20 membered heteroaryl;
and
R^{V-1} is selected from substituted or unsubstituted C₁₋₁₂ alkyl, C₃₋₁₀ cycloalkyl;
R^{V-2} is selected from the substituted or unsubstituted C₆₋₃₀ heteroaryl;
R^{V-3}, R^{V-4}, R^{V-5}, R^{V-6}, R^{V-7}, R^{V-8}, R^{V-9}, R^{V-10} are independently selected from hydrogen, hydroxyl, halogen, C₁₋₁₂ alkyl, C₃₋₁₂ cycloalkyl, C₃₋₁₀ heterocycloalkyl, C₂₋₁₀ alkenyl, C₆₋₃₀ aryl, 5-20 membered heteroaryl, C₁₋₁₀ alkoxyl, C₁₋₁₀ alkylamino, C₁₋₁₀ alkylamino, C₁₋₁₀ alkylthio; two adjacent R^{V-3}, R^{V-4}, R^{V-5}, R^{V-6}, R^{V-7}, R^{V-8}, R^{V-9}, R^{V-10} located on rings, together with the ring atoms to which they are attached, form a 4-8 membered carbocycle or heterocycle;
A if it is -L¹-A¹-L²-, may each independently be selected from chemical bonds, O, NH, S, substituted or unsubstituted C₁₋₁₂ alkyl, C₃₋₈ cycloalkyl, C₃₋₈ heterocycloalkyl, C₆₋₃₀ aryl, 5-20 membered heteroaryl, biphenyl, 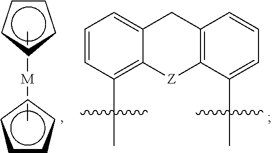
with the proviso that L¹, A¹ and L² are not all a chemical bond at the same time;
and
if the said aryl and heteroaryl are substituted by two or more substituents, any two of said substituents may, together with the ring atoms to which they are attached, form a 4-8 membered carbocycle or heterocycle;
M is selected from Fe, Co, Ru;
B is selected from substituted or unsubstituted methylene, ethylidene, ═CHR, ═NR, hydroxymethyl, carbonyl, thiocarbonyl, 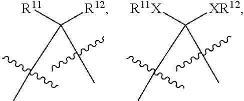
—C(O)—C(O)—; where R is selected from substituted or unsubstituted C₁₋₁₀ alkyl, C₆₋₃₀ aryl, and 5-20 membered heteroaryl;
B is selected from substituted or unsubstituted methylene, ethylidene, ═CHR, ═NR, hydroxymethyl, carbonyl, thiocarbonyl, 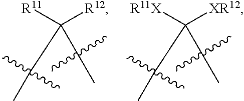
—C(O)—C(O)—; where R is selected from substituted or unsubstituted C₁₋₁₀ alkyl, C₆₋₃₀ aryl, 5-20 membered heteroaryl;
R¹¹, R¹² are independently selected from hedrogen, hydroxyl, substituted or unsubstituted C₁₋₁₀ alkyl, or 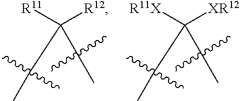
form 5-8 membered heterocycles, each may be unsubstituted or substituted by C₁₋₆ alkyl;
X is selected from O, S;
Z is selected from O, S, NH;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, benzyl, —CH(Ph)₂, unless expressly stated otherwise;
wherein the phosphine ligand of Formula VI has the following structure: 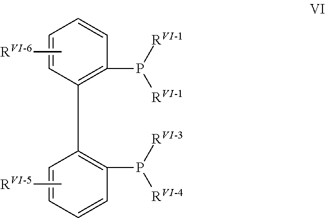
wherein
R^{VI-1}, R^{VI-4} are independently selected from substituted or unsubstituted C₁₋₁₀ alkyl, C₃₋₁₀ cycloalkyl, C₆₋₃₀ aryl;
R^{VI-2}, R^{VI-3} are independently selected from 5-20 membered heteroaryl;
R^{VI-5}, R^{VI-6} are independently selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, and 5-30 membered heteroaryl; two adjacent R^{VI-5}, R^{VI-6} located on rings, together with the ring atoms to which they are attached, form a 5-7 membered carbocycle or heterocycle; wherein the heterocycles contain 1-3 heteroatoms selected from N, O, S;
when used herein, substituted refers to replacement of one or more hydrogens by radicals independently selected from C₁₋₁₀ alkyl, C₁₋₁₀ alkoxy, C₂₋₁₀ ester, cyano, COOH, phenylsulfonyl, tri(C₁₋₄ alkyl)silyl, nitro, C₆₋₃₀ aryl, or 5-30 membered heteroaryl, unless expressly stated otherwise.

36. The phosphine ligand according to claim 35,
wherein in the formula I, A is selected from CH₂, 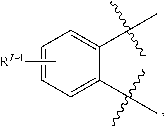
or A together with 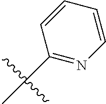 [SMILES: *c1ccccn1]
form 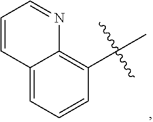 [SMILES: *c1cccc2cccnc12] [symbol]
s the attachment site linked to phosphorus;
wherein R^{I-4} is one or more substituents selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl.

37. The phosphine ligand according to claim 35,
wherein in the formula I,
R^{I-1} is selected from substituted or unsubstituted C₁₋₁₀ alkyl, C₃₋₁₀ cycloalkyl;
R^{I-2} is selected from the substituted or unsubstituted 5-10 membered heteroaryl;
R^{I-3} is selected from hydrogen, substituted or unsubstituted C₁₋₁₀ alkyl, C₁₋₁₀ alkoxy, C₂₋₁₀ ester, cyano, tri(C₁₋₄ alkyl)silyl, nitro, —CH(Ph)₂, C₆₋₃₀ aryl, 5-10 membered heteroaryl.

38. (canceled)

39. The phosphine ligand according to claim 35,
wherein the ligand of Formula I is selected from

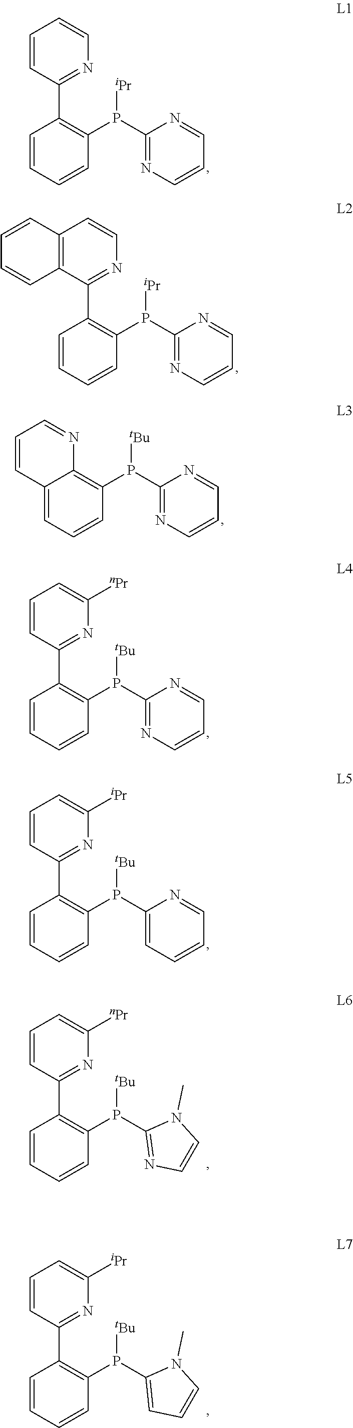

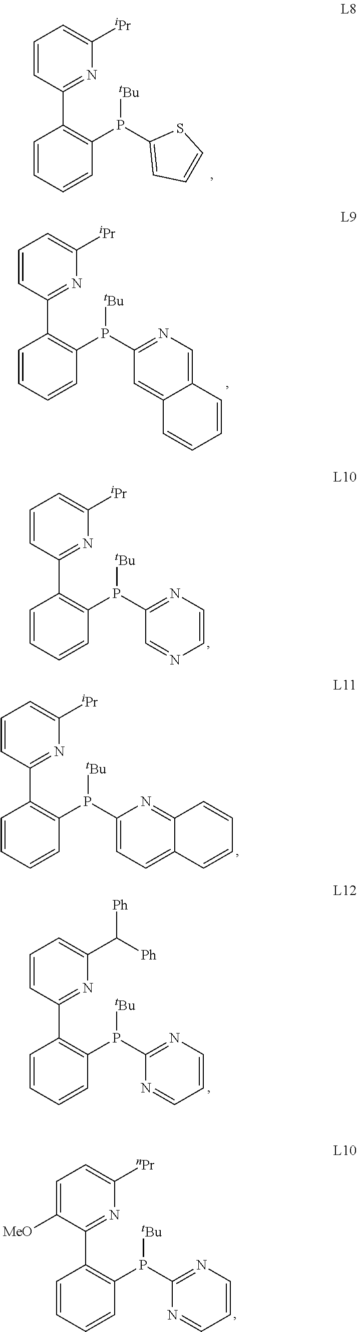

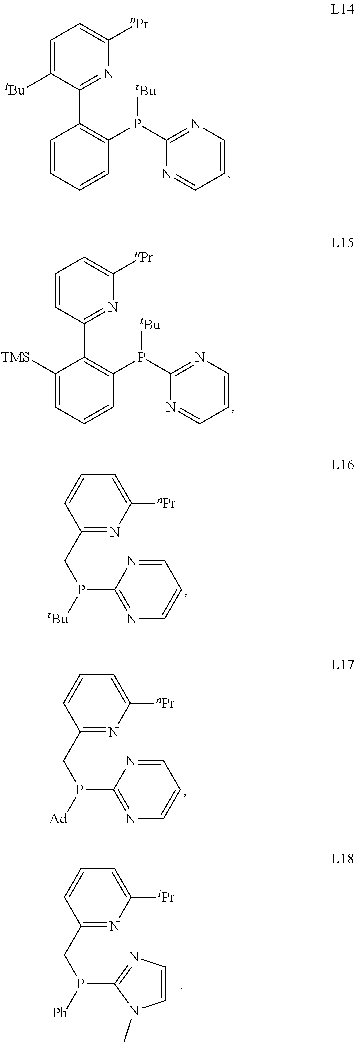

40. The phosphine ligand according to claim 35,
wherein in the formula V,
R^{V-1} is selected from substituted or unsubstituted C₁₋₁₂ alkyl, C₃₋₁₀ cycloalkyl, C₆₋₂₀ aryl;
R^{V-2} is selected from the substituted or unsubstituted 5-12 membered heteroaryl;
and
R^{V-1} is selected from substituted or unsubstituted C₁₋₁₂ alkyl, C₃₋₁₀ cycloalkyl;
R^{V-2} is selected from the substituted or unsubstituted C₆₋₂₀ heteroaryl.

41. The phosphine ligand according to claim 35,
wherein in the formula V,
R^{V-3}, R^{V-4}, R^{V-5}, R^{V-6}, R^{V-7}, R^{V-8}, R^{V-9}, R^{V-10} are independently selected from hydrogen, C₁₋₁₂ alkyl, C₃₋₁₂ cycloalkyl, C₃₋₁₀ heterocycloalkyl, C₂₋₁₀ alkenyl, C₁₋₁₀ alkoxyl, C₁₋₁₀ alkylamino, C₁₋₁₀ alkylamino, C₁₋₁₀ alkylthio; two adjacent R^{V-3}, R^{V-10} located on rings, together with the ring atoms to which they are attached, form a 4-8 membered carbocycle or heterocycle;
B is selected from substituted or unsubstituted methylene, ethylidene, ═NR, hydroxymethyl, carbonyl, 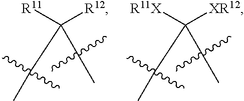
—C(O)—C(O)—; where R is selected from substituted or unsubstituted C₁₋₁₀ alkyl, phenyl;
R¹¹, R¹² are independently selected from hydrogen, hydroxyl, substituted or unsubstituted C₁₋₆ alkyl, or 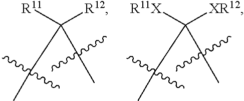
form 5-8 membered heterocycles, each may be unsubstituted or substituted by C₁₋₆ alkyl;
X is selected from O, S.

42. (canceled)

43. The phosphine ligand according to claim 35,
wherein the ligand of Formula V is selected from 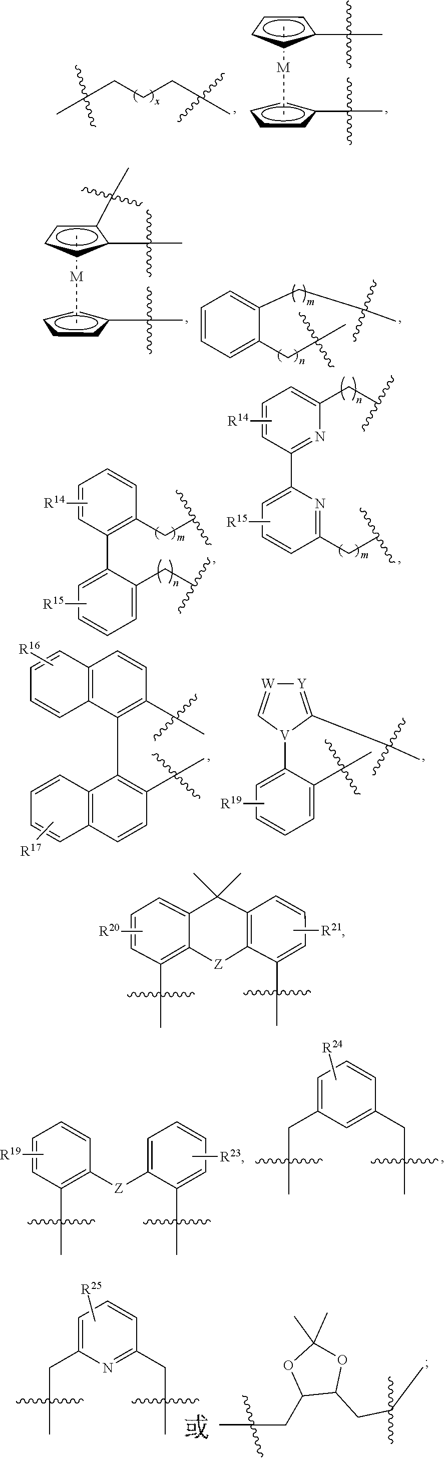
where
R¹³, R¹⁴, R¹⁵, R¹⁶, R¹⁷, R¹⁸, R¹⁹, R²⁰, R²¹, R²², R²³, R²⁴, R²⁵ are independently selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, and 5-30 membered heteroaryl; two adjacent R¹³, R¹⁴, R¹⁵, R¹⁶, R¹⁷, R¹⁸, R¹⁹, R²⁰, R²¹, R²², R²³ located on rings, together with the ring atoms to which they are attached, form a 5-7 membered carbocycle or heterocycle;
x is selected from 0, 1, 2, 3, 4, 5;
m, n are independently selected from 0, 1, 2, 3;
M is selected from Fe, Co, Ru;
V, W, Y are independently selected from N, CH; wherein any hydrogen atom on said CH may be substituted by R¹⁸; 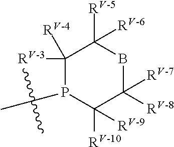
is selected from 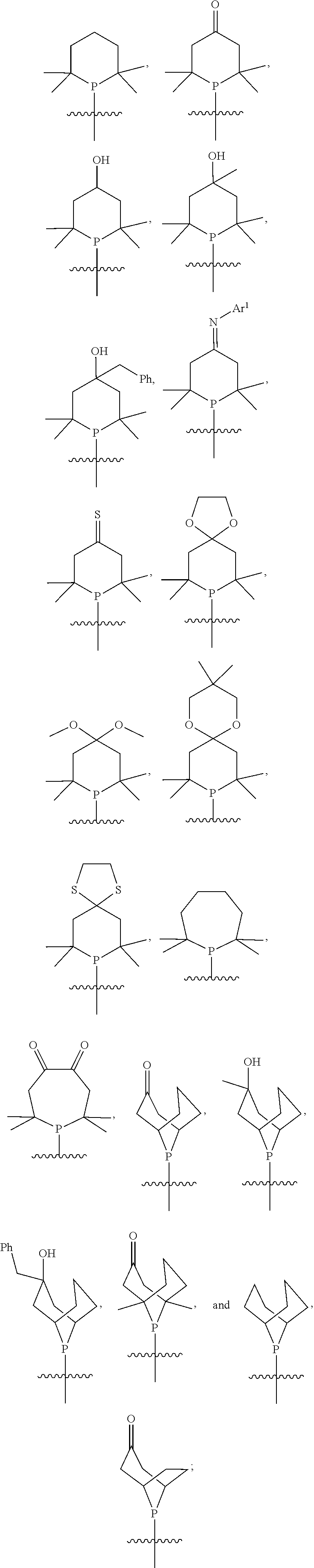
wherein
Ar¹ is selected from substituted or unsubstituted C₆₋₃₀ aryl, 5-20 membered heteroaryl.

44. The phosphine ligand according to claim 35,
wherein the ligand of Formula V is selected from

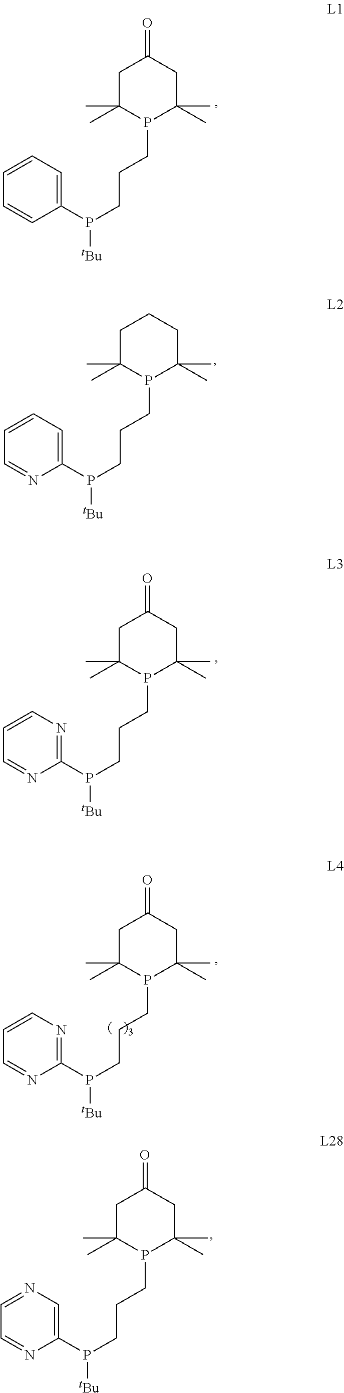

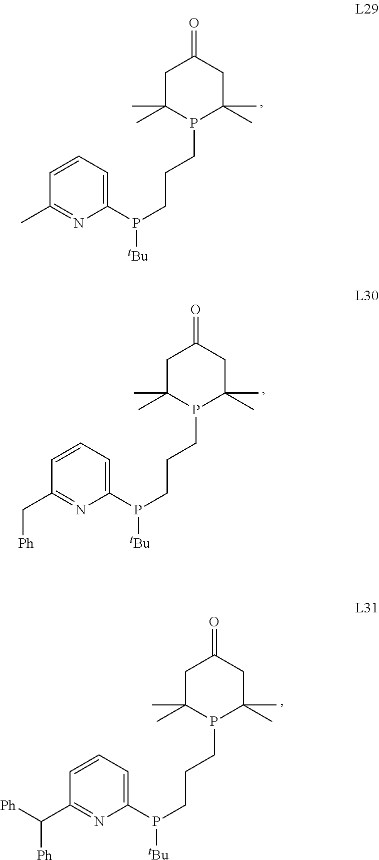

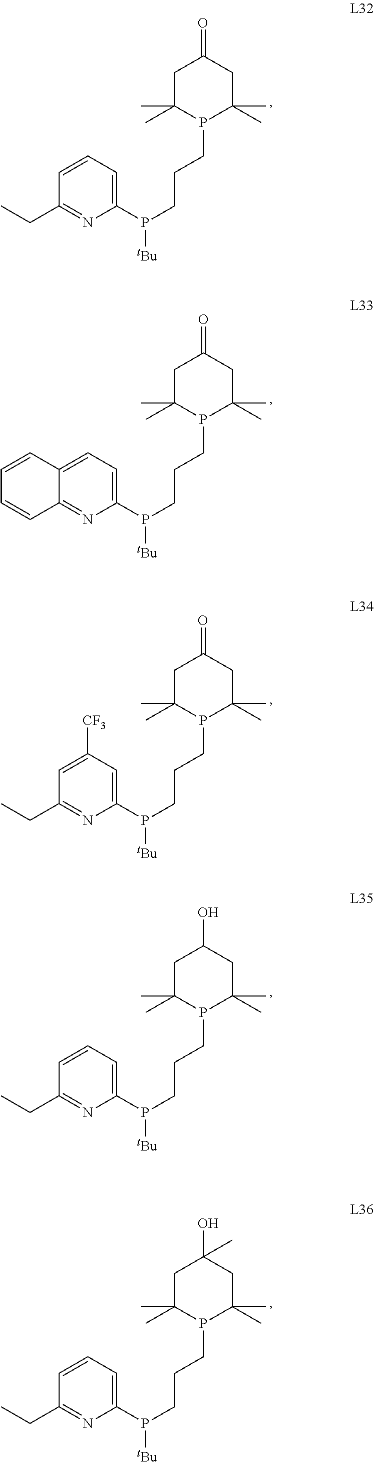

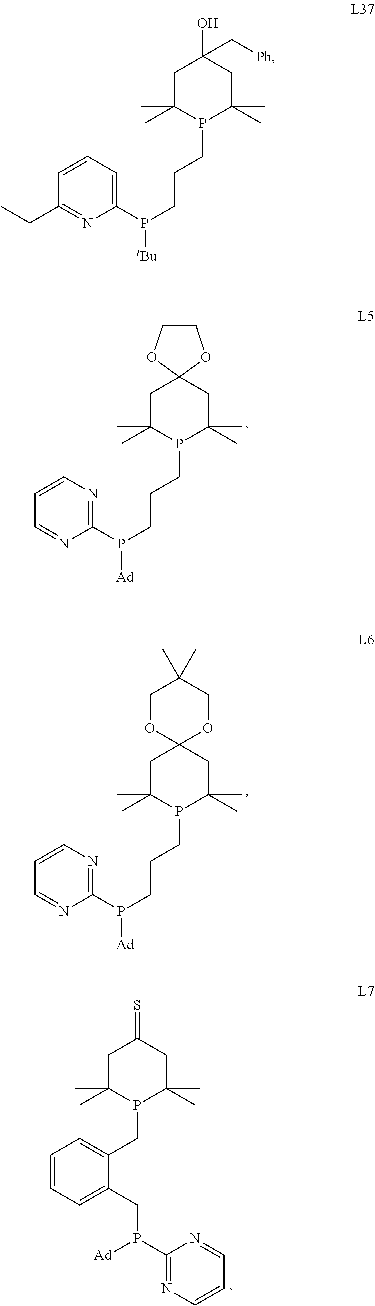

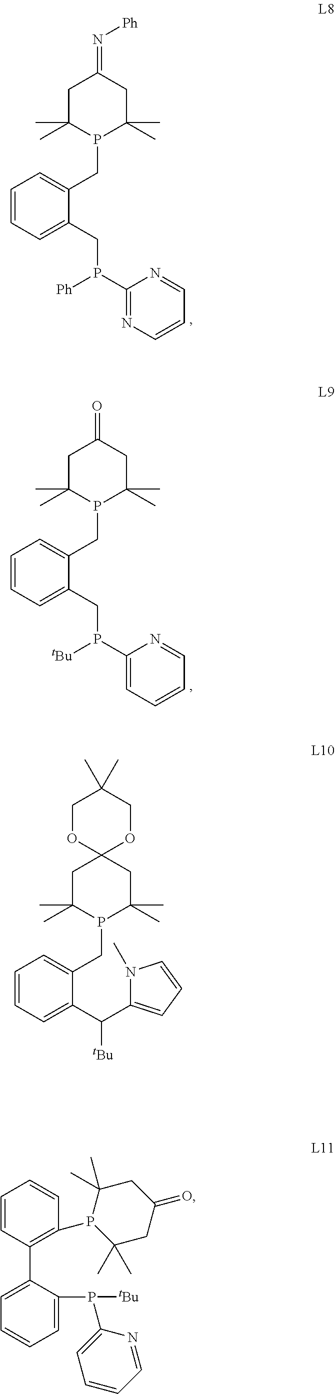 [SMILES: CC1(C)CC(=Nc2ccccc2)CC(C)(C)P1Cc1ccccc1CP(c1ccccc1)c1ncccn1 ; CC(C)(C)P(Cc1ccccc1CP1C(C)(C)CC(=O)CC1(C)C)c1ccccn1 ; Cn1cccc1C(c1ccccc1CP1C(C)(C)CC2(CC1(C)C)OCC(C)(C)CO2)C(C)(C)C ; CC(C)(C)P(c1ccccn1)c1ccccc1-c1ccccc1P1C(C)(C)CC(=O)CC1(C)C]

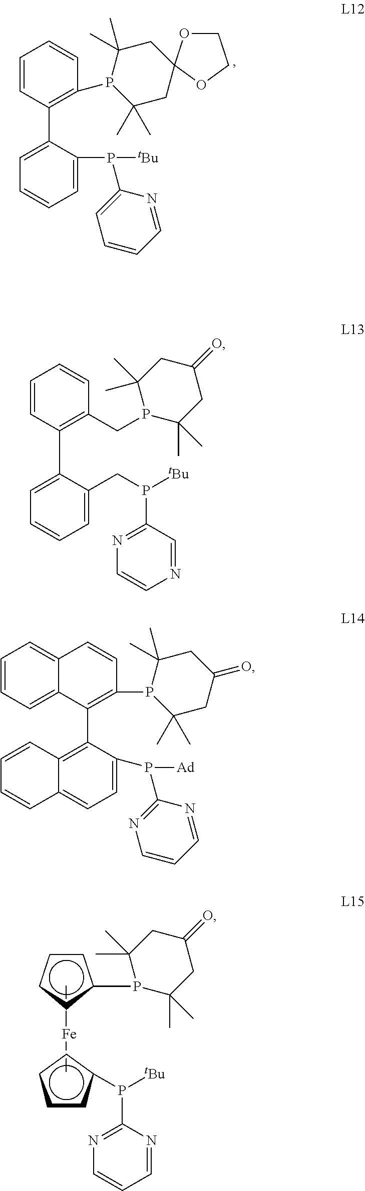

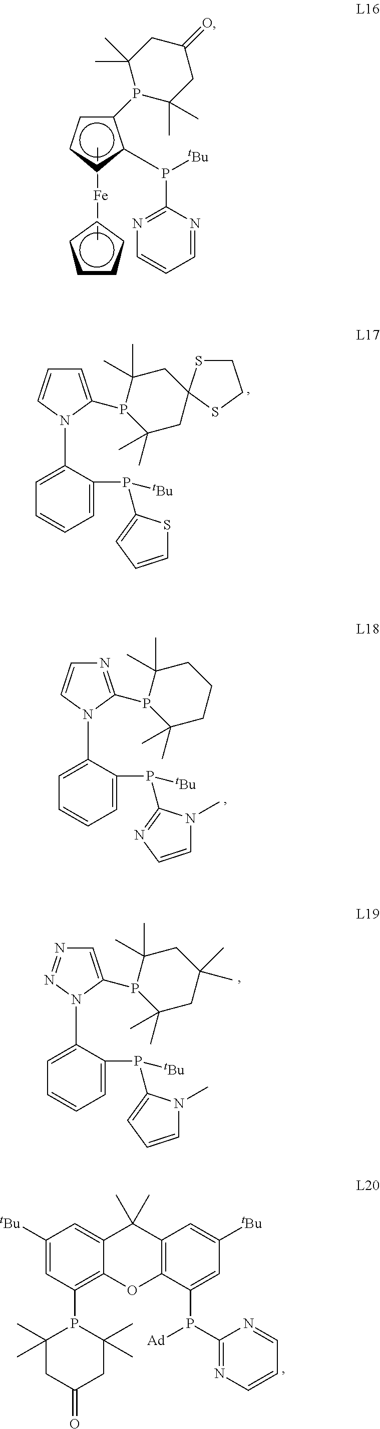

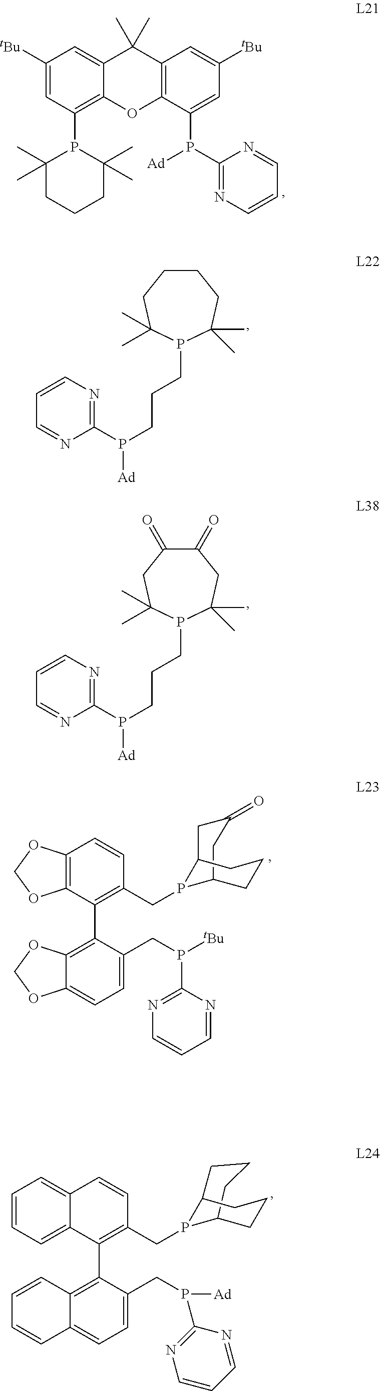

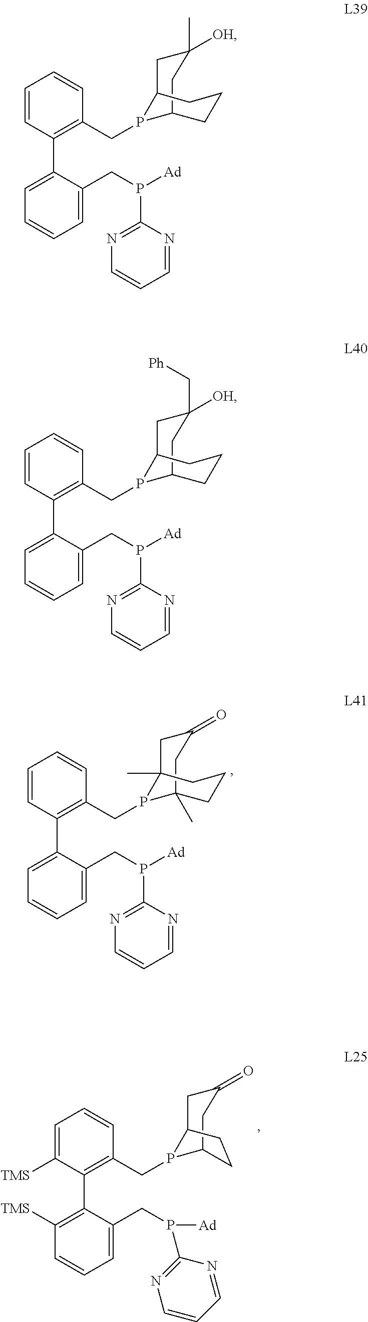

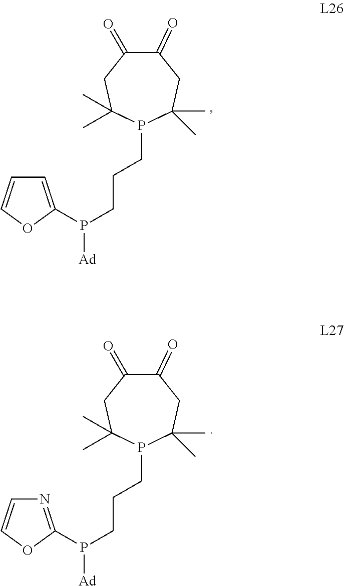

45. The phosphine ligand according to claim 35,
wherein in the formula VI,
R^{VI-1}, R^{VI-4} are independently selected from substituted or unsubstituted C₁₋₄ alkyl, C₃₋₆ cycloalkyl, C₆₋₁₄ aryl;
R^{VI-2}, R^{VI-3} are independently selected from 5-20 membered heteroaryl;
and
R^{VI-1}, R^{VI-2}, R^{VI-3}, R^{VI-4} are independently unsubstituted or substituted by one or more substituents selected from methyl, methoxyl, cyano, COOH, benzenesulfonyl, trimethylsilyl.

46. The phosphine ligand according to claim 35,
wherein in the formula VI,
R^{VI-1}, R^{VI-4} are independently selected from methyl, ethyl, n-propyl, isopropyl, n-butyl, tert-butyl, isobutyl, cyclopropyl, cyclobutyl, cyclopentyl, cyclohexyl, phenyl, adamantyl;
R^{VI-2}, R^{VI-3} are independently selected from pyridyl, pyrazinyl, pyrimidinyl, pyridazinyl, pyrrolyl, imidazolyl, pyrazolyl, thienyl, furyl, thiazolyl, triazolyl;
R^{VI-5}, R^{VI-6} are independently selected from hydrogen, C₁₋₁₀ alkyl, C₁₋₁₀ alkoxy, C₂₋₁₀ ester, cyano, COOH, benzenesulfonyl, tri(C₁₋₄ alkyl)silyl, nitro, C₆₋₃₀ aryl, 5-30 membered heteroaryl; or two adjacent R^{VI-5}, R^{VI-6} located on rings, together with the ring atoms to which they are attached, form a 5-7 membered carbocycle or heterocycle.

47. The phosphine ligand according to claim 35,
wherein the ligand of Formula VI is selected from

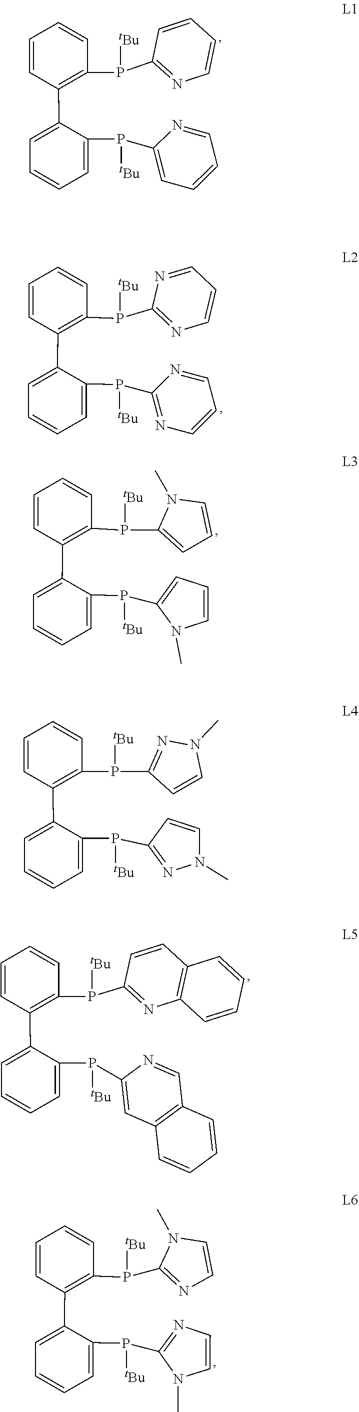

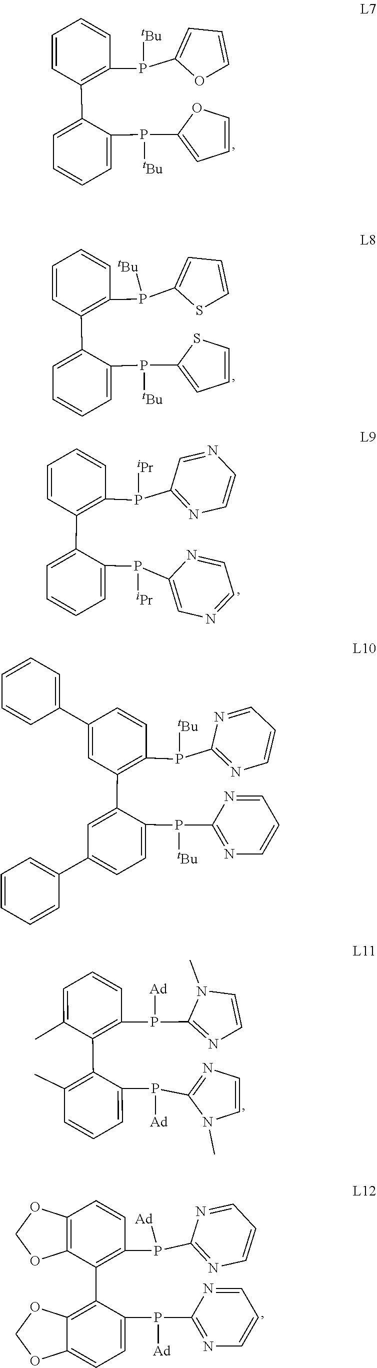

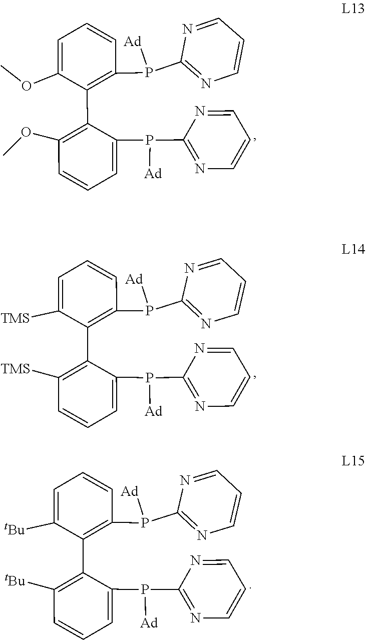

48-73. (canceled)

74. Process for synthesizing acrylate through acetylene carbonylation, comprising the following process steps:
a) dissolving a palladium catalyst, the phosphine ligand of claim 35, and an acid in an alcoholic solvent, optionally with additional solvent, in a reaction vessel;
b) charging acetylene into the reactor, followed by feeding in CO; and
c) heating the reaction mixture to convert acetylene to an acrylate, then isolation.

75. The process according to claim 74, wherein the alcohol is a C1-12 alkyl alcohol; and
the alcohol is selected from methanol, ethanol, propanol, butanol, octanol, and combinations thereof.

76. The process according to claim 74, wherein the palladium catalyst is selected from the group consisting of palladium acetate, palladium trifluoroacetate, palladium pivalate, palladium(II) tetraacetonitrile tetrafluoroborate, palladium(II) hexafluoroacetylacetonate, palladium(II) bis(acetylacetonate), palladium(II) tetraacetonitrile trifluoromethanesulfonate, palladium(II) neopentanoate, bis(dibenzylideneacetone)palladium(O), tris(dibenzylideneacetone)dipalladium(O), palladium(II) chloride, palladium(II) diacetonitrile dichloride, palladium(II) dibenzonitrile dichloride, and combinations thereof.

77. Process for synthesizing propionate esters through palladium-catalyzed ethylene carbonylation, comprising steps:
a) dissolving a palladium catalyst, the phosphine ligand of claim 35, and an acid in an alcoholic solvent, optionally with additional solvent, in a reaction vessel;
b) charging ethylene into the reactor, followed by feeding in CO; and
c) heating the reaction mixture to convert ethylene to propionate esters, then isolation.

78. The process according to claim 77, wherein the alcohol is a C1-12 alkyl alcohol; and the alcohol is selected from the group consisting of methanol, ethanol, propanol, butanol, octanol, and combinations thereof.

79. The process according to claim 77, wherein the palladium catalyst is selected from palladium acetate, palladium trifluoroacetate, palladium pivalate, palladium(II) tetraacetonitrile tetrafluoroborate, palladium(II) hexafluoroacetylacetonate, palladium(II) bis(acetylacetonate), palladium(II) tetraacetonitrile trifluoromethanesulfonate, palladium(II) neopentanoate, bis(dibenzylideneacetone)palladium(O), tris(dibenzylideneacetone)dipalladium(O), palladium(II) chloride, palladium(II) diacetonitrile dichloride, palladium(II) dibenzonitrile dichloride, and combinations thereof.
